(19) United States
(12) Patent Application Publication
(10) Pub. No.: US 2022/0056018 A1
(43) Pub. Date: Feb. 24, 2022

(54) SREBP INHIBITORS COMPRISING A THIOPHENE CENTRAL RING

(71) Applicant: Capulus Therapeutics, LLC, San Francisco, CA (US)

(72) Inventors: Michael John GREEN, Half Moon Bay, CA (US); Barry Patrick HART, Palo Alto, CA (US)

(21) Appl. No.: 17/385,484

(22) Filed: Jul. 26, 2021

Related U.S. Application Data
(63) Continuation of application No. PCT/US2020/015260, filed on Jan. 27, 2020.
(60) Provisional application No. 62/797,759, filed on Jan. 28, 2019.

(51) Int. Cl. C07D 409/04; C07D 409/14; C07D 413/14; A61P 1/16; A61P 35/00

(52) U.S. Cl. CPC C07D 409/04; C07D 409/14; A61P 35/00; A61P 1/16; C07D 413/14

(57) ABSTRACT

Provided herein are compounds comprising a three-ring core, such as compounds of Formula (I), Formula (I-i), Formula (I-A), Formula (I-A-i), Formula (I-A-i-1), Formula (I-B), Formula (I-B-i), Formula (I-B-i-1), Formula (II), Formula (II-i), Formula (II-A), Formula (II-A-i), Formula (II-A-i-1), Formula (II-B), Formula (II-B-i), and Formula (II-B-i-1) and pharmaceutically acceptable salts, solvates, tautomers, isotopes, or isomers thereof. Also provided herein are methods of inhibiting a component of the sterol regulatory element binding protein (SREBP) pathway, such as an SREBP or SREBP cleavage activating protein (SCAP), using these compounds, or pharmaceutically acceptable salts, solvates, tautomers, isotopes, or isomers thereof. Further provided are methods of treating a disorder in a subject in need thereof, such as liver disease, non-alcoholic steatohepatitis, insulin resistance, or cancer.

### CROSS REFERENCE TO RELATED APPLICATIONS

This application claims the benefit of U.S. Provisional Application No. 62/797,759, filed Jan. 28, 2019, the content of which is incorporated herein by reference in its entirety.

### FIELD OF THE INVENTION

The present disclosure relates to compounds comprising a three-ring core, their use for inhibiting components of the sterol regulatory element binding protein (SREBP) pathway, such as SREBP or SREBP cleavage activating protein (SCAP), and their use in therapeutic methods of treating conditions and disorders.

### BACKGROUND

SREBPs are membrane-bound transcription factors that regulate cholesterol, fatty acid, and triglyceride biosynthesis, and lipid uptake. Fatty acids and lipids are a source of energy and important components of many biological structures, such as lipid membranes of cells. Cholesterol is an important component of biological processes and structures. In mammals, there are three known SREBP isoforms: SREBP-1a, SREBP-1c, and SREBP-2. SREBP-1a controls a broad range of target genes that are involved in the production of fatty acids, triglycerides, phospholipids, and cholesterol. SREBP-1c primarily activates genes which control fatty acid and triglyceride synthesis. SREBP-2 activates genes involved in the synthesis of regulators of cholesterol metabolism, which has been demonstrated in mouse, human, and Drosophila studies. The activity of SREBPs is regulated by SREBP cleavage activating protein (SCAP), which transports SREBP(s) from the endoplasmic reticulum to the Golgi apparatus where the SREBP(s) are proteolytically cleaved, releasing the transcription factor domain.

The pathways regulated by SREBPs and SCAP have been implicated in disorders of metabolism, such as hypertension, dyslipidemia, obesity, type 2 diabetes, insulin resistance, fatty liver, and nonalcoholic steatohepatitis (NASH). NASH, for example, is liver inflammation and hepatocyte ballooning as a result of fat building up in the liver, which can lead to liver damage, such as cirrhosis. NASH can also be associated with other metabolism disorders, such as insulin resistance and metabolic syndrome.

The metabolism of fatty acids, cholesterol, and triglycerides may also be linked to hyperproliferative disorders, such as cancer. One characteristic of the oncogenic transformation of cancer cells is the shift of metabolism from catabolic to anabolic processes. Many cancers require synthesis of fatty acids and other lipids (such as cholesterol), and steroids (such as androgens). Thus, components of the SREBP pathway may play a role in hyperproliferative disorders, such as prostate cancer. SREBP-1c is the major transcriptional regulator of the biosynthesis of fatty acids, and expression of this transcription factor can be stimulated by androgens and epidermal growth factor in prostate cancer cells. Overexpression of SREBP-1c may drive tumorgenicity and invasion of prostate cancer cells. In addition to regulating androgen synthesis, SREBP-2 itself is also regulated by androgens in a direct feedback circuit of androgen production. However, prostate cancer cells have dysfunctional cholesterol homeostasis, resulting in accumulation of cholesterol and increased proliferation. This increase in cholesterol levels has been shown to be driven by regulated by increased SREBP-2 activity. SREBP-2 expression increases during disease progression, and is significantly higher after castration compared to prior.

Regulating components of the SREBP pathway, such as SCAP or SREBPs, is an important therapeutic approach for treating disorders, such as metabolic diseases and cancer. Thus, there is a need for compounds that can inhibit components of the SREBP pathway, such as SREBPs and SCAP.

### BRIEF SUMMARY

In some aspects, provided herein is a compound of Formula (II):

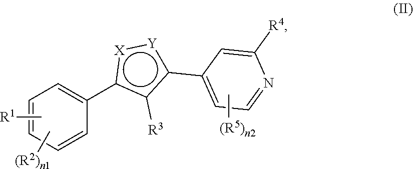

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   or R¹ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁴, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
      wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, —C(O)R¹⁴, and —OC(O)R²²,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
   R²² is independently —R²³N(R²⁴)₂ or —(CH₂CH₂—O—)ₙ₈CH₃,
   wherein each R²³ is (C₁-C₆)alkylene; each R²⁴ is independently H or —CH₃; and each n8 is independently an integer from 2 to 8;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, heterocycloalkyl, or heterocycloalkyl-alkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

In some aspects, provided herein is a compound of Formula (I):

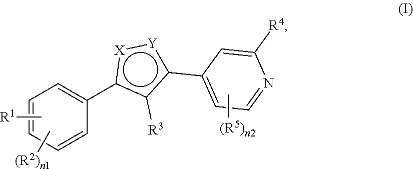

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
      wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹².
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, or heterocycloalkyl, or two R² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

In some aspects, the compound is of Formula (I-A):

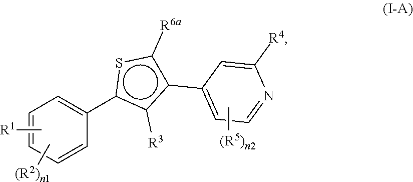

or a pharmaceutically acceptable salt, solvate, tautomer, isotope, or isomer thereof, wherein R¹, R², R³, R⁴, R⁵, R^{6a}, n1, and n2 are as defined for Formula (I).

In certain aspects, the compound is of Formula (I-A-i):

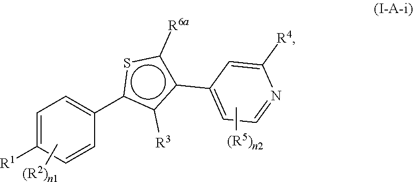

or a pharmaceutically acceptable salt, solvate, tautomer, isotope, or isomer thereof, wherein R¹, R², R³, R⁴, R⁵, R^{6a}, n1, and n2 are as defined for Formula (I).

In other aspects, the compound is of Formula (I-B):

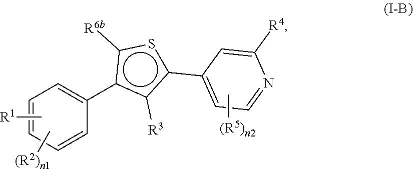

or a pharmaceutically acceptable salt, solvate, tautomer, isotope, or isomer thereof, wherein R¹, R², R³, R⁴, R⁵, R^{6b}, n1, and n2 are as defined for Formula (I).

In certain aspects, the compound is of Formula (I-B-i):

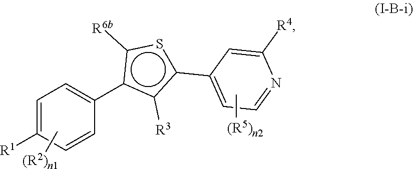

or a pharmaceutically acceptable salt, solvate, tautomer, isotope, or isomer thereof, wherein R¹, R², R³, R⁴, R⁵, R^{6b}, n1, and n2 are as defined for Formula (I).

In some aspects, the compound is of Formula (II-A):

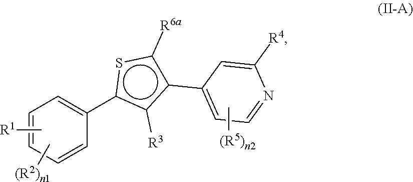

or a pharmaceutically acceptable salt, solvate, tautomer, isotope, or isomer thereof, wherein R¹, R², R³, R⁴, R⁵, R^{6a}, n1, and n2 are as defined for Formula (II).

In certain aspects, the compound is of Formula (II-A-i):

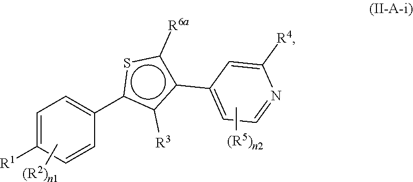

or a pharmaceutically acceptable salt, solvate, tautomer, isotope, or isomer thereof, wherein R¹, R², R³, R⁴, R⁵, R^{6a}, n1, and n2 are as defined for Formula (II).

In other aspects, the compound is of Formula (II-B):

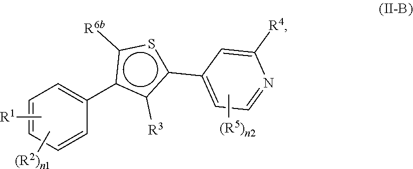

or a pharmaceutically acceptable salt, solvate, tautomer, isotope, or isomer thereof, wherein R¹, R², R³, R⁴, R⁵, R^{6b}, n1, and n2 are as defined for Formula (II).

In certain aspects, the compound is of Formula (II-B-i):

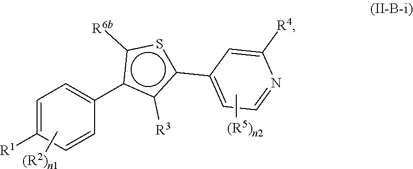

or a pharmaceutically acceptable salt, solvate, tautomer, isotope, or isomer thereof, wherein R¹, R², R³, R⁴, R⁵, R^{6b}, n1, and n2 are as defined for Formula (I).

In some aspects, R¹ is —NR⁷C(O)NR⁸R⁹, —NR⁷S(O)₂NR⁸R⁹, —NR⁷C(O)OR⁸, —S(O)₂R⁸, —NR⁷(SO)₂R⁹, or —NR⁸R⁹. In certain aspects, R¹ is —NR⁷C(O)NR⁸R⁹, —NR⁷S(O)₂NR⁸R⁹, —NR⁷C(O)OR⁸, —S(O)₂R⁸, or —NR⁸R⁹. In further aspects, R¹ is —NR⁷C(O)NR⁸R⁹ or —NR⁷S(O)₂NR⁸R⁹. In some aspects, R¹ is —NR⁷C(O)OR⁹ or —S(O)₂R⁹.

In some aspects, n1 is 0 or 1. In certain aspects, each R² is independently halo, alkyl or —OR¹¹, wherein each R¹¹ is independently hydrogen, unsubstituted alkyl, or haloalkyl. In some aspects, R³ and R⁶a or R^{6b} are both hydrogen.

In further aspects, provided herein is a pharmaceutical composition, comprising a compound as described herein, or a pharmaceutically acceptable salt, solvate, tautomer, isotope, or isomer thereof, and a pharmaceutically acceptable excipient.

In still other aspects, provided herein is a method of inhibiting a sterol regulatory element-binding protein (SREBP), comprising contacting the SREBP or contacting an SREBP cleavage activating-protein (SCAP) with an effective amount of a compound described herein, or a pharmaceutically acceptable salt, solvate, tautomer, isotope, or isomer thereof, or a pharmaceutical composition as described herein. In some aspects, provided herein is a compound as described herein, or a pharmaceutically acceptable salt, solvate, tautomer, isotope, or isomer thereof, for use in inhibiting a sterol regulatory element-binding protein (SREBP). In still further aspects, provided herein is the use of a compound as described herein, or a pharmaceutically acceptable salt, solvate, tautomer, isotope, or isomer thereof, in the manufacture of a medicament for inhibiting a sterol regulatory element-binding protein (SREBP).

In certain aspects, provided herein is a method of inhibiting the proteolytic activation of a sterol regulatory element-binding protein (SREBP), comprising contacting an SREBP cleavage activating-protein (SCAP) with an effective amount of a compound described herein, or a pharmaceutically acceptable salt, solvate, tautomer, isotope, or isomer thereof, or the pharmaceutical composition described herein. In some aspects, provided herein is a compound as described herein, or a pharmaceutically acceptable salt, solvate, tautomer, isotope, or isomer thereof, for use in inhibiting the proteolytic activation of a sterol regulatory element-binding protein (SREBP). In some aspects, provided herein is the use of a compound as described herein, or a pharmaceutically acceptable salt, solvate, tautomer, isotope, or isomer thereof, in the manufacture of a medicament for inhibiting the proteolytic activation of a sterol regulatory element-binding protein (SREBP).

In still further aspects, provided herein is a method of treating a disorder in a subject in need thereof, comprising administering to the subject in need thereof an effective amount of a compound as described herein, or a pharmaceutically acceptable salt, solvate, tautomer, isotope, or isomer thereof, or a pharmaceutical composition described herein. In some aspects, provided herein is a compound as described herein, or a pharmaceutically acceptable salt, solvate, tautomer, isotope, or isomer thereof, for use in treating a disorder in a subject in need thereof. In other aspects, provided herein is the use of a compound as described herein, or a pharmaceutically acceptable salt, solvate, tautomer, isotope, or isomer thereof, in the manufacture of a medicament for treating a disorder in a subject in need thereof.

In yet other aspects, provided herein is a method of treating a disorder in a subject in need thereof, wherein the disorder is mediated by a sterol regulatory element-binding protein (SREBP), comprising administering to the subject in need thereof an effective amount of a compound as described herein, or a pharmaceutically acceptable salt, solvate, tautomer, isotope, or isomer thereof, or a pharmaceutical composition as described herein. In some aspects, provided herein is a compound as described herein, or a pharmaceutically acceptable salt, solvate, tautomer, isotope, or isomer thereof, for use in treating a disorder in a subject in need thereof. In other aspects, provided herein is the use of a compound as described herein, or a pharmaceutically acceptable salt, solvate, tautomer, isotope, or isomer thereof, in the manufacture of a medicament for treating a disorder in a subject in need thereof.

In some aspects of the methods, compounds for use, or uses provided herein, the disorder is Metabolic Syndrome, type 2 diabetes, obesity, liver disease, insulin resistance, adiposopathy, or dyslipidemia. In some embodiments, the liver disease is nonalcoholic steatohepatitis, liver fibrosis, or liver inflammation, or a combination thereof. In other embodiments, the disorder is a hyperproliferative disorder, such as cancer, for example, breast cancer, liver cancer, ovarian cancer, pancreatic cancer, or prostate cancer.

In some aspects of the methods, compounds for use, or uses provided herein, the disorder is a hyperproliferative disorder, such as cancer, for example, soft tissue sarcoma, bladder cancer, endometrial cancer, skin cancer, colon cancer, hematologic cancer, placenta cancer, brain cancer, kidney cancer, lung cancer, or bone cancer.

### DETAILED DESCRIPTION

The following description sets forth numerous exemplary configurations, methods, parameters, and the like. It should be recognized, however, that such description is not intended as a limitation on the scope of the present disclosure, but is instead provided as a description of exemplary embodiments.

### I. Compounds

In some aspects, provided herein is a compound of Formula (II):

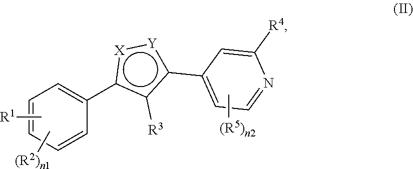

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰OR¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, —C(O)R¹⁴, and —OC(O)R²²,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R²² is independently —R²³N(R²⁴)₂ or —(CH₂CH₂—O—)ₙ₈CH₃,
   wherein each R²³ is (C₁-C₆)alkylene; each R²⁴ is independently H or —CH₃; and each n8 is independently an integer from 2 to 8;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, heterocycloalkyl, or heterocycloalkyl-alkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

In some embodiments, provided herein is a compound of Formula (I):

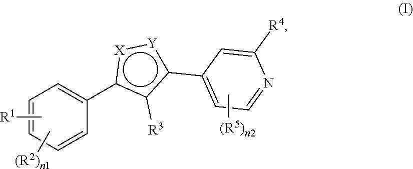

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
      wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, or heterocycloalkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹¹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

In some embodiments, the compound of Formula (I) is a compound of Formula (I-i):

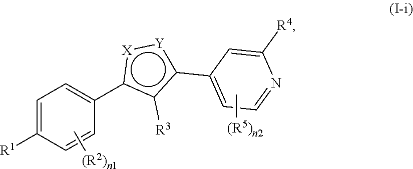

or a pharmaceutically acceptable salt, solvate, tautomer, isotope, or isomer thereof, wherein X, Y, R¹, R², R³, R⁴, R⁵, n1, and n2 are as described for Formula (I) above.

In some embodiments, the compound of Formula (II) is a compound of Formula (II-i):

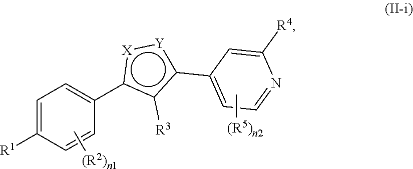

or a pharmaceutically acceptable salt, solvate, tautomer, isotope, or isomer thereof, wherein X, Y, R¹, R², R³, R⁴, R⁵, n1, and n2 are as described for Formula (II) above.

In some embodiments of the compound of Formula (I), X is S, Y is CR⁶, and the compound of Formula (I) is a compound of Formula (I-A):

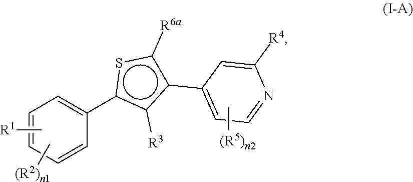

or a pharmaceutically acceptable salt, solvate, tautomer, isotope, or isomer thereof, where R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (I) above.

In some embodiments of the compound of Formula (II), X is S, Y is CR⁶, and the compound of Formula (I) is a compound of Formula (II-A):

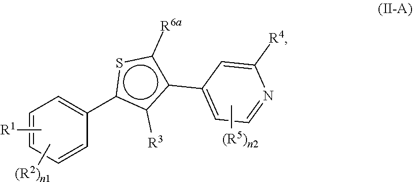

or a pharmaceutically acceptable salt, solvate, tautomer, isotope, or isomer thereof, where R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (II) above.

In certain embodiments, the compound of Formula (I) or Formula (I-A) is a compound of Formula (I-A-i):

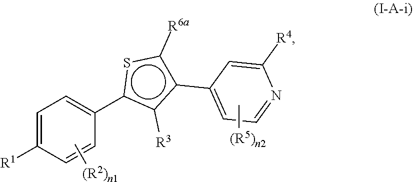

or a pharmaceutically acceptable salt, solvate, tautomer, isotope, or isomer thereof, where R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (I) above.

In certain embodiments, the compound of Formula (II) or Formula (II-A) is a compound of Formula (I-A-i):

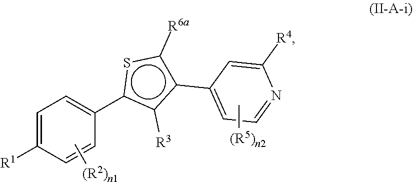

or a pharmaceutically acceptable salt, solvate, tautomer, isotope, or isomer thereof, where R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (II) above.

In even further embodiments, the compound of Formula (I), Formula (I-A), or Formula (I-A-i) is a compound of Formula (I-A-i-1):

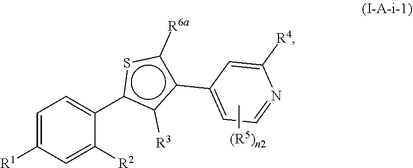

or a pharmaceutically acceptable salt, solvate, tautomer, isotope, or isomer thereof, where R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, and n2 are as described for Formula (I) above.

In even further embodiments, the compound of Formula (II), Formula (II-A), or Formula (II-A-i) is a compound of Formula (I-IA-i-1):

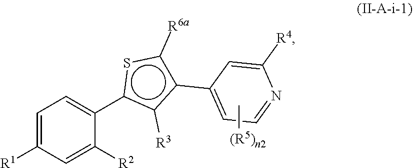

or a pharmaceutically acceptable salt, solvate, tautomer, isotope, or isomer thereof, where R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, and n2 are as described for Formula (II) above.

In other embodiments of the compound of Formula (I), Y is S, X is CR^{6b}, and the compound of Formula (I) is a compound of Formula (I-B):

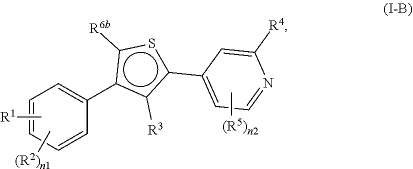

or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (I) above.

In other embodiments of the compound of Formula (II), Y is S, X is CR^{6b}, and the compound of Formula (II) is a compound of Formula (II-B):

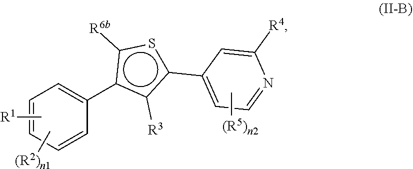

or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (II) above.

In certain embodiments, the compound of Formula (I) or Formula (I-B) is a compound of Formula (I-B-i):

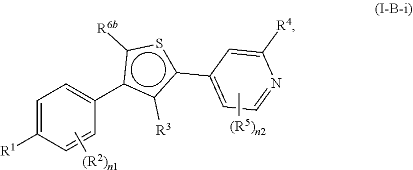

or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹; and R², R, R⁴, R⁵, R^{6a}, n1, and n2 are as described for Formula (I) above.

In certain embodiments, the compound of Formula (II) or Formula (II-B) is a compound of Formula (II-B-i):

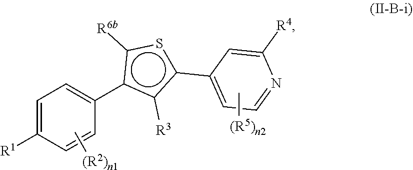

or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, n1, and n2 are as described for Formula (II) above.

In even further embodiments, the compound of Formula (I), Formula (I-B), or Formula (I-B-i) is a compound of Formula (I-B-i-1):

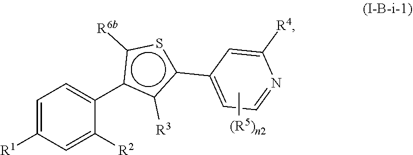

or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, and n2 are as described for Formula (I) above.

In even further embodiments, the compound of Formula (II), Formula (II-B), or Formula (II-B-i) is a compound of Formula (II-B-i-1):

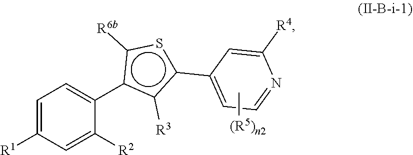

or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹; and R², R³, R⁴, R⁵, R^{6a}, and n2 are as described for Formula (II) above.

“Alkyl”, as used herein, refers to an unbranched or branched saturated hydrocarbon chain. Alkyl can be used alone, or as part of another radical, such as cycloalkyl-alkyl. In some embodiments, alkyl as used herein has 1 to 50 carbon atoms ((C₁₋₅₀)alkyl), 1 to 20 carbon atoms ((C₁₋₂₀)alkyl), 1 to 12 carbon atoms ((C₁₋₁₂)alkyl), 1 to 10 carbon atoms ((C₁₋₁₀)alkyl), 1 to 8 carbon atoms ((C₁₋₈)alkyl), 1 to 6 carbon atoms ((C₁₋₆)alkyl), or 1 to 4 carbon atoms ((C₁₋₄)alkyl). Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methyl pentyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

“Alkylene,” as used herein, refers to a divalent radical derived from a branched or unbranched alkyl, as exemplified, but not limited, by —CH₂CH₂CH₂CH₂—. In some embodiments, alkylene, as used herein, has 1 to 50 carbon atoms ((C₁₋₅₀)alkylene), 1 to 20 carbon atoms ((C₁₋₂₀)alkylene), 1 to 12 carbon atoms ((C₁₋₁₂)alkylene), 1 to 10 carbon atoms ((C₁₋₁₀)alkylene), 1 to 8 carbon atoms ((C₁₋₈)alkylene), 1 to 6 carbon atoms ((C₁₋₆)alkylene), or 1 to 4 carbon atoms ((C₁₋₄)alkylene).

“Alkenyl”, as used herein, refers to an unbranched or branched hydrocarbon chain containing at least one carbon-carbon double bond. Alkenyl can be used alone, or as part of another radical, such as cycloalkyl-alkenyl. In some embodiments, alkenyl as used herein has 1 to 50 carbon atoms ((C₁₋₅₀)alkenyl), 1 to 20 carbon atoms ((C₁₋₂₀)alkenyl), 1 to 12 carbon atoms ((C₁₋₁₂)alkenyl), 1 to 10 carbon atoms ((C₁₋₁₀)alkenyl), 1 to 8 carbon atoms ((C₁₋₈)alkenyl), 1 to 6 carbon atoms ((C₁₋₆)alkenyl), or 1 to 4 carbon atoms ((C₁₋₄)alkenyl). Alkenyl may have one, two, three, four, five, or more carbon-carbon double bonds, as valency permits. When an alkenyl residue having a specific number of carbons is named, all geometric isomers having that number of carbons may be encompassed.

“Carbocyclyl” refers to a monocyclic or polycyclic saturated or unsaturated hydrocarbon. Carbocyclyl includes cycloalkyl, aryl, and non-aromatic unsaturated carbocyclic groups such as cycloalkenyl. In some embodiments, carbocyclyl has 3 to 50 carbon atoms ((C₃₋₅₀)carbocyclyl), 3 to 20 carbon atoms ((C₃₋₂₀)carbocyclyl), 3 to 12 carbon atoms ((C₃₋₁₂)carbocyclyl), 3 to 10 carbon atoms ((C₃₋₁₀)carbocyclyl), 3 to 8 carbon atoms ((C₃₋₈)carbocyclyl), 3 to 6 carbon atoms ((C₃₋₆)carbocyclyl), or 3 to 5 carbon atoms ((C₃₋₄)carbocyclyl).

“Cycloalkyl”, as used herein, refers to a monocyclic or polycyclic saturated hydrocarbon. In some embodiments, cycloalkyl has 3 to 50 carbon atoms ((C₃₋₅₀)cycloalkyl), 3 to 20 carbon atoms ((C₃₋₂₀)cycloalkyl), 3 to 12 carbon atoms ((C₃₋₁₂)cycloalkyl), 3 to 10 carbon atoms ((C₃₋₁₀)cycloalkyl), 3 to 8 carbon atoms ((C₃₋₈)cycloalkyl), 3 to 6 carbon atoms ((C₃₋₆)cycloalkyl), or 3 to 5 carbon atoms ((C₃₋₄)cycloalkyl). Cycloalkyl includes monocyclic and polycyclic groups, such as fused bicycles, bridged rings, and spirocycles. Examples of cycloalkyl groups include cyclopropyl, cyclobutyl, cyclopentyl, cyclohexyl, octahydropentalenyl, octahydro-1H-indene, decahydronaphthalene, cubane, bicyclo[3.1.0]hexane, and bicyclo[1.1.1]pentane.

“Cycloalkenyl”, as used herein, refers to a non-aromatic monocyclic or polycyclic hydrocarbon containing at least one carbon-carbon double bond. In some embodiments, cycloalkenyl has 3 to 50 carbon atoms ((C₃₋₅₀)cycloalkenyl), 3 to 20 carbon atoms ((C₃₋₂₀)cycloalkenyl), 3 to 12 carbon atoms ((C₃₋₁₂)cycloalkenyl), 3 to 10 carbon atoms ((C₃₋₁₀)cycloalkenyl), 3 to 8 carbon atoms ((C₃₋₈)cycloalkenyl), 3 to 6 carbon atoms ((C₃₋₆)cycloalkenyl), or 3 to 5 carbon atoms ((C₃₋₄)cycloalkenyl). Cycloalkenyl includes monocyclic and polycyclic groups, and may have one, two, three, four, five, or more carbon-carbon double bonds, as valency permits.

“Cycloalkyl-alkyl” refers to a cycloalkyl group (as defined above) connected to an alkyl group (as defined above), wherein the alkyl group is attached to another moiety (such as the core structure of the molecule). Substituted cycloalkyl-alkyl can include one or more additional attachments to substituents at any point of the cycloalkyl or alkyl, as valency permits. The cycloalkyl-alkyl may comprise any combination of cycloalkyl and alkyl groups. In some embodiments, the cycloalkyl has 3 to 50 carbon atoms ((C₃₋₅₀)cycloalkyl-alkyl), 3 to 20 carbon atoms ((C₃₋₂₀)cycloalkyl-alkyl), 3 to 12 carbon atoms ((C₃₋₁₂)cycloalkyl-alkyl), 3 to 10 carbon atoms ((C₃₋₁₀)cycloalkyl-alkyl), 3 to 8 carbon atoms ((C₃₋₈)cycloalkyl-alkyl), 3 to 6 carbon atoms ((C₃₋₆)cycloalkyl-alkyl), or 3 to 5 carbon atoms ((C₃₋₄)cycloalkyl-alkyl). In some embodiments, the alkyl has 1 to 50 carbon atoms (cycloalkyl-(C₁₋₅₀)alkyl), 1 to 20 carbon atoms (cycloalkyl-(C₁₋₂₀)alkyl), 1 to 12 carbon atoms (cycloalkyl-(C₁₋₁₂)alkyl), 1 to 10 carbon atoms (cycloalkyl-(C₁₋₁₀)alkyl), 1 to 8 carbon atoms (cycloalkyl-(C₁₋₈)alkyl), 1 to 6 carbon atoms (cycloalkyl-(C₁₋₆)alkyl), or 1 to 4 carbon atoms (cycloalkyl-(C₁₋₄)alkyl). In certain embodiments, the cycloalkyl-alkyl is a (C₃₋₂₀)cycloalkyl(C₁₋₂₀)alkyl, (C₃₋₁₂)cycloalkyl(C₁₋₁₂)alkyl, (C₃₋₁₀)cycloalkyl(C₁₋₁₀)alkyl, (C₃₋₁₀)cycloalkyl(C₁₋₈)alkyl, (C₃₋₁₀)cycloalkyl(C₁₋₆)alkyl, (C₃₋₆)cycloalkyl(C₁₋₈)alkyl, (C₃₋₆)cycloalkyl(C₁₋₆)alkyl, or (C₃₋₆)cycloalkyl(C₁₋₄)alkyl.

“Heterocycloalkyl”, as used herein, refers to a saturated monocyclic or polycyclic ring containing carbon and at least one heteroatom selected from the group consisting of O, N, and S. The heterocycloalkyl group may comprise 3, 4, 5, 6, 7, 8, 9, 10, 11, 12, or more ring atoms (e.g., be a 3-membered, 4-membered, 5-membered, 6-membered, 7-membered, 8-membered, 9-membered, 10-membered, 11-membered, or 12-membered heterocycloalkyl). Heterocycloalkyl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. Each ring S atom, where present, may independently be unoxidized sulfur (e.g., —S—) or a sulfur oxide, such as —S(O)—, or —S(O)₂—. In certain examples, a heterocycloalkyl has 2 to 8 ring carbon atoms and with 1 to 3 ring heteroatoms independently selected from N, O, and S. In some embodiments, heterocycloalkyl is connected through an annular carbon atom, wherein the point of attachment of the heterocycloalkyl to another group is a ring carbon atom of the heterocycloalkyl. Heterocycloalkyl includes polycyclic systems, such as bridged, fused, and spirocycles comprising at least one heteroatom in at least one of the rings. Examples of heterocycloalkyl include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxinyl, piperidinyl, morpholinyl, thiomorpholinyl, thiomorpholinyl S-oxide, thiomorpholinyl S-dioxide, piperazinyl, azepinyl, oxepinyl, diazepinyl, and tropanyl.

“Heterocycloalkenyl”, as used herein, refers to a non-aromatic monocyclic or polycyclic ring containing carbon, at least one heteroatom selected from the group consisting of O, N, and S, and at least one double bond. Each ring S atom, where present, may independently be a sulfur oxide, such as —S(O)—, or —S(O)₂—. The heterocycloalkenyl group may comprise 3, 4, 5, 6, 7, 8, 9, 10, 11, 12, or more ring atoms (e.g., be a 3-membered, 4-membered, 5-membered, 6-membered, 7-membered, 8-membered, 9-membered, 10-membered, 11-membered, or 12-membered heterocycloalkenyl). Heterocycloalkenyl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In certain examples, a heterocycloalkenyl has 2 to 8 ring carbon atoms and with 1 to 3 ring heteroatoms independently selected from N, O, and S. In some embodiments, heterocycloalkenyl is connected through an annular carbon atom, wherein the point of attachment of the heterocycloalkenyl to another group is a ring carbon atom of the heterocycloalkenyl. Heterocycloalkenyl may have one, two, three, four, five, or more double bonds, as valency permits, and each double bond independently may be between two ring carbon atoms, two ring heteroatoms, or one ring carbon atom and one ring heteroatom, as valency permits.

“Heterocyclyl” refers to a saturated or unsaturated monocyclic or polycyclic ring containing carbon and at least one heteroatom selected from the group consisting of O, N, and S. Each ring S atom, where present, may independently be a sulfur oxide, such as —S(O)—, or —S(O)₂—. Heterocyclyl includes heterocycloalkyl, heteroaryl, and non-aromatic unsaturated heterocyclic groups such as heterocycloalkenyl. The heterocyclyl group may comprise 3, 4, 5, 6, 7, 8, 9, 10, 11, 12, or more ring atoms (e.g., be a 3-membered, 4-membered, 5-membered, 6-membered, 7-membered, 8-membered, 9-membered, 10-membered, 11-membered, or 12-membered heterocyclyl), and may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In some embodiments, heterocyclyl is connected through an annular carbon atom, wherein the point of attachment of the heterocyclyl to another group is a ring carbon atom of the heterocyclyl.

“Heterocycloalkyl-alkyl” refers to a heterocycloalkyl group (as defined above) connected to an alkyl group (as defined above), wherein the alkyl group is attached to another moiety (such as the core structure of the molecule). The alkyl group may be attached to the heterocycloalkyl through an annular carbon atom of the heterocycloalkyl, or through an annular heteroatom of the heterocycloalkyl (such as through a ring N atom). Substituted heterocycloalkyl-alkyl can include one or more additional attachments to substituents at any point of the heterocycloalkyl or alkyl, as valency permits. The heterocycloalkyl-alkyl may comprise any combination of heterocycloalkyl and alkyl groups. In some embodiments, the heterocycloalkyl comprises 3, 4, 5, 6, 7, 8, 9, 10, 11, 12, or more ring atoms. The heterocycloalkyl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In some embodiments, the alkyl has 1 to 50 carbon atoms (heterocycloalkyl-(C₁₋₅₀)alkyl), 1 to 20 carbon atoms (heterocycloalkyl-(C₁₋₂₀)alkyl), 1 to 12 carbon atoms (heterocycloalkyl-(C₁₋₁₂)alkyl), 1 to 10 carbon atoms (heterocycloalkyl-(C₁₋₁₀)alkyl), 1 to 8 carbon atoms (heterocycloalkyl-(C₁₋₈)alkyl), 1 to 6 carbon atoms (heterocycloalkyl-(C₁₋₆)alkyl), or 1 to 4 carbon atoms (heterocycloalkyl-(C₁₋₄)alkyl). In certain embodiments, the heterocycloalkyl-alkyl is a (3-20 membered)heterocycloalkyl(C₁₋₂₀)alkyl, (3-12 membered)heterocycloalkyl(C₁₋₁₂)alkyl, (3-12 membered)heterocycloalkyl(C₁₋₁₀)alkyl, (3-10 membered)heterocycloalkyl(C₁₋₈)alkyl, (3-10 membered)heterocycloalkyl(C₁₋₆)alkyl, (3-6 membered)heterocycloalkyl(C₁₋₈)alkyl, (3-6 membered)heterocycloalkyl(C₁₋₆)alkyl, or (3-6 membered)heterocycloalkyl(C₁₋₄)alkyl.

“Aryl”, as used herein, refers to an aromatic hydrocarbon monocyclic or polycyclic radical. Aryl may include groups with a single aromatic ring (e.g., phenyl) and multiple fused aromatic rings (e.g., naphthyl, anthryl). In some embodiments, aryl as used herein has from 6 to 14 annular carbon atoms ((C₆₋₁₄)aryl), or 6 to 10 annular carbon atoms ((C₆₋₁₀)aryl).

“Aryl-alkyl” refers to an aryl group (as defined above) connected to an alkyl group (as defined above), wherein the alkyl group is attached to another moiety (such as the core structure of the molecule). Substituted aryl-alkyl can include one or more additional attachments to substituents at any point of the aryl or alkyl, as valency permits. The aryl-alkyl may comprise any combination of aryl and alkyl groups. In some embodiments, the aryl has from 6 to 14 annular carbon atoms ((C₆₋₁₄)aryl-alkyl), or 6 to 10 annular carbon atoms ((C₆₋₁₀)aryl-alkyl). In some embodiments, the alkyl has 1 to 50 carbon atoms (aryl-(C₁₋₅₀)alkyl), 1 to 20 carbon atoms (aryl-(C₁₋₂₀)alkyl), 1 to 12 carbon atoms (aryl-(C₁₋₁₂)alkyl), 1 to 10 carbon atoms (aryl-(C₁₋₁₀)alkyl), 1 to 8 carbon atoms (aryl-(C₁₋₈)alkyl), 1 to 6 carbon atoms (aryl-(C₁₋₆)alkyl), or 1 to 4 carbon atoms (aryl-(C₁₋₄)alkyl). In certain embodiments, the aryl-alkyl is a (C₆₋₁₄)aryl(C₁₋₂₀)alkyl, (C₆₋₁₄)aryl(C₁₋₁₂)alkyl, (C₆₋₁₄)aryl(C₁₋₁₀)alkyl, (C₆₋₁₄)aryl(C₁₋₈)alkyl, (C₆₋₁₄)aryl(C₁₋₆)alkyl, (C₆₋₁₀)aryl(C₁₋₁₀)alkyl, (C₆₋₁₀)aryl(C₁₋₈)alkyl, (C₆₋₁₀)aryl(C₁₋₆)alkyl, or (C₆₋₁₀)aryl(C₁₋₄)alkyl.

“Heteroaryl”, as used herein, refers to a monocyclic or polycyclic radical comprising at least one aromatic ring, wherein the aromatic ring comprises at least one ring heteroatom independently selected from the group consisting of N, O, and S, (e.g., pyridine, pyrazine, furan, thiophene, quinoline). Each ring S atom, where present, may independently be unoxidized sulfur (e.g., —S—) or a sulfur oxide, such as —S(O)—, or —S(O)₂—. Heteroaryl may include groups comprising 1 to 5 ring heteroatoms, 1 to 4 heteroatoms, 1 to 3 ring heteroatoms, 1 or 2 ring heteroatoms, or 1 ring heteroatom, wherein each heteroatom is independently selected from the group consisting of N, O, and S. In certain examples, a heteroaryl has 3 to 8 ring carbon atoms, with 1 to 3 ring heteroatoms independently selected from N, O, and S. Heteroaryl may comprise 5, 6, 7, 8, 9, 10, 11, 12, or more annular atoms (e.g., be a 3-membered, 4-membered, 5-membered, 6-membered, 7-membered, 8-membered, 9-membered, 10-membered, 11-membered, or 12-membered heteroaryl), wherein the annular atoms are present in one or more rings. Heteroaryl may comprise, for example, 1 to 14 annular carbon atoms ((C₁₋₁₄)heteroaryl), 1 to 10 annular carbon atoms ((C₁₋₁₀)heteroaryl), 1 to 6 annular carbon atoms ((C₁₋₆)heteroaryl), 1 to 5 annular carbon atoms ((C₁₋₅)heteroaryl), or 2 to 5 annular carbon atoms ((C₂₋₅)heteroaryl). In some embodiments, heteroaryl is connected through an annular carbon atom, wherein the point of attachment of the heteroaryl to another group is a ring carbon atom of the heteroaryl. Examples of heteroaryl groups include pyridyl, pyridazinyl, pyrimidinyl, benzothiazolyl, furanyl, and pyrazolyl.

“Heteroaryl-alkyl” refers to a heteroaryl group (as defined above) connected to an alkyl group (as defined above), wherein the alkyl group is attached to another moiety (such as the core structure of the molecule). Substituted heteroaryl-alkyl can include one or more additional attachments to substituents at any point of the heteroaryl or alkyl, as valency permits. The alkyl group may be attached to the heteroaryl through an annular carbon atom of the heteroaryl, or through an annular heteroatom of the heteroaryl. The heteroaryl-alkyl may comprise any combination of heteroaryl and alkyl groups. The heteroaryl may have 3 to 8 ring carbon atoms, with 1 to 3 ring heteroatoms independently selected from N, O, and S. Heteroaryl may comprise 5, 6, 7, 8, 9, 10, 11, 12, or more annular atoms (e.g., be a 3-membered, 4-membered, 5-membered, 6-membered, 7-membered, 8-membered, 9-membered, 10-membered, 11-membered, or 12-membered heteroaryl), wherein the annular atoms are present in one or more rings. In some embodiments, the alkyl has 1 to 50 carbon atoms (heteroaryl-(C₁₋₅₀)alkyl), 1 to 20 carbon atoms (heteroaryl-(C₁₋₂₀)alkyl), 1 to 12 carbon atoms (heteroaryl-(C₁₋₁₂)alkyl), 1 to 10 carbon atoms (heteroaryl-(C₁₋₁₀)alkyl), 1 to 8 carbon atoms (heteroaryl-(C₁₋₈)alkyl), 1 to 6 carbon atoms (heteroaryl-(C₁₋₆)alkyl), or 1 to 4 carbon atoms (heteroaryl-(C₁₋₄)alkyl). In certain embodiments, the heteroaryl-alkyl is a (C₁₋₁₄)heteroaryl(C₁₋₂₀)alkyl, (C₁₋₁₀)heteroaryl(C₁₋₁₂)alkyl, (C₁₋₆)heteroaryl(C₁₋₁₀)alkyl, (C₁₋₅)heteroaryl(C₁₋₈)alkyl, (C₁₋₅)heteroaryl(C₁₋₆)alkyl, (C₁₋₅)heteroaryl (C₁₋₈)alkyl, (C₁₋₅)heteroaryl(C₁₋₆)alkyl, or (C₁₋₅)heteroaryl(C₁₋₄)alkyl.

It should be understood that when a range of values is listed, it is intended to encompass each value and sub-range within the range. For example, “(C₁₋₆)alkyl” (which may also be referred to as C₁-C₆ alkyl, C₁-C₆ alkyl, or C₁₋₆ alkyl) is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Hydroxy”, as used herein, refers to the radical —OH.

“Halo”, as used herein, refers to fluoro, chloro, bromo, or iodo radicals.

“Cyano” means the radical —CN.

“Oxo”, as used herein, refers to the radical ═O.

“Optional” or “optionally” means that the subsequently described event or circumstance may or may not occur, and that the description includes instances where the event or circumstance occurs and instances in which it does not.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), the compound is a solvate. In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), the solvate is a hydrate.

In some embodiments, provided is a pharmaceutically acceptable salt of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1).

“Pharmaceutically acceptable” includes that which is useful in preparing a pharmaceutical composition that is generally safe, non-toxic and not biologically or otherwise undesirable, and includes that which is acceptable for veterinary use as well as human pharmaceutical use. For example, provided herein is a pharmaceutical composition comprising a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, and a pharmaceutically acceptable excipient.

“Pharmaceutically acceptable salt” includes a salt which is generally safe, non-toxic and not biologically or otherwise undesirable, and includes that which is acceptable for veterinary use as well as human pharmaceutical use. Such salts may include acid addition salts and base addition salts. Acid addition salts may be formed with inorganic acid such as, but not limited to, hydrochloric acid, hydrobromic acid, sulfuric acid, nitric acid, phosphoric acid and the like; or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, or undecylenic acid. Salts derived from inorganic bases may include, but are not limited to, sodium, potassium, lithium, ammonium, calcium, magnesium, iron, zinc, copper, manganese, and aluminum salts. Salts derived from organic bases may include, but are not limited to, salts of primary, secondary, or tertiary amines; substituted amines including naturally occurring substituted amines; cyclic amines; ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, or N-ethylpiperidine.

In some embodiments, provided is an isotope of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1).

Unless otherwise stated, structures depicted herein, such as compounds of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, or isomer thereof, are also meant to include compounds which differ only in the presence of one or more isotopically enriched atoms. The compounds herein may contain unnatural proportions of atomic isotopes at one or more of the atoms that constitute such compounds. In some embodiments, the compound is isotopically-labeled, such as an isotopically-labeled compound of Formula (I), (I-i), (I-A), (I-A-i), (I-A-i-1), (I-B), (I-B-i), (I-B-i-1), (II), (II-i), (II-A), (II-A-i), (I-A-i-1), (II-B), (II-B-i), or (II-B-i-1), or a pharmaceutically acceptable salt, solvate, tautomer, or isomer thereof, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³⁵S, ¹⁸F, ³⁶Cl. Certain isotope labeled compounds (e.g. ³H and ¹⁴C) may be useful in compound or substrate tissue distribution study. Incorporation of heavier isotopes such as deuterium (²H) may, in some embodiments, afford certain therapeutic advantages resulting from greater metabolic stability, for example, increased in vivo half-life, or reduced dosage requirements.

The compounds disclosed herein, such as compounds of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, or isotope thereof, may contain one or more asymmetric centers and thus may give rise to one or more isomers.

In some embodiments, provided is a tautomer of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, or isotope thereof genus, n2 is 0. In some embodiments, R³ is hydrogen. In certain embodiments, R⁶a or R^{6b} is hydrogen. In some embodiments, n1 is 0 or 1. In some embodiments, R² is halo.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, or isotope thereof:
R⁷, R⁸, and R⁹ are independently selected from the group consisting of hydrogen, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, (C₆₋₁₀)aryl, aryl-(C₁₋₁₀)alkyl, heteroaryl, and heteroaryl-(C₁₋₁₀)alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹, is independently unsubstituted or substituted with one or more substituents independently selected from the group consisting of halo, (C₁₋₁₀)alkyl, (C₁₋₁₀)haloalkyl, cyano, oxo, —OR¹⁴, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
or R⁸ and R⁹, together with the nitrogen atom to which they are attached, form a heterocycloalkyl, which is unsubstituted or substituted with one or more substituents independently selected from the group consisting of halo, cyano, oxo, (C₁₋₁₀)alkyl, (C₂₋₁₀)alkenyl, (C₂₋₁₀)alkynyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, aryl, aryl-(C₁₋₁₀)alkyl, heteroaryl, heteroaryl-(C₁₋₁₀)alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   wherein each (C₁₋₁₀)alkyl, (C₂₋₁₀)alkenyl, (C₂₋₁₀)alkynyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, aryl, aryl-(C₁₋₁₀)alkyl, heteroaryl, and heteroaryl-(C₁₋₁₀)alkyl is independently unsubstituted or substituted with one or more substituents independently selected from the group consisting of halo, oxo, (C₁₋₁₀)alkyl, (C₁₋₁₀)haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, (C₁₋₁₀)alkyl, (C₃₋₁₀)cycloalkyl, (C₃₋₁₀)cycloalkyl-(C₁₋₁₀)alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, (C₁₋₁₀)alkyl, (C₃₋₁₀)cycloalkyl, (C₃₋₁₀)cycloalkyl-(C₁₋₁₀)alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is (C₁₋₁₀)alkyl, (C₂₋₁₀)alkenyl, (C₃₋₁₀)cycloalkyl, (C₃₋₁₀)cycloalkyl-(C₁₋₁₀)alkyl, (C₃₋₁₀)cycloalkenyl, heterocycloalkyl, heterocycloalkyl-(C₁₋₁₀)alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, —OR¹¹, —C(O)NR¹³R¹³, —S(O)₂NR¹³R¹³, —S(O)ₘ₄R¹³, or —C(O)R¹³;
or R⁴ and one R⁵, together with the atoms to which they are attached, form a (C₄₋₁₀)carbocyclyl or heterocyclyl,
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, (C₁₋₁₀)alkyl, (C₃₋₁₀)cycloalkyl, (C₃₋₁₀)cycloalkyl-(C₁₋₁₀)alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴,
   wherein each (C₁₋₁₀)alkyl, (C₃₋₁₀)cycloalkyl, (C₃₋₁₀)cycloalkyl-(C₁₋₁₀)alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, (C₁₋₁₀)alkyl, or (C₁₋₁₀)haloalkyl; each n4 is independently 0, 1, or 2; each n5 is independently an integer from 0 to 5; and each R¹⁸ is independently (C₁₋₁₀)alkylene or (C₁₋₁₀)haloalkylene;
R³, R^{6a}, and R^{6b} are independently selected from the group consisting of hydrogen, halo, cyano, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, (C₁₋₁₀)alkyl, (C₃₋₁₀)cycloalkyl, (C₃₋₁₀)cycloalkyl-(C₁₋₁₀)alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, (C₁₋₁₀)alkyl, (C₃₋₁₀)cycloalkyl, (C₃₋₁₀)cycloalkyl-(C₁₋₁₀)alkyl, or heterocycloalkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, (C₁₋₁₀)alkyl, (C₁₋₁₀)haloalkyl, —C(O)OR¹⁹, —C(O)NR¹⁹R¹⁹, —NR¹⁹C(O)R¹¹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, (C₁₋₁₀)alkyl, or (C₁₋₁₀)haloalkyl; each n6 is independently 0, 1, or 2; each n7 is independently an integer from 0 to 5; and each R²⁰ is independently (C₁₋₁₀)alkylene or (C₁₋₁₀)haloalkylene;
each R²¹ is independently (C₁₋₁₀)alkylene or (C₁₋₁₀)haloalkylene;
each n6 is independently an integer from 1 to 5; and
each m1, m2, m3, and m4 is independently 0, 1, or 2.

In some embodiments of the genus in the preceding paragraph, n2 is 0 or 1. In some embodiments, n2 is 0. In some embodiments, R³ is hydrogen. In certain embodiments, R^{6a} or Rb is hydrogen. In some embodiments, n1 is 0 or 1. In some embodiments, R² is halo.

In some embodiments of the compound of Formula (II), (II-i), (II-A), (II-A-i), (II-A-i-1), (II-B), (II-B-i), or (II-B-i-1), or a pharmaceutically acceptable salt, solvate, tautomer, or isotope thereof:
R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, —C(O)R¹⁴, and —OC(O)R²²,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R²² is independently —R²³N(R²⁴)₂ or —(CH₂CH₂—O—)ₙ₈CH₃, wherein each R²³ is (C₁-C₆)alkyl; each R²⁴ is independently H or —CH₃; and each n8 is independently an integer from 2 to 8;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, heterocycloalkyl, or heterocycloalkyl-alkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

In some embodiments of the genus in the preceding paragraph, n2 is 0 or 1. In some embodiments, n2 is 0. In some embodiments, R³ is hydrogen. In certain embodiments, R^{6a} or R^{6b} is hydrogen. In some embodiments, n1 is 0 or 1. In some embodiments, R² is halo.

In certain embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, or isotope thereof:
R⁷ is hydrogen;
R⁸ and R⁹ are independently hydrogen, (C₁₋₁₀)alkyl, (C₃₋₁₀)cycloalkyl, (C₃₋₁₀)cycloalkyl-(C₁₋₁₀)alkyl, heterocycloalkyl, or heterocycloalkyl-(C₁₋₁₀)alkyl; wherein each alkyl, cycloalkyl, cycloalkyl-alkyl, heterocycloalkyl, and heterocycloalkyl-alkyl is independently unsubstituted or substituted with one or more substituents independently selected from the group consisting of halo and —OR¹⁰;
or R⁸ and R⁹, together with the nitrogen atom to which they are attached, form a heterocycloalkyl, which is unsubstituted or substituted with one or more substituents independently selected from the group consisting of halo, oxo, (C₁₋₁₀)alkyl, (C₁₋₁₀)haloalkyl, and —OR¹⁰;
n1 is 0 or 1;
R² is halo;
R⁴ is (C₁₋₁₀)alkyl, (C₃₋₁₀)cycloalkyl, (C₃₋₁₀)cycloalkyl-(C₁₋₁₀)alkyl, heterocycloalkyl, or heterocycloalkyl-(C₁₋₁₀)alkyl,
   wherein R⁴ may be unsubstituted or substituted with one or more substituents independently selected from the group consisting of halo, (C₁₋₁₀)alkyl, (C₃₋₁₀)cycloalkyl, —OR¹⁴, and —C(O)OR¹⁴,
      wherein the (C₁₋₁₀)alkyl and (C₃₋₁₀)cycloalkyl may independently be unsubstituted or substituted with one or more halo, —OH, —O(C₁₋₁₀)alkyl, or —O(C₁₋₁₀)haloalkyl, or any combinations thereof,
or R⁴ and R⁵, if present, together with the atoms to which they are attached, form a 5- or 8-membered heterocyclyl, unsubstituted or substituted with one or more substituents independently selected from the group consisting of halo, —OH, —O(C₁₋₁₀)alkyl, and —O(C₁₋₁₀)haloalkyl;
n2 is 0 or 1;
R³, R^{6a}, and R^{6b} are independently selected from the group consisting of hydrogen, halo, (C₁₋₁₀)alkyl, and (C₁₋₁₀)haloalkyl;
each R¹⁰ and R¹⁴ is independently hydrogen, (C₁₋₁₀)alkyl, (C₁₋₁₀)haloalkyl, (C₃₋₁₀)cycloalkyl, or (C₃₋₁₀)halocycloalkyl.

In certain embodiments of the compound of Formula (II), (II-i), (II-A), (II-A-i), (II-A-i-1), (II-B), (II-B-i), or (II-B-i-1), or a pharmaceutically acceptable salt, solvate, tautomer, or isotope thereof:
R⁷ is hydrogen;
R⁸ and R⁹ are independently hydrogen, (C₁₋₁₀)alkyl, (C₃₋₁₀)cycloalkyl, (C₃₋₁₀)cycloalkyl-(C₁₋₁₀)alkyl, heterocycloalkyl, or heterocycloalkyl-(C₁₋₁₀)alkyl; wherein each alkyl, cycloalkyl, cycloalkyl-alkyl, heterocycloalkyl, and heterocycloalkyl-alkyl is independently unsubstituted or substituted with one or more substituents independently selected from the group consisting of halo and —OR¹⁰;
or R⁸ and R⁹, together with the nitrogen atom to which they are attached, form a heterocycloalkyl, which is unsubstituted or substituted with one or more substituents independently selected from the group consisting of halo, oxo, (C₁₋₁₀)alkyl, (C₁₋₁₀)haloalkyl, and —OR¹⁰; n1 is 0 or 1;
R² is halo;
R⁴ is (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, or —OR¹²,
   wherein R⁴ may be unsubstituted or substituted with one or more substituents independently selected from the group consisting of halo, (C₁₋₁₀)alkyl, (C₃₋₁₀)cycloalkyl, —OR¹⁴, —C(O)OR¹⁴, and —OC(O)R²²,
      wherein the (C₁₋₁₀)alkyl and (C₃₋₁₀)cycloalkyl may independently be unsubstituted or substituted with one or more halo, —OH, —O(C₁₋₁₀)alkyl, or —O(C₁₋₁₀)haloalkyl, or any combinations thereof,
or R⁴ and R⁵, if present, together with the atoms to which they are attached, form a 5- or 8-membered heterocyclyl, unsubstituted or substituted with one or more substituents independently selected from the group consisting of halo, —OH, —O(C₁₋₁₀)alkyl, and —O(C₁₋₁₀)haloalkyl;
n2 is 0 or 1;
R³, R^{6a}, and R^{6b} are independently selected from the group consisting of hydrogen, halo, (C₁₋₁₀)alkyl, and (C₁₋₁₀)haloalkyl;
each R¹⁰ and R¹⁴ is independently hydrogen, (C₁₋₁₀)alkyl, (C₁₋₁₀)haloalkyl, (C₃₋₁₀)cycloalkyl, or (C₃₋₁₀)halocycloalkyl;
R¹² is independently hydrogen, alkyl, cycloalkyl, cycloalkyl-alkyl, heterocycloalkyl, or heterocycloalkyl-alkyl,
   wherein each R¹² is independently unsubstituted or substituted with one or more substituents independently selected from the group consisting of cyano, —OH, —OCH₃, and —NH₂;
R²² is independently —R²³N(R²⁴)₂ or —(CH₂CH₂—O—)ₙ₈CH₃,
   wherein each R²³ is (C₁-C₆)alkyl; each R²⁴ is independently H or —CH₃; and each n8 is independently an integer from 2 to 8.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R¹ is —NR⁷C(O)NR⁸R⁹, —NR⁷S(O)₂NR⁸R⁹, —NR⁷C(O)OR⁸, —S(O)₂R⁸, or —NR⁸R⁹. In certain embodiments, R¹ is —NR⁷C(O)NR⁸R⁹ or —NR⁷S(O)₂NR⁸R⁹. In some embodiments, R¹ is —NR⁷C(O)OR⁹ or —S(O)₂R⁹.

In some embodiments of the compound of Formula (I), (I-i), (I-A), (I-A-i), (I-A-i-1), (II), (II-i), (II-A), (II-A-i), or (II-A-i-1), or a pharmaceutically acceptable salt, solvate, tautomer, isotope, or isomer thereof, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹. In certain embodiments, R¹ is —NR⁷C(O)NR⁸R⁹, —NR⁷S(O)₂NR⁸R⁹, —NR⁷C(O)OR⁹, —NR⁷S(O)₂R⁹, —S(O)₂R⁹, —NR⁷C(S)NR⁸R⁹, or —NR⁷C(O)SR⁹. In still further embodiments, R¹ is —NR⁷C(O)NR⁸R⁹, —NR⁷S(O)₂NR⁸R⁹, —NR⁷C(O)OR⁹, —NR⁷S(O)₂R⁹, or —S(O)₂R⁹. In certain embodiments, R¹ is —NR⁷C(O)NR⁸R⁹, —NR⁷S(O)₂NR⁸R⁹, —NR⁷C(O)OR⁸, —S(O)₂R⁸, —NR⁷(SO)₂R⁹, or —NR⁸R⁹.

In some embodiments of the compound of Formula (I), (I-i), (I-B), (I-B-i), (I-B-i-1), (II), (II-i), (II-B), (II-B-i), or (II-B-i-1), or a pharmaceutically acceptable salt, solvate, tautomer, isotope, or isomer thereof, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹. In certain embodiments, R¹ is —NR⁷C(O)NR⁸R⁹, —NR⁷S(O)₂NR⁸R⁹, —NR⁷C(O)OR⁹, —S(O)₂R⁹, —NR⁷C(S)NR⁸R⁹, or —NR⁷C(O)SR⁹. In still further embodiments, R¹ is —NR⁷C(O)NR⁸R⁹, —NR⁷S(O)₂NR⁸R⁹, —NR⁷C(O)OR⁹, or —S(O)₂R⁹.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R⁷ is hydrogen, alkyl, cycloalkyl, or cycloalkyl-alkyl, wherein the alkyl, cycloalkyl, or cycloalkyl-alkyl is unsubstituted or substituted with one or more substituents independently selected from the group consisting of halo, cyano, oxo, —OR¹⁰, —C(O)NR¹⁰R¹⁰, —NR¹⁰C(O)R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, and —C(O)R¹⁰. In some embodiments, R⁷ is hydrogen, alkyl, cycloalkyl, or cycloalkyl-alkyl, wherein the alkyl, cycloalkyl, or cycloalkyl-alkyl is unsubstituted or substituted with one or more substituents independently selected from the group consisting of halo and —OR¹⁰. In certain embodiments, each R¹⁰ is independently hydrogen, alkyl, or haloalkyl. In still further embodiments, each R¹⁰ is independently hydrogen, (C₁₋₁₀)alkyl, or (C₁₋₁₀)haloalkyl. In some embodiments, R⁷ is hydrogen. In certain embodiments, wherein R⁷ is alkyl, cycloalkyl, or cycloalkyl-alkyl, unsubstituted or substituted, R⁷ is (C₁₋₁₀)alkyl, (C₃₋₈)cycloalkyl, or (C₃₋₈)cycloalkyl-(C₁₋₁₀)alkyl.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R⁸ hydrogen, alkyl, cycloalkyl, or cycloalkyl-alkyl, wherein the alkyl, cycloalkyl, or cycloalkyl-alkyl is unsubstituted or substituted with one or more substituents independently selected from the group consisting of halo, cyano, oxo, —OR¹⁰, —C(O)NR¹⁰R¹⁰, —NR¹⁰C(O)R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, and —C(O)R¹⁰. In some embodiments, R⁸ is hydrogen, alkyl, cycloalkyl, or cycloalkyl-alkyl, wherein the alkyl, cycloalkyl, or cycloalkyl-alkyl is unsubstituted or substituted with one or more substituents independently selected from the group consisting of halo and —OR¹⁰. In certain embodiments, each R¹⁰ is independently hydrogen, alkyl, or haloalkyl. In still further embodiments, each R¹⁰ is independently hydrogen, (C₁₋₁₀)alkyl, or (C₁₋₁₀)haloalkyl. In some embodiments, R⁸ is hydrogen. In certain embodiments, wherein R⁸ is alkyl, cycloalkyl, or cycloalkyl-alkyl, unsubstituted or substituted, R⁷ is (C₁₋₁₀)alkyl, (C₃₋₈)cycloalkyl, or (C₃₋₈)alkyl, (C₁₋₁₀)alkyl.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R⁹ is hydrogen, alkyl, cycloalkyl, cycloalkyl-alkyl, heterocycloalkyl, or heterocycloalkyl-alkyl; wherein the alkyl, cycloalkyl, cycloalkyl-alkyl, heterocycloalkyl, or heterocycloalkyl-alkyl is unsubstituted or substituted with one or more substituents independently selected from the group consisting of halo, cyano, oxo, —OR¹⁰, —C(O)NR¹⁰R¹⁰, —NR¹⁰C(O)R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, and —C(O)R¹⁰. In some embodiments, R⁹ is alkyl, cycloalkyl, cycloalkyl-alkyl, heterocycloalkyl, or heterocycloalkyl-alkyl, and is unsubstituted or substituted with one or more substituents independently selected from the group consisting of halo and —OR¹⁰. In certain embodiments, each R¹⁰ is independently hydrogen, alkyl, or haloalkyl. In still further embodiments, each R¹⁰ is independently hydrogen, (C₁₋₁₀)alkyl, or (C₁₋₁₀)haloalkyl. In still further embodiments, R⁹ is (C₁₋₁₀)alkyl, (C₃₋₈)cycloalkyl, (C₃₋₈)cycloalkyl-(C₁₋₁₀)alkyl, (5- to 7-membered)heterocycloalkyl, or (5- to 7-membered)heterocycloalkyl-(C₁₋₁₀) alkyl, which is unsubstituted or substituted with one or more substituents independently selected from the group consisting of halo and —OR¹⁰. In some embodiments, R¹⁰ is hydrogen, alkyl, or haloalkyl. In still further embodiments, R⁹ is (C₁₋₁₀)alkyl, unsubstituted or substituted with one or more halo, —OH, —O(C₁₋₁₀)alkyl, or —O(C₁₋₁₀)haloalkyl. In some embodiments, R⁹ is (C₃₋₈)cycloalkyl, unsubstituted or substituted with one or more halo, —OH, —O(C₁₋₁₀)alkyl, or —O(C₁₋₁₀)haloalkyl. In some embodiments, R⁹ is (C₃₋₈)cycloalkyl-(C₁₋₁₀)alkyl, unsubstituted or substituted with one or more halo, —OH, —O(C₁₋₁₀)alkyl, or —O(C₁₋₁₀)haloalkyl. In some embodiments, R⁹ is (5- to 7-membered)heterocycloalkyl, unsubstituted or substituted with one or more halo, —OH, —O(C₁₋₁₀)alkyl, or —O(C₁₋₁₀)haloalkyl. In still further embodiments, R⁹ is (5- to 7-membered)heterocycloalkyl-(C₁₋₁₀) alkyl, unsubstituted or substituted with one or more halo, —OH, —O(C₁₋₁₀)alkyl, or —O(C₁₋₁₀)haloalkyl. In certain embodiments, R⁹ is selected from the group consisting of methyl, ethyl, propyl, butyl, cyclopropyl, cyclopentyl, cyclohexyl, cyclopentane-methyl, cyclopentane-ethyl, cyclohexane-methyl, cyclohexane-ethyl, pyrrolidinyl, pyrrolidine-ethyl, pyrrolidine-methyl, piperidinyl, piperidinyl-methyl, or piperidinyl-ethyl, wherein each of the foregoing is independently unsubstituted or substituted with one or more halo, one or more —OH, or any combinations thereof.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R⁸ and R⁹, together with the nitrogen atom to which they are attached, form a heterocycloalkyl, wherein the heterocycloalkyl is unsubstituted or substituted with one or more substituents selected from the group consisting of halo, cyano, oxo, alkyl, alkenyl, alkynyl, cycloalkyl, cycloalkyl-alkyl, heterocycloalkyl, heterocycloalkyl-alkyl, —C(O)NR¹⁰R¹⁰, —NR¹⁰C(O)R¹⁰, —OR¹⁰, —NR¹⁰R¹⁰, —C(O)OR¹⁰, and —C(O)R¹⁰. In some embodiments, the heterocycloalkyl is a 4- to 8-membered heterocycloalkyl. In certain embodiments, the heterocycloalkyl a 5- to 7-membered heterocycloalkyl. In some embodiments, the heterocycloalkyl comprises, in addition to the one nitrogen attached to both R⁸ and R⁹, between 0 to 3 heteroatoms selected from the group consisting of O, N, and S. In other embodiments, the heterocycloalkyl comprises 0, 1, or 2 heteroatoms selected from the group consisting of O, N, and S, in addition to the one N attached to both R^{x} and R⁹. In some embodiments, R⁸ and R⁹, together with the nitrogen atom to which they are attached, form a 5- to 7-membered heterocycloalkyl, wherein the heterocycloalkyl comprises 0, 1, or 2 heteroatoms selected from the group consisting of O, N, and S, in addition to the one N attached to both R⁸ and R⁹. In some embodiments, the heterocycloalkyl is unsubstituted or substituted with one to three substituents independently selected from the group consisting of halo, cyano, oxo, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, —C(O)NR¹⁰R¹⁰, —NR¹⁰C(O)R¹⁰, —OR¹⁰, —NR¹⁰R¹⁰, —C(O)OR¹⁰, and —C(O)R¹⁰. In further embodiments, the heterocycloalkyl is unsubstituted or substituted with one to three substituents independently selected from the group consisting of halo, oxo, and —OR¹⁰. In some embodiments, each R¹⁰ is independently hydrogen, (C₁₋₁₀)alkyl, or (C₁₋₁₀)haloalkyl.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, such as embodiments described herein, R¹ is —NR⁷C(O)NR⁸R⁹. In some embodiments, R⁷ is hydrogen, alkyl, or haloalkyl. In certain embodiments, R⁷ is hydrogen. In some embodiments, R⁸ and R⁹ are independently hydrogen, alkyl, cycloalkyl, cycloalkyl-alkyl, heterocycloalkyl, or heterocycloalkyl-alkyl; wherein each alkyl, cycloalkyl, cycloalkyl-alkyl, heterocycloalkyl, and heterocycloalkyl-alkyl is independently unsubstituted or substituted with one or more substituents independently selected from the group consisting of halo and —OR¹⁰. In some embodiments, R⁸ is hydrogen. In certain embodiments, R⁹ is (C₁₋₁₀)alkyl, (C₃₋₁₀)cycloalkyl, (C₃₋₁₀)cycloalkyl-(C₁₋₁₀)alkyl, heterocycloalkyl, or heterocycloalkyl-(C₁₋₁₀)alkyl. In some embodiments, R⁹ is 5- or 6-membered heterocycloalkyl. In other embodiments, R⁹ is 5- or 6-membered heterocycloalkyl-(C₁₋₁₀)alkyl. In some embodiments, R⁹ is methyl, ethyl, propyl, butyl, pentyl, hexyl, cyclopropyl, cyclobutyl, cyclopentyl, cyclohexyl, cyclobutane-ethyl, cyclopentane-methyl, cyclopentane-ethyl, cyclohexane-methyl, cyclohexane-ethyl, pyrrolidinyl, piperidinyl, pyrrolidine-methyl, pyrrolidine-ethyl, pyrrolidine-propyl, piperidine-methyl, piperidine-ethyl, or piperidine-propyl. In other embodiments, R⁸ and R⁹, together with the nitrogen atom to which they are attached, form a heterocycloalkyl. For example, in some embodiments, R⁸ and R⁹, together with the nitrogen atom to which they are attached, form a 5- or 6-membered heterocycloalkyl. In certain embodiments, each of the foregoing moieties of R⁹, or the heterocycloalkyl formed by R⁸ and R⁹ together, is unsubstituted or substituted with one to three substituents independently selected from the group consisting of halo and —OR¹⁰. In certain embodiments, each R¹⁰ is independently hydrogen, (C₁₋₁₀)alkyl, or (C₁₋₁₀)haloalkyl. In some embodiments, R¹⁰ is —OH. In certain embodiments, R¹ is:

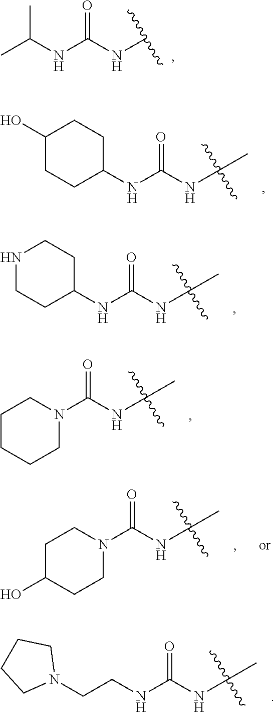

In other embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, such as embodiments described herein, R¹ is —NR⁷C(O)OR⁹. In some embodiments, R⁷ is hydrogen, alkyl, or haloalkyl. In certain embodiments, R⁷ is hydrogen. In certain embodiments, R⁹ is alkyl, cycloalkyl, cycloalkyl-alkyl, heterocycloalkyl, or heterocycloalkyl-alkyl, wherein each of the foregoing is unsubstituted or substituted with one or more substituents independently selected from the group consisting of halo and —OR¹⁰. In some embodiments, R⁹ is 5- or 6-membered heterocycloalkyl. In other embodiments, R⁹ is (5- or 6-membered)heterocycloalkyl-(C₁₋₁₀)alkyl. In some embodiments, R⁹ is methyl, ethyl, propyl, butyl, pentyl, hexyl, cyclopropyl, cyclobutyl, cyclopentyl, cyclohexyl, cyclobutane-ethyl, cyclopentane-methyl, cyclopentane-ethyl, cyclohexane-methyl, cyclohexane-ethyl, pyrrolidinyl, piperidinyl, pyrrolidine-methyl, pyrrolidine-ethyl, pyrrolidine-propyl, piperidine-methyl, piperidine-ethyl, or piperidine-propyl. In certain embodiments, each of the foregoing moieties of R⁹ is unsubstituted or substituted with one to three substituents independently selected from the group consisting of halo and —OR¹⁰. In certain embodiments, each R¹⁰ is independently hydrogen, (C₁₋₁₀)alkyl, or (C₁₋₁₀)haloalkyl. In some embodiments, R¹⁰ is —OH. In some embodiments, R¹ is:

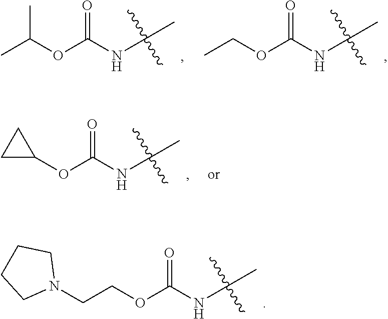

In other embodiments of the compound of Formula (I), (I-i), (I-A), (I-A-i), (I-A-i-1), (I-B), (I-B-i), (I-B-i-1), (II), (II-i), (II-A), (I1-A-i), (IT-A-i-1), (IT-B), (II-B-i), or (II-B-i-1), or a pharmaceutically acceptable salt, solvate, tautomer, isotope, or isomer thereof, such as embodiments described herein, R¹ is —NR⁷S(O)₂NR⁸R⁹. In some embodiments, R⁷ is hydrogen, alkyl, or haloalkyl. In certain embodiments, R⁷ is hydrogen. In some embodiments, R⁸ and R⁹ are independently hydrogen, alkyl, cycloalkyl, cycloalkyl-alkyl, heterocycloalkyl, or heterocycloalkyl-alkyl; wherein each alkyl, cycloalkyl, cycloalkyl-alkyl, heterocycloalkyl, and heterocycloalkyl-alkyl is independently unsubstituted or substituted with one or more substituents independently selected from the group consisting of halo and —OR¹⁰. In some embodiments, R⁸ is hydrogen. In certain embodiments, R⁹ is (C₁₋₁₀)alkyl, (C₃₋₁₀)cycloalkyl, (C₃₋₁₀)cycloalkyl-(C₁₋₁₀)alkyl, heterocycloalkyl, or heterocycloalkyl-(C₁₋₁₀)alkyl. In some embodiments, R⁹ is 5- or 6-membered heterocycloalkyl. In other embodiments, R⁹ is 5- or 6-membered heterocycloalkyl-(C₁₋₁₀)alkyl. In some embodiments, R⁹ is methyl, ethyl, propyl, butyl, pentyl, hexyl, cyclopropyl, cyclobutyl, cyclopentyl, cyclohexyl, cyclobutane-ethyl, cyclopentane-methyl, cyclopentane-ethyl, cyclohexane-methyl, cyclohexane-ethyl, pyrrolidinyl, piperidinyl, pyrrolidine-methyl, pyrrolidine-ethyl, pyrrolidine-propyl, piperidine-methyl, piperidine-ethyl, or piperidine-propyl. In other embodiments, R⁸ and R⁹, together with the nitrogen atom to which they are attached, form a heterocycloalkyl. For example, in some embodiments, R⁸ and R⁹, together with the nitrogen atom to which they are attached, form a 5- or 6-membered heterocycloalkyl. In certain embodiments, each of the foregoing moieties of R⁹, or the heterocycloalkyl formed by R⁸ and R⁹ together, is unsubstituted or substituted with one to three substituents independently selected from the group consisting of halo and —OR¹⁰. In certain embodiments, each R¹⁰ is independently hydrogen, (C₁₋₁₀)alkyl, or (C₁₋₁₀)haloalkyl. In some embodiments, R¹⁰ is —OH. In some embodiments, R⁷ and R⁸ are hydrogen and R⁹ is (C₁₋₄)alkyl or (C₃₋₆)cycloalkyl. In some embodiments, R⁷ and R⁸ are hydrogen and R⁹ is cyclopropyl. In some embodiments, R⁷ and R⁸ are hydrogen and R⁹ is propyl, such as n-propyl or isopropyl. In certain embodiments, R¹ is:

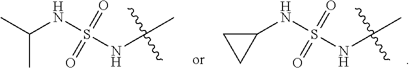

In still further embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, such as embodiments described herein, R¹ is —NR⁸R⁹. In some embodiments, R⁸ and R⁹ are independently hydrogen, alkyl, cycloalkyl, cycloalkyl-alkyl, heterocycloalkyl, or heterocycloalkyl-alkyl; wherein each alkyl, cycloalkyl, cycloalkyl-alkyl, heterocycloalkyl, and heterocycloalkyl-alkyl is independently unsubstituted or substituted with one or more substituents independently selected from the group consisting of halo and —OR¹⁰. In some embodiments, R⁸ and R⁹ are independently hydrogen, (C₁₋₁₀)alkyl, (C₁₋₁₀)haloalkyl, (C₃₋₁₀)cycloalkyl, or (C₃₋₁₀)halocycloalkyl. In some embodiments, both R⁸ and R⁹ are hydrogen, and R¹ is —NH₂. In still further embodiments, R⁸ and R⁹, together with the nitrogen atom to which they are attached, form a heterocycloalkyl, which may be unsubstituted or substituted. In some embodiments, the heterocycloalkyl is a 5- to 7-membered heterocycloalkyl. In certain embodiments, R⁸ and R⁹, together with the nitrogen atom to which they are attached, form 5- to 7-membered heterocycloalkyl, wherein the heterocycloalkyl comprises 0, 1, or 2 heteroatoms selected from the group consisting of O, N, and S, in addition to the one N attached to both R⁸ and R⁹, and wherein the heterocycloalkyl is unsubstituted or substituted with one to three substituents independently selected from the group consisting of halo, oxo, and —OR¹⁰, wherein each R¹⁰ is independently hydrogen, (C₁₋₁₀)alkyl, or (C₁₋₁₀)haloalkyl. In certain embodiments, R¹ is:

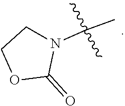 [SMILES: *N1CCOC1=O]

In still further embodiments of the compound of Formula (I), (I-i), (I-A), (I-A-i), (I-A-i-1), (II), (II-i), (II-A), (II-A-i), or (II-A-i-1), or a pharmaceutically acceptable salt, solvate, tautomer, isotope, or isomer thereof, such as embodiments described herein, R¹ is —NR⁷S(O)₂R⁹. In some embodiments, R⁷ is hydrogen, alkyl, or haloalkyl. In certain embodiments, R⁷ is hydrogen. In certain embodiments, R⁹ is alkyl, cycloalkyl, cycloalkyl-alkyl, heterocycloalkyl, or heterocycloalkyl-alkyl, wherein each of the foregoing is unsubstituted or substituted with one or more substituents independently selected from the group consisting of halo and —OR¹⁰. In some embodiments, R⁹ is 5- or 6-membered heterocycloalkyl. In other embodiments, R⁹ is 5- or 6-membered heterocycloalkyl-(C₁₋₁₀)alkyl. In some embodiments, R⁹ is methyl, ethyl, propyl, butyl, pentyl, hexyl, cyclopropyl, cyclobutyl, cyclopentyl, cyclohexyl, cyclobutane-ethyl, cyclopentane-methyl, cyclopentane-ethyl, cyclohexane-methyl, cyclohexane-ethyl, pyrrolidinyl, piperidinyl, pyrrolidine-methyl, pyrrolidine-ethyl, pyrrolidine-propyl, piperidine-methyl, piperidine-ethyl, or piperidine-propyl. In certain embodiments, each of the foregoing moieties of R⁹ is unsubstituted or substituted with one to five substituents independently selected from the group consisting of halo and —OR¹⁰. In certain embodiments, each R¹⁰ is independently hydrogen, (C₁₋₁₀)alkyl, or (C₁₋₁₀)haloalkyl. In some embodiments, R¹⁰ is —OH. In some embodiments, R⁷ is hydrogen and R⁹ is methyl or halomethyl, such as trifluoromethyl. In some embodiments, R¹ is

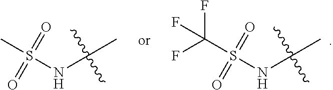

In still further embodiments of the compound of Formula (I), (I-i), (I-A), (I-A-i), (I-A-i-1), (II), (II-i), (II-A), (II-A-i), or (II-A-i-1), or a pharmaceutically acceptable salt, solvate, tautomer, isotope, or isomer thereof, such as embodiments described herein, R¹ is —NR⁷S(O)₂R⁹. In some embodiments, R⁷ is hydrogen, alkyl, or haloalkyl. In certain embodiments, R⁷ is methyl. In certain embodiments, R⁹ is alkyl, cycloalkyl, cycloalkyl-alkyl, heterocycloalkyl, or heterocycloalkyl-alkyl, wherein each of the foregoing is unsubstituted or substituted with one or more substituents independently selected from the group consisting of halo and —OR¹⁰. In some embodiments, R⁹ is 5- or 6-membered heterocycloalkyl. In other embodiments, R⁹ is 5- or 6-membered heterocycloalkyl-(C₁₋₁₀)alkyl. In some embodiments, R⁹ is methyl, ethyl, propyl, butyl, pentyl, hexyl, cyclopropyl, cyclobutyl, cyclopentyl, cyclohexyl, cyclobutane-ethyl, cyclopentane-methyl, cyclopentane-ethyl, cyclohexane-methyl, cyclohexane-ethyl, pyrrolidinyl, piperidinyl, pyrrolidine-methyl, pyrrolidine-ethyl, pyrrolidine-propyl, piperidine-methyl, piperidine-ethyl, or piperidine-propyl. In certain embodiments, each of the foregoing moieties of R⁹ is unsubstituted or substituted with one to five substituents independently selected from the group consisting of halo and —OR¹⁰. In certain embodiments, each R¹⁰ is independently hydrogen, (C₁₋₁₀)alkyl, or (C₁₋₁₀)haloalkyl. In some embodiments, R¹⁰ is —OH. In some embodiments, R⁷ is hydrogen and R⁹ is methyl or halomethyl, such as trifluoromethyl. In some embodiments, R¹ is

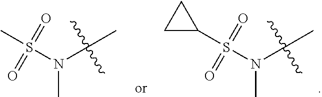

In still further embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, such as embodiments described herein, R¹ is —S(O)₂R⁹. In certain embodiments, R⁹ is alkyl, cycloalkyl, cycloalkyl-alkyl, heterocycloalkyl, or heterocycloalkyl-alkyl, wherein each of the foregoing is unsubstituted or substituted with one or more substituents independently selected from the group consisting of halo and —OR¹⁰. In some embodiments, R⁹ is 5- or 6-membered heterocycloalkyl. In other embodiments, R⁹ is 5- or 6-membered heterocycloalkyl-(C₁₋₁₀)alkyl. In some embodiments, R⁹ is methyl, ethyl, propyl, butyl, pentyl, hexyl, cyclopropyl, cyclobutyl, cyclopentyl, cyclohexyl, cyclobutane-methyl, cyclobutane-ethyl, cyclopentane-methyl, cyclopentane-ethyl, cyclohexane-methyl, cyclohexane-ethyl, pyrrolidinyl, piperidinyl, pyrrolidine-methyl, pyrrolidine-ethyl, pyrrolidine-propyl, piperidine-methyl, piperidine-ethyl, or piperidine-propyl. In certain embodiments, each of the foregoing moieties of R⁹ is unsubstituted or substituted with one to five substituents independently selected from the group consisting of halo and —OR¹⁰. In certain embodiments, each R¹⁰ is independently hydrogen, (C₁₋₁₀)alkyl, or (C₁₋₁₀)haloalkyl. In some embodiments, R¹⁰ is —OH. In certain embodiments, R⁹ is methyl, propyl, or cyclopentane-ethyl. In some embodiments, R¹ is

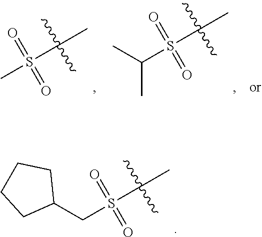

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, n1 is 0, 1, or 2. In some embodiments, n1 is 0 or 1. In certain embodiments, n1 is 1. In some embodiments, wherein n1 is 1 or 2 (for example, when n1 is 1), each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo. In some embodiments, each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹R¹¹, —S(O)ₘ₂R¹¹, —C(O)OR¹¹, and —C(O)R¹¹, wherein each alkyl, cycloalkyl, and cycloalkyl-alkyl is independently unsubstituted or substituted with one or more halo. In still further embodiments, each R² is independently halo, alkyl, haloalkyl, or —OR¹¹, wherein each R¹¹ is independently hydrogen, alkyl, or haloalkyl. In still other embodiments, each R² is independently halo. In some embodiments, at least one R² is halo. In certain embodiments, at least one R² is chloro. In still further embodiments, n1 is 1, and R² is chloro.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R³ is hydrogen, halo, cyano, alkyl, cycloalkyl, cycloalkyl-alkyl, heterocycloalkyl, heterocycloalkyl-alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. For example, in some embodiments, R³ is hydrogen, halo, cyano, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. In certain embodiments, R³ is hydrogen, halo, cyano, (C₁₋₁₀)alkyl, (C₁₋₁₀)haloalkyl, or —OR¹⁵, wherein R¹⁵ is hydrogen, (C₁₋₁₀)alkyl, or (C₁₋₁₀)haloalkyl. In some embodiments, R³ is hydrogen, halo, (C₁₋₁₀)alkyl, (C₁₋₁₀)haloalkyl. In still further embodiments, R³ is hydrogen.

In some embodiments of the compound of Formula (I), (I-i), (I-A), (I-A-i), (I-A-i-1), (II), (II-i), (II-A), (II-A-i), or (II-A-i-1), or a pharmaceutically acceptable salt, solvate, tautomer, isotope, or isomer thereof, R⁶a is hydrogen, halo, cyano, alkyl, cycloalkyl, cycloalkyl-alkyl, heterocycloalkyl, heterocycloalkyl-alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. For example, in some embodiments, R⁶a is hydrogen, halo, cyano, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. In certain embodiments, R⁶a is hydrogen, halo, cyano, (C₁₋₁₀)alkyl, (C₁₋₁₀)haloalkyl, or —OR¹⁵, wherein R¹⁵ is hydrogen, (C₁₋₁₀)alkyl, or (C₁₋₁₀)haloalkyl. In some embodiments, R⁶a is hydrogen, halo, (C₁₋₁₀)alkyl, or (C₁₋₁₀)haloalkyl. In still further embodiments, R^{6a} is hydrogen.

In some embodiments of the compound of Formula (I), (I-i), (I-B), (I-B-i), (I-B-i-1), (11), (II-i), (II-B), (II-B-i), or (II-B-i-1), or a pharmaceutically acceptable salt, solvate, tautomer, isotope, or isomer thereof, R^{6b} is hydrogen, halo, cyano, alkyl, cycloalkyl, cycloalkyl-alkyl, heterocycloalkyl, heterocycloalkyl-alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. For example, in some embodiments, R^{6b} is hydrogen, halo, cyano, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. In certain embodiments, R^{6b} is hydrogen, halo, cyano, (C₁₋₁₀)alkyl, (C₁₋₁₀)haloalkyl, or —OR¹⁵, wherein R¹⁵ is hydrogen, (C₁₋₁₀)alkyl, or (C₁₋₁₀)haloalkyl. In some embodiments, R^{6b} is hydrogen, halo, (C₁₋₁₀)alkyl, or (C₁₋₁₀)haloalkyl. In still further embodiments, R^{6b} is hydrogen.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R³, R^{6a}, and R^{6b} are independently hydrogen, halo, cyano, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, or —OR¹⁵, wherein each alkyl, cycloalkyl, cycloalkyl-alkyl, heterocycloalkyl, and heterocycloalkyl-alkyl is independently unsubstituted or substituted with one or more halo. In certain embodiments, R³, R^{6a}, and R^{6b} are independently hydrogen, halo, cyano, (C₁₋₁₀)alkyl, (C₁₋₁₀)haloalkyl, or —OR¹⁵, wherein R¹⁵ is hydrogen, (C₁₋₁₀)alkyl, or (C₁₋₁₀)haloalkyl. In some embodiments, R³, R^{6a}, and R^{6b} are independently hydrogen, halo, (C₁₋₁₀)alkyl, or (C₁₋₁₀)haloalkyl. In still further embodiments, R³, R^{6a}, and R^{6b} are all hydrogen.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, n2 is 0, 1, 2, or 3. In some embodiments, n2 is 0, 1, or 2. In certain embodiments, n2 is 0 or 1. In some embodiments, n2 is 0. In other embodiments, n2 is 1. In some embodiments, each R⁵ is independently halo, alkyl, cycloalkyl, cycloalkyl-alkyl, heterocycloalkyl, heterocycloalkyl-alkyl, —OR¹⁰, —C(O)NR¹³R¹³, —S(O)₂NR¹³R¹³, —S(O)ₘ₄R¹³, or —C(O)R¹³; wherein each alkyl, cycloalkyl, cycloalkyl-alkyl, heterocycloalkyl, and heterocycloalkyl-alkyl is independently unsubstituted or substituted with one or more substituents independently selected from the group consisting of halo, cyano, oxo, alkyl, cycloalkyl, cycloalkyl-alkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴, wherein each alkyl, cycloalkyl, and cycloalkyl-alkyl is independently unsubstituted or substituted with one or more halo. In some embodiments, each R⁵ is independently halo, (C₁₋₁₀)alkyl, (C₃₋₁₀)cycloalkyl, (C₃₋₁₀)cycloalkyl-(C₁₋₁₀)alkyl, heterocycloalkyl, heterocycloalkyl-(C₁₋₁₀)alkyl, —OR¹³, —C(O)NR¹³R¹³, —S(O)₂NR¹³R¹³, —S(O)ₘ₄R¹³, or —C(O)R¹³; wherein each alkyl, cycloalkyl, cycloalkyl-alkyl, heterocycloalkyl, and heterocycloalkyl-alkyl is independently unsubstituted or substituted with one or more substituents independently selected from the group consisting of halo, cyano, oxo, (C₁₋₁₀)alkyl, (C₃₋₁₀)cycloalkyl, (C₃₋₁₀)cycloalkyl-(C₁₋₁₀)alkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴, wherein each alkyl, cycloalkyl, and cycloalkyl-alkyl is independently unsubstituted or substituted with one or more halo. In still further embodiments, each R⁵ is independently halo, (C₁₋₁₀)alkyl, (C₃₋₁₀)cycloalkyl, (C₃₋₁₀)cycloalkyl-(C₁₋₁₀)alkyl, (5- to 7-membered)heterocycloalkyl, (5- to 7-membered)heterocycloalkyl-(C₁₋₁₀)alkyl, or —OR¹³; wherein each alkyl, cycloalkyl, cycloalkyl-alkyl, heterocycloalkyl, and heterocycloalkyl-alkyl is independently unsubstituted or substituted with one or more substituents independently selected from the group consisting of halo, cyano, oxo, (C₁₋₁₀)alkyl, (C₁₋₁₀)haloalkyl, (C₃₋₁₀)cycloalkyl, (C₃₋₁₀)halocycloalkyl, —OR¹⁴, or —NR¹⁴R¹⁴. In still further embodiments, each R⁵ is independently halo, (C₁₋₁₀)alkyl, or (C₁₋₁₀)haloalkyl.

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²; wherein each alkyl, alkenyl, cycloalkyl, cycloalkyl-alkyl, cycloalkenyl, heterocycloalkyl, heterocycloalkyl-alkyl, and heterocycloalkenyl is independently unsubstituted or substituted with one or more substituents independently selected from the group consisting of halo, cyano, oxo, alkyl, cycloalkyl, cycloalkyl-alkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴, wherein each alkyl, cycloalkyl, and cycloalkyl-alkyl is independently unsubstituted or substituted with one or more halo.

In some embodiments of the compound of Formula (II), (II-i), (II-A), (II-A-i), (II-A-i-1), (II-B), (II-B-i), or (II-B-i-1), or a pharmaceutically acceptable salt, solvate, tautomer, isotope, or isomer thereof, R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²; wherein each alkyl, alkenyl, cycloalkyl, cycloalkyl-alkyl, cycloalkenyl, heterocycloalkyl, heterocycloalkyl-alkyl, and heterocycloalkenyl is independently unsubstituted or substituted with one or more substituents independently selected from the group consisting of halo, cyano, oxo, alkyl, cycloalkyl, cycloalkyl-alkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, —C(O)R¹⁴, and —OC(O)R²², wherein each alkyl, cycloalkyl, and cycloalkyl-alkyl is independently unsubstituted or substituted with one or more halo.

In some embodiments, R⁴ is alkyl, alkenyl, cycloalkyl, cycloalkyl-alkyl, cycloalkenyl, heterocycloalkyl, heterocycloalkyl-alkyl, heterocycloalkenyl, —OR², —C(O)NR¹²R¹², —NR¹²C(O)NR¹²R¹², —S(O)₂NR¹²R¹², —S(O)ₘ₃R², or —C(O)R¹²; wherein each alkyl, alkenyl, cycloalkyl, cycloalkyl-alkyl, cycloalkenyl, heterocycloalkyl, heterocycloalkyl-alkyl, and heterocycloalkenyl is independently unsubstituted or substituted with one or more —OC(O)R²².

In some embodiments, R⁴ is alkyl substituted with one or more —OC(O)R²². In some embodiments, R⁴ is (C₃-C₄)alkyl substituted with one or more —OC(O)R²². In some embodiments, R²² is independently —R²³N(R²⁴)₂ or —(CH₂CH₂—O—)ₙ₈CH₃, wherein each R²³ is (C₁-C₆)alkylene; each R²⁴ is independently H or —CH₃; and each n8 is independently an integer from 2 to 8. In some embodiments, R⁴ is:

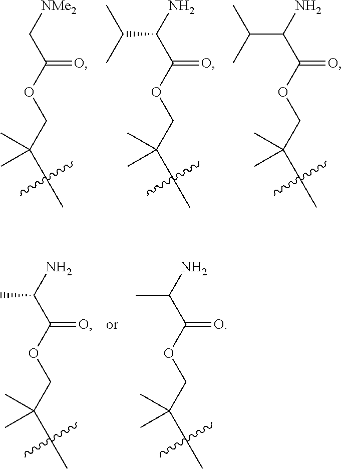

In some embodiments, R⁴ is

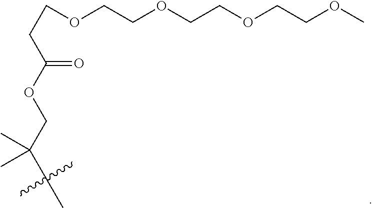 [SMILES: *C(C)(C)COC(=O)CCOCCOCCOCCOC]

In some embodiments, R⁴ is —OR¹². In some embodiments, R¹² is hydrogen, alkyl, cycloalkyl, cycloalkyl-alkyl, heterocycloalkyl, or heterocycloalkyl-alkyl; wherein each of the foregoing is independently unsubstituted or substituted with one or more substituents independently selected from the group consisting of halo, cyano, oxo, alkyl, haloalkyl,

—C(O)OR¹⁹, —C(O)NR¹⁹R¹⁹, —NR¹⁹C(O)R¹¹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene. In some embodiments, R⁴ is:

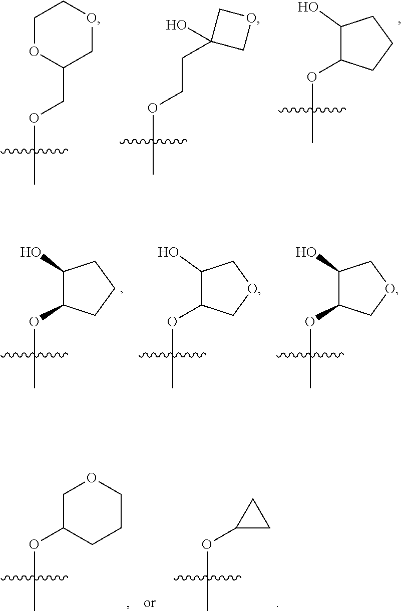

In some embodiments, R⁴ is (C₁₋₁₀)alkyl, (C₂₋₁₀)alkenyl, (C₃₋₁₀)cycloalkyl, (C₃₋₁₀)cycloalkyl-(C₁₋₁₀)alkyl, (C₃₋₁₀)cycloalkenyl, (5- to 7-membered)heterocycloalkyl, (5- to 7-membered)heterocycloalkyl-(C₁₋₁₀)alkyl, (5- to 7-membered)heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²; wherein each alkyl, alkenyl, cycloalkyl, cycloalkyl-alkyl, cycloalkenyl, heterocycloalkyl, heterocycloalkyl-alkyl, and heterocycloalkenyl is independently unsubstituted or substituted with one or more substituents independently selected from the group consisting of halo, cyano, oxo, (C₁₋₁₀)alkyl, (C₃₋₁₀)cycloalkyl, (C₃₋₁₀)cycloalkyl-(C₁₋₁₀)alkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴, wherein each alkyl, cycloalkyl, and cycloalkyl-alkyl is independently unsubstituted or substituted with one or more halo.

In some embodiments, R⁴ is (C₁₋₁₀)alkyl, (C₁₋₁₀)alkenyl, (C₃₋₁₀)cycloalkyl, (C₃₋₁₀)cycloalkenyl, (5- to 7-membered)heterocycloalkyl, or (5- to 7-membered)heterocycloalkenyl. In certain embodiments, R⁴ is (C₁₋₁₀)alkyl, (C₁₋₁₀)alkenyl, (C₃₋₁₀)cycloalkyl, (5- to 7-membered)heterocycloalkyl, or (5- to 7-membered)heterocycloalkenyl. In still further embodiments, R⁴ is (C₁₋₁₀)alkyl, (C₃₋₁₀)cycloalkyl, or (5- to 7-membered)heterocycloalkyl. In some embodiments, R⁴ is (C₁₋₈)alkyl, (C₁₋₆)alkyl, or (C₁₋₄)alkyl, such as hexyl, pentyl, butyl, propyl, ethyl, or methyl, which may be unsubstituted or substituted. In some embodiments, R⁴ is (C₁₋₈)alkenyl, (C₁₋₆)alkenyl, or (C₁₋₄)alkenyl, wherein the alkenyl comprises one or two C═C double bonds, and wherein the alkenyl may be unsubstituted or substituted. In other embodiments, R⁴ is a 4-, 5-, or 6-membered heterocycloalkyl comprising one to three heteroatoms independently selected from O and N. In some embodiments, wherein R⁴ is a heterocycloalkyl, the heterocycloalkyl is connected through an annular carbon atom. For example, in some embodiments R⁴ is a C₃₋₅ cycloalkyl comprising one or two O atoms. In still further embodiments, R⁴ is a 5- or 6-membered heterocycloalkenyl comprising one to three heteroatoms independently selected from 0 and N. In some embodiments, wherein R⁴ is a heterocycloalkenyl, the heterocycloalkenyl is connected through an annular carbon atom. In still further embodiments, R⁴ is (C₃₋₁₀)cycloalkyl, for example (C₃₋₈)cycloalkyl, (C₃₋₆)cycloalkyl, or (C₄₋₆)cycloalkyl. In some embodiments, R⁴ is (C₁₋₆)alkyl or (C₃₋₆)cycloalkyl, wherein the alkyl or cycloalkyl is unsubstituted or substituted with one to six substituents selected from the group consisting of halo, (C₁₋₁₀)alkyl, (C₃₋₁₀)cycloalkyl, and —OR¹⁴, wherein the (C₁₋₁₀)alkyl and (C₃₋₁₀)cycloalkyl are independently unsubstituted or substituted with one or more halo or —(OR⁸)ₙ₅OR¹⁷, or a combination thereof. Thus, in some embodiments, R⁴ is (C₁₋₆)alkyl or (C₃₋₆)cycloalkyl, wherein the alkyl or cycloalkyl is unsubstituted or substituted with one to six substituents selected from the group consisting of halo; (C₁₋₁₀)alkyl; (C₁₋₁₀)alkyl substituted with —(OR¹⁸)ₙ₅OR¹⁷; (C₁₋₁₀)haloalkyl substituted with —(OR¹⁸)ₙ₅OR¹⁷; (C₃₋₁₀)cycloalkyl; (C₃₋₁₀)halocycloalkyl; and —OR¹⁴. In some embodiments, n5 is 0 or 1. In certain embodiments, R¹⁸ is alkylene, such as (C₁₋₄)alkylene. In still further embodiments, R¹⁷ is hydrogen, (C₁₋₆)alkyl, or (C₁₋₆)haloalkyl. In some embodiments, R⁴ is methyl, ethyl, propyl, butyl, pentyl, or hexyl, each of which is unsubstituted or substituted with one to six substituents selected from the group consisting of halo, (C₃₋₁₀)cycloalkyl, (C₃₋₁₀)halocycloalkyl, and —OR¹⁴. In other embodiments, R⁴ is cyclopropyl, cyclobutyl, or cyclopentyl, each of which is unsubstituted or substituted with one to six substituents selected from the group consisting of halo, (C₁₋₁₀)alkyl, (C₁₋₁₀)alkyl substituted with —(OR¹⁸)ₙ₅OR¹⁷, (C₁₋₁₀)haloalkyl, (C₁₋₁₀)haloalkyl substituted with —(OR¹⁸)ₙ₅OR¹⁷, and —OR¹⁴. In still further embodiments, R⁴ is cyclopropyl, cyclobutyl, or cyclopentyl, each of which is unsubstituted or substituted with one to six substituents selected from the group consisting of: halo; (C₁₋₁₀)alkyl; (C₁₋₁₀)alkyl substituted with —O, —O(C₁₋₆)alkyl, or —O(C₁₋₆)haloalkyl; (C₁₋₁₀)haloalkyl; (C₁₋₁₀)haloalkyl substituted with —O, —O(C₁₋₆)alkyl, or —O(C₁₋₆)haloalkyl; and —OR¹⁴ In some embodiments, R⁴ is:

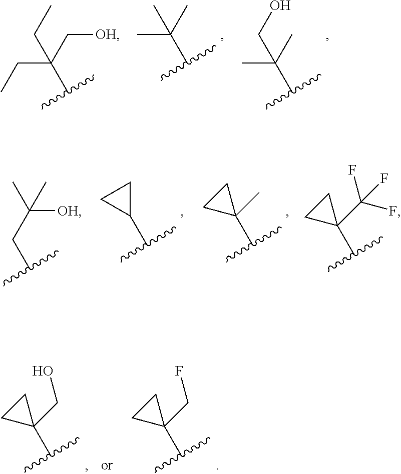

In some embodiments, R⁴ is:

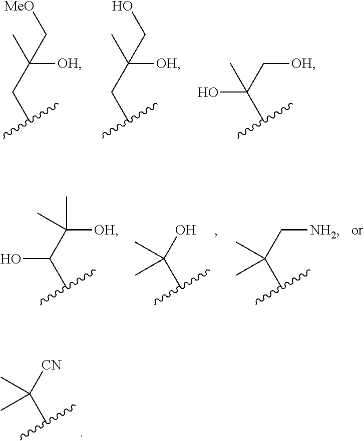

In still further embodiments, n2 is 1, 2, or 3, and R⁴ and one R⁵, together with the atoms to which they are attached, form a carbocyclyl or heterocyclyl. In some embodiments, R⁴ and R⁵, together with the atoms to which they are attached, form a 5- to 8-membered carbocyclyl or heterocyclyl. In certain embodiments, the heterocyclyl comprises one or two heteroatoms independently selected from the group consisting of O, N, and S. In certain embodiments, the heterocyclyl comprises one or two O heteroatoms. In some embodiments, the heterocyclyl is a 5- or 6-membered heterocyclyl comprising one or two O heteroatoms. In certain embodiments:

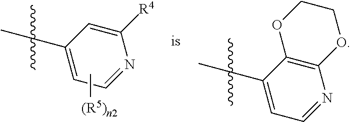

In some embodiments of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, each R¹⁰, R¹¹, R¹², R¹³, R¹⁴, R¹⁵, and R¹⁶ is independently hydrogen, alkyl, cycloalkyl, cycloalkyl-alkyl, or heterocycloalkyl; or hydrogen, (C₁₋₁₀)alkyl, (C₃₋₈)cycloalkyl, (C₃₋₁₀)cycloalkyl-(C₁₋₁₀)alkyl, or (5- to 7-membered)heterocycloalkyl; or hydrogen, (C₁₋₁₀)alkyl, or (C₃₋₈)cycloalkyl; or hydrogen or (C₁₋₁₀)alkyl; wherein each of the foregoing is independently unsubstituted or substituted with one or more halo. In some embodiments, two R¹⁰ together with the nitrogen atom to which they are attached may form a heterocycloalkyl (such as (5- to 7-membered)heterocycloalkyl); two R¹¹ together with the nitrogen atom to which they are attached may form a heterocycloalkyl (such as (5- to 7-membered)heterocycloalkyl); two R¹² together with the nitrogen atom to which they are attached may form a heterocycloalkyl (such as (5- to 7-membered)heterocycloalkyl); two R¹³ together with the nitrogen atom to which they are attached may form a heterocycloalkyl (such as (5- to 7-membered)heterocycloalkyl); or two R¹⁴ together with the nitrogen atom to which they are attached may form a heterocycloalkyl (such as (5- to 7-membered)heterocycloalkyl); wherein each of the foregoing moieties is independently unsubstituted or substituted with one or more halo.

In certain embodiments, the compound of Formula (I), Formula (I-i), Formula (I-A), Formula (I-A-i), Formula (II), Formula (II-i), Formula (II-A), or Formula (II-A-i) is:

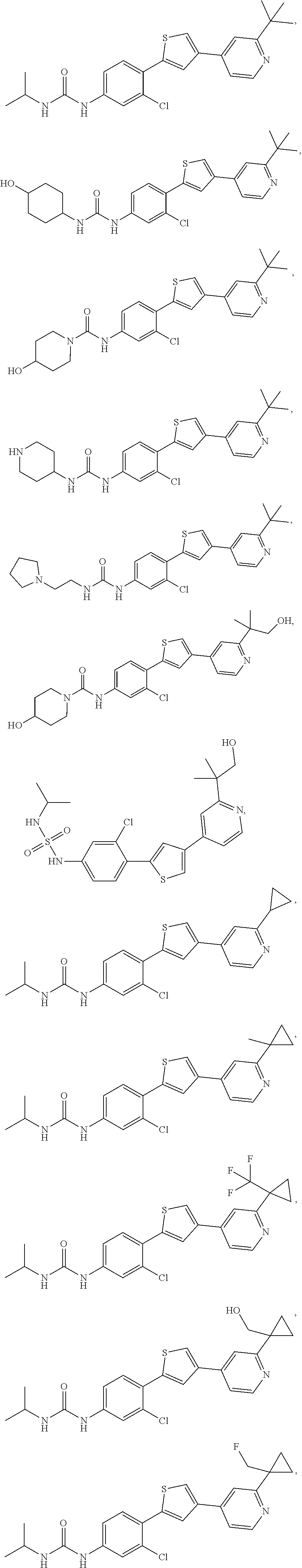

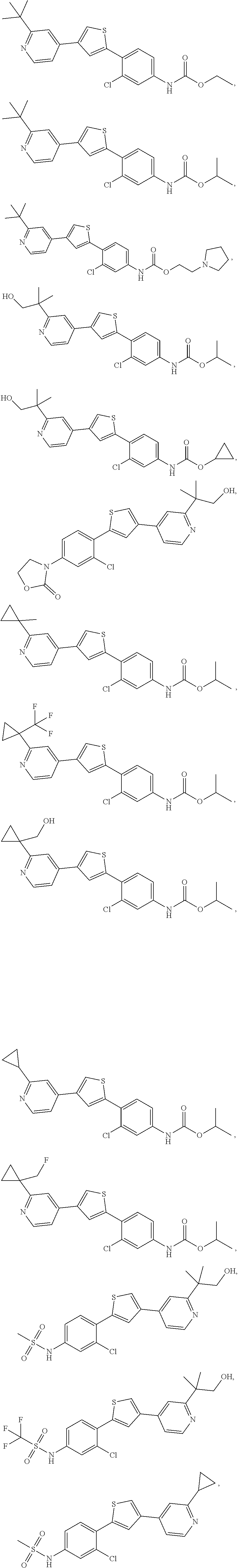

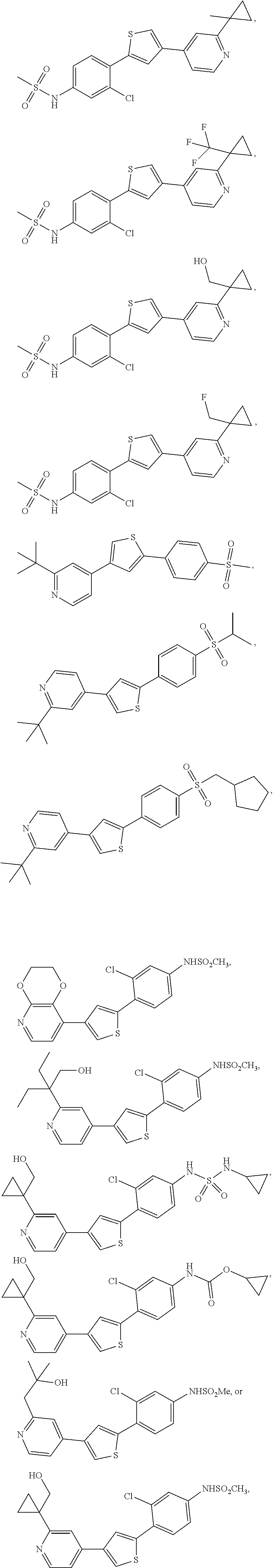

or a pharmaceutically acceptable salt, solvate, tautomer, isotope, or isomer of any of the foregoing.

In certain embodiments, the compound of Formula (I), Formula (I-i), Formula (I-A), Formula (I-A-i), Formula (II), Formula (II-i), Formula (II-A), or Formula (II-A-i) is:

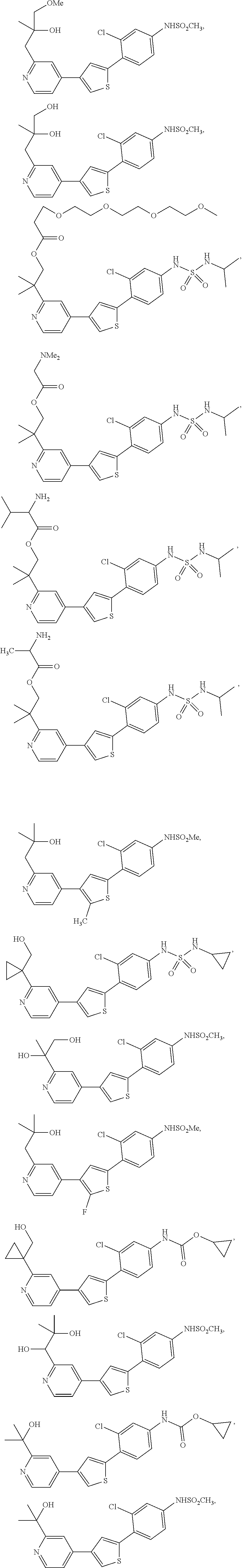

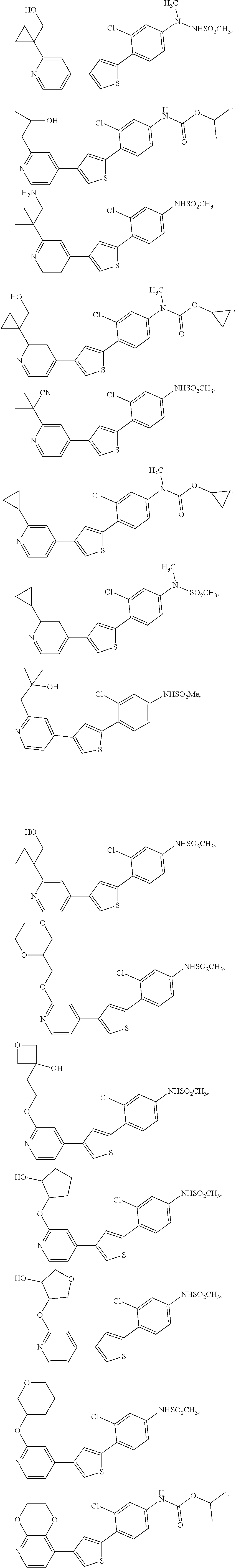

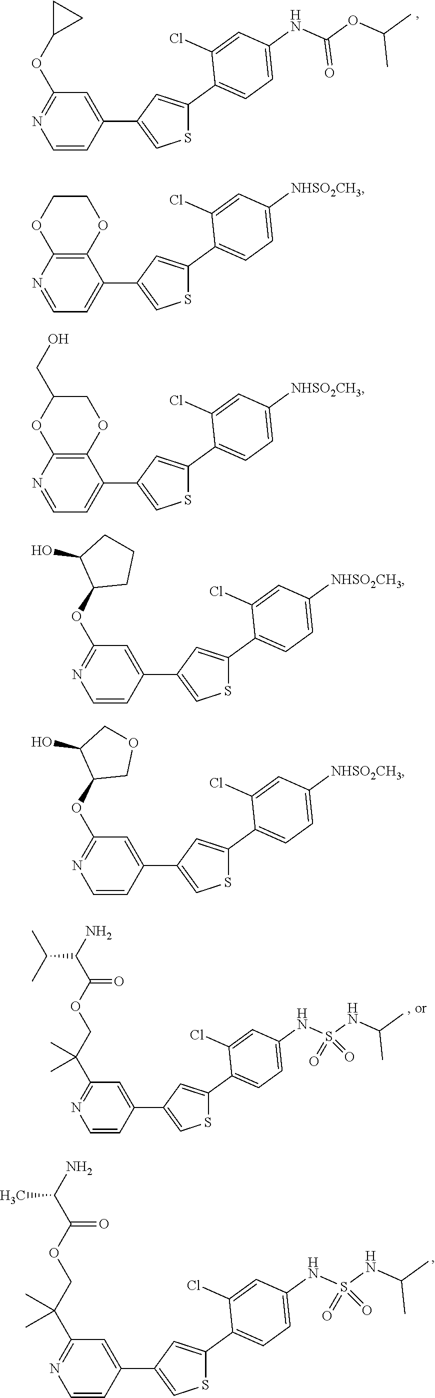

or a pharmaceutically acceptable salt, solvate, tautomer, isotope, or isomer of any of the foregoing.

Further provided are pharmaceutical compositions comprising any of the compounds disclosed herein, such as a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, and a pharmaceutically acceptable excipient.

The compounds disclosed herein, such as a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1) or a pharmaceutically acceptable salt, solvate, tautomer, isotope, or isomer thereof, may be prepared, for example, through the reaction routes depicted in General Schemes I and II.

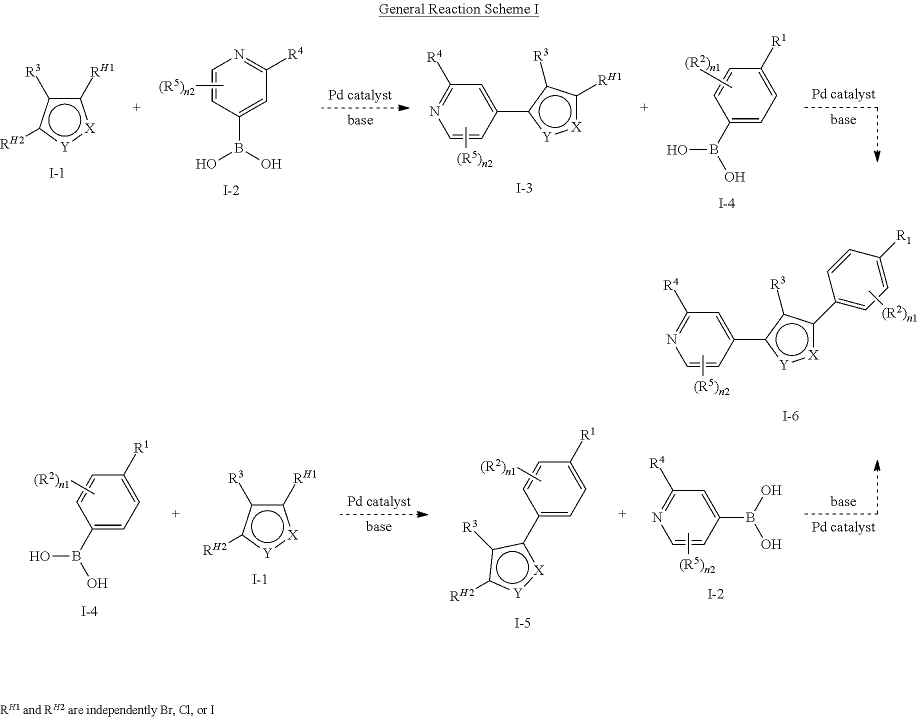

General Reaction Scheme I provides two routes to compound I-6, which is an example of a compound of Formula (I), (I-i), (I-A), (I-A-i), (I-B), (I-B-i), (II), (II-i), (II-A), (II-A-i), (II-B), or (II-B-i), or a pharmaceutically acceptable salt, solvate, tautomer, isotope, or isomer thereof as described herein. In the top route, compound I-1 is coupled with compound I-2 in the presence of a palladium catalyst and base to produce compound I-3. In the next step, compound I-3 is reacted with compound I-4 in the presence of a palladium catalyst and a base to produce compound I-6. In the second route (bottom), compound I-4 is coupled with compound I-1 to produce compound I-5, which is then coupled with compound I-2 to produce compound I-6. Suitable palladium catalysts for the first step of either route may include, for example, tetrakis(triphenylphosphine)palladium(0). Suitable palladium catalysts for this second step of either route may include, for example, bis(di-tert-butyl(4-dimethylaminophenyl)phosphine)dichloropalladium(II); [1,1′-bis(diphenylphosphino)ferrocene]dichloropalladium(II); or tetrakis(triphenylphosphine) palladium(O). Suitable bases for any of the steps of both routes may include, for example, aqueous sodium carbonate or potassium carbonate. Any of the steps depicted in General Reaction Scheme I may further include a solvent, for example, dioxane or dimethoxyethane. In some embodiments, the reactions are carried out between 60° C. to 120° C., for between 8 h to 24 h. In some embodiments of General Reaction Scheme I, X is S and Y is CR^{6a}. In other embodiments, X is CR^{6b} and Y is S.

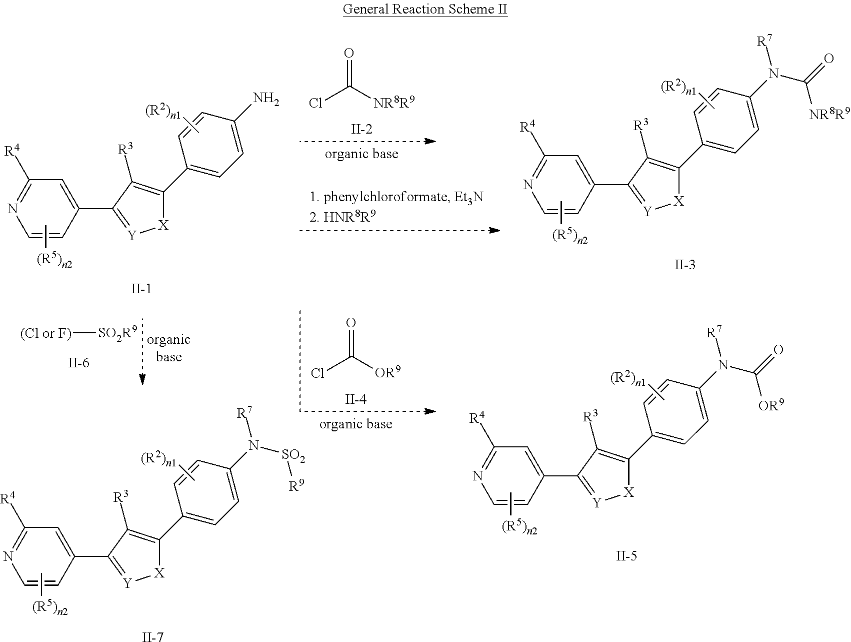

Compounds of Formula (I), (I-i), (I-A), (I-A-i), (II), (II-i), (II-A), or (II-A-i), or a pharmaceutically acceptable salt, solvate, tautomer, isotope, or isomer thereof, wherein R¹ is —NR⁷SO₂R⁹, —NR⁷C(O)NR⁸R⁹, or —NR⁷C(O)OR⁹ may in some embodiments be prepared following the various routes provided in General Reaction Scheme II, above, beginning with compound II-1. Compound II-1 may be prepared, for example, as described in General Reaction Scheme I above, wherein R¹ is —NH₂. Compound II-3 may be prepared, for example, following the top route, reacting compound II-1 with a carbamoyl chloride compound II-2 in the presence of an organic base, such as diisopropylethyl amine or triethylamine. The reaction may be carried out in a solvent, such as dichloromethane, at room temperature for 4 h to 24 h. Alternatively, compound 11-3 may be prepared by reacting compound II-1 with phenylchloroformate in dichloromethane in the presence of triethylamine (Et₃N) for approximately 16 h at room temperature, and then treating resulting phenylcarbamate with an amine HNR⁸R⁹ in tetrahydrofuran at 0° C. to room temperature for 4 h to 24 h. Compound II-5 may be prepared, for example, by reacting compound II-1 with a chloroformate compound 1-4, in the presence of an organic base (such as diisopropylethyl amine or triethylamine). The reaction may be carried out in a solvent, such as dichloromethane, at room temperature for 4 h to 24 h. Compound II-7 may be prepared, for example, by reacting compound II-1 with a sulfonyl chloride or sulfonyl fluoride compound II-6. This reaction may be carried out in the presence of an organic base, such as triethylamine, and in solvent such as pyridine for 4 h to 24 h at room temperature. In compounds 11-3, 11-5, and II-7, the moiety R⁷ may be hydrogen or may be another group as defined herein for Formula (I). When R⁷ is not hydrogen, it may be introduced, for example, by replacing one hydrogen on the free amine in compound II-1 with R⁷ prior to the coupling reactions depicted in General Reaction Scheme II, or it may be introduced into an intermediate compound after the coupling reactions depicted in General Scheme II.

The variables R¹, R², R³, R⁴, R⁵, R^{6a}, n1, and n2 in the compounds of General Reaction Schemes I and II are as described for Formula (I) herein. While General Reaction Schemes I and II depict the preparation of compounds of Formulae (I), (I-i), (I-A), and (I-A-i), or a pharmaceutically acceptable salt, solvate, tautomer, isotope, or isomer thereof, compounds of Formulae (I-B) and (I-B-i), or a pharmaceutically acceptable salt, solvate, tautomer, isotope, or isomer thereof, may also in some embodiments be prepared following analogous reaction schemes. The reactants, solvents, coupling agents, catalysts, and other compounds used to prepare compounds of Formulae (I), (I-i), (I-A), (I-A-i), (I-B), or (I-B-i), (II), (II-i), (II-A), (II-A-i), (II-B), or (II-B-i), or a pharmaceutically acceptable salt, solvate, tautomer, isotope, or isomer thereof, by following General Reaction Schemes I or 11, or by another route, may be commercially available may be prepared following organic chemical techniques.

### II. Methods of Using the Compounds and Pharmaceutical Compositions Comprising the Compounds

Provided herein are methods of using the compounds disclosed herein, such as compounds of Formula (I), (I-i), (I-A), (I-A-i), (I-A-i-1), (I-B), (I-B-i), (I-B-i-1), (II), (II-i), (II-A), (II-A-i), (II-A-i-1), (TI-B), (TI-B-i), or (TI-B-i-1), or a pharmaceutically acceptable salt, solvate, tautomer, isotope, or isomer thereof, or a pharmaceutical composition comprising any of the foregoing and a pharmaceutically acceptable excipient. These include methods of inhibiting a component of the SREBP pathway, such as an SREBP or SCAP; and methods of treating a disorder in a subject in need thereof. In some embodiments, the disorder is mediated by an SREBP or SCAP.

The terms “treat,” “treating,” or “treatment” refers to any indicia of success in the amelioration of a disorder (such as injury, disease pathology, or condition), including any objective or subjective parameter such as abatement; remission; diminishing of symptoms or making the disorder more tolerable to the subject; slowing or stopping the rate of degeneration, decline, or development; slowing the progression of disorder; making the final point of degeneration less debilitating; improving a subject's physical or mental well-being; or relieving or causing regression of the disorder. The treatment of symptoms, including the amelioration of symptoms, can be based on objective or subjective parameters, which may include the results of a physical examination, a neuropsychiatric exam, and/or a psychiatric evaluation. Certain methods and uses disclosed herein may treat cancer by, for example, decreasing the incidence of cancer, causing remission of cancer, slowing the rate of growth of cancer cells, slowing the rate of spread of cancer cells, reducing metastasis, or reducing the growth of metastatic tumors, reducing the size of one or more tumors, reducing the number of one or more tumors, or any combinations thereof.

The embodiments described herein for methods of treatment should also be considered to apply to the use of compounds of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the forgoing, for the treatment of disorders; and the use of compounds of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (IT-B-i-1), or a pharmaceutically acceptable salt, solvate, tautomer, isotope, or isomer thereof, or a pharmaceutical composition comprising any of the forgoing, for inhibiting an SREBP or inhibiting the proteolytic activation of an SREBP; and other uses of compounds of Formula (I), (I-i), (I-A), (I-A-i), (I-A-i-1), (I-B), (I-B-i), (I-B-i-1), (11), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the forgoing, as described herein; and the use of compounds of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, in the manufacture of medicaments.

A. Inhibiting SREBP or SCAP

Provided herein are uses and methods of inhibiting a component of the SREBP pathway, such as an SREBP or SCAP. In some embodiments, a combination of an SREBP and SCAP is inhibited. Such methods may include contacting an SREBP with a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the forgoing and a pharmaceutically acceptable excipient. Such uses and methods may also include contacting SCAP with a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the forgoing and a pharmaceutically acceptable excipient.

In certain embodiments, a compound of Formula (I), (I-i), (I-A), (I-A-i), (I-A-i-1), (I-B), (I-B-i), (I-B-i-1), (II), (II-i), (II-A), (I1-A-i), (IT-A-i-1), (IT-B), (II-B-i), or (II-B-i-1), or a pharmaceutically acceptable salt, solvate, tautomer, isotope, or isomer thereof is administered to a subject in need thereof to inhibit a component of the SREBP pathway. In other embodiments, a pharmaceutical composition comprising a pharmaceutically acceptable excipient and a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (I-B-i-1), or a pharmaceutically acceptable salt, solvate, tautomer, isotope, or isomer thereof, is administered to the subject in need thereof. In certain embodiments, the amount of the compound or pharmaceutically acceptable salt, solvate, tautomer, isotope, or isomer thereof, relative to the subject's body mass, is between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof is administered to a subject in need thereof to inhibit a component of the SREBP pathway. In certain embodiments, the compound or pharmaceutically acceptable salt, solvate, tautomer, isotope, or isomer thereof, is administered as a pharmaceutical composition, as described herein.

The component of the SREBP pathway that is inhibited by the methods and uses described herein may be an SREBP or SCAP. In some embodiments, an SREBP is inhibited. The SREBP may be, for example, an SREBP-1 (such as SREBP-1a or SREBP-1c) or SREBP-2. In certain variations, two or three of SREBP-1a, SREBP-1c, and SREBP-2 are inhibited. In some embodiments, the component is an SREBP-1. In other embodiments, the SREBP is SREBP-1a. In certain embodiments, the component is SREBP-1c. In still other embodiments, the SREBP is SREBP-2. In other embodiments, the component of the SREBP pathway is SCAP. In some embodiments, both an SREBP and SCAP are inhibited. In certain embodiments, two or three of SREBP-1a, SREBP-1c, and SREBP-2 are inhibited, and SCAP is inhibited.

Inhibition of a component of the SREBP pathway, such as an SREBP or SCAP, may include partial inhibition or full inhibition. Partial inhibition may include reducing the activity of a component of the SREBP pathway to a level that is still detectable. Full inhibition may include stopping all activity of a component of the SREBP pathway (such as stopping the activity of an SREBP or SCAP), or reducing the activity of a component of the SREBP pathway to a level below detection. Inhibition of a component of the SREBP pathway may be measured directly or indirectly, using any methods known in the art.

In some embodiments, inhibition of a component of the SREBP pathway is measured directly, for example by measuring the product of a reaction catalyzed by an SREBP pathway component. Inhibition of SREBP activation (for example, by inhibiting SCAP) may in some embodiments be demonstrated by western blotting and quantitatively assessing the levels of full-length and cleaved SREBP-1 and/or SREBP-2 proteins from a cell line (such as a hepatic cell lines) or primary cells (such as primary hepatocytes of mouse, rat or human origin).

In some embodiments, inhibition of a component of the SREBP pathway is measured indirectly, for example by measuring the level of expression of one or more genes that are regulated by SREBP. The inhibition of a component of the SREBP pathway, such as an SREBP or SCAP, may reduce the expression of one or more genes that are regulated by an SREBP, for example an SREBP-1 (such as SREBP-1a or SREBP-1c) or SREBP-2. SCAP plays a role in activating SREBPs, thus inhibiting the activity of SCAP may reduce the expression of one or more genes that are regulated by an SREBP. SREBP pathway inhibition may also be determined by assessing gene transcription levels of one or more target genes of SREBP-1 and/or SREBP-2, such as one or more of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, or ACACB. The transcription levels may be assessed, for example, by transcriptomic analysis, including but not limited to q-PCR. A reduction in one, two, three, four, five, or more of these genes may indicate inhibition of SREBP activation. This evaluation of endogenous SREBP gene expression may be assessed in cell lines (such as hepatic cell lines) or primary cells (such as primary hepatocytes of mouse, rat, or human origin). In some embodiments, the gene transcription levels of PCSK9 or PNPLA3, or a combination thereof, are evaluated.

Therefore, provided herein are uses and methods of reducing the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB, comprising contacting an SREBP or SCAP with a compound of Formula (I), (I-i), (I-A), (I-A-i), (I-A-i-1), (I-B), (I-B-i), (I-B-i-1), (I1), (I1-i), (II-A), (II-A-i), (II-A-i-1), (I1-B), (I1-B-i), or (I1-B-i-1), or a pharmaceutically acceptable salt, solvate, tautomer, isotope, or isomer thereof. In some embodiments, the expression of PCSK9 is reduced. In other embodiments, the expression of PNPLA3 is reduced. In still further embodiments, both the expression of PCSK9 and PNPLA3 are reduced. In certain embodiments, one or more SREBP is contacted, for example an SREBP-1 (such as SREBP-1a or SREBP-1c) or SREBP-2, or any combinations thereof. In other embodiments, SCAP is contacted. In still further embodiments, one or more of SREBP-1a, SREBP-1c, SREBP-2, and SCAP is contacted. In certain embodiments, inhibition of a component of the SREBP pathway may treat a disorder mediated by an SREBP, such as the disorders as described herein. Thus, in certain embodiments, expression of one or more genes as described above is reduced in a subject in need thereof.

Another method of indirectly detecting SREBP pathway inhibition may include: Serum-starving a hepatic cell line (HepG2) expressing luciferase under the control of the LSS-promoter to induce SREBP activation and increased luciferase expression. The cells may then be treated with a compound, such as a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof. Following treatment, a reduction of luciferase activity reflects inhibition of SREBP activation, and non-cytotoxicity of the compound can be assessed by LDH release.

B. Treating a Disorder

In other aspects, provided herein are uses and methods of treating a disorder in a subject in need thereof, comprising administering to the subject in need thereof a compound of Formula (I), (I-i), (I-A), (I-A-i) (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof. In certain aspects, provided herein are uses and methods of treating a disorder in a subject in need thereof, comprising administering to the subject in need thereof a pharmaceutical composition comprising a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, and a pharmaceutically acceptable excipient. In certain embodiments, the compound is a compound of Formula (I), or a pharmaceutically acceptable salt, solvate, tautomer, isotope, or isomer thereof. In some embodiments, the compound is a compound of Formula (I-i), or a pharmaceutically acceptable salt, solvate, tautomer, isotope, or isomer thereof. In other embodiments, the compound is a compound of Formula (I-A), or a pharmaceutically acceptable salt, solvate, tautomer, isotope, or isomer thereof. In certain embodiments, the compound is a compound of Formula (I-A-i), or a pharmaceutically acceptable salt, solvate, tautomer, isotope, or isomer thereof. In still further embodiments, the compound is a compound of Formula (I-A-i-1), or a pharmaceutically acceptable salt, solvate, tautomer, isotope, or isomer thereof. In certain embodiments, the compound is a compound of Formula (I-B), or a pharmaceutically acceptable salt, solvate, tautomer, isotope, or isomer thereof. In other embodiments, the compound is a compound of Formula (I-B-i), or a pharmaceutically acceptable salt, solvate, tautomer, isotope, or isomer thereof. In certain embodiments, the compound is a compound of Formula (I-B-i-1), or a pharmaceutically acceptable salt, solvate, tautomer, isotope, or isomer thereof. In certain embodiments, the compound is a compound of Formula (II), or a pharmaceutically acceptable salt, solvate, tautomer, isotope, or isomer thereof. In some embodiments, the compound is a compound of Formula (II-i), or a pharmaceutically acceptable salt, solvate, tautomer, isotope, or isomer thereof. In other embodiments, the compound is a compound of Formula (II-A), or a pharmaceutically acceptable salt, solvate, tautomer, isotope, or isomer thereof. In certain embodiments, the compound is a compound of Formula (II-A-i), or a pharmaceutically acceptable salt, solvate, tautomer, isotope, or isomer thereof. In still further embodiments, the compound is a compound of Formula (II-A-i-1), or a pharmaceutically acceptable salt, solvate, tautomer, isotope, or isomer thereof. In certain embodiments, the compound is a compound of Formula (II-B), or a pharmaceutically acceptable salt, solvate, tautomer, isotope, or isomer thereof. In other embodiments, the compound is a compound of Formula (II-B-i), or a pharmaceutically acceptable salt, solvate, tautomer, isotope, or isomer thereof. In certain embodiments, the compound is a compound of Formula (II-B-i-1), or a pharmaceutically acceptable salt, solvate, tautomer, isotope, or isomer thereof. In some embodiments, the disorder is mediated by an SREBP.

The uses and methods of treatment describe herein may use a compound of Formula (I), (I-i), (I-A), (I-A-i), (T-A-i-1), (I-B), (I-B-i), (I-B-i-1), (II), (II-i), (II-A), (II-A-i), (IT-A-i-1), (II-B), (II-B-i), or (II-B-i-1), or a pharmaceutically acceptable salt, solvate, tautomer, isotope, or isomer thereof; or a pharmaceutical composition comprising a compound of Formula (I), (I-i), (I-A), (I-A-i), (I-A-i-1, (I-B), (I-B-i), (I-B-i-1), (II), (II-i), (II-A), (II-A-i), (II-A-i-1), (II-B), (II-B-i, or (II-B-i-1), or a pharmaceutically acceptable salt, solvate, tautomer, isotope, or isomer thereof, and a pharmaceutically acceptable excipient.

1. Metabolic Disorders

In some embodiments, the disorder is a metabolic disorder, such as a disorder that affects lipid metabolism, cholesterol metabolism, or insulin metabolism. In certain embodiments, the disorder is related to lipid metabolism, cholesterol metabolism, or insulin metabolism, for example, liver disease as a result of the buildup of fat in the liver, or cardiovascular disease.

In some embodiments, the disorder is a liver disease, such as chronic liver disease. In some embodiments, the liver disease is mediated by a component of the SREBP pathway, such as an SREBP or SCAP. In some embodiments, the liver disease is mediated by an SREBP. In certain embodiments, the liver disease is mediated by a downstream gene target of an SREBP, such as PNPLA-3. In other embodiments, the liver disease is mediated by SCAP. Thus, in some aspects, provided herein are uses and methods of treating a liver disease in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. The chronic liver disease may be, for example, primary alcoholic liver disease, nonalcoholic fatty liver disease (NAFLD), or nonalcoholic steatohepatitis (NASH). In some embodiments, the liver disease is liver fat, liver inflammation, or liver fibrosis, or a combination thereof.

In certain embodiments, the liver disease is non-alcoholic fatty liver disease (NAFLD). NAFLD is a group of conditions that are related to fat buildup in the liver. Non-alcoholic steatohepatitis (NASH) is a form of NAFLD which includes liver inflammation. In NASH, the liver inflammation may lead to liver damage and scarring, which can be irreversible, and it can also progress to cirrhosis and liver failure. NAFLD and NASH are associated with metabolic disorders such as obesity, dyslipidemia, insulin resistance, and type 2 diabetes. Other disorders associated with NAFLD and NASH include increased abdominal fat and high blood pressure. In some embodiments, NASH is mediated by a component of the SREBP pathway, such as an SREBP or SCAP.

In other aspects, provided herein are uses and methods of treating NASH in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. Treatment of NASH may include reduction in average liver fat content, which may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation as evaluated through histological scoring of liver biopsy; reduction of liver fibrosis as evaluated through histological scoring of liver biopsy; reduction of liver fat content as evaluated through histological scoring of liver biopsy; or any combinations thereof. Treatment of NASH may be evaluated using the NAFLD activity score (NAS); or steatosis, activity, and fibrosis score (SAF); or other NASH diagnostic and/or scoring metrics (such as FIB4 or ELF).

Further provided herein are uses and methods of treating a disorder in a subject in need thereof, wherein the disorder is liver fibrosis associated with NASH, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. In some embodiments, the liver fibrosis is mediated by SREBP. Treatment of liver fibrosis may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation and/or fibrosis as evaluated through histological scoring of liver biopsy; or any combinations thereof

Further provided herein are uses and methods of treating a disorder in a subject in need thereof, wherein the disorder is fatty liver disease, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. In some embodiments, the fatty liver disease is mediated by SREBP. In certain embodiments, a subject may have fatty liver disease when the fat content of the subject's liver is 5% or greater. In some embodiments, a subject with fatty liver disease has NASH, or liver fibrosis associated with NASH. In certain embodiments, a subject with fatty liver disease has not been diagnosed with NASH or liver fibrosis associated with NASH. Treatment of fatty liver disease may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation as evaluated through histological scoring of liver biopsy; reduction of liver fibrosis as evaluated through histological scoring of liver biopsy; reduction of liver fat content as evaluated through histological scoring of liver biopsy; or any combinations thereof.

In some embodiments of the uses and methods of treating liver disease provided herein, such as methods of treating liver fibrosis, fatty liver disease, or NASH, the subject is administered between about 0.01 mg/kg to about 100 mg/kg of compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (II), (II-i), (II-A), (II-A-i), (II-A-i-1), (TI-B), (II-B-i), or (I-B-i-1), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

Other metabolic disorders which may be treated with the compounds or pharmaceutical compositions described herein may include, for example, insulin resistance, hyperglycemia, diabetes mellitus, dyslipidemia, adiposopathy, obesity, and Metabolic Syndrome. In some embodiments, the metabolic disorder is mediated by a genetic factor. In other embodiments, the metabolic disorder is mediated by one or more environmental factors, such as a diet rich in fat, or a diet rich in sugar, or a combination thereof. In some embodiments, the metabolic disorder is mediated by SREBP. In some embodiments, the diabetes mellitus is type I diabetes. In certain embodiments, the diabetes mellitus is type II diabetes.

Provided herein are uses and methods of treating diabetes in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. Diabetes (also known as diabetes mellitus) refers to a disease or condition that is generally characterized by metabolic defects in production and utilization of glucose, which result in the failure to maintain appropriate blood sugar levels in the body. In some embodiments, the diabetes is type II diabetes, which is characterized by insulin resistance, in which insulin loses its ability to exert its biological effects across a broad range of concentrations. In some embodiments, the diabetes is mediated by a component of the SREBP pathway, such as an SREBP or SCAP.

Further provided herein are uses and methods of treating insulin resistance in a subject in need thereof, comprising administering to the subject in need thereof a compound of Formula (I), (I-i), (I-A), (I-A-i) (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. Insulin resistance has been hypothesized to unify the clustering of hypertension, glucose intolerance, hyperinsulinemia, increased levels of triglyceride, decreased HDL cholesterol, and central and overall obesity. “Metabolic Syndrome” refers to a similar clustering of conditions, which may include abdominal obesity, hypertension, high blood sugar, high serum triglycerides (such as elevated fasting serum triglycerides), and low HDL levels, and is associated with a risk of developing cardiovascular disease and/or type II diabetes. Further provided herein are uses and methods of treating Metabolic Syndrome in a subject in need thereof, comprising administering to the subject in need thereof a compound of Formula (J), (J-i), (I-A), (I-A-i), (I-A-i-1), (I-B), (J-B-i), (I-B-i-1), (II), (II-i), (II-A), (II-A-i), (II-A-i-1), (II-B), (II-B-i), or (JI-B-i-1), or a pharmaceutically acceptable salt, solvate, tautomer, isotope, or isomer thereof; or a pharmaceutical composition comprising any of the foregoing and a pharmaceutically acceptable excipient. In some embodiments, the Metabolic Syndrome or insulin resistance is mediated by a component of the SREBP pathway, such as an SREBP or SCAP.

In some embodiments of the uses and methods of treating insulin resistance, hyperglycemia, diabetes mellitus, obesity, or Metabolic Syndrome provided herein, the subject is administered between about 0.01 mg/kg to about 100 mg/kg of compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

In other embodiments, the metabolic disorder is dyslipidemia. Thus, in other aspects, provided herein are uses and methods of treating dyslipidemia in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B3-i-1), or a pharmaceutically acceptable salt, solvate, tautomer, isotope, or isomer thereof; or a pharmaceutical composition comprising any of the foregoing and a pharmaceutically acceptable excipient. Dyslipidemia refers to abnormal blood plasma levels of one or more lipids or one or more lipoproteins, or any combinations thereof. Dyslipidemia may include depressed levels or elevated levels of one or more lipids and/or one or more lipoproteins, or a combination of depressed and elevated levels (for example, elevated levels of one type of lipid and depressed levels of another type of lipid and/or lipoprotein). Dyslipidemia may include, but is not limited to, elevated low density lipoprotein cholesterol (LDL), elevated apolipoprotein B, elevated triglycerides (TGs), elevated lipoprotein(a), elevated apolipoprotein A, reduced high density lipoprotein cholesterol (HDL), or reduced apolipoprotein A1, or any combinations thereof. Dyslipidemia, such as abnormal cholesterol or abnormal TG levels, is associated with an increased risk for vascular disease (such as heart attack or stroke), atherosclerosis, and coronary artery disease. In some embodiments of the uses and methods provided herein, the dyslipidemia is hyperlipidemia. Hyperlipidemia refers to the presence of an abnormally elevated level of lipids in the blood, and may include (1) hypercholesterolemia (an elevated cholesterol level); (2) hypertriglyceridemia, (an elevated triglyceride level); and (3) combined hyperlipidemia, (a combination of hypercholesterolemia and hypertriglyceridemia). Dyslipidemia may arise from a combination of genetic predisposition and diet, and may be associated with being overweight, diabetes, or Metabolic Syndrome. Lipid disorders may also arise as the result of certain medications (such as those used for anti-rejection regimens in people who have had organ or tissue transplants). In some embodiments, the dyslipidemia, such as hyperlipidemia, is mediated by a component of the SREBP pathway, such as an SREBP or SCAP. Thus, in some aspects, provided herein are uses and methods of reducing cholesterol levels, modulating cholesterol metabolism, modulating cholesterol catabolism, modulating the absorption of dietary cholesterol, reversing cholesterol transport, or lowering triglycerides in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient.

In some embodiments of the uses and methods of treating dyslipidemia provided herein, such as reducing cholesterol levels, modulating cholesterol metabolism, modulating cholesterol catabolism, modulating the absorption of dietary cholesterol, reversing cholesterol transport, or lowering triglycerides in a subject in need thereof as provided herein, the subject is administered between about 0.01 mg/kg to about 100 mg/kg of compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

In still other aspects, provided herein are uses and methods of treating adiposopathy in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the foregoing and a pharmaceutically acceptable excipient. In some embodiments, the adiposopathy is associated with Metabolic Syndrome. In some embodiments, the adiposopathy is mediated by a component of the SREBP pathway, such as an SREBP or SCAP.

In certain aspects, provided herein are uses and methods of treating gallstones in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (IT-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient. Gallstones may be associated with gallbladder inflammation, pancreas inflammation, or liver inflammation. In certain embodiments, the gallstones are cholesterol gallstones, which may form when bile contains a high concentration of cholesterol and not enough bile salts. In some embodiments, the gallstones, which may include cholesterol gallstone disease, is mediated by a component of the SREBP pathway, such as an SREBP or SCAP.

In other embodiments, the disorder is pancreatitis. In yet other embodiments, the disorder is endotoxic shock, systemic inflammation, or xanthoma. In still further embodiments, the disorder is atherosclerosis, coronary artery disease, angina pectoris, carotid artery disease, stroke, or cerebral arteriosclerosis. In certain embodiments, any of the foregoing disorders are mediated by a component of the SREBP pathway, such as an SREBP or SCAP.

In some embodiments of the uses and methods of treating gall stones, pancreatitis, endotoxic shock, systemic inflammation, xanthoma, atherosclerosis, coronary artery disease, angina pectoris, carotid artery disease, stroke, or cerebral arteriosclerosis provided herein, the subject is administered between about 0.01 mg/kg to about 100 mg/kg of compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof is administered to the subject in need thereof. In certain embodiments, the compound or pharmaceutically acceptable salt, solvate, tautomer, isotope, or isomer thereof, is administered as a pharmaceutical composition, as described herein.

In some embodiments of any of the above embodiments, the subject is overweight, obese, has insulin resistance, is pre-diabetic or has type II diabetes. In certain embodiments of any of the preceding embodiments, the subject has NASH.

2. Hyperproliferative Disorders

In another embodiment, the disorder is a hyperproliferative disorder. Thus, in some aspects, provided herein are uses and methods of treating a hyperproliferative disorder in a subject in need thereof, comprising administering to the subject in need thereof a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof; or a pharmaceutical composition comprising any of the foregoing and a pharmaceutically acceptable excipient.

As described above, the metabolism of fatty acids, cholesterol, and triglycerides may play a role in hyperproliferative disorders, such as cancer. Often, during transformation of non-cancerous cells to cancerous cell, cell metabolism shifts from catabolic to anabolic processes. Depending on the type of tumor, the tumor cells may synthesize up to 95% of the saturated and mono-unsaturated fatty acids. Some cancers exhibit increased synthesis of fatty acids and other lipids (such as cholesterol), and steroids (such as androgens). Elevated fatty acid synthase (FAS) expression may induce progression to S phase in cancer cells, and inhibition of FAS expression may reduce cell growth and may induce apoptosis. Thus, components of the SREBP pathway may play a role in hyperproliferative disorders.

Hyperproliferative disorders, which are disorders associated with some degree of abnormal cell proliferation, may be benign or malignant. Benign hyperproliferative disorders may include pre-cancerous disorders.

In some embodiments of the uses and methods provided herein, the disorder is a benign hyperproliferative disorder. In some embodiments, the benign hyperproliferative disorder is mediated by a component of the SREBP pathway, such as an SREBP or SCAP. In other embodiments, the disorder is a malignant hyperproliferative disorder. In some embodiments, the malignant hyperproliferative disorder is mediated by a component of the SREBP pathway, such as an SREBP or SCAR

In some embodiments, the hyperproliferative disorder is breast cancer, liver cancer, ovarian cancer, pancreatic cancer, or prostate cancer.

In some embodiments, the hyperproliferative disorder is a soft tissue sarcoma, bladder cancer, endometrial cancer, skin cancer, colon cancer, hematologic cancer, placenta cancer, brain cancer, kidney cancer, lung cancer, or bone cancer. Sarcoma can include cancers that begin in the bones and in the soft tissues. Sarcoma includes, for example, connective tissue cancers, such as muscle cancers.

In some embodiments of the uses and methods of treating a hyperproliferative disorder in a subject in need thereof, as described herein, between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, relative to the body mass of the subject, is administered to the subject in need thereof. In certain embodiments, the compound or pharmaceutically acceptable salt, solvate, tautomer, isotope, or isomer thereof, is administered as a pharmaceutical composition, as described herein.

### III. Dosing and Methods of Administration

The dose of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, administered to a subject in need thereof according to any of the disclosed methods may vary with the particular compound or pharmaceutically acceptable salt, solvate, tautomer, isotope, or isomer thereof; the method of administration; the particular disorder being treated; and the characteristics of the subject (such as weight, sex, and/or age). In some embodiments, the amount of the compound or pharmaceutically acceptable salt, solvate, tautomer, isotope, or isomer thereof is a therapeutically effective amount.

The effective amount of the compound or pharmaceutically acceptable salt, solvate, tautomer, isotope, or isomer thereof, relative to the subject's body mass, may in some embodiments be between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of the compound of Formula (I), (I-i), (I-A), (I-A-i), (I-A-i-1), (I-B), (I-B-i), (I-B-i-1), (11), (IT-i), (II-A), (I1-A-i), (II-A-i-1), (II-B), (II-B-i), or (I-B-i-1), or a pharmaceutically acceptable salt, solvate, tautomer, isotope, or isomer thereof is administered to a subject in need thereof. In certain embodiments, the compound or pharmaceutically acceptable salt, solvate, tautomer, isotope, or isomer thereof, is administered as a pharmaceutical composition, as described herein.

Any of the uses and methods provided herein may comprise administering to a subject in need therein a pharmaceutical composition that comprises an effective amount of a compound provided herein, such as a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a corresponding amount of a pharmaceutically acceptable salt, solvate, tautomer, isotope, or isomer thereof, and a pharmaceutically acceptable excipient.

The compounds of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof as provided herein, or a pharmaceutical composition comprising any of these and a pharmaceutically acceptable excipient as provided herein, may be administered to a subject via any suitable route, including, for example, intravenous, intramuscular, subcutaneous, oral, or transdermal routes.

In certain aspects, the invention provides a method of treating a disorder in subject in need thereof by parenterally administering to the subject in need thereof an effective amount of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof as provided herein, or a pharmaceutical composition comprising an effective amount of any of the foregoing and a pharmaceutically acceptable excipient as provided herein. In some embodiments, the disorder is a hyperproliferative disorder. In certain embodiments, the hyperproliferative disorder is cancer. In other embodiments, the disorder is fatty liver disease. In certain embodiments, the disorder is NASH. In some embodiments, the route of administration is intravenous, intra-arterial, intramuscular, or subcutaneous. In some embodiments, the route of administration is transdermal.

In some aspects, provided herein are pharmaceutical compositions comprising a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, and a pharmaceutically acceptable excipient, for the use in treating a disorder as described herein. In some embodiments, the disorder is prevented, or the onset delayed, or the development delayed. In some embodiments, the disorder is a hyperproliferative disorder. In certain embodiments, the hyperproliferative disorder is cancer. In some embodiments, the disorder is fatty liver disease. In certain embodiments, the disorder is NASH. In certain embodiments, the composition comprises a pharmaceutical formulation, which is present in a one or more unit dosage forms, for example one, two, three, four, or more unit dosage forms.

### IV. Kits

Also provided are articles of manufacture comprising a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or pharmaceutical compositions comprising any of the foregoing, or unit dosages comprising any of these, as described herein in suitable packaging for use in the methods described herein. Suitable packaging may include, for example, vials, vessels, ampules, bottles, jars, flexible packaging, and the like. An article of manufacture may further be sterilized and/or be sealed kits.

Further provided herein are kits comprising a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the foregoing and a pharmaceutically acceptable excipient. The kits may be used in any of the uses and methods described herein. In some embodiments, the kit further comprises instructions. The kits may be used for any one or more of the uses described herein, and, accordingly, may contain instructions for the treatment of a hyperproliferative disorder (such as cancer), fatty liver disease, or NASH. The kits may comprise one or more containers. Each component (if there is more than one component) may be packaged in separate containers or some components may be combined in one container where cross-reactivity and shelf life permit.

The kits may be in unit dosage forms, bulk packages (e.g., multi-dose packages) or subunit doses. For example, kits may be provided that contain sufficient dosages of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the foregoing and a pharmaceutically acceptable excipient, as disclosed herein and/or a second pharmaceutically active compound useful for a disorder detailed herein to provide effective treatment of a subject for an extended period, such as one week, 2 weeks, 3 weeks, 4 weeks, 6 weeks, 8 weeks, 3 months, 4 months, 5 months, 7 months, 8 months, 9 months, or more. Kits may also include multiple unit doses of a compound of Formula (I), (I-i), (I-A), (I-A-i), (I-A-i-1), (I-B), (I-B-i), (I-B-i-1), (II), (II-i), (II-A), (II-A-i), (II-A-i-1), (II-B), (II-B-i), or (II-B-i-1), or a pharmaceutically acceptable salt, solvate, tautomer, isotope, or isomer thereof, or a pharmaceutical composition comprising any of the foregoing and a pharmaceutically acceptable excipient, and instructions for use, and be packaged in quantities sufficient for storage and use in pharmacies (e.g., hospital pharmacies or compounding pharmacies).

The kits may optionally include a set of instructions, generally written instructions, although electronic storage media (e.g., magnetic diskette or optical disk) containing instructions are also acceptable, relating to the use of component(s) of the uses and methods as described herein. The instructions included with the kit may include information as to the components and their administration to an individual.

### ENUMERATED EMBODIMENTS

### Embodiment I-1

A compound of Formula (I):

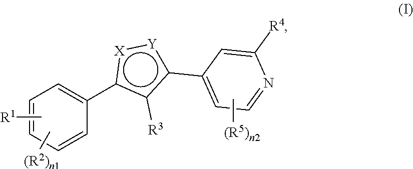

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
      wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹¹, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, or heterocycloalkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

### Embodiment I-2

The compound of embodiment I-1, wherein the compound is of Formula (I-A):

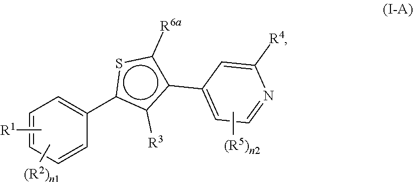

or a pharmaceutically acceptable salt, solvate, tautomer, isotope, or isomer thereof, wherein R¹, R², R³, R⁴, R⁵, R^{6a}, n1, and n2 are as defined for Formula (I).

### Embodiment I-3

The compound of embodiment I-1 or I-2, wherein the compound is of Formula (I-A-i):

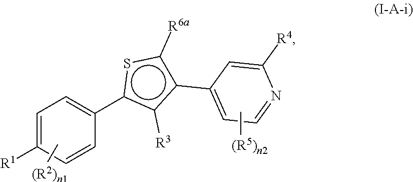

or a pharmaceutically acceptable salt, solvate, tautomcr, isotope, or isomer thereof, wherein R¹, R², R³, R⁴, R⁵, R^{6a}, n1, and n2 are as defined for Formula (1).

### Embodiment I-4. The compound of embodiment I-1, wherein the compound is of Formula (I-B):

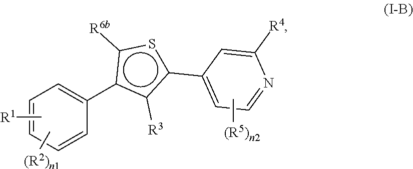

or a pharmaceutically acceptable salt, solvate, tautomer, isotope, or isomer thereof, wherein R¹, R², R³, R⁴, R⁵, R^{6b}, n1, and n2 are as defined for Formula (I).

### Embodiment I-5

The compound of embodiment I-1 or I-4, wherein the compound is of Formula (I-B-i):

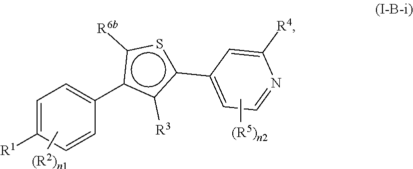

or a pharmaceutically acceptable salt, solvate, tautomer, isotope, or isomer thereof, wherein R¹, R², R³, R⁴, R⁵, R^{6b}, n1, and n2 are as defined for Formula (I).

### Embodiment I-6

The compound of any one of embodiments I-1 to I-3, or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁸, —S(O)₂R⁸, —NR⁷(SO)₂R⁹, or —NR⁸R⁹.

### Embodiment I-7

The compound of any one of embodiments I-1 to I-6, or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁸, —S(O)₂R⁸, or —NR⁸R⁹.

### Embodiment I-8

The compound of any one of embodiments I-1 to I-7, or a pharmaceutically acceptable salt, solvate, tautomer, isotope, or isomer thereof, wherein R¹ is —NR⁷C(O)NR⁸R⁹ or —NR⁷S(O)₂NR⁸R⁹.

### Embodiment I-9

The compound of any one of embodiments 1-1 to I-7, or a pharmaceutically acceptable salt, solvate, tautomer, isotope, or isomer thereof, wherein R¹ is —NR⁷C(O)OR⁹, —NR⁷(SO)₂R⁹, or —S(O)₂R⁹.

### Embodiment I-10

The compound of any one of embodiments I-1 to I-9, or a pharmaceutically acceptable salt, solvate, tautomer, isotope, or isomer thereof, wherein R⁷ and R⁸ are both hydrogen, and R⁹ is alkyl, cycloalkyl, cycloalkyl-alkyl, heterocycloalkyl, or heterocycloalkyl-alkyl, wherein the alkyl, cycloalkyl, cycloalkyl-alkyl, heterocycloalkyl, or heterocycloalkyl-alkyl is unsubstituted or substituted with one or more substituents independently selected from the group consisting of halo and —OR¹⁰, wherein each R¹⁰ is independently hydrogen, alkyl, or haloalkyl.

### Embodiment I-11

The compound of any one of embodiments I-1 to I-8, or a pharmaceutically acceptable salt, solvate, tautomer, isotope, or isomer thereof, wherein R⁸ and R⁹, together with the nitrogen atom to which they are attached, form a heterocycloalkyl, wherein the heterocycloalkyl is unsubstituted or substituted with 1 to 3 substituents independently selected from the group consisting of halo, oxo, and —OR¹⁰, wherein each R¹⁰ is independently hydrogen, unsubstituted alkyl, or haloalkyl.

### Embodiment I-12

The compound of any one of embodiments I-1 to I-11, or a pharmaceutically acceptable salt, solvate, tautomer, isotope, or isomer thereof, wherein n1 is 0 or 1.

### Embodiment I-13

The compound of any one of embodiments I-1 to I-12, or a pharmaceutically acceptable salt, solvate, tautomer, isotope, or isomer thereof, wherein each R² is independently halo, alkyl or —OR¹¹, wherein each R¹¹ is independently hydrogen, unsubstituted alkyl, or haloalkyl.

### Embodiment I-14

The compound of any one of embodiments I-1 to I-13, or a pharmaceutically acceptable salt, solvate, tautomer, isotope, or isomer thereof, wherein each R² is independently halo.

### Embodiment I-15

The compound of any one of embodiments 1-1 to I-14, or a pharmaceutically acceptable salt, solvate, tautomer, isotope, or isomer thereof, wherein each R² is chloro.

### Embodiment I-16

The compound of any one of embodiments I-1 to I-15, or a pharmaceutically acceptable salt, solvate, tautomer, isotope, or isomer thereof, wherein R³ and R⁶a or R^{6b} are both hydrogen.

### Embodiment I-17

The compound of any one of embodiments I-1 to I-16, or a pharmaceutically acceptable salt, solvate, tautomer, isotope, or isomer thereof, wherein n2 is 0.

### Embodiment I-18

The compound of any one of embodiments I-1 to I-17, or a pharmaceutically acceptable salt, solvate, tautomer, isotope, or isomer thereof, wherein R⁴ is alkyl or cycloalkyl, wherein the alkyl or cycloalkyl is unsubstituted or substituted with 1 to 3 substituents independently selected from the group consisting of halo, alkyl, alkyl substituted with —(OR¹⁸)ₙ₅OR¹⁷, haloalkyl, haloalkyl substituted with —(OR¹⁸)ₙ₅OR¹⁷, cycloalkyl, and —OR¹⁴, wherein each R¹⁴ and R¹⁷ is independently hydrogen, unsubstituted alkyl, or haloalkyl, and each R¹⁸ is independently alkylene.

### Embodiment I-19

The compound of any one of embodiments I-1 to I-3 or I-6 to I-18, selected from the group consisting of:

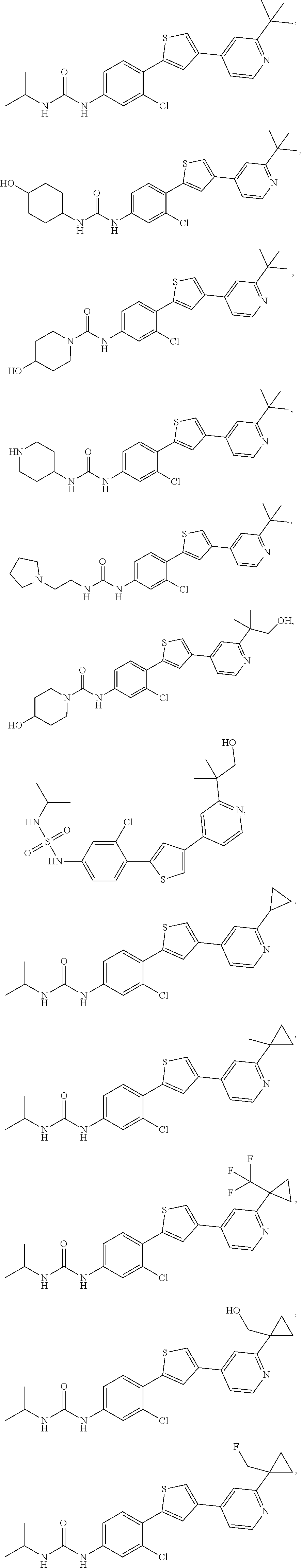

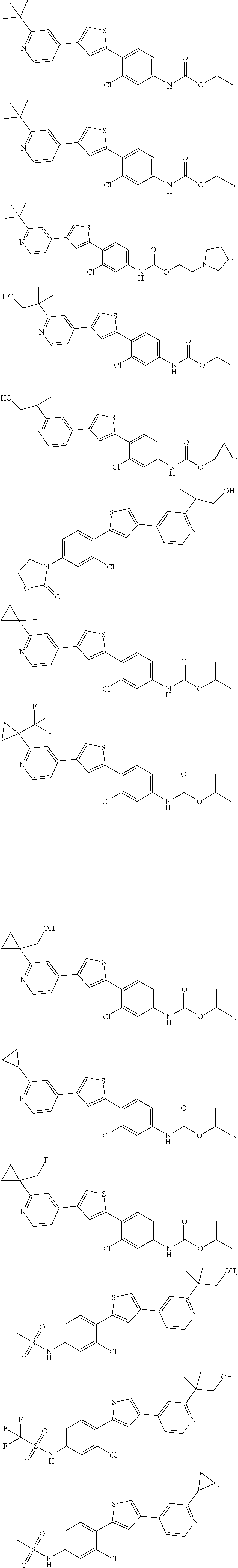

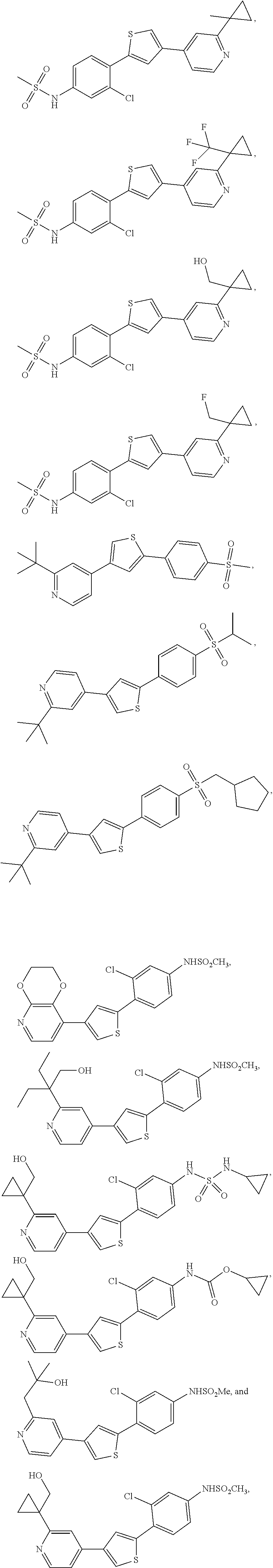

or a pharmaceutically acceptable salt, solvate, tautomer, isotope, or isomer thereof.

### Embodiment I-20

A pharmaceutical composition, comprising the compound of any one of embodiments I-1 to I-19, or a pharmaceutically acceptable salt, solvate, tautomer, isotope, or isomer thereof, and a pharmaceutically acceptable excipient.

### Embodiment I-21

A method of inhibiting a sterol regulatory element-binding protein (SREBP), comprising contacting the SREBP or contacting an SREBP cleavage activating-protein (SCAP) with an effective amount of a compound of any one of embodiments I-1 to I-19, or a pharmaceutically acceptable salt, solvate, tautomer, isotope, or isomer thereof, or the pharmaceutical composition of embodiment I-20.

### Embodiment I-22

A method of inhibiting the proteolytic activation of a sterol regulatory element-binding protein (SREBP), comprising contacting an SREBP cleavage activating-protein (SCAP) with an effective amount of a compound of any one of embodiments I-1 to I-19, or a pharmaceutically acceptable salt, solvate, tautomer, isotope, or isomer thereof, or the pharmaceutical composition of embodiment I-20.

### Embodiment 1-23

The method of embodiment 1-21 or 1-22, wherein the SREBP is an SREBP-1.

### Embodiment I-24

The method of embodiment I-23, wherein the SREBP-1 is SREBP-1a.

### Embodiment I-25

The method of embodiment I-23, wherein the SREBP-1 is SREBP-1c.

### Embodiment I-26

The method of embodiment I-21 or I-22, wherein the SREBP is SREBP-2.

### Embodiment I-27

The method of any one of embodiments I-21 to I-26, wherein SREBP is inhibited in a subject in need thereof.

### Embodiment I-28

The method of any one of embodiments 1-21 to 1-27, wherein SCAP is inhibited in a subject in need thereof.

### Embodiment I-29

The method of any one of embodiments 1-21 to 1-28, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof, or the pharmaceutical composition.

### Embodiment I-30

A method of treating a disorder in a subject in need thereof, comprising administering to the subject in need thereof an effective amount of a compound of any one of embodiments I-1 to I-19, or a pharmaceutically acceptable salt, solvate, tautomer, isotope, or isomer thereof, or the pharmaceutical composition of embodiment I-20.

Embodiment I-31. A method of treating a disorder in a subject in need thereof, wherein the disorder is mediated by a sterol regulatory element-binding protein (SREBP), comprising administering to the subject in need thereof an effective amount of a compound of any one of embodiments I-1 to I-19, or a pharmaceutically acceptable salt, solvate, tautomer, isotope, or isomer thereof, or the pharmaceutical composition of embodiment I-20.

### Embodiment I-32

The method of embodiment I-30 or I-31, wherein the disorder is Metabolic Syndrome, type 2 diabetes, obesity, liver disease, insulin resistance, adiposopathy, or dyslipidemia.

### Embodiment I-33

The method of embodiment I-32, wherein the dyslipidemia is hypertriglyceridemia or elevated cholesterol levels.

### Embodiment I-34

The method of embodiment I-32, wherein the liver disease is nonalcoholic steatohepatitis, liver fibrosis, or liver inflammation, or a combination thereof.

### Embodiment I-35

The method of embodiment I-30 or I-31, wherein the disorder is a hyperproliferative disorder.

### Embodiment I-36

The method of embodiment I-35, wherein the hyperproliferative disorder is cancer.

### Embodiment I-37

The method of embodiment I-36, wherein the cancer is breast cancer, liver cancer, ovarian cancer, pancreatic cancer, or prostate cancer.

### Embodiment I-38

The method of embodiment I-30 or I-31, wherein the disorder is endotoxic shock, systemic inflammation, or atherosclerosis.

### Embodiment I-39

A compound of any one of embodiments I-1 to I-19, or a pharmaceutically acceptable salt, solvate, tautomer, isotope, or isomer thereof, for use in inhibiting a sterol regulatory element-binding protein (SREBP).

### Embodiment I-40

A compound of any one of embodiments I-1 to I-19, or a pharmaceutically acceptable salt, solvate, tautomer, isotope, or isomer thereof, for use in inhibiting the proteolytic activation of a sterol regulatory element-binding protein (SREBP).

### Embodiment I-41

The compound for use of embodiment I-39 or I-40, wherein the SREBP is an SREBP-1.

### Embodiment I-42

The compound for use of embodiment I-41, wherein the SREBP-1 is SREBP-1a.

### Embodiment I-43

The compound for use of embodiment I-42, wherein the SREBP-1 is SREBP-1c.

### Embodiment I-44

The compound for use of embodiment I-39 or I-40, wherein the SREBP is SREBP-2.

### Embodiment I-45

The compound for use of any one of embodiments I-39 to I-44, wherein SREBP is inhibited in a subject in need thereof.

### Embodiment I-46

The compound for use of any one of embodiments I-39 to I-45, wherein SCAP is inhibited in a subject in need thereof.

### Embodiment I-47

The compound for use of any one of embodiments I-39 to I-46, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

### Embodiment I-48

A compound of any one of embodiments I-1 to I-19, or a pharmaceutically acceptable salt, solvate, tautomer, isotope, or isomer thereof, for use in treating a disorder in a subject in need thereof.

### Embodiment I-49

A compound of any one of embodiments I-1 to I-19, or a pharmaceutically acceptable salt, solvate, tautomer, isotope, or isomer thereof, for use in treating a disorder in a subject in need thereof, wherein the disorder is mediated by a sterol regulatory element-binding protein (SREBP).

### Embodiment I-50

The compound for use of embodiment I-48 or I-49, wherein the disorder is Metabolic Syndrome, type 2 diabetes, obesity, liver disease, insulin resistance, adiposopathy, or dyslipidemia.

### Embodiment I-51

The compound for use of embodiment I-50, wherein the dyslipidemia is hypertriglyceridemia or elevated cholesterol levels.

### Embodiment I-52

The compound for use of embodiment I-50, wherein the liver disease is nonalcoholic steatohepatitis, liver fibrosis, or liver inflammation, or a combination thereof.

### Embodiment I-53

The compound for use of embodiment I-48 or I-49, wherein the disorder is a hyperproliferative disorder.

### Embodiment I-54

The compound for use of embodiment I-53, wherein the hyperproliferative disorder is cancer.

### Embodiment I-55

The compound for use of embodiment I-54, wherein the cancer is breast cancer, liver cancer, ovarian cancer, pancreatic cancer, or prostate cancer.

### Embodiment I-56

The compound for use of embodiment I-48 or I-49, wherein the disorder is endotoxic shock, systemic inflammation, or atherosclerosis.

### Embodiment I-57

Use compound of any one of embodiments I-1 to I-19, or a pharmaceutically acceptable salt, solvate, tautomer, isotope, or isomer thereof, in the manufacture of a medicament for inhibiting a sterol regulatory element-binding protein (SREBP).

### Embodiment I-58

Use of a compound of any one of embodiments I-1 to I-19, or a pharmaceutically acceptable salt, solvate, tautomer, isotope, or isomer thereof, in the manufacture of a medicament for inhibiting the proteolytic activation of a sterol regulatory element-binding protein (SREBP).

### Embodiment I-59

The use of embodiment I-57 or I-58, wherein the SREBP is an SREBP-1.

### Embodiment I-60

The use of embodiment I-59, wherein the SREBP-1 is SREBP-1a.

### Embodiment I-61

The use of embodiment I-59, wherein the SREBP-1 is SREBP-1c.

### Embodiment I-62

The use of embodiment I-57 or I-58, wherein the SREBP is SREBP-2.

### Embodiment I-63

The use of any one of embodiments I-57 to I-62, wherein SREBP is inhibited in a subject in need thereof.

### Embodiment I-64

The use of any one of embodiments I-57 to I-63, wherein SCAP is inhibited in a subject in need thereof.

### Embodiment I-65

The use of any one of embodiments I-57 to I-64, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

### Embodiment I-66

Use of a compound of any one of embodiments I-1 to I-19, or a pharmaceutically acceptable salt, solvate, tautomer, isotope, or isomer thereof, in the manufacture of a medicament for treating a disorder in a subject in need thereof.

### Embodiment I-67

Use of a compound of any one of embodiments I-1 to I-19, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

### Embodiment I-68

The use of embodiment I-66 or I-67, wherein the disorder is Metabolic Syndrome, type 2 diabetes, obesity, liver disease, insulin resistance, adiposopathy, or dyslipidemia.

### Embodiment I-69

The use of embodiment I-68, wherein the dyslipidemia is hypertriglyceridemia or elevated cholesterol levels.

### Embodiment I-70

The use of embodiment I-68, wherein the liver disease is nonalcoholic steatohepatitis, liver fibrosis, or liver inflammation, or a combination thereof.

### Embodiment I-71

The use of embodiment I-66 or I-67, wherein the disorder is a hyperproliferative disorder.

### Embodiment I-72

The use of embodiment I-71, wherein the hyperproliferative disorder is cancer.

### Embodiment I-73

The use of embodiment I-72, wherein the cancer is breast cancer, liver cancer, ovarian cancer, pancreatic cancer, or prostate cancer.

### Embodiment I-74

The use of embodiment I-66 or I-67, wherein the disorder is endotoxic shock, systemic inflammation, or atherosclerosis.

### Embodiment II-1

A compound of Formula (II):

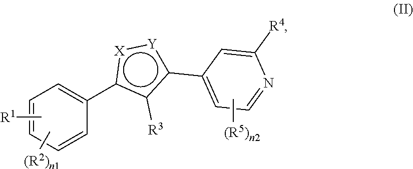

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹OS(O)₂R¹⁰, —S(O)ₘ₁R¹⁰, —C(O)OR¹⁰, —C(O)R¹⁰, and —(OR²¹)ₙ₆OR¹⁰;
   or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
      wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, —C(O)R¹⁴, and —OC(O)R²²,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R²² is independently —R²³N(R²⁴)₂ or —(CH₂CH₂—O—)ₙ₈CH₃,
   wherein each R²³ is (C₁-C₆)alkylene; each R²⁴ is independently H or —CH₃; and each n8 is independently an integer from 2 to 8;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, heterocycloalkyl, or heterocycloalkyl-alkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

### Embodiment II-2

A compound of Formula (I):

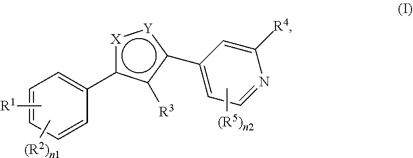

or a pharmaceutically acceptable salt, solvate, tautomer, isotope, or isomer thereof, wherein:
X is S and Y is —CR^{6a}, or Y is S and X is —CR^{6b};
   wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
   wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
   R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
   or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
      wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹².
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³; or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, and —C(O)R¹⁴,
   wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, or heterocycloalkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹, —C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

### Embodiment II-3

The compound of embodiment II-1, wherein the compound is of Formula (II-A):

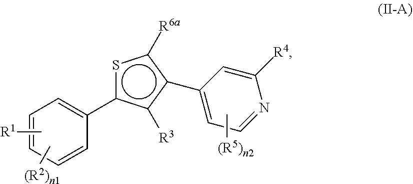

or a pharmaceutically acceptable salt, solvate, tautomer, isotope, or isomer thereof.

### Embodiment II-4

The compound of embodiment II-2, wherein the compound is of Formula (I-A):

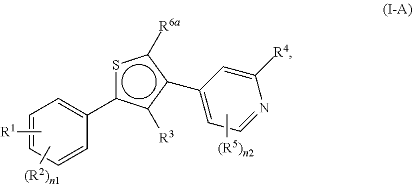

or a pharmaceutically acceptable salt, solvate, tautomer, isotope, or isomer thereof.

### Embodiment II-5

The compound of embodiment II-1 or II-3, wherein the compound is of Formula (II-A-i):

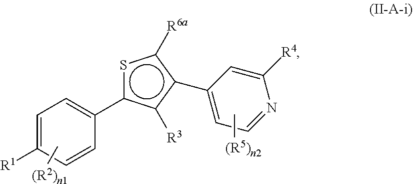

or a pharmaceutically acceptable salt, solvate, tautomer, isotope, or isomer thereof.

### Embodiment II-6

The compound of embodiment II-2 or II-4, wherein the compound is of Formula (I-A-i):

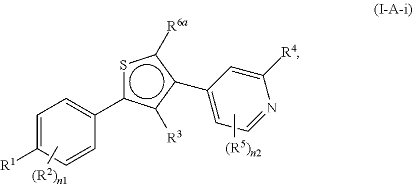

or a pharmaceutically acceptable salt, solvate, tautomer, isotope, or isomer thereof.

### Embodiment II-7

The compound of embodiment II-1, wherein the compound is of Formula (II-B):

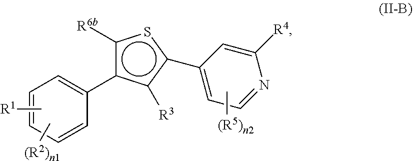

or a pharmaceutically acceptable salt, solvate, tautomer, isotope, or isomer thereof.

### Embodiment II-8

The compound of embodiment II-2, wherein the compound is of Formula (I-B):

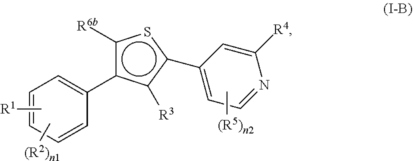

or a pharmaceutically acceptable salt, solvate, tautomer, isotope, or isomer thereof.

### Embodiment II-9

The compound of embodiment II-1 or II-7, wherein the compound is of Formula (II-B-i):

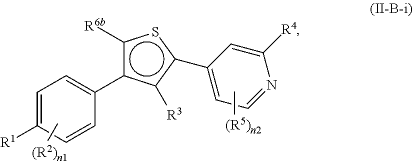

or a pharmaceutically acceptable salt, solvate, tautomer, isotope, or isomer thereof.

### Embodiment II-10

The compound of embodiment II-2 or 11-8, wherein the compound is of Formula (I-B-i):

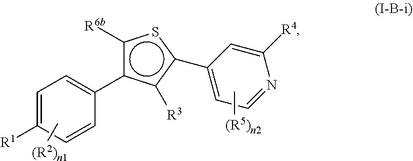

or a pharmaceutically acceptable salt, solvate, tautomer, isotope, or isomer thereof.

### Embodiment II-11

The compound of any one of embodiments II-1 to 11-10, or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)OR⁸, —S(O)₂R⁸, —NR⁷(SO)₂R⁹, or —NR⁸R⁹.

### Embodiment II-12

The compound of any one of embodiments II-1 to II-11, or a pharmaceutically acceptable salt, solvate, tautomer, isotope, or isomer thereof, wherein R¹ is —NR⁷C(O)NR⁸R⁹, —NR⁷S(O)₂NR⁸R⁹, —NR⁷C(O)ORB, —S(O)₂R⁸, or —NR⁸R⁹.

### Embodiment II-13

The compound of any one of embodiments II-1 to II-12, or a pharmaceutically acceptable salt, solvate, tautomer, isotope, or isomer thereof, wherein R¹ is —NR⁷C(O)NR⁸R⁹ or —NR⁷S(O)₂NR⁸R⁹.

Embodiment I1-14. The compound of any one of embodiments II-1 to II-12, or a pharmaceutically acceptable salt, solvate, tautomer, isotope, or isomer thereof, wherein R¹ is —NR⁷C(O)OR⁹, —NR⁷(SO)₂R⁹, or —S(O)₂R⁹.

### Embodiment II-15

The compound of any one of embodiments II-1 to II-14, or a pharmaceutically acceptable salt, solvate, tautomer, isotope, or isomer thereof, wherein R⁷ and R⁸ are both hydrogen, and R⁹ is alkyl, cycloalkyl, cycloalkyl-alkyl, heterocycloalkyl, or heterocycloalkyl-alkyl, wherein the alkyl, cycloalkyl, cycloalkyl-alkyl, heterocycloalkyl, or heterocycloalkyl-alkyl is unsubstituted or substituted with one or more substituents independently selected from the group consisting of halo and —OR¹⁰, wherein each R¹⁰ is independently hydrogen, alkyl, or haloalkyl.

### Embodiment II-16

The compound of any one of embodiments II-1 to II-13, or a pharmaceutically acceptable salt, solvate, tautomer, isotope, or isomer thereof, wherein R⁸ and R⁹, together with the nitrogen atom to which they are attached, form a heterocycloalkyl, wherein the heterocycloalkyl is unsubstituted or substituted with 1 to 3 substituents independently selected from the group consisting of halo, oxo, and —OR¹⁰, wherein each R¹⁰ is independently hydrogen, unsubstituted alkyl, or haloalkyl.

### Embodiment II-17

The compound of any one of embodiments II-1 to 11-16, or a pharmaceutically acceptable salt, solvate, tautomer, isotope, or isomer thereof, wherein n1 is 0 or 1.

### Embodiment II-18

The compound of any one of embodiments II-1 to II-17, or a pharmaceutically acceptable salt, solvate, tautomer, isotope, or isomer thereof, wherein each R² is independently halo, alkyl or —OR¹¹, wherein each R¹¹ is independently hydrogen, unsubstituted alkyl, or haloalkyl.

### Embodiment II-19

The compound of any one of embodiments II-1 to II-18, or a pharmaceutically acceptable salt, solvate, tautomer, isotope, or isomer thereof, wherein each R² is independently halo.

### Embodiment II-20

The compound of any one of embodiments II-1 to II-19, or a pharmaceutically acceptable salt, solvate, tautomer, isotope, or isomer thereof, wherein each R² is chloro.

### Embodiment II-21

The compound of any one of embodiments II-1 to II-20, or a pharmaceutically acceptable salt, solvate, tautomer, isotope, or isomer thereof, wherein R³ and R⁶a or R^{6b} are both hydrogen.

### Embodiment II-22

The compound of any one of embodiments II-1 to II-21, or a pharmaceutically acceptable salt, solvate, tautomer, isotope, or isomer thereof, wherein n2 is 0.

### Embodiment II-23

The compound of any one of embodiments II-1 to II-22, or a pharmaceutically acceptable salt, solvate, tautomer, isotope, or isomer thereof, wherein R⁴ is alkyl or cycloalkyl, wherein the alkyl or cycloalkyl is unsubstituted or substituted with 1 to 3 substituents independently selected from the group consisting of halo, alkyl, alkyl substituted with —(OR¹⁸)ₙ₅OR¹⁷, haloalkyl, haloalkyl substituted with —(OR¹⁸)ₙ₅OR¹⁷, cycloalkyl, and —OR¹⁴, wherein each R¹⁴ and R¹⁷ is independently hydrogen, unsubstituted alkyl, or haloalkyl, and each R¹⁸ is independently alkylene.

### Embodiment II-24

The compound of any one of embodiments II-1 to II-22, or a pharmaceutically acceptable salt, solvate, tautomer, isotope, or isomer thereof, wherein R⁴ is —OR¹² and R¹² is heterocycloalkyl-alkyl.

### Embodiment II-25

The compound of any one of embodiments II-1 to II-22 or a pharmaceutically acceptable salt, solvate, tautomer, isotope, or isomer thereof, wherein R⁴ is alkyl substituted with one or more —OC(O)R²²; wherein R²² is —R²³N(R²⁴)₂ or —(CH₂CH₂—O—)ₙ₈CH₃.

### Embodiment II-26

The compound of embodiment II-25, or a pharmaceutically acceptable salt, solvate, tautomer, isotope, or isomer thereof, wherein R²² is —R²³N(R²⁴)₂.

### Embodiment II-27

The compound of embodiment II-25, or a pharmaceutically acceptable salt, solvate, tautomer, isotope, or isomer thereof, wherein R²² is —(CH₂CH₂—O—)ₙ₈CH₃.

### Embodiment II-28

The compound of any one of embodiments II-1 to II-6 or II-11 to II-27, selected from the group consisting of:

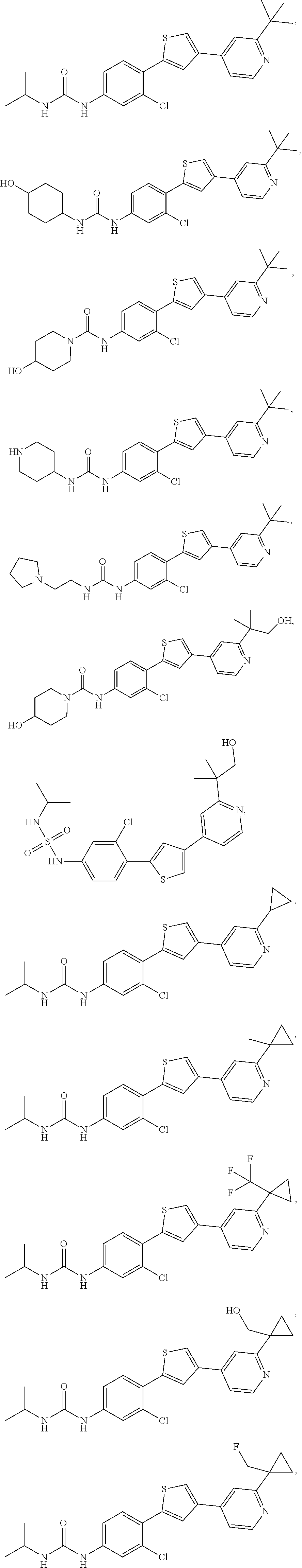

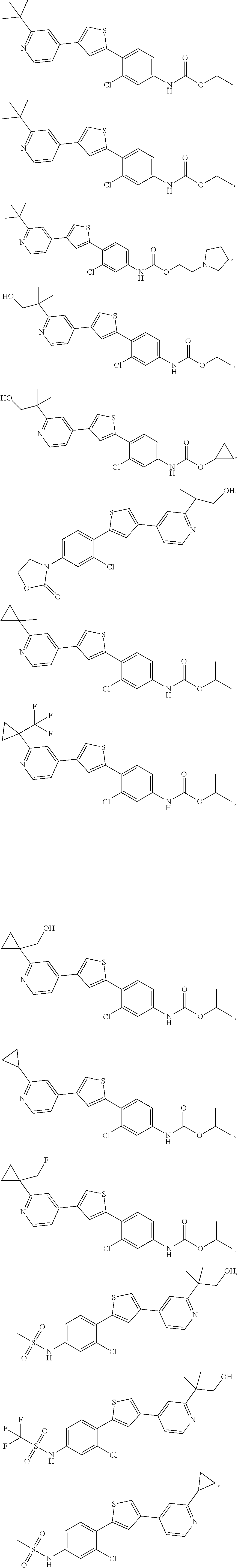

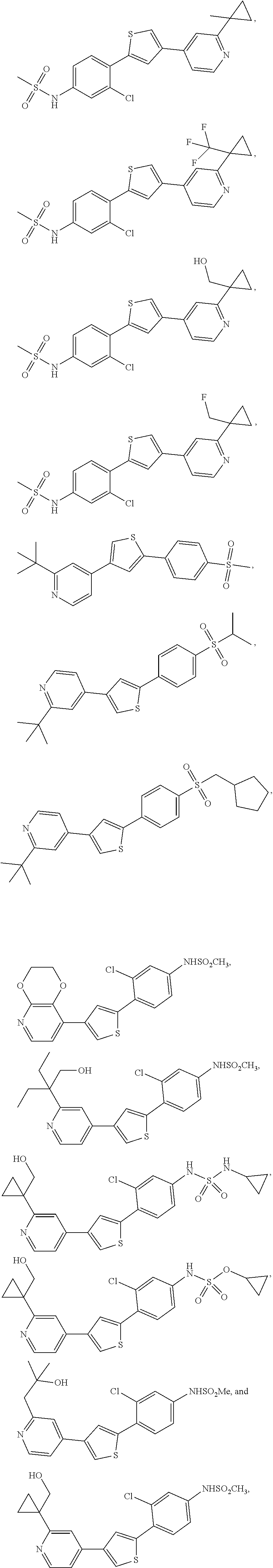

or a pharmaceutically acceptable salt, solvate, tautomer, isotope, or isomer of any of the foregoing.

### Embodiment II-29

The compound of any one of embodiments II-1 to II-27, selected from the group consisting of:

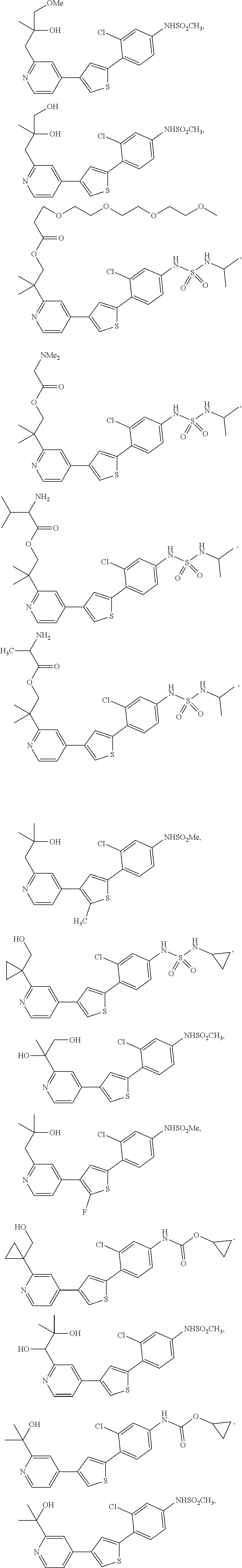

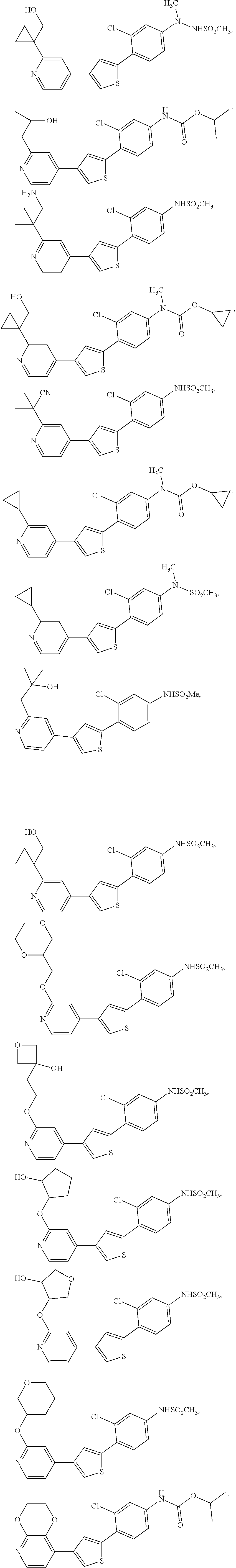

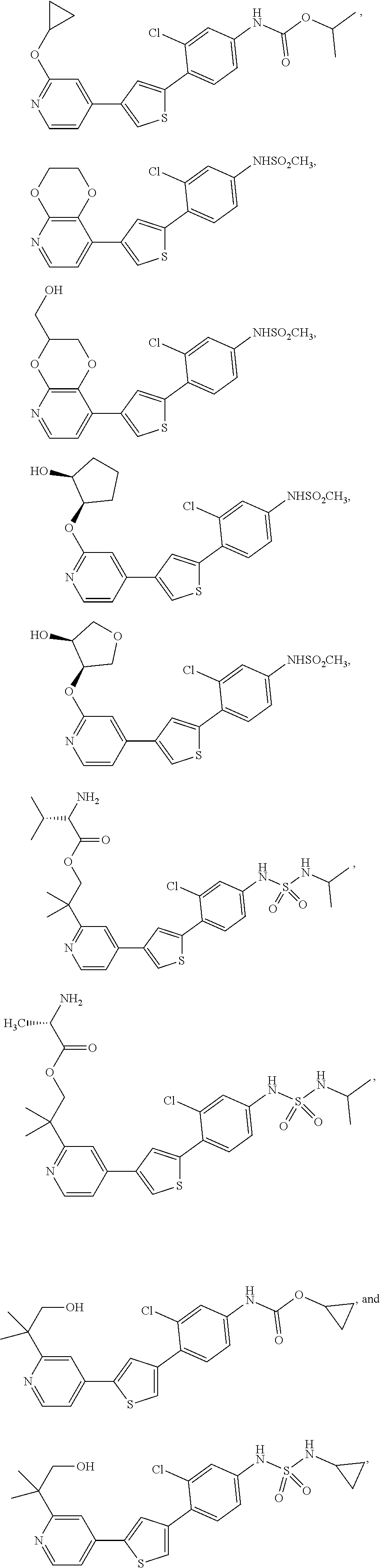

or a pharmaceutically acceptable salt, solvate, tautomer, isotope, or isomer of any of the foregoing.

### Embodiment II-30

The compound of any one of embodiments II-1 to II-6 or II-11 to II-27, selected from the group consisting of:

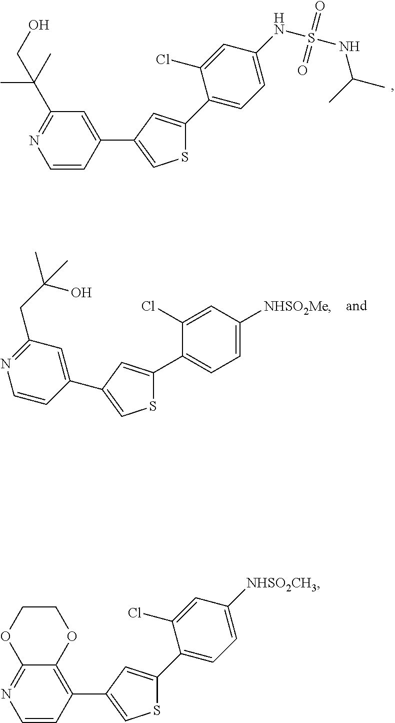

or a pharmaceutically acceptable salt, solvate, tautomer, isotope, or isomer of any of the foregoing.

### Embodiment II-31

A pharmaceutical composition, comprising the compound of any one of embodiments II-1 to II-30, or a pharmaceutically acceptable salt, solvate, tautomer, isotope, or isomer thereof, and a pharmaceutically acceptable excipient.

### Embodiment II-32

A method of inhibiting a sterol regulatory element-binding protein (SREBP), comprising contacting the SREBP or contacting an SREBP cleavage activating-protein (SCAP) with an effective amount of a compound of any one of embodiments II-1 to II-30, or a pharmaceutically acceptable salt, solvate, tautomer, isotope, or isomer thereof, or the pharmaceutical composition of embodiment II-31.

### Embodiment II-33

A method of inhibiting the proteolytic activation of a sterol regulatory element-binding protein (SREBP), comprising contacting an SREBP cleavage activating-protein (SCAP) with an effective amount of a compound of any one of embodiments II-1 to II-30, or a pharmaceutically acceptable salt, solvate, tautomer, isotope, or isomer thereof, or the pharmaceutical composition of embodiment II-31.

### Embodiment II-34

The method of embodiment II-32 or II-33, wherein the SREBP is an SREBP-1.

### Embodiment II-35

The method of embodiment II-34, wherein the SREBP-1 is SREBP-1a.

### Embodiment II-36

The method of embodiment II-34, wherein the SREBP-1 is SREBP-1c.

### Embodiment II-37

The method of embodiment II-32 or II-33, wherein the SREBP is SREBP-2.

### Embodiment II-38

The method of any one of embodiments II-32 to II-37, wherein SREBP is inhibited in a subject in need thereof.

### Embodiment II-39

The method of any one of embodiments II-32 to II-38, wherein SCAP is inhibited in a subject in need thereof.

### Embodiment II-40

The method of any one of embodiments II-32 to II-39, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof, or the pharmaceutical composition.

### Embodiment II-41

A method of treating a disorder in a subject in need thereof, comprising administering to the subject in need thereof an effective amount of a compound of any one of embodiments II-1 to II-30, or a pharmaceutically acceptable salt, solvate, tautomer, isotope, or isomer thereof, or the pharmaceutical composition of embodiment II-31.

### Embodiment II-42

A method of treating a disorder in a subject in need thereof, wherein the disorder is mediated by a sterol regulatory element-binding protein (SREBP), comprising administering to the subject in need thereof an effective amount of a compound of any one of embodiments II-1 to II-30, or a pharmaceutically acceptable salt, solvate, tautomer, isotope, or isomer thereof, or the pharmaceutical composition of embodiment II-31.

### Embodiment II-43

The method of embodiment II-41 or II-42, wherein the disorder is Metabolic Syndrome, type 2 diabetes, obesity, liver disease, insulin resistance, adiposopathy, or dyslipidemia.

### Embodiment II-44

The method of embodiment II-43, wherein the dyslipidemia is hypertriglyceridemia or elevated cholesterol levels.

### Embodiment II-45

The method of embodiment II-43, wherein the liver disease is nonalcoholic steatohepatitis, liver fibrosis, or liver inflammation, or a combination thereof.

### Embodiment II-46

The method of embodiment II-41 or II-42, wherein the disorder is a hyperproliferative disorder.

### Embodiment II-47

The method of embodiment II-46, wherein the hyperproliferative disorder is cancer.

### Embodiment II-48

The method of embodiment II-47, wherein the cancer is breast cancer, liver cancer, ovarian cancer, pancreatic cancer, prostate cancer, soft tissue sarcoma, bladder cancer, endometrial cancer, skin cancer, colon cancer, hematologic cancer, placenta cancer, brain cancer, kidney cancer, lung cancer, or bone cancer.

### Embodiment II-49

The method of embodiment II-41 or II-42, wherein the disorder is endotoxic shock, systemic inflammation, or atherosclerosis.

### Embodiment II-50

A compound of any one of embodiments II-1 to II-30, or a pharmaceutically acceptable salt, solvate, tautomer, isotope, or isomer thereof, for use in inhibiting a sterol regulatory element-binding protein (SREBP).

### Embodiment II-51

A compound of any one of embodiments II-1 to II-30, or a pharmaceutically acceptable salt, solvate, tautomer, isotope, or isomer thereof, for use in inhibiting the proteolytic activation of a sterol regulatory element-binding protein (SREBP).

### Embodiment II-52

The compound for use of embodiment II-50 or II-51, wherein the SREBP is an SREBP-1.

### Embodiment II-53

The compound for use of embodiment II-52, wherein the SREBP-1 is SREBP-1a.

### Embodiment II-54

The compound for use of embodiment II-53, wherein the SREBP-1 is SREBP-1c.

### Embodiment II-55

The compound for use of embodiment II-50 or II-51, wherein the SREBP is SREBP-2.

### Embodiment II-56

The compound for use of any one of embodiments II-50 to II-55, wherein SREBP is inhibited in a subject in need thereof.

### Embodiment II-57

The compound for use of any one of embodiments II-50 to II-56, wherein SCAP is inhibited in a subject in need thereof.

### Embodiment II-58

The compound for use of any one of embodiments II-50 to II-57, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

### Embodiment II-59

A compound of any one of embodiments II-1 to II-30, or a pharmaceutically acceptable salt, solvate, tautomer, isotope, or isomer thereof, for use in treating a disorder in a subject in need thereof.

### Embodiment II-60

A compound of any one of embodiments II-1 to II-30, or a pharmaceutically acceptable salt, solvate, tautomer, isotope, or isomer thereof, for use in treating a disorder in a subject in need thereof, wherein the disorder is mediated by a sterol regulatory element-binding protein (SREBP).

### Embodiment II-61

The compound for use of embodiment II-59 or II-60, wherein the disorder is Metabolic Syndrome, type 2 diabetes, obesity, liver disease, insulin resistance, adiposopathy, or dyslipidemia.

### Embodiment II-62

The compound for use of embodiment II-61, wherein the dyslipidemia is hypertriglyceridemia or elevated cholesterol levels.

### Embodiment II-63

The compound for use of embodiment II-61, wherein the liver disease is nonalcoholic steatohepatitis, liver fibrosis, or liver inflammation, or a combination thereof.

### Embodiment II-64

The compound for use of embodiment II-59 or II-60, wherein the disorder is a hyperproliferative disorder.

### Embodiment II-65

The compound for use of embodiment II-64, wherein the hyperproliferative disorder is cancer.

### Embodiment II-66

The compound for use of embodiment II-65, wherein the cancer is breast cancer, liver cancer, ovarian cancer, pancreatic cancer, prostate cancer, soft tissue sarcoma, bladder cancer, endometrial cancer, skin cancer, colon cancer, hematologic cancer, placenta cancer, brain cancer, kidney cancer, lung cancer, or bone cancer.

### Embodiment II-67

The compound for use of embodiment II-59 or II-60, wherein the disorder is endotoxic shock, systemic inflammation, or atherosclerosis.

### Embodiment II-68

Use compound of any one of embodiments II-1 to II-30, or a pharmaceutically acceptable salt, solvate, tautomer, isotope, or isomer thereof, in the manufacture of a medicament for inhibiting a sterol regulatory element-binding protein (SREBP).

### Embodiment II-69

Use of a compound of any one of embodiments II-1 to II-30, or a pharmaceutically acceptable salt, solvate, tautomer, isotope, or isomer thereof, in the manufacture of a medicament for inhibiting the proteolytic activation of a sterol regulatory element-binding protein (SREBP).

### Embodiment II-70

The use of embodiment II-68 or II-69, wherein the SREBP is an SREBP-1.

### Embodiment II-71

The use of embodiment II-70, wherein the SREBP-1 is SREBP-1a.

### Embodiment II-72

The use of embodiment II-70, wherein the SREBP-1 is SREBP-1c.

### Embodiment II-73

The use of embodiment II-68 or II-69, wherein the SREBP is SREBP-2.

### Embodiment II-74

The use of any one of embodiments II-68 to II-73, wherein SREBP is inhibited in a subject in need thereof.

### Embodiment II-75

The use of any one of embodiments II-68 to II-74, wherein SCAP is inhibited in a subject in need thereof.

### Embodiment II-76

The use of any one of embodiments II-68 to II-75, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

### Embodiment II-77

Use of a compound of any one of embodiments II-1 to II-30, or a pharmaceutically acceptable salt, solvate, tautomer, isotope, or isomer thereof, in the manufacture of a medicament for treating a disorder in a subject in need thereof.

### Embodiment II-78

Use of a compound of any one of embodiments II-1 to II-30, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

### Embodiment II-79

The use of embodiment II-77 or II-78, wherein the disorder is Metabolic Syndrome, type 2 diabetes, obesity, liver disease, insulin resistance, adiposopathy, or dyslipidemia.

### Embodiment II-80

The use of embodiment II-79, wherein the dyslipidemia is hypertriglyceridemia or elevated cholesterol levels.

### Embodiment II-81

The use of embodiment II-79, wherein the liver disease is nonalcoholic steatohepatitis, liver fibrosis, or liver inflammation, or a combination thereof.

### Embodiment II-82

The use of embodiment II-77 or II-78, wherein the disorder is a hyperproliferative disorder.

### Embodiment II-83

The use of embodiment II-82, wherein the hyperproliferative disorder is cancer.

### Embodiment II-84

The use of embodiment II-83, wherein the cancer is breast cancer, liver cancer, ovarian cancer, pancreatic cancer, prostate cancer, soft tissue sarcoma, bladder cancer, endometrial cancer, skin cancer, colon cancer, hematologic cancer, placenta cancer, brain cancer, kidney cancer, lung cancer, or bone cancer.

### Embodiment II-85

The use of embodiment II-77 or II-78, wherein the disorder is endotoxic shock, systemic inflammation, or atherosclerosis.

### EXAMPLES

The following Examples are merely illustrative and are not meant to limit any aspects of the present disclosure in any way.

### Synthesis Example 1: 2-(tert-butyl)-4-(5-(4-(isopropylsulfonyl)phenyl)thiophen-3-yl)pyridine (Compound I-8728)

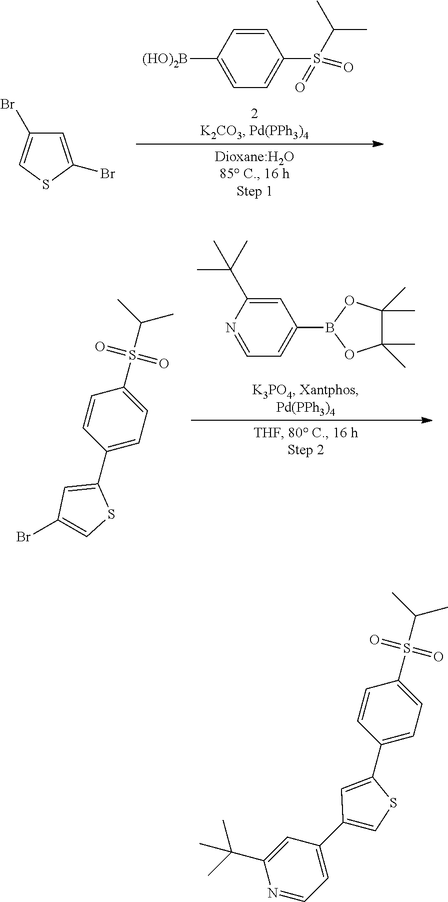 [SMILES: CC(C)S(=O)(=O)c1ccc(B(O)O)cc1 ; Brc1csc(Br)c1 ; CC(C)S(=O)(=O)c1ccc(-c2cc(Br)cs2)cc1 ; CC(C)(C)c1cc(B2OC(C)(C)C(C)(C)O2)ccn1 ; CC(C)S(=O)(=O)c1ccc(-c2cc(-c3ccnc(C(C)(C)C)c3)cs2)cc1]

### Step 1: 4-bromo-2-(4-(isopropylsulfonyl)phenyl)thiophen

2,4-Dibromothiophene (0.150 g, 0.620 mmol), (4-(isopropylsulfonyl)phenyl)boronic acid (0.169 g, 0.744 mmol), potassium carbonate (0.171 g, 1.24 mmol) in dioxane:water (2.4:0.6 mL) were charged in 10 mL glass seal tube and purged with N2 gas for 10 min. Palladium tetrakis (0.071 g, 0.062 mmol) was added and the mixture was gain purged with nitrogen gas for 10 minutes. The tube was sealed and heated to 85° C. for 16h. After completion, the reaction mixture was cooled to room temperature (RT) and the solvent evaporated. 5 mL water was added and the product was extracted into ethyl acetate (EtOAc; 3×15 mL). The extracts were combined and washed with brine (3×10 mL) and dried over anhydrous sodium sulfate and concentrated under reduced pressure to give a crude product which was purified by column chromatography (silica gel: #100-200) using 0-20% EtOAc in hexane as eluent to afford 4-bromo-2-(4-(isopropylsulfonyl)phenyl)thiophene (0.155 g, 72.42%) as pale yellow semi solid. ¹H NMR (400 MHz, CDCl₃: δ (ppm): 7.89 (2H, J=6.4 Hz d) 7.72 (2H, J=8.8 Hz, d), 7.35 (1H, J 1.6 Hz d), 7.30 (1H, J=1.2 Hz, d), 3.24-3.17 (1H, m), 1.32-1.31 (6H, d); LCMS 93% 347.15[M+2H].

### Step 2: 2-(tert-butyl)-4-(5-(4-(isopropylsulfonyl)phenyl)thiophen-3-yl)pyridine

The product of Step 1 (0.080 g, 0.2316 mmol), 2-tert butyl pyridine 4-boronic acid pinacol ester (0.079 g, 0.2779 mmol) and K₃PO₄ (0.148 g, 0.6948 mmol, 2 eq) in tetrahydrofuran (THF; 1.6 mL) were charged in 10 mL glass seal tube and purged with nitrogen gas for 10 minutes. After adding xantphos (0.013 g, 0.0231 mmol) and palladium tetrakis (0.027 g, 0.0231 mmol) it was again purged with nitrogen gas for 10 minutes, then the tube was sealed and heated to 80° C. for 16 h. After completion, the reaction mass was cooled, the solvent was evaporated, 10 mL water added, and the product extracted into EtOAc (3×20 mL). The extracts were combined, dried over anhydrous sodium sulfate and concentrated under reduced pressure to give a crude that was purified by column chromatography (silica gel: #100-200) using 0-20% EtOAc in hexane as eluent to afford 2-(tert-butyl)-4-(5-(4-(isopropylsulfonyl)phenyl)thiophen-3-yl)pyridine, (0.060 g, 65.21%) as an off-white solid. ¹H NMR (400 MHz, CDCl₃): δ (ppm): 8.62-8.60 (1H, J=0.8 Hz, dd), 7.92 (2H, J=1.6 Hz, dd), 7.84 (2H, J=2 Hz, dd), 7.74 (1H, J=1.6 Hz, d) 7.68 (1H, J=1.6 Hz, d), 7.54-7.53 (1H, J=0.8 Hz, dd), 7.32-7.30 (1H, J=1.6 Hz, dd), 3.26-3.19 (1H, m), 1.43 (9H, s), 1.34-1.32 (6H, d), LCMS 99.56% (m/z 400.1 [M+H]).

### Synthesis Example 2: 2-(tert-butyl)-4-(5-(4-(methylsulfonyl)phenyl)thiophen-3-yl)pyridine (Compound I-8723)

This compound was prepared following a procedure similar to that in Example 1. ¹H NMR (400 MHz, DMSO): (8.55 (d. J=5.2 Hz, 1H), 8.38 (d, J=1.2 Hz, 1H), 8.355 (s, 1H), 8.057 (d. J=8.8 Hz, 2H), 7.98 (d. J=8.4 Hz, 2H), 7.806 (s, 1H), 7.61-7.603 (m, 1H), 3.26 (s, 3H), 1.38 (s, 9H); LCMS: 96.08% (m/z=372.20 [M+H]).

### Synthesis Example 3: 2-(tert-butyl)-4-(5-(4-((cyclopentylmethyl)sulfonyl)phenyl)thiophen-3-yl)pyridine (Compound I-8729)

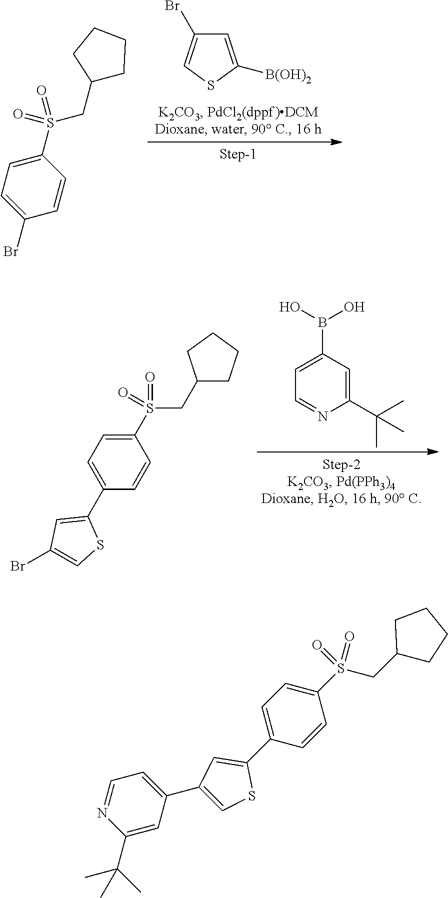 [SMILES: O=S(=O)(CC1CCCC1)c1ccc(Br)cc1 ; OB(O)c1cc(Br)cs1 ; O=S(=O)(CC1CCCC1)c1ccc(-c2cc(Br)cs2)cc1 ; CC(C)(C)c1cc(B(O)O)ccn1 ; CC(C)(C)c1cc(-c2csc(-c3ccc(S(=O)(=O)CC4CCCC4)cc3)c2)ccn1]

### Step 1: 4-bromo-2-(4-((cyclopentylmethyl)sulfonyl)phenyl)thiophene

To a stirred solution of 1-bromo-4-((cyclopentylmethyl)sulfonyl)benzene (0.5 g, 1.64 mmol) in dioxane: water (4:1 mL) in a glass tube was added K₂CO₃ (0.56 g, 4.1 mmol) followed by (4-bromothiophen-2-yl)boronic acid (0.48 g, 1.97 mmol) at room temperature under a nitrogen atmosphere. The reaction mass was purged for 15 minutes with nitrogen, then added PdCl₂(dppf).dichloromethane (DCM) (0.13 g, 0.164 mmol) and again purged with nitrogen for 10 minutes. The reaction tube was sealed and stirred at 90° C. for 16 h. The reaction mixture was cooled to 25° C., water (50 mL) was added and the product extracted into ethyl acetate; the organic layer was washed with brine, dried over sodium sulfate, filtered and concentrated under reduced pressure to give a crude product which was purified by silica gel column chromatography eluting with 0-30% ethyl acetate in pet ether to afford 4-bromo-2-(4-((cyclopentylmethyl)sulfonyl)phenyl)thiophene (80 mg, 13%). ¹H NMR (400 MHz, CDCl₃): δ 7.93-7.91 (m, 2H), 7.81-7.61 (m, 2H), 7.43 (d, J=1.2 Hz, 1H), 7.35 (d, J=1.2 Hz, 1H), 3.18-3.12 (m, 2H), 2.28-2.24 (m, 1H), 1.92-1.88 (m, 2H), 1.64-1.51 (m, 4H), 1.28-1.20 (m, 2H); LCMS: 45.91% (m/z=387.21 [M+2]).

### Step 2: 2-(tert-butyl)-4-(5-(4-((cyclopentylmethyl)sulfonyl)phenyl)thiophen-3-yl)pyridine

To a stirred solution of 4-bromo-2-(4-((cyclopentylmethyl)sulfonyl)phenyl)thiophene (0.075 g, 0.19 mmol) in dioxane/H₂O (0.8:0.2 mL) in a glass tube was added (2-(tert-butyl)pyridin-4-yl)boronic acid (0.039 g, 0.21 mmol), K₂CO₃ (0.08 g, 0.58 mmol) at room temperature under a nitrogen atmosphere. The reaction mass was purged for 15 minutes with nitrogen followed by addition of palladium tetrakis (0.023 g, 0.019 mmol) and again purged for 10 minutes with nitrogen. The reaction vessel was sealed and stirred at 90° C. for 16 h. The reaction mixture was cooled to 25° C., water (50 mL) was added and the product was extracted into ethyl acetate. The organic layer was washed with brine, dried over sodium sulfate, filtered and concentrated under reduced pressure to give a crude product which was purified by prep HPLC (preparative HPLC) to give 2-(tert-butyl)-4-(5-(4-((cyclopentylmethyl)sulfonyl)phenyl)thiophen-3-yl)pyridine (10 mg; 12%). ¹H NMR (400 MHz, DMSO): δ 8.55 (d, J=5.2 Hz, 1H), 8.39 (d, J=1.6 Hz, 1H), 8.35 (d, J=1.2 Hz, 1H), 8.05 (d, J=8.4 Hz, 2H), 7.98 (d, J=8.8 Hz, 2H), 7.80 (bs, 1H), c7.62-7.60 (m, 1H), 3.39 (d, J=6.8 Hz, 2H), 2.11-2.07 (m, 1H), 1.76-1.71 (m, 2H), 1.57-1.53 (m, 2H), 1.51-1.38 (m, 11H), 1.23-1.22 (m, 2H); LCMS: 97.59% (m/z=440.77 [M+H]).

### Synthesis Example 4: 1-(4-(4-(2-(tert-butyl)pyridin-4-yl)thiophen-2-yl)-3-chlorophenyl)-3-isopropylurea (Compound I-8731)

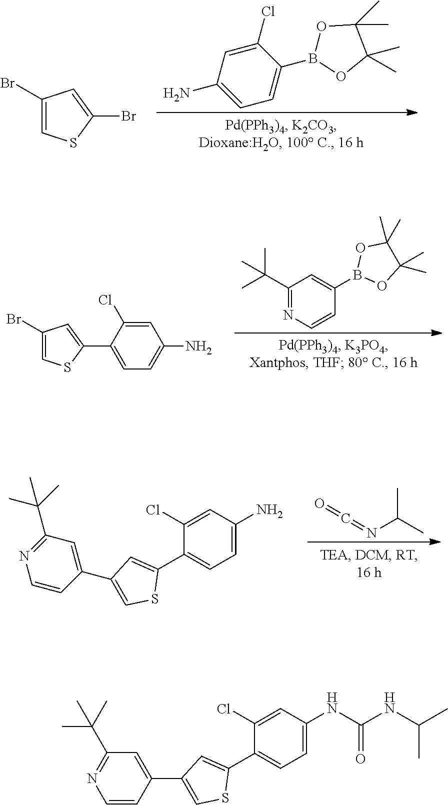

### Step 1: 4-(4-bromothiophen-2-yl)-3-chloroaniline

2,4-Dibromo thiophene (0.800 g, 3.307 mmol, 1.0 eq), 3-chloro-4-(4,4,5,5-tetramethyl-1,3,2-dioxaborolan-2-yl)aniline (0.923 g, 3.637 mmol, 1.1 eq), potassium carbonate (1.14 g, 8.267 mmol, 2.5 eq) in water (2 mL) and dioxane (10 mL) were charged in a glass seal tube and purged with nitrogen gas for 15 minutes. After adding tetrakistriphenylphosphine palladium (0.382 g, 0.330 mmol, 0.1 eq), the tube was purged again with nitrogen gas for 10 min and then the reaction tube was sealed and heated to 100° C. for 16h. After completion, the dioxane was removed under reduced pressure and the residue was dissolved in water (10 mL) and extracted with EtOAc (3×15 mL). The combined extracts were dried over anhydrous sodium sulfate and concentrated under reduced pressure to obtain crude, which was purified by column chromatography (Silica 60-120 mesh) and eluted with 10% EtOAc in petroleum (pet) ether. The collected fractions were concentrated under reduced pressure to obtain 4-(4-bromothiophen-2-yl)-3-chloroaniline as a pale yellow liquid which was used as such for next step.

### Step 2: 4-(4-(2-(tert-butyl)pyridin-4-yl)thiophen-2-yl)-3-chloroaniline

To a stirred solution of 4-(4-bromothiophen-2-yl)-3-chloroaniline (0.550 g, 1.906 mmol, 1.0 eq) in THF (10 mL) in a glass tube, was added 2-(tert-butyl)-4-(4,4,5,5-tetramethyl-1,3,2-dioxaborolan-2-yl)pyridine (0.409 g, 2.287 mmol, 1.2 eq), K₃PO₄ (1.010 g, 4.766 mmol, 2.5 eq) at room temperature under a nitrogen atmosphere. The reaction mass was purged for 15 min with nitrogen. Then was added tetrakistriphenylphosphine palladium (0.220 g, 0.190 mmol, 0.1 eq) and xantphos (0.111 g, 0.190 mmol, 0.1 eq), and the tube again purged for 10 min with nitrogen. The reaction tube was sealed and stirred at 80° C. for 16 h. After completion, the residue was dissolved in water (10 mL) and extracted with EtOAc (3×15 mL). The extracts were combined, dried over anhydrous sodium sulfate and concentrated under reduced pressure to obtain crude which was purified by column chromatography, eluting with 15% EtOAc in pet ether. Product fractions were concentrated under reduced pressure to afford 4-(4-(2-(tert-butyl)pyridin-4-yl)thiophen-2-yl)-3-chloroaniline (0.220 g, 35%) as a pale yellow liquid. ¹H NMR (400 MHz, DMSO: δ (ppm): 8.51-8.50 (dd, J=0.4 Hz, J=0.4 Hz, 1H), 8.13 (d, J=1.2 Hz, 1H), 7.74-7.70 (dd, J=1.6 Hz, J=0.8 Hz, 2H), 7.53-7.51 (dd, J=1.6 Hz, J=1.6 Hz, 1H), 7.38 (d, J=8.4 Hz, 1H), 6.72 (d, J=2.0 Hz, 1H), 6.61-6.58 (dd, J=2.4 Hz, J=2.4 Hz, 1H), 5.67 (s, 2H), 1.36 (s, 9H).

### Step 3: Synthesis of 1-(4-(4-(2-(tert-butyl)pyridin-4-yl)thiophen-2-yl)-3-chlorophenyl)-3-isopropylurea

To a stirred solution of 4-(4-(2-(tert-butyl)pyridin-4-yl)thiophen-2-yl)-3-chloroaniline (0.100 g, 0.292 mmol, 1.0 eq) in dichloromethane (DCM; 5 mL) was added TEA (0.061 mL, 0.438 mmol, 1.5 eq) and isopropylisocyanate (0.037 g, 0.438 mmol, 1.5 eq). The reaction mixture was stirred at RT for 16 h. After completion, the solvent was evaporated and the residue dissolved in water (10 mL) and extracted with DCM (3×10 mL). The extracts were combined, dried over anhydrous sodium sulfate and concentrated under reduced pressure to obtain crude product, which was purified by prep HPLC. The product fraction was concentrated under reduced pressure to afford 1-(4-(4-(2-(tert-butyl)pyridin-4-yl)thiophen-2-yl)-3-chlorophenyl)-3-isopropylurea. (0.035 g, 28%) as a white solid. ¹H NMR (400 MHz, DMSO: δ (ppm): 8.83 (s, 1H), 8.52-8.51 (dd, J=0.4 Hz, J=0.4 Hz, 1H), 8.23 (d, J=1.6 Hz, 1H), 7.88 (d, J=1.6 Hz, 1H), 7.81 (d, J=2.4 Hz, 1H), 7.73 (d, J=0.8 Hz, 1H), 7.60 (d, J=8.8 Hz, 1H), 7.55-7.54 (dd, J=0.8 Hz, J=1.6 Hz, 1H), 7.31-7.29 (dd, J=2.0 Hz, J=2.4 Hz, 1H), 6.32 (d, J=7.6 Hz, 1H), 3.79-3.74 (q, 1H), 1.36 (s, 9H), 1.10 (d, J=6.4 Hz, 6H); LCMS: (428.38[M+H].

### Synthesis Example 5: N-(4-(4-(2-(tert-butyl)pyridin-4-yl)thiophen-2-yl)-3-chlorophenyl)-4-hydroxypiperidine-1-carboxamide (Compound I-8736)

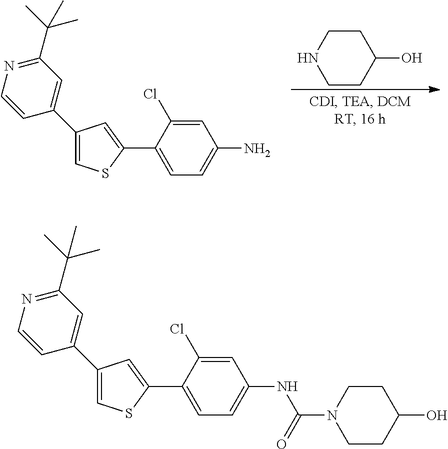 [SMILES: CC(C)(C)c1cc(-c2csc(-c3ccc(N)cc3Cl)c2)ccn1 ; OC1CCNCC1 ; CC(C)(C)c1cc(-c2csc(-c3ccc(NC(=O)N4CCC(O)CC4)cc3Cl)c2)ccn1]

To a stirred solution of 4-(4-(2-(tert-butyl)pyridin-4-yl)thiophen-2-yl)-3-chloroaniline (0.060 g, 0.093 mmol, 1.0 eq) in DCM (2 mL) was added triethylamine (TEA; 0.061 mL, 0.186 mmol, 2.0 eq) and CDI (0.056 g, 0.093 mmol, 1.0 eq). After 30 min, 4-hydroxypiperidine (0.035 g, 0.186 mmol, 2.0 eq) was added. The reaction mixture was stirred at RT for 16 h, with progress monitored by thin layer chromatography (TLC). After completion, the residue was dissolved in water (10 mL) and extracted with DCM (3×10 mL), extracts combined, dried over anhydrous sodium sulfate and concentrated under reduced pressure to obtain crude product, which was purified by prep HPLC. Product fraction was concentrated under reduced pressure to afford N-(4-(4-(2-(tert-butyl)pyridin-4-yl)thiophen-2-yl)-3-chlorophenyl)-4-hydroxypiperidine-1-carboxamide (0.025 g, 30%) as an off-white solid. ¹H NMR (400 MHz, DMSO: δ (ppm): 8.80 (s, 1H), 8.52-8.51 (dd, J=0.4 Hz, J=0.4 Hz, 1H), 8.24 (d, J=1.6 Hz, 1H), 7.91 (d, J=1.6 Hz, 1H), 7.82 (d, J=2.0 Hz, 1H), 7.74 (d, J=0.8 Hz, 1H), 7.64 (d, J=8.4 Hz, 1H), 7.56-7.51 (m, 2H), 4.72 (d, J=4.4 Hz, 1H), 3.85-3.80 (m, 2H), 3.70-3.66 (q, 1H), 3.12-3.06 (m, 2H), 1.77-1.73 (m, 2H), 1.36 (s, 11H); LCMS: (470.42[M+H].

### Synthesis Example 6: 1-(4-(4-(2-(tert-butyl)pyridin-4-yl)thiophen-2-yl)-3-chlorophenyl)-3-(4-hydroxycyclohexyl)urea (Compound I-8742)

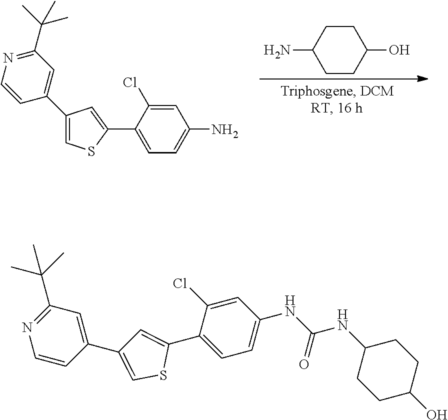 [SMILES: CC(C)(C)c1cc(-c2csc(-c3ccc(N)cc3Cl)c2)ccn1 ; NC1CCC(O)CC1 ; CC(C)(C)c1cc(-c2csc(-c3ccc(NC(=O)NC4CCC(O)CC4)cc3Cl)c2)ccn1]

To a stirred solution of 4-(4-(2-(tert-butyl)pyridin-4-yl)thiophen-2-yl)-3-chloroaniline (0.080 g, 0.233 mmol, 1.0 eq) in DCM (3 mL) was added triphosgene (0.068 g, 0.233 mmol, 1.0 eq) at 0° C. The reaction mixture was stirred at RT for 3h. Then 4-aminocyclohexan-1-ol (0.053 g, 0.46 mmol, 2.0 eq) was added. The reaction mixture was stirred at RT for 16h. After completion, the solvent was evaporated and the residue was dissolved in water (10 mL) and extracted with DCM (3×10 mL). The extracts were combined, dried over anhydrous sodium sulfate and concentrated under reduced pressure to obtain crude compound, which was purified by prep HPLC. The product fraction was concentrated under reduced pressure to afford 1-(4-(4-(2-(tert-butyl)pyridin-4-yl)thiophen-2-yl)-3-chlorophenyl)-3-(4-hydroxycyclohexyl)urea (0.011 g, 10%) as a white solid. ¹H NMR (400 MHz, DMSO: δ (ppm): 8.68 (s, 1H), 8.52-8.51 (dd, J=0.8 Hz, J=0.8 Hz, 1H), 8.23 (d, J=1.6 Hz, 1H), 7.88 (d, J=1.2 Hz, 1H), 7.79 (d, J=2.4 Hz, 1H), 7.73 (d, J=0.8 Hz, 1H), 7.61 (d, J=8.4 Hz, 1H), 7.55-7.54 (dd, J=1.6 Hz, J=1.6 Hz, 1H), 7.30-7.27 (dd, J=2.0 Hz, J=2.4 Hz, 1H), 6.18 (d, J=7.6 Hz, 1H), 4.53 (d, J=4.4 Hz, 1H), 3.41-3.39 (m, 2H), 1.85-1.79 (m, 4H), 1.36 (s, 9H), 1.24-1.22 (m, 4H); LCMS: (484.47[M+H].

### Synthesis Example 7: 1-(4-(4-(2-(tert-butyl)pyridin-4-yl)thiophen-2-yl)-3-chlorophenyl)-3-(piperidin-4-yl)urea (Compound I-8755)

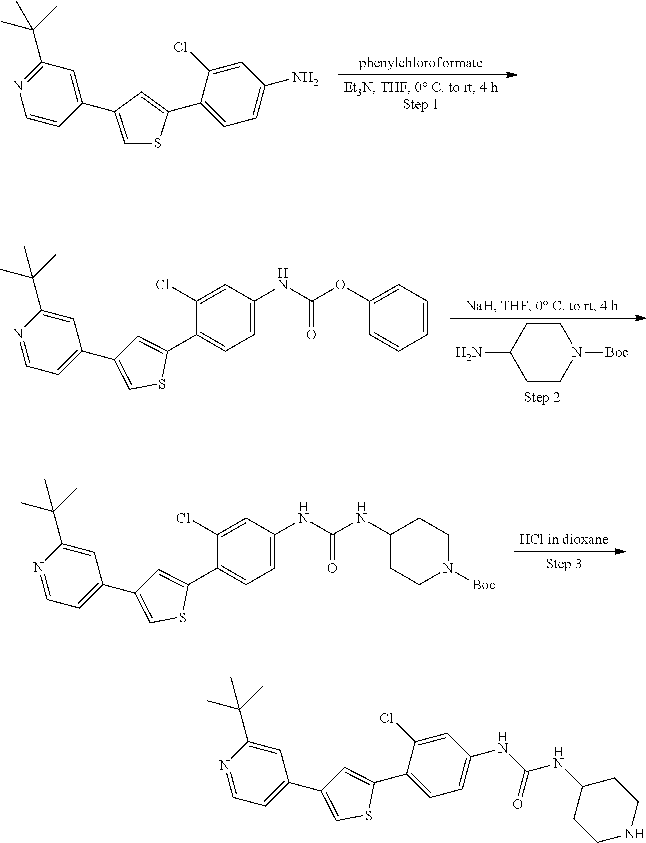 [SMILES: CC(C)(C)c1cc(-c2csc(-c3ccc(N)cc3Cl)c2)ccn1 ; CC(C)(C)c1cc(-c2csc(-c3ccc(NC(=O)Oc4ccccc4)cc3Cl)c2)ccn1 ; CC(C)(C)OC(=O)N1CCC(N)CC1 ; CC(C)(C)OC(=O)N1CCC(NC(=O)Nc2ccc(-c3cc(-c4ccnc(C(C)(C)C)c4)cs3)c(Cl)c2)CC1 ; CC(C)(C)c1cc(-c2csc(-c3ccc(NC(=O)NC4CCNCC4)cc3Cl)c2)ccn1]

### Step 1: Synthesis of Phenyl (4-(4-(2-(tert-butyl)pyridin-4-yl)thiophen-2-yl)-3-chlorophenyl)carbamate

To a stirred solution of 4-(4-(2-(tert-butyl)pyridin-4-yl)thiophen-2-yl)-3-chloroaniline (0.100 g, 0.292 mmol, 1.0 eq) in THF (5 mL) was added Et₃N (0.059 g, 0.584 mmol, 1.2 eq) at 0° C. and then phenylchloroformate (0.053 g, 0.350 mmol, 1.2 eq). The reaction mixture was stirred at RT for 4h. After completion, ice-cold water (10 mL) was the product was and extracted into EtOAc (3×10 mL). The extracts were combined, dried over anhydrous sodium sulfate and concentrated under reduced pressure to obtain crude phenyl (4-(4-(2-(tert-butyl)pyridin-4-yl)thiophen-2-yl)-3-chlorophenyl)carbamate (0.135 g) as a pale yellow gummy solid which was used for next step without purification.

### Step 2: Synthesis of tert-butyl 4-(3-(4-(4-(2-(tert-butyl)pyridin-4-yl)thiophen-2-yl)-3-chlorophenyl)ureido)piperidine-1-carboxylate

To a stirred solution of tert-butyl 4-aminopiperidine-1-carboxylate (0.084 g, 0.421 mmol, 1.5 eq) in THF (5 mL) was added NaH (0.020 g, 0.421 mmol, 1.5 eq) at 0° C. After 10 min, phenyl (4-(4-(2-(tert-butyl)pyridin-4-yl)thiophen-2-yl)-3-chlorophenyl)carbamate (0.135 g, 0.281 mmol, 1.5 eq) was added at 0° C. and stirred at RT for 4h. After completion, the residue was dissolved in water (10 mL) and extracted with EtOAc (3×10 mL). The extracts were combined, dried over anhydrous sodium sulfate and concentrated under reduced pressure to obtain crude product, which was purified by column chromatography to afford tert-butyl 4-(3-(4-(4-(2-(tert-butyl)pyridin-4-yl)thiophen-2-yl)-3-chlorophenyl)ureido)piperidine-1-carboxylate (0.073 g) as a pale yellow gummy solid.

### Step 3: Synthesis of 1-(4-(4-(2-(tert-butyl)pyridin-4-yl)thiophen-2-yl)-3-chlorophenyl)-3-(piperidin-4-yl)urea

HCl in dioxane (4M) (2.5 mL) was added to tert-butyl 4-(3-(4-(4-(2-(tert-butyl)pyridin-4-yl)thiophen-2-yl)-3-chlorophenyl)ureido)piperidine-1-carboxylate (0.062 g, 0.108 mmol, 1.0 eq) at 0° C. The reaction mixture was stirred at RT for 16h. After completion, the dioxane was removed under reduced pressure to obtain a residue, which was basified with NaHCO₃ solution and extracted with 10% MeOH in DCM (4×10 mL). The extracts were combined, dried over anhydrous sodium sulfate and concentrated under reduced pressure to obtain crude compound, which was purified by prep HPLC. The product fraction was concentrated under reduced pressure to afford 1-(4-(4-(2-(tert-butyl)pyridin-4-yl)thiophen-2-yl)-3-chlorophenyl)-3-(piperidin-4-yl)urea (0.027 g, 52%) as an off-white solid. ¹H NMR (400 MHz, DMSO: δ (ppm): 8.74 (s, 1H), 8.52 (d, J=5.2 Hz, 1H), 8.24 (d, J=1.6 Hz, 1H), 7.89 (d, J=1.6 Hz, 1H), 7.81 (d, J=2.4 Hz, 1H), 7.74 (d, J=0.8 Hz, 1H), 7.61 (d, J=8.4 Hz, 1H), 7.56-7.54 (dd, J=1.6 Hz, J=1.6 Hz, 1H), 7.31-7.28 (dd, J=2.0 Hz, J=2.4 Hz, 1H), 6.32 (d, J=7.2 Hz, 1H), 3.53-3.48 (m, 1H), 2.91-2.88 (m, 2H), 2.50-2.49 (m, 2H), 1.77-1.74 (m, 2H), 1.36 (s, 9H), 1.28-1.23 (m, 2H); LCMS: (469.2[M+H].

### Synthesis Example 8: 1-(4-(4-(2-(tert-butyl)pyridin-4-yl)thiophen-2-yl)-3-chlorophenyl)-3-(2-(pyrrolidin-1-yl)ethyl)urea (Compound I-8761)

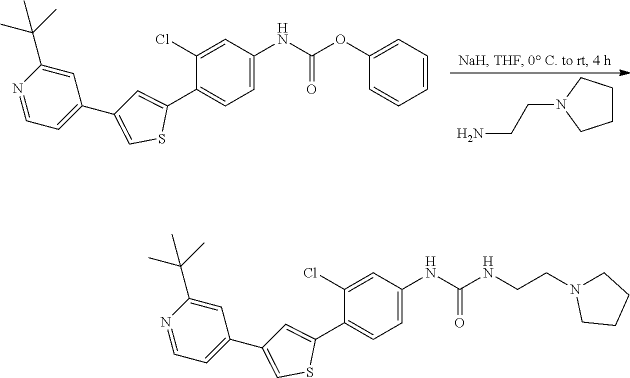 [SMILES: CC(C)(C)c1cc(-c2csc(-c3ccc(NC(=O)Oc4ccccc4)cc3Cl)c2)ccn1 ; NCCN1CCCC1 ; CC(C)(C)c1cc(-c2csc(-c3ccc(NC(=O)NCCN4CCCC4)cc3Cl)c2)ccn1]

To a stirred solution of phenyl (4-(4-(2-(tert-butyl)pyridin-4-yl)thiophen-2-yl)-3-chlorophenyl)carbamate (0.052 g, 0.454 mmol, 1.5 eq) in THF (5 mL) was added NaH (0.021 g, 0.454 mmol, 1.5 eq) at 0° C. After 10 minutes 2-(pyrrolidin-1-yl)ethan-1-amine (0.140 g, 0.303 mmol, 1.5 eq) was added at 0° C. The reaction mixture was stirred at RT for 4h. After completion, water (10 mL) was added and the product extracted into EtOAc (3×10 mL). The extracts were combined, dried over anhydrous sodium sulfate and concentrated under reduced pressure to obtain crude compound, which was purified by prep HPLC. The product fraction was concentrated under reduced pressure to afford 1-(4-(4-(2-(tert-butyl)pyridin-4-yl)thiophen-2-yl)-3-chlorophenyl)-3-(2-(pyrrolidin-1-yl)ethyl)urea (0.038 g, 26%) as an off white solid. ¹H NMR (400 MHz, DMSO: δ (ppm): 9.02 (s, 1H), 8.52-8.51 (dd, J=0.4 Hz, J=0.8 Hz, 1H), 8.24 (d, J=1.6 Hz, 1H), 7.88 (d, J=1.2 Hz, 1H), 7.81 (d, J=2.4 Hz, 1H), 7.73 (d, J=0.8 Hz, 1H), 7.61 (d, J=8.0 Hz, 1H), 7.55-7.54 (dd, J=1.6 Hz, J=1.6 Hz, 1H), 7.31-7.28 (dd, J=2.4 Hz, J=2.0 Hz, 1H), 6.26-6.24 (t, J=5.2 Hz, 1H), 3.23-3.19 (q, 2H), 2.49-2.47 (m, 6H), 1.72-1.69 (m, 4H), 1.36 (s, 9H); LCMS: (483.2[M+H].

### Synthesis Example 9: Isopropyl (4-(4-(2-(tert-butyl)pyridin-4-yl)thiophen-2-yl)-3-chlorophenyl)carbamate (Compound I-8741)

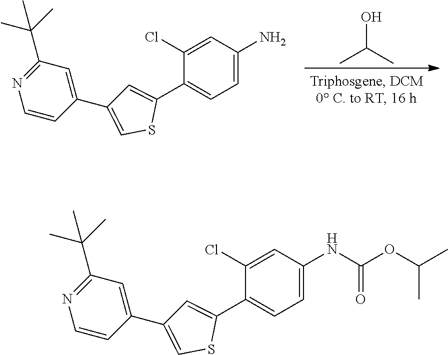

To a stirred solution of 4-(4-(2-(tert-butyl)pyridin-4-yl)thiophen-2-yl)-3-chloroaniline (0.250 g, 0.730 mmol, 1.0 eq) in DCM (5 mL) was added triphosgene (0.216 g, 0.730 mmol, 1.0 eq) at 0° C. After 1h, IPA (0.065 g, 1.096 mmol, 1.5 eq) was added 0° C. Then the reaction mixture was stirred at RT for 16h. After completion, the solvent was evaporated from the reaction mixture, ice-cold water (10 mL) was added and the product was extracted into DCM (3×10 mL). The extracts were combined, dried over anhydrous sodium sulfate and concentrated under reduced pressure to obtain crude compound, which was purified by prep HPLC. The product fraction was concentrated under reduced pressure to afford isopropyl (4-(4-(2-(tert-butyl)pyridin-4-yl)thiophen-2-yl)-3-chlorophenyl)carbamate (0.176 g, 56%) as a white solid. ¹H NMR (400 MHz, DMSO-d₆): δ (ppm): 9.94 (s, 1H), 8.53-8.51 (dd, J=0.4 Hz, J=0.4 Hz, 1H), 8.26 (d, J=0.8 Hz, 1H), 7.91 (d, J=1.2 Hz, 1H), 7.76 (d, J=2.0 Hz, 1H), 7.73 (d, J=0.8 Hz, 1H), 7.68 (d, J=8.4 Hz, 1H), 7.56-7.54 (dd, J=1.6 Hz, J=1.6 Hz, 1H), 7.50-7.48 (dd, J=2.4 Hz, J=2.0 Hz, 1H), 4.95-4.89 (m, 1H), 1.36 (s, 9H), 1.27 (d, J=6.0 Hz, 6H); LCMS: (429.53[M+H]).

### Synthesis Example 10: Ethyl (4-(4-(2-(tert-butyl)pyridin-4-yl)thiophen-2-yl)-3-chlorophenyl)carbamate (Compound I-8735)

This compound was prepared following a procedure similar to Example 9 above. ¹H NMR (400 MHz, DMSO: δ (ppm): 10.0 (s, 1H), 8.53-8.51 (dd, J=0.8 Hz, J=0.8 Hz, 1H), 8.26 (d, J=1.2 Hz, 1H), 7.91 (d, J=1.2 Hz, 1H), 7.76-7.73 (dd, J=2.0 Hz, J=0.8 Hz, 2H), 7.69 (d, J=8.8 Hz, 1H), 7.56-7.54 (dd, J=1.6 Hz, J=1.6 Hz, 1H), 7.51-7.48 (m, 1H), 4.19-4.13 (q, 2H), 1.36 (s, 9H), 1.28-1.24 (t, J=7.2 Hz, 3H); LCMS: (415.45[M+H].

### Synthesis Example 11: 2-(pyrrolidin-1-yl)ethyl (4-(4-(2-(tert-butyl)pyridin-4-yl)thiophen-2-yl)-3-chlorophenyl)carbamate (Compound I-8762)

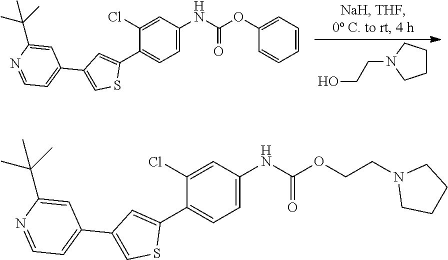 [SMILES: CC(C)(C)c1cc(-c2csc(-c3ccc(NC(=O)Oc4ccccc4)cc3Cl)c2)ccn1 ; OCCN1CCCC1 ; CC(C)(C)c1cc(-c2csc(-c3ccc(NC(=O)OCCN4CCCC4)cc3Cl)c2)ccn1]

To a stirred solution of 2-(pyrrolidin-1-yl)ethan-1-ol (0.056 g, 0.486 mmol, 1.5 eq) in THF (5 mL) was added NaH (60% moisture) (0.023 g, 0.486 mmol, 1.5 eq) at 0° C. After 10 min, phenyl (4-(4-(2-(tert-butyl)pyridin-4-yl)thiophen-2-yl)-3-chlorophenyl)carbamate (0.150 g, 0.324 mmol, 1.5 eq) at 0° C. was added. Then the reaction mixture was stirred at RT for 4h. After completion water (10 mL) was added and the product was extracted into EtOAc (3×10 mL). The extracts were combined, dried over anhydrous sodium sulfate and concentrated under reduced pressure to obtain crude compound, which was purified by prep HPLC. The product fraction was concentrated under reduced pressure to afford 2-(pyrrolidin-1-yl)ethyl (4-(4-(2-(tert-butyl)pyridin-4-yl)thiophen-2-yl)-3-chlorophenyl)carbamate (0.019 g, 16%) as a white solid. ¹H NMR (400 MHz, DMSO: δ (ppm): 10.07 (s, 1H), 8.52 (d, J=4.8 Hz, 1H), 8.26 (d, J=1.6 Hz, 1H), 7.91 (d, J=0.3 Hz, 1H), 7.77-7.68 (m, 3H), 7.56-7.49 (m, 2H), 4.22-4.19 (t, J=1.6 Hz, 2H), 2.70 (bs, 2H), 2.51-2.50 (m, 4H), 1.69 (bs, 4H), 1.36 (s, 9H); LCMS: (484.42[M+H].

### Synthesis Example 12: Preparation of Reagents 2-(4-bromopyridin-2-yl)-2-methylpropan-1-ol and 2-methyl-2-(4-(4,4,5,5-tetramethyl-1,3,2-dioxaborolan-2-yl)pyridin-2-yl)propan-1-ol

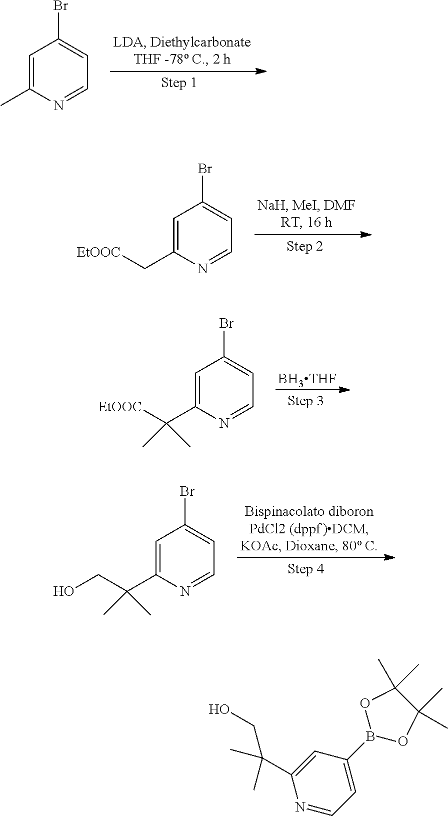

### Step 1: Ethyl 2-(4-bromopyridin-2-yl)acetate

To a stirred solution of 4-bromo-2-methylpyridine (1.2 g, 4.93 mmol, 1.0 eq) in THF (15 mL), was added diethyl carbonate (0.698 mL, 5.92 mmol, 1.2 eq) and the mixture stirred at −78° C. under nitrogen atmosphere. LDA (2M) (2.96 mL, 5.92 mmol) was added dropwise over 15 min and the reaction was stirred at −78° C. for 2h. The reaction was then quenched with saturated sodium chloride solution and the product extracted with EtOAc (2×50 mL). The combined organic layers were washed with brine (50 mL), dried over anhydrous sodium sulfate and concentrated under reduced pressure, to give a crude compound, which was purified by column chromatography to give ethyl 2-(4-bromopyridin-2-yl)acetate (0.500 g, 30%) as a pale yellow liquid.

### Step 2: Ethyl 2-(4-bromopyridin-2-yl)-2-methylpropanoate

To a stirred solution of ethyl 2-(4-bromopyridin-2-yl)acetate (0.5 g, 2.05 mmol, 1.0 eq) in DMF (5 mL) at 0° C. under nitrogen atmosphere was added NaH (60%) (0.246 g, 6.10 mmol, 3 eq). The reaction mass was stirred at RT for 30 min, then cooled to 0° C. followed by the addition of methyl iodide (1.486 g, 10.25 mmol, 5.0 eq) under nitrogen atmosphere and stirred at RT for 16h. The DMF was removed under reduced pressure, water (10 mL) was added and the product extracted into EtOAc (3×15 mL). The combined organic extracts were dried over anhydrous sodium sulfate, concentrated under reduced pressure to give a crude product which was purified by column chromatography to give ethyl 2-(4-bromopyridin-2-yl)-2-methylpropanoate (0.140 g, 25%) as a pale yellow liquid.

### Step 3: 2-(4-bromopyridin-2-yl)-2-methylpropan-1-ol

To a stirred solution of ethyl 2-(4-bromopyridin-2-yl)-2-methylpropanoate (600 mg, 2.2 mmol, 1 eq) in THF (10 mL) under nitrogen atmosphere, at 0° C., was added 1M BH₃. THE (3.3 mL, 3.31 mmol) dropwise. The mixture was stirred at room temperature for 16 h and then quenched with methanol (10 mL) and concentrated under reduced pressure to afford crude product which was diluted with water and extracted into ethyl acetate. The ethyl acetate layer was dried over sodium sulfate and concentrated under reduced pressure to afford 2-(4-bromopyridin-2-yl)-2-methylpropan-1-ol (280 mg) as a pale yellow liquid.

### Step 4: 2-methyl-2-(4-(4,4,5,5-tetramnethyl-1,3,2-dioxaborolan-2-yl)pyridin-2-yl)propan-1-ol

A stirred solution of 2-(4-bromopyridin-2-yl)-2-methylpropan-1-ol (200 mg, 0.8733 mmol), bispinacolato diboron (243.95 mg, 0.9606 mmol) and potassium acetate (257.14 mg, 2.6199) in 1,4-dioxane (4 mL) in 14 ml glass seal tube was purged with nitrogen for 15 min. After adding PdCl₂(dppf).DCM adduct (71 mg, 0.0873 mmol), the reaction tube was again purged with nitrogen gas for 20 min, then sealed and heated at 80° C. for 16 h. After completion of the reaction (monitored by TLC), the mixture was cooled to RT and filtered through a Celite® bed. The bed was washed with ethyl acetate (10 mL), and the combined organic solutions were concentrated under reduced pressure to afford crude product (300 mg). The crude compound was co-distilled with methanol (3×10 mL) and stirred with 5% ethyl acetate in pet ether. The suspension was filtered and the filtrate was evaporated completely to give the desired boronate as a brown gum, which was used without further purification.

### Synthesis Example 13: N-(3-chloro-4-(4-(2-(1-hydroxy-2-methylpropan-2-yl)pyridin-4-yl)thiophen-2-yl)phenyl)methanesulfonamide (Compound I-8909)

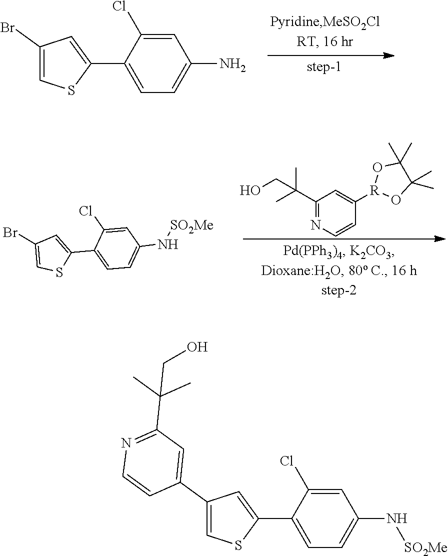

### Step 1: N-(4-(4-bromothiophen-2-yl)-3-chlorophenyl)methanesulfonamide

To a stirred solution of 4-(4-bromothiophen-2-yl)-3-chloroaniline (0.1 g, 0.34 mmol) in pyridine, was added methanesulfonylchloride (0.08 g, 0.696 mmol) at room temperature. The reaction mass was stirred at RT for 16 h. After completion (monitored by TLC), the residue was dissolved in water (10 mL) and the product extracted into EtOAc (3×15 mL). The extracts were combined, dried over anhydrous sodium sulfate and concentrated under reduced pressure. The obtained crude was purified by column chromatography (neutral silica gel), eluted with 15 EtOAc in pet ether to give N-(4-(4-bromothiophen-2-yl)-3-chlorophenyl)methanesulfonamide as a white solid (0.1 g, 79%).

### Step 2: N-(3-chloro-4-(4-(2-(1-hydroxy-2-methylpropan-2-yl)pyridin-4-yl)thiophen-2-yl)phenyl)methanesulfonamide

To a stirred solution of N-(4-(4-bromothiophen-2-yl)-3-chlorophenyl)methanesulfonamide (0.10 g, 0.273 mmol) in dioxane/water (5:1, 12 mL) in a glass tube, was added K₂CO₃ (0.113 g, 0.819 mmol) and 2-methyl-2-(4-(4,4,5,5-tetramethyl-1,3,2-dioxaborolan-2-yl)pyridin-2-yl)propan-1-ol (0.076 g, 0.273 mmol); the reaction mixture was purged with nitrogen gas for 5 min, and then Pd(PPh₃)₄ was added and again purged with nitrogen gas for 5 min. The tube was sealed and heated at 80° C. for 16 hr. After completion (monitored by TLC), the solvent was evaporated and the residue was diluted with ethyl acetate and washed with water (3×10 mL). The organic layer was dried over anhydrous sodium sulfate and concentrated under reduced pressure. The obtained crude was purified by prep HPLC to give N-(3-chloro-4-(4-(2-(1-hydroxy-2-methylpropan-2-yl)pyridin-4-yl)thiophen-2-yl)phenyl)methanesulfonamide as an off-white solid (0.19 g, 40%). ¹H NMR (400 MHz, DMSO-d₆): δ 10.187 (s, 1H), 8.53 (d, J=5.20 Hz, 1H), 8.27 (d, J=1.20 Hz, 1H), 7.91 (d, J=1.60 Hz, 1H), 7.74 (d, J=8.40 Hz, 1H), 7.70 (s, 1H), 7.55 (dd, J=1.60, 5.20 Hz, 1H), 7.38 (d, J=2.40 Hz, 1H), 7.26 (dd, J=2.40, 8.40 Hz, 1H), 4.67 (t, J=5.60 Hz, 1H), 3.60 (d, J=5.20 Hz, 2H), 3.10 (s, 3H), 1.30 (s, 6H); LCMS: 98.4600 (437.32[M+H]).

The following compounds were prepared in a similar fashion:

| | |
|---|---|
| 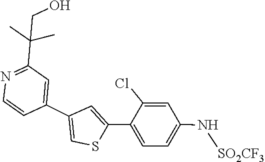 | N-(3-chloro-4-(4-(2-(1-hydroxy-2-methylpropan-2-yl)pyridin-4-yl)thiophen- 2-yl)phenyl)-1,1,1-trifluoromethanesulfonamide (Compound I-8940) ¹H NMR: 400 MHz, DMSO-d6: δ 8.51 (d, J = 5.20 Hz, 1H), 8.18 (s, 1H), 7.82 (s, 1H), 7.70 (s, 1H), 7.55 (d, J = 4.40 Hz, 1H), 7.43 (d, J = 8.40 Hz, 1H), 7.17 (d, J = 2.00 Hz, 1H), 6.97 (dd, J = 2.00, 8.40 Hz, 1H), 4.68 (br s, 1H), 3.60 (s, 2H), 1.30 (s, 6H); LCMS: 98.81% (491.25[M + H]). |
| I-8940 | |
| 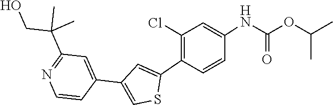 [SMILES: CC(C)OC(=O)Nc1ccc(-c2cc(-c3ccnc(C(C)(C)CO)c3)cs2)c(Cl)c1] | Isopropyl (3-chloro-4-(4-(2-(1-hydroxy-2-methylpropan-2-yl)pyridin-4- yl)thiophen-2-yl)phenyl)carbamate (Compound I-8907) ¹H NMR: 400 MHz, DMSO-d6: δ 9.94 (s, 1H), 8.52 (d, J = 5.20 Hz, 1H), 8.25 (s, 1H), 7.90 (s, 1H), 7.77 (s, 1H), 7.69-7.67 (m, 2H), 7.56 (d, J = 4.80 Hz, 1H), 7.50-7.48 (dd, J = 2.40, 8.40 Hz, 1H), 4.95-4.89 (m, 1H), 4.67 (br s, 1H), 3.60 (s, 2H), 1.30 (s, 6H), 1.27 (d, J = 6.0 Hz, 6H); LCMS: 99.31% (445.0, M + H). |
| I-8907 | |
| 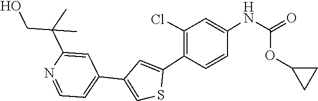 [SMILES: CC(C)(CO)c1cc(-c2csc(-c3ccc(NC(=O)OC4CC4)cc3Cl)c2)ccn1] I-8918 | Cyclopropyl (3-chloro-4-(4-(2-(1-hydroxy-2-methylpropan-2-yl)pyridin- 4-yl)thiophen-2-yl)phenyl)carbamate (Compound I-8918) ¹H NMR: (400 MHz, DMSO-d6): δ 10.05 (s, 1H), 8.52 (d, J = 5.20 Hz, 1H), 8.25 (d, J = 1.20 Hz, 1H), 7.91 (d, J = 1.20 Hz, 1H), 7.75 (d, J = 2.00 Hz, 1H), 7.71-7.67 (m, 2H), 7.57-7.52 (m, 1H), 7.52-7.46 (m, 1H), 4.67 (t, J = 5.60 Hz, 1H), 4.15-4.08 (m, 1H), 3.60 (d, J = 5.20 Hz, 2H), 1.30 (s, 6H), 0.75-0.65 (m, 4H); LCMS: 98.61% (m/z = 443.00[M + H]). |
| 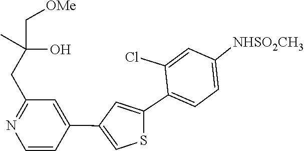 | N-(3-chloro-4-(4-(2-(2-hydroxy-3-methoxy-2-methylpropyl)pyridin-4-yl) thiophen-2-yl)phenyl)methanesulfonamide (Compound I-9129) H¹ NMR (400 MHz, DMSO): δ 10.20 (s, 1H), 8.50 (d, J = 5.2 Hz, 1H), 8.24 (d, J = 1.2 Hz, 1H), 7.88 (d, J = 1.2 Hz, 1H), 7.70 (d, J = 8.4 Hz, 1H), 7.66 (s, 1H), 7.61 (dd, J = 2.4 Hz, 1H), 7.37 (d, J = 2.4 Hz, 1H), 7.25 (q, J = 3.6 Hz, 1H), 4.97 (s, 1H), 3.28 (s, 3H), 3.15 (d, J = 4.4 Hz, 2H), 3.09 (s, 3H), 2.90 (d, J = 4.0 Hz, 2H), 1.05 (s, 3H); LCMS: 99.93% (m/z = 467.70 [M + H])⁺ |
| I-9129 | |
| 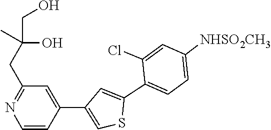 | N-(3-chloro-4-(4-(2-(2,3-dihydroxy-2-methylpropyl)pyridin-4-yl)thiophen-2- yl)phenyl)methanesulfonamide (Compound I-9119 H NMR (400 MHz, DMSO) δ 10.20 (s, 1H), 8.50 (d, J = 5.2 Hz, 1H), 8.25 (d, J = 1.2 Hz, 1H), 7.88 (d, J = 1.2 Hz, 1H), 7.72 (d, J = 8.4 Hz, 1H), 7.68 (s, 1H), 7.61 (dd, J = 1.6, 5.2 Hz, 1H), 7.39 (d, J = 2.4 Hz, 1H), 7.27 (dd, J = 2.4, 8.4 Hz, 1H), 4.79 (s, 1H), 4.71 (t, J = 5.6 Hz, 1H), 3.22-3.20 (m, 2H), 3.10 (s, 3H), 2.90 (d, J = 4.4 Hz, 2H), 1.01 (s, 3H); LCMS: 99.47% (m/z = 451.0 [M − H]) |
| I-9119 | |
| 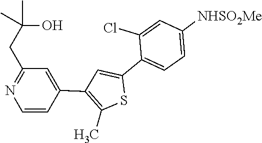 | N-(3-chloro-4-(4-(2-(2-hydroxy-2-methylpropyl)pyridin-4-yl)-5- methylthiophen-2-yl)phenyl)methanesulfonamide (Compound I-9091) ¹H NMR (400 MHz, DMSO) δ 10.16 (s, 1H), 8.53 (d, J = 5.2 Hz, 1H), 7.68 (d, J = 8.4 Hz, 1H), 7.46-7.44 (m, 2H), 7.37-7.36 (m, 2H), 7.25 (dd, J = 2.4, 8.8 Hz, 1H), 4.75 (s, 1H), 3.10 (s, 3H), 2.90 (s, 2H), 2.58 (s, 3H), 1.13 (s, 6H); LCMS: 99.68% ((M + H) 451.42) |
| I-9091 | |
| 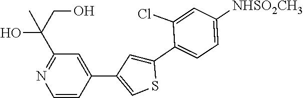 I-9088 | N-(3-chloro-4-(4-(2-(1,2-dihydroxypropan-2-yl)pyridin-4- yl)thiophen-2-yl)phenyl)methanesulfonamide (Compound I- 9088) ¹H NMR (400 MHz, DMSO): δ 10.20 (s, 1H), 8.52 (t, J = 2.8 Hz, 1H), 8.25 (d, J = 1.5 Hz, 1H), 7.95 (d, J = 1.0 Hz, 1H), 7.88 (d, J = 1.5 Hz, 1H), 7.73 (d, J = 8.5 Hz, 1H), 7.61 (q, J = 2.3 Hz, 1H), 7.39 (d, J = 2.3 Hz, 1H), 7.27 (q, J = 3.6 Hz, 1H), 5.16 (s, 1H), 4.63 (t, J = 6.0 Hz, 1H), 3.59 (m, J = 5.1 Hz, 2H), 3.11 (s, 3H), 1.42 (s, 3H); LCMS: 99.05% (m/z = 439.17 [M + H]+) |
| 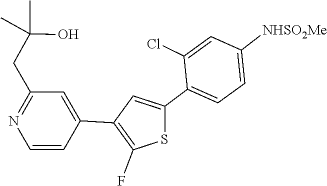 | N-(3-chloro-4-(5-fluoro-4-(2-(2-hydroxy-2-methylpropyl)pyridin- 4-yl)thiophen-2-yl)phenyl)methanesulfonamide (Compound I- 9085) ¹H NMR (400 MHz, DMSO) δ 10.22 (s, 1H), 8.56 (d, J = 5.2 Hz, 1H), 7.74 (d, J = 8.8 Hz, 1H), 7.60-5.8(m, 2H), 7.53 (dd, J = 1.2, 5.2 Hz, 1H), 7.37 (d, J = 2.4 Hz, 1H), 7.27 (dd, J = 2.4, 8.4 Hz, 1H), 4.73 (s, 1H), 3.11 (s, 3H), 2.89 (s, 2H), 1.12 (s, 6H); LCMS: 99.84% ((M + H) 455.22) |
| I-9085 | |
| 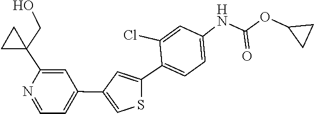 [SMILES: O=C(Nc1ccc(-c2cc(-c3ccnc(C4(CO)CC4)c3)cs2)c(Cl)c1)OC1CC1] | cyclopropyl (3-chloro-4-(4-(2-(1-(hydroxymethyl)cyclopropyl)pyridin-4-yl) thiophen-2-yl)phenyl)carbamate (Compound I-9082) ¹H NMR (400 MHz, DMSO): δ 10.04 (s, 1H), 8.45 (d, J = 5.1 Hz, 1H), 8.23 (d, J = 1.4 Hz, 1H), 7.88 (d, J = 1.5 Hz, 1H), 7.75 (s, 2H), 7.69 (d, J = 8.6 Hz, 1H), 7.49 (m, J = 2.0 Hz, 2H), 4.80 (t, J = 5.6 Hz, 1H), 4.11 (m, J = 3.1 Hz, 1H), 3.83 (d, J = 5.6 Hz, 2H), 1.15 (q, J = 3.3 Hz, 2H), 0.91 (q, J = 3.3 Hz, 2H), 0.72 (m, J = 2.3 Hz, 4H); LCMS: 99.43% (M + H = 414.33) |
| I-9082 | |
| 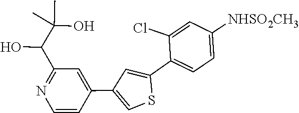 | N-(3-chloro-4-(4-(2-(1,2-dihydroxy-2-methylpropyl)pyridin-4-yl)thiophen-2-yl) phenyl)methanesulfonamide (Compound I-9075) ¹H NMR (400 MHz, DMSO): δ 10.20 (s, 1H), 8.51 (d, J = 5.1 Hz, 1H), 8.25 (d, J = 1.4 Hz, 1H), 7.85 (t, J = 6.1 Hz, 1H), 7.73 (d, J = 8.5 Hz, 1H), 7.66 (q, J = 2.3 Hz, 1H), 7.39 (d, J = 2.2 Hz, 1H), 7.27 (q, J = 3.6 Hz, 1H), 5.43 (d, J = 5.5 Hz, 1H), 4.74 (s, 1H), 4.41 (d, J = 5.5 Hz, 1H), 3.11 (s, 3H), 1.08 (s, 3H), 1.03 (s, 3H); LCMS: 98.15% (m/z = 453.24 [M + H]⁺ |
| I-9075 | |
| 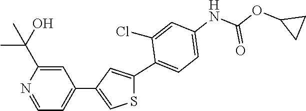 [SMILES: CC(C)(O)c1cc(-c2csc(-c3ccc(NC(=O)OC4CC4)cc3Cl)c2)ccn1] I-9065 | cyclopropyl (3-chloro-4-(4-(2-(2-hydroxypropan-2-yl)pyridin-4- yl)thiophen-2-yl)phenyl)carbamate (Compound I-9065) ¹H NMR (400 MHz, DMSO): δ 10.04 (s, 1H), 8.51 (dd, J = 0.8 Hz and 5.2 Hz, 1H), 8.23 (d, J = 1.2 Hz, 1H), 7.96 (d, J = 1.2 Hz, 1H), 7.87 (d, J = 1.2 Hz, 1H), 7.75 (d, J = 2.0 Hz, 1H), 7.69 (d, J = 8.8 Hz, 1H), 7.59 (dd, J = 1.6 Hz and 5.2 Hz, 1H), 7.49 (dd, J = 2.0 Hz and 8.8 Hz, 1H), 5.27 (s, 1H), 4.11 (m, 1H), 1.48 (s, 6H), 0.72 (m, 4H); LCMS: 99.75% (m/z = 427.28[M + H]). |
| 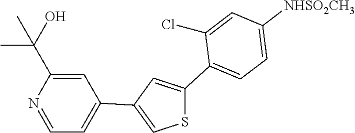 | N-(3-chloro-4-(4-(2-(2-hydroxypropan-2-yl)pyridin-4- yl)thiophen-2-yl)phenyl)methanesulfonamide ¹H NMR (400 MHz, DMSO) δ 10.19 (s, 1H), 8.51 (d, J = 4.8 Hz, 1H), 8.25 (d, J = 1.6 Hz, 1H), 7.97 (d, J = 0.8 Hz, 1H), 7.88 (d, J = 1.6 Hz, 1H), 7.73 (d, J = 8.4 Hz, 1H), 7.59 (dd, J = 2.0 Hz and 5.2 Hz, 1H), 7.39 (d, J = 2.0 Hz, 1H), 7.27 (dd, J = 2.4 Hz and 8.4 Hz, 1H), 5.27 (s, 1H), 3.11 (s, 3H), 1.48 (s, 6H); LCMS: 99.66% (m/z = 423.25[M + H]). |
| I-9064 | |
| 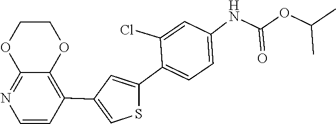 [SMILES: CC(C)OC(=O)Nc1ccc(-c2cc(-c3ccnc4c3OCCO4)cs2)c(Cl)c1] | isopropyl (3-chloro-4-(4-(2,3-dihydro-[1,4]dioxino[2,3-b]pyridin- 8-yl)thiophen-2-yl)phenyl)carbamate (Compound I-9062) ¹H NMR (400 MHz, DMSO) δ 9.93 (s, 1H), 8.13 (d, J = 1.6 Hz, 1H), 7.81 (d, J = 1.6 Hz, 1H), 7.76 (t, J = 2.6 Hz, 2H), 7.62 (d, J = 8.6 Hz, 1H), 7.48 (q, J = 3.6 Hz, 1H), 7.29 (d, J = 5.2 Hz, 1H), 4.92 (m, J = 6.2 Hz, 1H), 4.46 (m, J = 2.0 Hz, 2H), 4.36 (m, J = 2.0 Hz, 2H), 1.27 (d, J = 6.2 Hz, 6H); LCMS: 98.91% (m/z = 429.1 [M + H]) |
| I-9062 | |
| 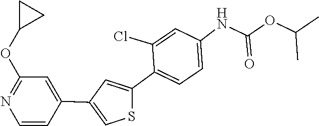 [SMILES: CC(C)OC(=O)Nc1ccc(-c2cc(-c3ccnc(OC4CC4)c3)cs2)c(Cl)c1] | isopropyl (3-chloro-4-(4-(2-cyclopropoxypyridin-4-yl)thiophen- 2-yl)phenyl)carbamate (Compound I-9060) ¹H NMR 400 MHz, DMSO-d₆: δ 9.97 (s, 1H), 8.26 (d, J = 1.60 Hz, 1H), 8.22 (d, J = 5.2 Hz, 1H), 7.89 (d, J = 1.60 Hz, 1H), 7.76 (d, J = 2.00 Hz, 1H), 7.67 (d, J = 8.40 Hz, 1H), 7.48 (dd, J = 2.00 Hz, 8.80 Hz, 1H), 7.41 (dd, J = 1.60 Hz, 5.40 Hz, 1H), 7.22 (d, J = 0.80 Hz, 1H), 4.95-4.88 (m, 1H), 4.29-4.24 (m, 1H), 1.27 (d, J = 6.40 Hz, 6H), 0.79 (m, 2H), 0.69 (t, J = 1.60 Hz, 2H); LCMS: 99.91% (m/z = 429.28 [M + H]) |
| I-9060 | |
| 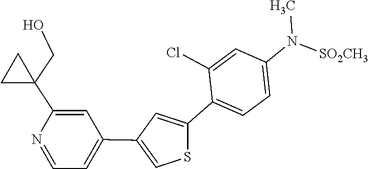 [SMILES: CN(c1ccc(-c2cc(-c3ccnc(C4(CO)CC4)c3)cs2)c(Cl)c1)S(C)(=O)=O] | N-(3-chloro-4-(4-(2-(1-(hydroxymethyl)cyclopropyl)pyridin-4- yl)thiophen-2-yl)phenyl)-N-methylmethanesulfonamide (Compound I-9056) ¹H NMR 400 MHz, DMSO-d6: δ 8.46 (d, J = 5.20 Hz, 1H), 8.30 (d, J = 1.20 Hz, 1H), 7.97 (d, J = 1.60 Hz, 1H), 7.80-7.76 (m, 2H), 7.67 (d, J = 2.40 Hz, 1H), 7.52-7.48 (m, 2H), 4.79 (t, J = 5.60 Hz, 1H), 3.83 (d, J = 5.60 Hz, 2H), 3.30 (s, 3H), 3.04 (s, 3H), 1.16-1.13 (m, 2H), 0.92-0.90 (m, 2H); LCMS: 99.30% (m/z = 449.34 [M + H]) |
| I-9056 | |
| 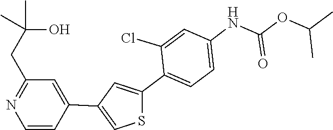 [SMILES: CC(C)OC(=O)Nc1ccc(-c2cc(-c3ccnc(CC(C)(C)O)c3)cs2)c(Cl)c1] | isopropyl (3-chloro-4-(4-(2-(2-hydroxy-2-methylpropyl)pyridin- 4-yl)thiophen-2-yl)phenyl)carbamate (Compound I-9051) ¹H NMR 400 MHz, DMSO-d6: δ 9.94 (s, 1H), 8.50 (d, J = 5.20 Hz, 1H), 8.22 (d, J = 1.20 Hz, 1H), 7.87 (d, J = 1.20 Hz, 1H), 7.81 (d, J = 1.60 Hz, 1H), 7.68-7.65 (m, 2H), 7.54 (d, J = 5.20 Hz, 1H), 7.49 (dd, J = 2.40 Hz, 8.60 Hz, 1H), 4.95-4.89 (m, 1H), 4.82 (s, 1H), 2.88 (s, 2H), 1.28 (d, J = 6.00 Hz, 6H), 1.12 (s, 6H); LCMS: 99.85% (m/z = 445.30[M + H]). |
| I-9051 | |
| 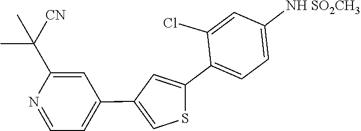 | N-(3-chloro-4-(4-(2-(2-cyanopropan-2-yl)pyridin-4-yl)thiophen- 2-yl)phenyl)methanesulfonamide (Compound I-9039) ¹H NMR 400 MHz, DMSO-d6: δ 8.41 (d, J = 4.80 Hz, 1H), 8.30 (d, J = 1.60 Hz, 1H), 7.99 (d, J = 1.60 Hz, 1H), 7.73 (d, J = 8.40 Hz, 1H), 7.68 (d, J = 1.20 Hz, 1H), 7.61 (d, J = 2.00 Hz, 1H), 7.50 (dd, J = 2.00, 5.20 Hz, 1H), 7.40 (dd, J = 2.40, 8.40 Hz, 1H), 4.07-4.04 (m, 1H), 3.24 (s, 3H), 2.16-2.10 (m, 1H), 0.97- 0.95 (m, 4H), 0.68-0.65 (m, 4H); LCMS: 99.91% (425.26[M + H]⁺ |
| I-9039 | |
| 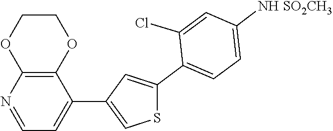 | N-(3-chloro-4-(4-(2,3-dihydro-[1,4]dioxino[2,3-b]pyridin-8- yl)thiophen-2-yl)phenyl)methanesulfonamide (Compound I-9017) ¹H NMR 400 MHz, DMSO-d6: δ 10.20 (s, 1H), 8.16 (d, J = 1.20 Hz, 1H), 7.82 (d, J = 1.20 Hz, 1H), 7.76 (d, J = 5.20 Hz, 1H), 7.68 (d, J = 8.40 Hz, 1H), 7.38 (d, J = 2.00 Hz, 1H), 7.29- 7.24 (m, 2H), 4.47-4.45 (m, 2H), 4.36-4.34 (m, 2H), 3.11 (s, 3H); LCMS: 99.8% (M + H = 423.2) |
| I-9017 | |
| 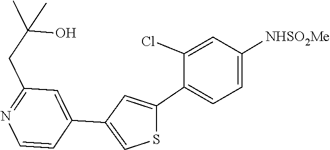 | N-(3-chloro-4-(4-(2-(2-hydroxy-2-methylpropyl)pyridin-4- yl)thiophen-2-yl)phenyl)methanesulfonamide (Compound I-9004) ¹H NMR 400 MHz, DMSO-d6: δ 10.19 (s, 1H), 8.50 (d, J = 5.20 Hz, 1H), 8.25 (d, J = 1.60 Hz, 1H), 7.89 (d, J = 1.60 Hz, 1H), 7.72 (d, J = 8.40 Hz, 1H), 7.66 (br s, 1H), 7.60 (dd, J = 1.60, 5.20 Hz, 1H), 7.39 (d, J = 2.40 Hz, 1H), 7.27 (dd, J = 2.40, 8.40 Hz, 1H), 4.82 (s, 1H), 3.11 (s, 3H), 2.88 (s, 2H), 1.12 (s, 6H); LCMS: 99.36% (M + H = 437.23) |
| I-9004 | |
| 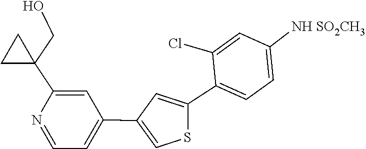 | N-(3-chloro-4-(4-(2-(1-(hydroxymethyl)cyclopropyl)pyridin-4- yl)thiophen-2-yl)phenyl)methanesulfonamide (Compound I-9002) ¹H NMR (400 MHz, DMSO): δ 10.19 (s, 1H), 8.45 (d, J = 5.20 Hz, 1H), 8.25 (d, J = 1.20 Hz, 1H), 7.89 (d, J = 1.60 Hz, 1H), 7.74-7.71 (m, 2H), 7.50 (dd, J = 1.60, 5.20 Hz, 1H), 7.39 (d, J = 2.40 Hz, 1H), 7.27 (dd, J = 2.40, 8.40 Hz, 1H), 4.80 (t, J = 5.60 Hz, 1H), 3.83 (d, J = 5.60 Hz, 2H), 3.10 (s, 3H), 1.14-1.14 (m, 2H), 0.91-0.90 (m, 2H); LCMS: 99.66% ((M − H) 433.0) |
| I-9002 | |

### Synthesis Example 14: N-(3-chloro-4-(4-(2-(1-hydroxy-2-methylpropan-2-yl)pyridin-4-yl)thiophen-2-yl)phenyl)-4-hydroxypiperidine-1-carboxamide (Compound I-8926)

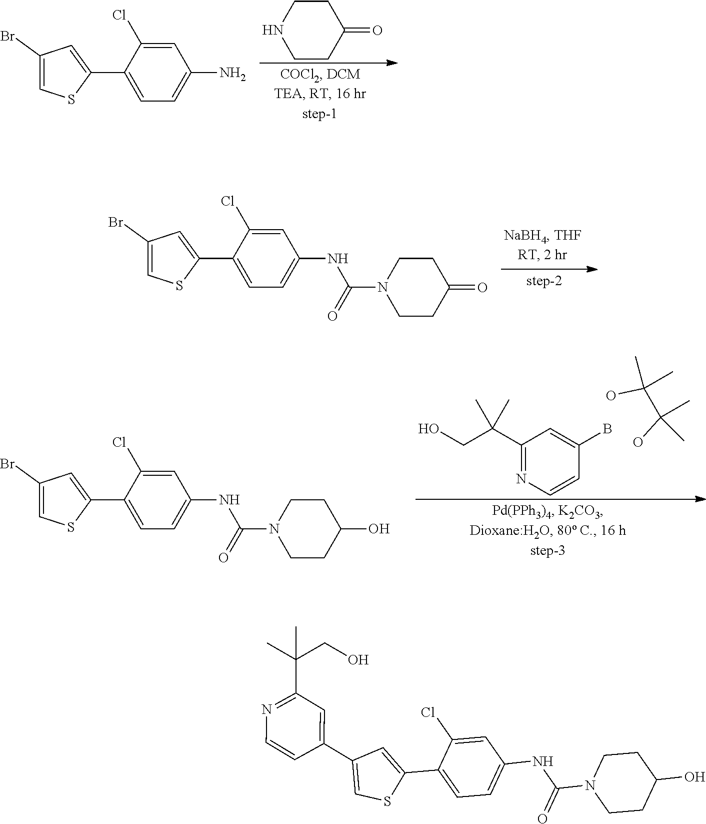

### Step 1: N-(4-(4-bromothiophen-2-yl)-3-chlorophenyl)-4-oxopiperidine-1-carboxamide

To a solution of 4-(4-bromothiophen-2-yl)-3-chloroaniline (0.2 g, 0.696 mmol), 4-piperidone (0.141 g, 1.04 mmol) and TEA (0.21 g, 2.088 mmol) in DCM, was added COCl₂ solution at 0° C. The reaction mixture was stirred at RT for 16 hr. After completion (monitored by TLC), the reaction mixture was quenched with saturated NaHCO₃ and the product extracted into DCM. The extracts were combined, dried over anhydrous sodium sulfate and concentrated under reduced pressure. The crude product was purified by column chromatography on neutral alumina to give N-(4-(4-bromothiophen-2-yl)-3-chlorophenyl)-4-oxopiperidine-1-carboxamide as a pale yellow solid.

### Step 2: N-(4-(4-bromothiophen-2-yl)-3-chlorophenyl)-4-hydroxypiperidine-1-carboxamide

To a stirred solution of N-(4-(4-bromothiophen-2-yl)-3-chlorophenyl)-4-oxopiperidine-1-carboxamide (0.25 g, 0.606 mmol) in THF was added NaBH₄ (0.046, 1.21 mmol) at 0° C. The reaction mass was stirred at RT for 16 h. After completion (monitored by TLC), the solvent was evaporated under reduced pressure and the residue was dissolved in water (10 mL) and extracted with EtOAc (3×15 mL). The extracts were combined, dried over anhydrous sodium sulfate and concentrated under reduced pressure to give crude product which was purified by column chromatography (neutral alumina) to give N-(4-(4-bromothiophen-2-yl)-3-chlorophenyl)-4-hydroxypiperidine-1-carboxamide as a pale yellow solid (0.25 g).

### Step 3: N-(3-chloro-4-(4-(2-(1-hydroxy-2-methylpropan-2-yl)pyridin-4-yl)thiophen-2-yl)phenyl)-4-hydroxypiperidine-1-carboxamide

To a stirred solution of N-(4-(4-bromothiophen-2-yl)-3-chlorophenyl)-4-hydroxypiperidine-1-carboxamide (0.250 g, 0.603 mmol) in dioxane/water (5:1, 12 mL) in a glass tube, was added K₂CO3 (0.250 g, 1.809 mmol) and 2-methyl-2-(4-(4,4,5,5-tetramethyl-1,3,2-dioxaborolan-2-yl)pyridin-2-yl)propan-1-ol (0.167 g, 0.603 mmol) and the reaction mixture was purged with nitrogen for 5 min. Pd(PPh₃)₄ (0.069 g, 0.06 mmol) was added and again purged with nitrogen for 5 min. The reaction tube was sealed and heated at 80° C. for 16 hr. After completion (monitored by TLC), the solvent was evaporated under reduced pressure and the residue was diluted with ethyl acetate (30 mL) and washed with water (10 mL). The organic layer was dried over anhydrous sodium sulfate and concentrated under reduced pressure to give a crude product which was purified by prep HPLC to give N-(3-chloro-4-(4-(2-(1-hydroxy-2-methylpropan-2-yl)pyridin-4-yl)thiophen-2-yl)phenyl)-4-hydroxypiperidine-i-carboxamide as an off-white solid (41 mg). ¹H NMR (400 MHz, DMSO-d₆): δ 8.81 (s, 1H), 8.52 (d, J=5.20 Hz, 1H), 8.23 (d, J=1.60 Hz, 1H), 7.89 (d, J=1.60 Hz, 1H), 7.83 (d, J=2.40 Hz, 1H), 7.70 (s, 1H), 7.64 (d, J=8.4 Hz, 1H), 7.56-7.52 (m, 2H), 4.73 (d, J=4.40 Hz, 1H), 4.67 (t, J=5.20 Hz, 1H), 3.86-3.81 (m, 2H), 3.70-3.67 (m, 1H), 3.60 (d, J=5.20 Hz, 2H), 3.13-3.06 (m, 2H), 1.80-1.70 (m, 2H), 1.45-1.25 (m, 8H): LCMS: 97.78% (486[M+H]).

### Synthesis Example 15: 3-(3-chloro-4-(4-(2-(1-hydroxy-2-methylpropan-2-yl)pyridin-4-yl)thiophen-2-yl)phenyl)oxazolidin-2-one (Compound I-8939)

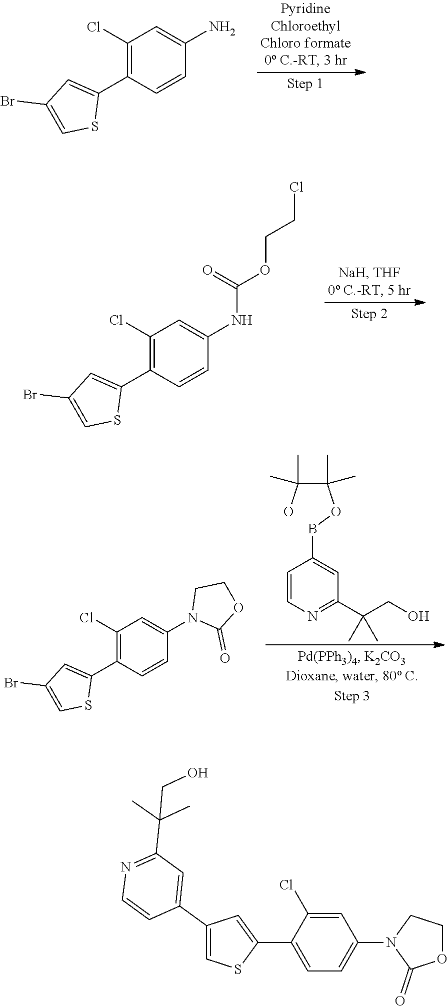

### Step 1: 2-chloroethyl (4-(4-bromothiophen-2-yl)-3-chlorophenyl)carbamate

To a stirred solution of 4-(4-bromothiophen-2-yl)-3-chloroaniline (0.5 g, 1.74 mmol) in pyridine was added chloroethylchloroformate (0.374 g, 2.61 mmol) at room temperature. The reaction mass was stirred at RT for 16 h. After completion (monitored by TLC), the reaction mixture was diluted with water (10 mL) and extracted with EtOAc (3×15 mL). The extracts were combined, dried over anhydrous sodium sulfate and concentrated under reduced pressure. The obtained crude was purified by column chromatography (neutral silica gel), eluting with 20% EtOAc in pet ether, to give 2-chloroethyl (4-(4-bromothiophen-2-yl)-3-chlorophenyl)carbamate as a yellow solid (0.5 g).

### Step 2: 3-(4-(4-bromothiophen-2-yl)-3-chlorophenyl)oxazolidin-2-one

To a stirred solution of 2-chloroethyl (4-(4-bromothiophen-2-yl)-3-chlorophenyl)carbamate (0.5 g, 1.265 mmol) in toluene was added NaH (0.091 g, 3.79 mmol) at 0° C. The reaction mass was stirred at RT for 2 h. After completion (monitored by TLC), the reaction mixture was quenched with ice-cold water and extracted with EtOAc (3×15 mL). The extracts were combined, dried over anhydrous sodium sulfate and concentrated under reduced pressure. The obtained crude was purified by column chromatography (neutral silica gel), eluted with 30% EtOAc in pet ether, to give 3-(4-(4-bromothiophen-2-yl)-3-chlorophenyl)oxazolidin-2-one as a yellow solid (0.2 g).

### Step 3: 3-(3-chloro-4-(4-(2-(1-hydroxy-2-methylpropan-2-yl)pyridin-4-yl)thiophen-2-yl)phenyl)oxazolidin-2-one

To a stirred solution of 3-(4-(4-bromothiophen-2-yl)-3-chlorophenyl)oxazolidin-2-one (0.300 g, 0.83 mmol) in dioxane/water (5:1; 14 mL) in a glass tube was added K₂CO₃ (0.344 g, 2.49 mmol) and 2-methyl-2-(4-(4,4,5,5-tetramethyl-1,3,2-dioxaborolan-2-yl)pyridin-2-yl)propan-1-ol (0.164 g, 0.83 mmol). The reaction mixture was purged with nitrogen for 15 min, Pd(PPh₃)₄ (0.096 g 0.083 mmol) was added, and the mixture again purged with nitrogen for 15 min. The tube was sealed and heated at 80° C. for 16 hr. After completion, the solvent was evaporated under reduced pressure and the residue was diluted with water and extracted with DCM (3×10 mL). The extracts were combined, dried over anhydrous sodium sulfate and concentrated under reduced pressure. The obtained crude compound was purified by prep HPLC to give 3-(3-chloro-4-(4-(2-(1-hydroxy-2-methylpropan-2-yl)pyridin-4-yl)thiophen-2-yl)phenyl)oxazolidin-2-one as an off-white solid (94 mg). ¹H NMR: 400 MHz, DMSO-d6: δ 8.55 (d, J=5.20 Hz, 1H), 8.34 (br s, 1H), 7.99 (s, 1H), 7.87 (d, J=2.00 Hz, 1H), 7.82-7.76 (m, 2H), 7.64-7.58 (m, 2H), 4.70 (s, 1H), 4.49 (t, J=8.40 Hz, 2H), 4.12 (t, J=8.40 Hz, 2H), 3.61 (s, 2H), 1.32 (s, 6H); LCMS: 98.81% (429.31 [M+H]).

### Synthesis Example 16: Compound I-8941

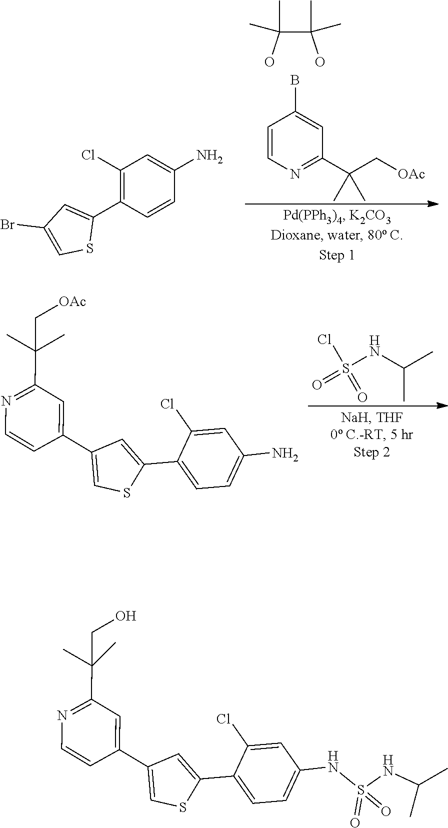

### Step 1: 2-(4-(5-(4-amino-2-chlorophenyl)thiophen-3-yl)pyridin-2-yl)-2-methylpropyl Acetate

To a stirred solution of 4-(4-bromothiophen-2-yl)-3-chloroaniline (0.200 g, 0.734 mmol) in dioxane/water (5:1; 14 mL) in a glass tube, was added K₂CO₃ (0.304 g, 2.2 mmol) and 2-methyl-2-(4-(4,4,5,5-tetramethyl-1,3,2-dioxaborolan-2-yl)pyridin-2-yl)propyl acetate (0.247 g, 0.734 mmol). After purging the reaction mixture with nitrogen for 15 min Pd(PPh₃)₄ (0.085 g 0.073 mmol) was added and the reaction was again purged with nitrogen for 15 min. The tube was sealed and heated at 80° C. for 16 hr. After completion (monitored by TLC), the solvent was evaporated under reduced pressure and the residue was quenched with saturated sodium bicarbonate solution and the product was extracted into DCM (3×10 mL). The combined extracts were dried over anhydrous sodium sulfate and concentrated under reduced pressure to give a crude product which was purified by column chromatography (100-200 silica gel) to give 2-(4-(5-(4-amino-2-chlorophenyl)thiophen-3-yl)pyridin-2-yl)-2-methylpropyl acetate as a brown liquid (25 g, 90%).

### Step 2

To a stirred solution of 2-(4-(5-(4-amino-2-chlorophenyl)thiophen-3-yl)pyridin-2-yl)-2-methylpropyl acetate as (0.230 g, 0.57 mmol) in THF was added NaH (0.041 g, 1.71 mmol) at 0° C. After 30 min at 0° C., isopropylsulfamoyl chloride (0.109 g, 0.68 mmol) was added and the reaction was stirred for a further 2 h at 0° C. The reaction was quenched with ice-cold water and a stirred reaction mixture at RT for 10 min and the product was extracted into extracted into ethyl acetate (3×10 mL). The extracts were combined, dried over anhydrous sodium sulfate and concentrated under reduced pressure to give crude compound which was purified by prep HPLC, to give Compound I-8941 as an off-white solid (0.2 g, 66%). ¹H NMR (400 MHz, DMSO-d6): 10.09 (s, 1H), δ 8.52 (d, J=5.20 Hz, 1H), 8.25 (s, 1H), 7.90 (s, 1H), 7.75-7.65 (m, 3H), 7.55 (dd, J=1.20, 5.20 Hz, 1H), 7.35 (d, J=2.00 Hz, 1H), 7.17 (dd, J=2.00, 8.40 Hz, 1H), 4.67 (t, J=5.20 Hz, 1H), 3.60 (d, J=5.20 Hz, 2H), 3.35 (m, 1H), 1.30 (s, 6H), 1.03 (d, J=6.4 Hz, 6H); LCMS: 98.27% (480.0[M+H]).

The following compounds were prepared in a similar fashion:

| | |
|---|---|
| 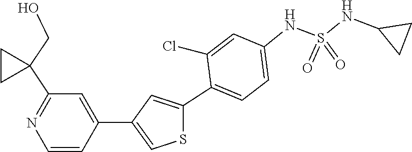 [SMILES: O=S(=O)(Nc1ccc(-c2cc(-c3ccnc(C4(CO)CC4)c3)cs2)c(Cl)c1)NC1CC1] | (1-(4-(5-(2-chloro-4-(((cyclopropylamino)thio)amino)phenyl)thiophen- 3-yl)pyridin-2-yl)cyclopropyhmethanol, S,S-dioxide (Compound I-9090) ¹H NMR (400 MHz, DMSO) δ 10.29 (s, 1H), 8.45 (d, J = 5.2 Hz, 1H), 8.22 (d, J = 1.2 Hz, 1H), 8.13 (s, 1H), 7.87 (d, J = 1.2 Hz, 1H), 7.74 (s, 1H), 7.66 (d, J = 8.8 Hz, 1H), 7.50 (dd, J = 1.6, 5.2 Hz, 1H), 7.35 (d, J = 2.4 Hz, 1H), 7.19 (dd, J = 2.0, 8.4 Hz, 1H), 4.80 (t, J = 5.6 Hz, 1H), 3.83 (d, J = 5.6 Hz, 2H), 2.28 (m, 1H), 1.15-1.13 (m, 2H), 0.91-0.89 (m, 2H), 0.58-0.55 (m, 2H), 0.42 (m, 2H); LCMS: 97.72% (476.26 [M + H]) |
| I-9090 | |

### Synthesis Example 17: Isopropyl (3-chloro-4-(4-(2-cyclopropylpyridin-4-yl)thiophen-2-yl)phenyl)carbamate (Compound I-8966)

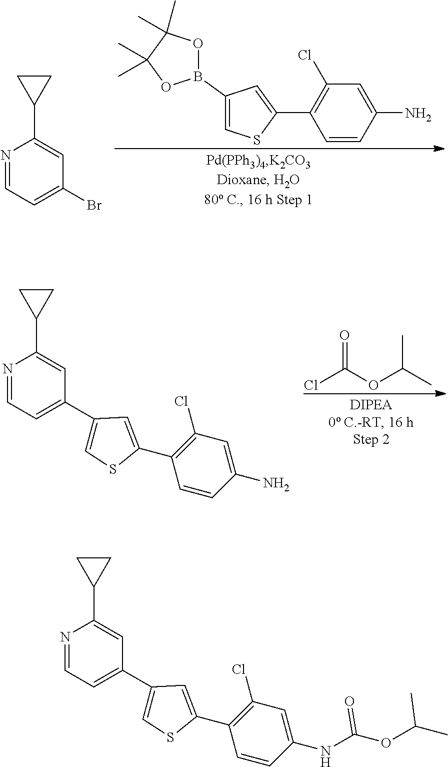

### Step 1: 3-chloro-4-(4-(2-cyclopropylpyridin-4-yl)thiophen-2-yl)aniline

A stirred solution of 4-bromo-2-cyclopropylpyridine (150 mg, 0.75 mmol), 3-chloro-4-(4-(4,4,5,5-tetramethyl-1,3,2-dioxaborolan-2-yl)thiophen-2-yl)aniline (380 mg, 1.13 mmol), and potassium carbonate (314 mg, 2.27 mmol) in 1,4-dioxane (5.6 mL) and water (1.4 mL) was purged with nitrogen for 15 min. After adding palladium Pd(PPh₃)₄ (87 mg, 0.07 mmol) and again purging with nitrogen, the reaction mass was heated at 80° C. for 16 h. After completion of the reaction (monitored by TLC), the reaction mixture was concentrated under reduced pressure to give crude product which was purified by column chromatography (neutral alumina) to give 3-chloro-4-(4-(2-cyclopropylpyridin-4-yl)thiophen-2-yl)aniline (0.15 g, 60%) as a pale yellow gum.

### Step 2: Isopropyl (3-chloro-4-(4-(2-cyclopropylpyridin-4-yl)thiophen-2-yl)phenyl)carbamate

To a stirred solution of 3-chloro-4-(4-(2-cyclopropylpyridin-4-yl)thiophen-2-yl)aniline (150 mg, 0.46 mmol) and DIPEA (0.2 mL) in DCM (5 mL), was added isopropyl carbonochloridate (78 mg, 0.64 mmol) drop wise at 0° C. The reaction mass was slowly allowed to come to RT and stirred for 16 h. After completion of the reaction (monitored by TLC), the reaction was quenched with water (10 mL) and the product was extracted into DCM (3×10 mL), the extracts were combined, dried over anhydrous sodium sulfate and concentrated under reduced pressure to obtain crude product which was purified by prep HPLC to give isopropyl (3-chloro-4-(4-(2-cyclopropylpyridin-4-yl)thiophen-2-yl)phenyl)carbamate as an off-white solid (17 mg). ¹H-NMR (400 MHz, DMSO-d6): δ 0.94 (s, 1H), 8.40 (d, J=4.80 Hz, 1H), 8.24 (d, J=1.20 Hz, 1H), 7.90 (d, J=1.60 Hz, 1H), 7.77 (d, J=2.40 Hz, 1H), 7.68-7.66 (m, 2H), 7.50-7.48 (m, 2H), 4.95-4.87 (m, 1H), 2.16-2.10 (m, 1H), 1.28 (d, J=6.00 Hz, 6H), 0.96 (d, J=6.00 Hz, 4H); LCMS: 98.95% (413.26[M+H]⁺.

### Synthesis Example 18: Isopropyl (3-chloro-4-(4-(2-(1-(hydroxymethyl)cyclopropyl)pyridin-4-yl)thiophen-2-yl)phenyl)carbamate (Compound I-8980)

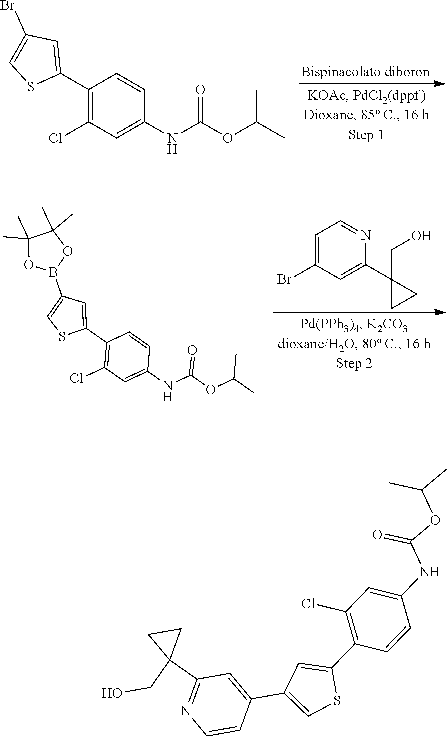

### Step 1: Isopropyl (3-chloro-4-(4-(4,4,5,5-tetramethyl-1,3,2-dioxaborolan-2-yl)thiophen-2-yl)phenyl)carbamate

A stirred solution of isopropyl (4-(4-bromothiophen-2-yl)-3-chlorophenyl)carbamate (250 mg, 0.67 mmol), B₂Pin₂ (254 mg, 1.00 mmol) and potassium acetate (197 mg, 2.00 mmol) in 1,4-dioxane (5 mL) was purged with nitrogen gas for 15 min. After adding palladium Pd(dppf)Cl₄ (48 mg, 0.067 mmol), the reaction mass was heated at 80° C. for 16 h. After completion of the reaction (monitored by TLC), the reaction mixture was filtered through Celite® and the filtrate was concentrated under reduced pressure to give crude isopropyl (3-chloro-4-(4-(4,4,5,5-tetramethyl-1,3,2-dioxaborolan-2-yl)thiophen-2-yl)phenyl)carbamate (370 mg) as a brown gum which was used in next step without purification.

### Step 2: Isopropyl (3-chloro-4-(4-(2-(1-(hydroxymethyl)cyclopropyl)pyridin-4-yl)thiophen-2-yl)phenyl)carbamate

A stirred solution of isopropyl (3-chloro-4-(4-(4,4,5,5-tetramethyl-1,3,2-dioxaborolan-2-yl)thiophen-2-yl)phenyl)carbamate (370 mg, 0.88 mmol), (1-(4-bromopyridin-2-yl)cyclopropyl)methanol (200 mg, 0.88 mmol) and potassium carbonate (366 mg, 2.65 mmol) in 1,4-dioxane (8 mL) and water (2 mL) was purged with nitrogen gas for 15 minutes. After adding palladium Pd(PPh₃)₄ (102 mg, 0.088 mmol), the reaction mixture was purged again with nitrogen and then heated at 80° C. for 16 h. After completion of the reaction (monitored by TLC), the reaction mixture was filtered through Celite® and the filtrate was concentrated under reduced pressure to give crude compound which was purified by prep HPLC to give isopropyl (3-chloro-4-(4-(2-(1-(hydroxymethyl)cyclopropyl)pyridin-4-yl)thiophen-2-yl)phenyl)carbamate (22 mg, 17%) as an off white solid. 1H NMR (400 MHz, DMSO-d6): δ 9.94 (s, 1H), 8.44 (dd, J=0.40, 5.20 Hz, 1H), 8.22 (d, J=1.60 Hz, 1H), 7.88 (d, J=1.60 Hz, 1H), 7.77-7.74 (m, 2H), 7.67 (d, J=8.80 Hz, 1H), 7.51-7.48 (m, 2H), 4.95-4.85 (m, 1H), 4.80 (t, J=5.60 Hz, 1H), 3.83 (d, J=5.60 Hz, 1H), 1.28 (d, J=6.00 Hz, 6H), 1.15-1.14 (m, 2H), 0.91-0.90 (m, 2H); LCMS: 99.67% ((M+H) 443.29).

### Synthesis Example 19: 3-chloro-4-(4-(2-((tetrahydro-2H-pyran-3-yl)oxy)pyridin-4-yl)thiophen-2-yl)aniline (Compound I-9107)

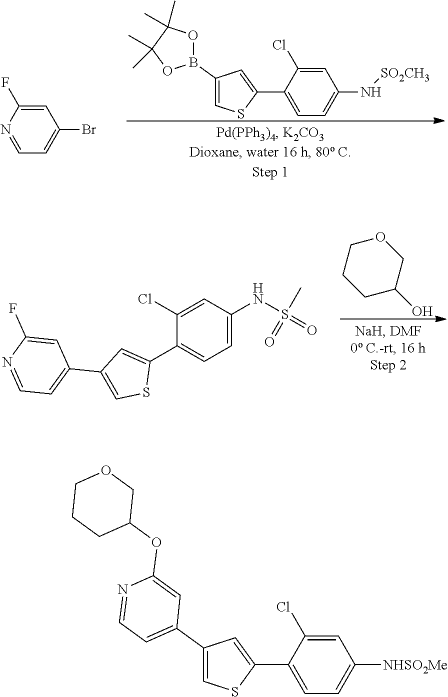

### Step 1: N-(3-chloro-4-(4-(2-fluoropyridin-4-yl)thiophen-2-yl)phenyl)methanesulfonamide

To a stirred solution of 4-bromo-2-fluoropyrdidine (1.5 g, 0.008 mol) in 1,4-dioxane:H₂O (30 mL) was added N-(3-chloro-4-(4-(4,4,5,5-tetramethyl-1,3,2-dioxaborolan-2-yl)thiophen-2-yl)phenyl)methanesulfonamide (5.28 g, 0.0012 mol) and K₂CO₃ (3.312 g, 0.024 mmol). The reaction mixture was purged with N₂, Pd(PPh₃)₄ (0.924 g, 0.0008 mmol) was added and then heated at 80° C. while stirring for 16 h. After completion of the reaction (TLC and LCMS), the mixture was cooled to RT, filtered through a bed of celite and concentrated under reduced pressure to afford crude product which was purified by column chromatography to give N-(3-chloro-4-(4-(2-fluoropyridin-4-yl)thiophen-2-yl)phenyl)methanesulfonamide as a brown solid (1.3 g, 60%); LCMS: 70% (m/z=383.20 [M+H]B).

### Step 2: 3-chloro-4-(4-(2-((tetrahydro-2H-pyran-3-yl)oxy)pyridin-4-yl)thiophen-2-yl)aniline

A mixture of the product of Step 1 (300 mg, 0.783 mmol) and tetrahydro-2H-pyran-3-ol (0.32 g, 3.1 mmol) in DMF (5 mL) was cooled to 0-5° C. NaH (75.16 mg, 3.132 mmol) was added slowly and the reaction mass was stirred at RT for 16 h. After completion of the reaction (TLC and LCMS), the mixture was quenched with ice and the product was extracted into DCM. The organic layer was washed with water, dried over sodium sulfate and concentrated under reduced pressure to afford a crude product which was purified by prep HPLC to give 3-chloro-4-(4-(2-((tetrahydro-2H-pyran-3-yl)oxy)pyridin-4-yl)thiophen-2-yl)aniline as an off white solid (20 mg, 8%). ¹H NMR (400 MHz, DMSO): δ 10.19 (s, 1H), 8.31 (d, J=1.6 Hz, 1H), 8.17 (d, J=5.6 Hz, 1H), 7.93 (d, J=1.6 Hz, 1H), 7.74 (d, J=8.4 Hz, 1H), 7.38-7.36 (m, 2H), 7.26-7.24 (m, 1H), 7.21 (d, J=0.8 Hz, 1H), 5.05-5.02 (m, 1H), 3.89-3.85 (m, 1H), 3.66-3.62 (m, 1H), 3.56-3.49 (m, 2H), 3.10 (s, 3H), 2.07-2.03 (m, 1H), 1.81-1.75 (m, 2H), 1.57-1.54 (m, 1H); LCMS: 98.25% (m/z=465.25 [M+H]⁺).

The following compounds were prepared in a similar fashion:

| | |
|---|---|
| 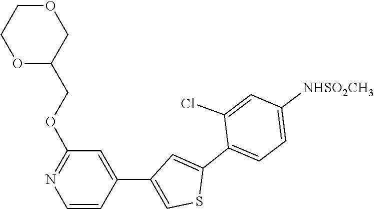 | N-(4-(4-(2-((1,4-dioxan-2-yl)methoxy)pyridin-4- yl)thiophen-2- yl)-3-chlorophenyl)methanesulfonamide (Compound I-9113) ¹H NMR (400 MHz, DMSO) δ 10.18 (s, 1H), 8.28 (d, J = 1.2 Hz, 1H), 8.18 (d, J = 5.6 Hz, 1H), 7.90 (d, J = 1.2 Hz, 1H), 7.70 (d, J = 8.4 Hz, 1H), 7.38-7.35 (m, 2H), 7.22 (dd, J = 2.0, 8.4 Hz, 1H), 7.18 (s, 1H), 5.77 (s, 1H), 4.50-4.45 (m, 4H), 4.41 (t, J = 6.8 Hz, 2H), 3.07 (s, 3H), 2.20 (s, 2H); LCMS: 99.88% (m/z = 481.22[M + H]) |
| I-9113 | |
| 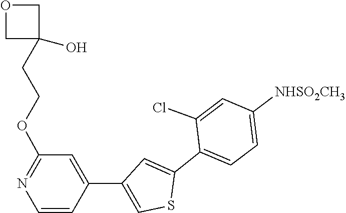 | N-(3-chloro-4-(4-(2-(2-(3-hydroxyoxetan-3-yl)ethoxy)pyridin-4- yl)thiophen-2-yl)phenyl)methanesulfonamide (Compound I- 9112) ¹H NMR (400 MHz, DMSO) δ 10.18 (s, 1H), 8.31 (d, J = 1.6 Hz, 1H), 8.17 (d, J = 5.2 Hz, 1H), 7.93 (d, J = 1.6 Hz, 1H), 7.72(d, J = 8.4 Hz, 1H), 7.40 (dd, J = 1.6, 5.6 Hz, 1H), 7.37 (d, J = 2.0 Hz, 1H), 7.25 (s, 1H), 7.23 (d, J = 2.4 Hz, 1H), 4.30- 4.23 (m, 2H), 3.89-3.86 (m, J = 2.0 Hz, 1H), 3.87-3.80 (m, 1H), 3.78-3.75 (m, 1H), 3.68-3.61 (m, 1H), 3.61-3.58 (q, J = 4.4 Hz, 1H), 3.53-3.47 (m, 1H), 3.43-3.38 (m, 1H), 3.09 (s, 3H); LCMS: 98.57% (m/z = 481.37[M + H]) |
| I-9112 | |
| 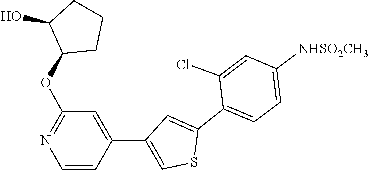 | N-(3-chloro-4-(4-(2-(((1R,2S)-2- hydroxycyclopentyl)oxy)pyridin-4-yl)thiophen-2- yl)phenyl)methanesulfonamide ¹H NMR (401 MHz, DMSO) δ 10.19 (s, 1H), 8.28 (d, J = 1.2 Hz, 1H), 8.15 (d, J = 5.2 Hz, 1H), 7.91 (d, J = 1.2 Hz, 1H), 7.74 (d, J = 8.4 Hz, 1H), 7.38 (d, J = 2.0 Hz, 1H), 7.34 (q, J = 2.4 Hz, 1H), 7.26 (q, J = 3.6 Hz, 1H), 7.18 (s, 1H), 5.14 (m, J = 3.6 Hz, 1H), 4.54 (d, J = 4.8 Hz, 1H), 4.17 (m, 1H), 3.11 (s, 3H), 1.97 (m, 1H), 1.79 (m, 3H), 1.65 (m, 1H), 1.52 (m, 1H); LCMS: 98.77% (m/z = 465.22 [M + H]) |
| I-9111 | |
| 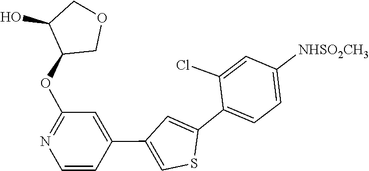 | N-(3-chloro-4-(4-(2-(((3R,4S)-4-hydroxytetrahydrofuran-3- yl)oxy)pyridin-4-yl)thiophen-2-yl)phenyl)methanesulfonamide (Compound I-9108) ¹H NMR (401 MHz, DMSO) δ 10.20 (s, 1H), 8.30 (d, J = 1.2 Hz, 1H), 8.16 (d, J = 5.2 Hz, 1H), 7.92 (s, 1H), 7.72 (d, J = 8.4 Hz, 1H), 7.39-7.36 (m,, 2H), 7.24-7.21 (m, 2H), 5.38-5.34 (m, 1H), 5.05 (d, J = 5.2 Hz, 1H), 4.43-4.38 (m, 1H), 4.09-4.05 (m, 1H), 3.94-3.90 (m, 1H), 3.76-3.72(m, 1H), 3.62-3.59 (m, 1H), 3.08 (s, 3H); LCMS: 96.86% (m/z = 467.23 [M + H]) |
| I-9108 | |

### Synthesis Example 20: (R)-1-(2-(4-(5-(2-chloro-4-((N-isopropylsulfamoyl)amino)phenyl)thiophen-3-yl)pyridin-2-yl)-2-methylpropoxy)-3-methyl-1-oxobutan-2-aminium chloride (I-9094)

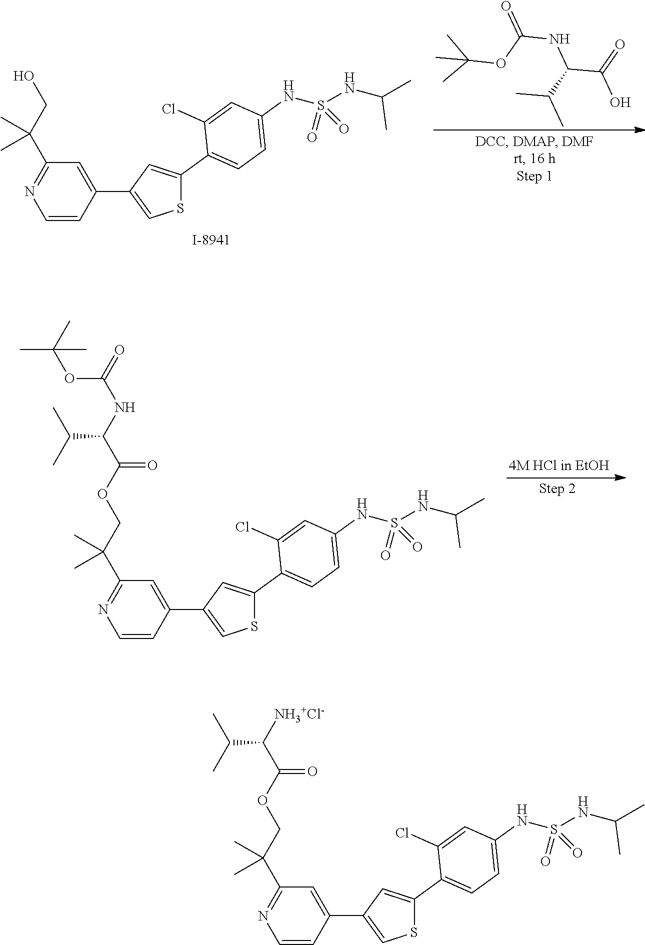

### Step 1: 2-(4-(5-(2-chloro-4-((N-isopropylsulfamoyl)amino)phenyl)thiophen-3-yl)pyridin-2-yl)-2-methylpropyl (tert-butoxycarbonyl)-L-valinate

To a mixture of I-8941 (250 mg, 0.52 mmol), (tert-butoxycarbonyl)-L-valine (169 mg, 0.78 mmol) and DCC (161 mg, 0.78 mmol) in DMF (5 mL) was added DMAP (6 mg, 0.05 mmol). The reaction was stirred at rt for 16 h and then diluted with ethyl acetate (30 mL) and washed with cold water (3×20 mL). The organic layer was dried over anhydrous over sodium sulfate and concentrated under reduced pressure to give a crude product which was purified by reverse phase column chromatography to give 2-(4-(5-(2-chloro-4-((N-isopropylsulfamoyl)amino)phenyl)thiophen-3-yl)pyridin-2-yl)-2-methylpropyl(tert-butoxycarbonyl)-L-valinate (200 mg; 57%) as an off white solid. LCMS: 80.57% (m/z=678.1 [M−H])⁺

### Step 2: (R)-1-(2-(4-(5-(2-chloro-4-((N-isopropyisulfamoyl)amino)phenyl)thiophen-3-yl)pyridin-2-yl)-2-methylpropoxy)-3-methyl-1-oxobutan-2-aminium Chloride

To a solution of the product of Step 1 (200 mg, 0.29 mmol) in EtOH, was added 4M HCl in EtOH (4 mL) drop wise at 0° C. The reaction was stirred at rt for 3 h and then concentrated under reduced pressure to give a crude product which was purified by prep HPLC (0.01 M % HCL in water and ACN) to give (R)-1-(2-(4-(5-(2-chloro-4-((N-isopropylsulfamoyl)amino)phenyl)thiophen-3-yl)pyridin-2-yl)-2-methylpropoxy)-3-methyl-1-oxobutan-2-aminium chloride (50 mg; 28%) as an off white solid. ¹H NMR (401 MHz, DMSO) δ 10.13 (s, 1H), 8.62 (d, J=4.8 Hz, 1H), 8.46-8.35 (m, 4H), 8.01-7.82 (m, 3H), 7.74 (d, J=7.6 Hz, 1H), 7.68 (d, J=8.4 Hz, 1H) 7.36 (d, J=2.4 Hz, 1H), 7.17 (m, J=20.4 Hz, 1H), 4.62 (d, J=10.6 Hz, 1H), 4.39 (d, J=10.6 Hz, 1H), 3.85 (t, J=4.8 Hz, 1H), 3.41-3.32 (m, 1H), 2.01 (m, J=4.6 Hz, 1H), 1.50-1.46 (m, 6H), 1.03 (d, J=6.8 Hz, 6H), 0.76 (t, J=6.4 Hz, 6H); LCMS: 99.85% (579.43[M+H]⁺ ion present.

The following compounds were prepared in a similar fashion:

| | |
|---|---|
| 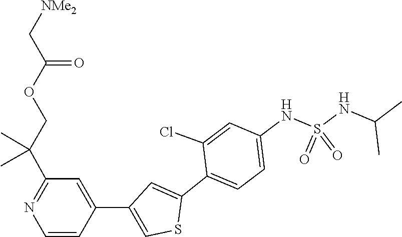 [SMILES: CC(C)NS(=O)(=O)Nc1ccc(-c2cc(-c3ccnc(C(C)(C)COC(=O)CN(C)C)c3)cs2)c(Cl)c1] | 2-(4-(5-(2-chloro-4-((N- isopropylsulfamoyl)amino)phenyl)thiophen-3-yl)pyridin- 2-yl)-2-methylpropyl dimethylglycinate hydrochloride (Compound I- 9095, isolated as the HCl salt) ¹H NMR (400 MHz, DMSO) δ 10.13 (s, 2H), 8.61 (d, J = 5.2 Hz, 1H), 8.45 (s, 1H), 8.01 (s, 1H), 7.92 (s, 1H), 7.80 (s, 1H), 7.74 (d, J = 7.6 Hz, 1H), 7.68 (d, J = 8.4 Hz, 1H), 7.36 (d, J = 2.0 Hz, 1H), 7.18 (dd, J = 2.4, 8.8 Hz, 1H), 4.52 (s, 2H), 4.17 (s, 2H), 3.41-3.34 (m, 1H), 2.76 (s, 6H), 1.46 (s, 6H), 1.03 (d, J = 6.4 Hz, 6H); LCMS: 99.59% (m/z = 565.35 [M + H]) ⁺ |
| I-9095 | |
| 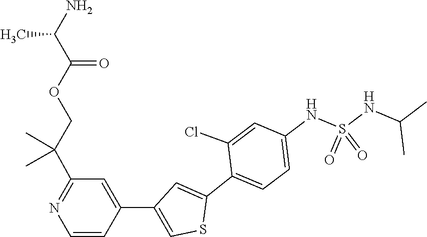 [SMILES: CC(C)NS(=O)(=O)Nc1ccc(-c2cc(-c3ccnc(C(C)(C)COC(=O)[C@H](C)N)c3)cs2)c(Cl)c1] | 2-(4-(5-(2-chloro-4-((N- isopropylsulfamoyl)amino)phenyl)thiophen-3-yl)pyridin- 2-yl)-2-methylpropyl L-alaninate hydrochloride (Compound I-9093, isolated as the HCl salt) ¹H NMR (401 MHz, DMSO) δ 10.15 (s, 1H), 8.66-8.56 (m, 2H), 8.40 (brs, 3H), 8.07-7.94 (m, 3H), 7.75 (d, J = 7.6 Hz, 1H), 7.68 (d, J = 8.4 Hz, 1H), 7.36 (d, J = 2.4 Hz, 1H), 7.19 (dd, J = 2.0, 8.4 Hz, 1H), 4.57 (d, J = 10.6 Hz, 1H), 4.41 (d, J = 10.6 Hz, 1H), 4.05-4.03 (m, 1H), 3.39-3.36 (m, 1H), 1.51-1.49 (m, 6H), 1.26 (d, J = 7.2 Hz, 3H), 1.04-1.02 (m, 6H). |
| I-9093 | |
| 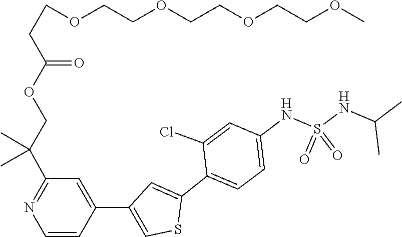 [SMILES: COCCOCCOCCOCCC(=O)OCC(C)(C)c1cc(-c2csc(-c3ccc(NS(=O)(=O)NC(C)C)cc3Cl)c2)ccn1] | 2-(4-(5-(2-chloro-4-((N- isopropylsulfamoyl)amino)phenyl)thiophen-3-yl)pyridin-2- yl)-2-methylpropyl 2,5,8,11-tetraoxatetradecan-14-oate (Compound I-9104) ¹H NMR (401 MHz, DMSO) δ 10.10 (s, 1H), 8.55 (d, J = 4.8 Hz, 1H), 8.29 (d, J = 1.2 Hz, 1H), 7.93 (d, J = 1.6 Hz, 1H), 7.63 (s, 2H), 7.67 (d, J = 7.6 Hz, 1H), 7.61-759 (m, 1H), 7.35 (d, J = 2.4 Hz, 1H), 7.17 (dd, J = 2.0 Hz, 8.4 Hz, 1H), 4.31 (s, 2H), 3.53-3.20 (m, 15H), 3.21 (s, 3H), 2.50-2.44 (m, 2H), 1.37 (s, 6H), 1.03 (d, J = 6.5 Hz, 6H); LCMS: 96.36% (M + H = 696.04) |
| I-9104 | |

### Synthesis Example 21: N-(4-(4-(2-(1-amino-2-methylpropan-2-yl)pyridin-4-yl)thiophen-2-yl)-3-chlorophenyl)methanesulfonamide (I-9049)

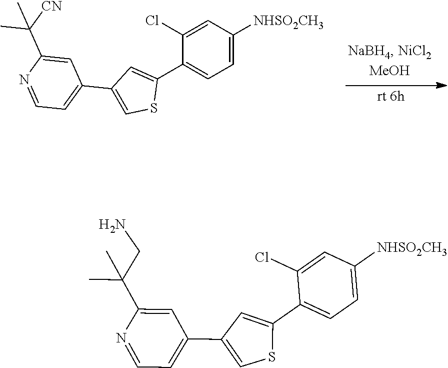

Nickel (II) chloride hexahydrate (172 mg, 0.7218 mmol) was added to a stirred solution of N-(3-chloro-4-(4-(2-(2-cyanopropan-2-yl)pyridin-4-yl)thiophen-2-yl)phenyl)methanesulfonamide (260 mg, 0.6015 mmol) in methanol at 0-5° C. Sodium borohydride (68.27 mg, 1.8045 mmol) was added portion wise over 40 mins and the reaction mixture was stirred for 6 h at RT. The reaction mixture was then filtered through a bed of celite and concentrated under reduced pressure to afford a crude product which was purified by prep H-PLC to give N-(4-(4-(2-(1-amino-2-methylpropan-2-yl)pyridin-4-yl)thiophen-2-yl)-3-chlorophenyl)methanesulfonamide (25 mg, 13.5%) as an off-white solid. ¹H NMR 400 MHz, DMSO-d6: δ 8.53 (d, J=5.20 Hz, 1H), 8.19 (d, J=1.60 Hz, 1H), 7.82 (d, J=1.60 Hz, 1H), 7.71 (s, 1H), 7.57 (dd, J=1.60, 5.20 Hz, 1H), 7.49 (d, J=8.40 Hz, 1H), 7.14 (d, J=2.40 Hz, 1H), 6.96 (dd, J=2.40, 8.60 Hz, 1H), 4.92 (br s, 2H), 2.90 (s, 2H), 2.81 (s, 3H), 1.32 (s, 6H); LCMS: 96.31% (m/z=436.00 [M+H]).

### Synthesis Example 22: Cyclopropyl (3-chloro-4-(4-(2-(1-(hydroxymethyl)cyclopropyl)pyridin-4-yl)thiophen-2-yl)phenyl)(methyl)carbamate (I-9042)

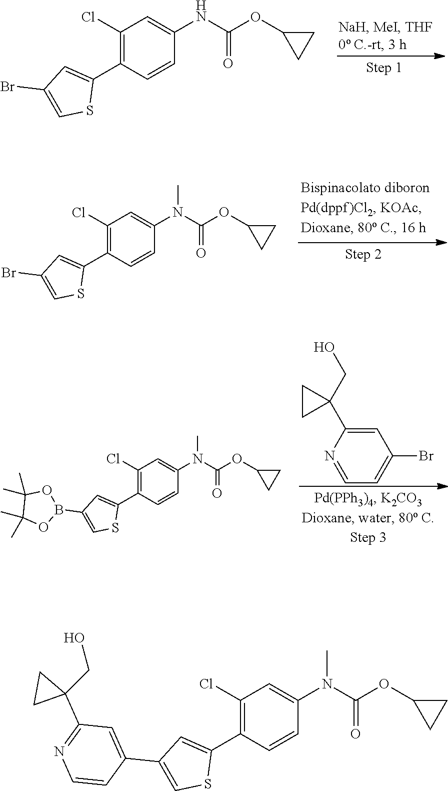 [SMILES: O=C(Nc1ccc(-c2cc(Br)cs2)c(Cl)c1)OC1CC1 ; CN(C(=O)OC1CC1)c1ccc(-c2cc(Br)cs2)c(Cl)c1 ; CN(C(=O)OC1CC1)c1ccc(-c2cc(B3OC(C)(C)C(C)(C)O3)cs2)c(Cl)c1 ; OCC1(c2cc(Br)ccn2)CC1 ; CN(C(=O)OC1CC1)c1ccc(-c2cc(-c3ccnc(C4(CO)CC4)c3)cs2)c(Cl)c1]

### Step 1: Cyclopropyl (4-(4-bromothiophen-2-yl)-3-chlorophenyl)(methyl)carbamate

To a stirred solution of cyclopropyl (4-(4-bromothiophen-2-yl)-3-chlorophenyl)carbamate (300 mg, 0.80 mmol) in THF (10 mL) at 0° C. was added NaH (64 mg, 1.61 mmol) and stirring was continued for 15 min. Mel (171 mg, 1.21 mmol) was added to the reaction drop wise and stirred at RT for 3 h. The reaction was quenched with ice-cold water (30 mL) and the product extracted into EtOAc (3×20 mL). The combined extracts were dried over anhydrous sodium sulfate and concentrated under reduced pressure to afford cyclopropyl (4-(4-bromothiophen-2-yl)-3-chlorophenyl)(methyl)carbamate (300 mg crude) which was used in the next step without purification.

### Step 2: Cyclopropyl (3-chloro-4-(4-(4,4,5,5-tetramethyl-1,3,2-dioxaborolan-2-yl)thiophen-2-yl)phenyl)(methyl)carbamate

A stirred solution of the product of Step 1 (300 mg, 0.77 mmol), bis(pinacolato-diboron) (296 mg, 1.16 mmol) and potassium acetate (230 mg, 2.33 mmol) in 1,4-dioxane (6 mL) was purged with nitrogen gas for 5 minutes. After adding PdCl₂(dppf) (57 mg, 0.07 mmol) the reaction was then heated to 80° C. for 16h. The reaction mixture was cooled to RT, filtered through a bed of celite and concentrated under reduced pressure to afford cyclopropyl (3-chloro-4-(4-(4,4,5,5-tetramethyl-1,3,2-dioxaborolan-2-yl)thiophen-2-yl)phenyl)(methyl)carbamate (800 mg crude) which was used in the next step without purification.

Step 3: Cyclopropyl (3-chloro-4-(4-(2-(1-(hydroxymethyl)cyclopropyl)pyridin-4-yl)thiophen-2-yl)phenyl)(methyl)carbamate. A solution of the product of Step 2 (150 mg, 0.66 mmol), (1-(4-bromopyridin-2-yl)cyclopropyl)methanol (343 mg, 0.79 mmol), K₂CO₃ (273 mg, 1.98 mmol) in 1,4-dioxane: water (8 mL:2 mL) was purged with nitrogen gas for 5 minutes. Tetrakis palladium (76 mg, 0.06 mmol) was then added and the reaction mixture was stirred at 80° C. for 16 h. The mixture was filtered through a bed of celite and concentrated under reduced pressure to obtain a crude product which was purified by prep HPLC to give cyclopropyl (3-chloro-4-(4-(2-(1-(hydroxymethyl)cyclopropyl)pyridin-4-yl)thiophen-2-yl)phenyl)(methyl)carbamate (0.045 g; 15%) as an off white solid. ¹H NMR 400 MHz, DMSO-d6: δ 8.45 (d, J=5.20 Hz, 1H), 8.28 (d, J=1.60 Hz, 1H), 7.96 (d, J=1.60 Hz, 1H), 7.75-7.72 (m, 2H), 7.61 (d, J=2.00 Hz, 1H), 7.51 (dd, J=1.60, 5.20 Hz, 1H), 7.40 (dd, J=2.40, 8.40 Hz, 1H), 4.80 (t, J=5.60 Hz, 1H), 4.07-4.04 (m, 1H), 3.83 (d, J=5.60 Hz, 2H), 3.24 (s, 3H), 1.16-1.13 (m, 2H), 0.92-0.90 (m, 2H), 0.68-0.66 (m, 4H); LCMS: 99.77% (m/z=455.34 [M+H]).

### Synthesis Example 23: N-(3-chloro-4-(4-(2-cyclopropylpyridin-4-yl)thiophen-2-yl)phenyl)-N-methylmethanesulfonamide (I-9037)

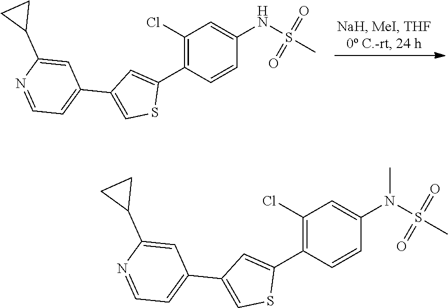 [SMILES: CS(=O)(=O)Nc1ccc(-c2cc(-c3ccnc(C4CC4)c3)cs2)c(Cl)c1 ; CN(c1ccc(-c2cc(-c3ccnc(C4CC4)c3)cs2)c(Cl)c1)S(C)(=O)=O]

To a stirred solution of N-(3-chloro-4-(4-(2-cyclopropylpyridin-4-yl)thiophen-2-yl)phenyl)methanesulfonamide (200 mg, 0.49 mmol) in THF (5 mL) at 0° C. was added NaH (40 mg, 0.99 mmol). Mel (104 mg, 0.74 mmol) was then added drop wise and stirring continued at rt for 24 h. The reaction was cooled to 0° C. and quenched by the addition of ice water and the product was extracted into EtOAc (3×20 mL). The combined extracts were dried over anhydrous sodium sulfate and concentrated under reduced pressure to give a crude product which was purified by prep HPLC to give N-(3-chloro-4-(4-(2-cyclopropylpyridin-4-yl)thiophen-2-yl)phenyl)-N-methylmethanesulfonamide (0.085 mg; 41%) as an off white solid. ¹H NMR 400 MHz, DMSO-d6: δ 8.41 (d, J=4.80 Hz, 1H), 8.32 (d, J=1.20 Hz, 1H), 8.00 (d, J=1.60 Hz, 1H), 7.80 (d, J=8.40 Hz, 1H), 7.68-7.66 (m, 2H), 7.51-7.48 (m, 2H), 3.32 (s, 3H), 3.04 (s, 3H), 2.16-2.07 (m, 1H), 0.97-0.96 (m, 4H); LCMS: 99.84% (419.17[M+H]⁺ ion present.

The following compounds were prepared in a similar fashion:

| | |
|---|---|
| 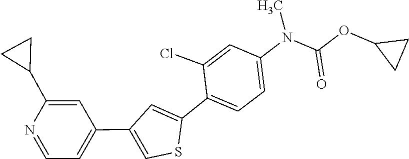 [SMILES: CN(C(=O)OC1CC1)c1ccc(-c2cc(-c3ccnc(C4CC4)c3)cs2)c(Cl)c1] | cyclopropyl (3-chloro-4-(4-(2-cyclopropylpyridin-4-yl)thiophen- 2-yl)phenyl)(methyl)carbamate ¹H NMR 400 MHz, DMSO-d6: δ 8.41 (d, J = 4.80 Hz, 1H), 8.30 (d, J = 1.60 Hz, 1H), 7.99 (d, J = 1.60 Hz, 1H), 7.73 (d, J = 8.40 Hz, 1H), 7.68 (d, J = 1.20 Hz, 1H), 7.61 (d, J = 2.00 Hz, 1H), 7.50 (dd, J = 2.00, 5.20 Hz, 1H), 7.40 (dd, J = 2.40, 8.40 Hz, 1H), 4.07-4.04 (m, 1H), 3.24 (s, 3H), 2.16-2.10 (m, 1H), 0.97-0.95 (m, 4H), 0.68-0.65 (m, 4H); LCMS: 99.91% (425.26[M + H]⁺ |
| I-9038 | |

### Synthesis Example 24: Cyclopropyl(3-chloro-4-(5-(2-(1-hydroxy-2-methylpropan-2-yl)pyridin-4-yl)thiophen-3-yl)phenyl)carbamate (I-9161)

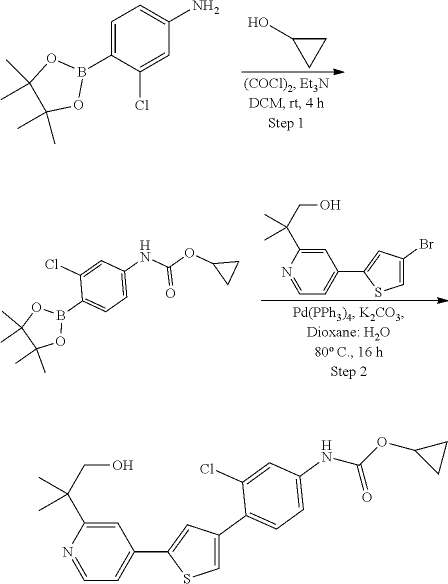

### Step 1: Cyclopropyl (3-chloro-4-(4,4,5,5-tetramethyl-1,3,2-dioxaborolan-2-yl)phenyl)carbamate

To a stirred solution of 3-chloro-4-(4,4,5,5-tetramethyl-1,3,2-dioxaborolan-2-yl)aniline (0.600 g, 2.37 mmol) and cyclopropanol (0.45 mL, 7.1146 mmol) in DCM cooled to 0-5° C., was added Et₃N (0.71 g, 7.1146 mmol). After 15 min COCl₂ (25% in toluene) (1.408 g, 2.817 mmol) was added dropwise and the reaction was stirred at RT for 6 h. The reaction was quenched with sodium bicarbonate and the product extracted into ethyl acetate. The organic layer was concentrated to give a crude product (0.670 g) as a brown gum which was purified by column chromatography to give cyclopropyl (3-chloro-4-(4,4,5,5-tetramethyl-1,3,2-dioxaborolan-2-yl)phenyl)carbamate as yellow liquid (0.3 g; 38%).

### Step 2: Cyclopropyl(3-chloro-4-(5-(2-(1-hydroxy-2-methylpropan-2-yl)pyridin-4-yl)thiophen-3-yl)phenyl)carbamate (I-9161)

To a stirred solution of the product of Step 1 (0.3 g, 0.8902 mmol) in 1,4-dioxane, H2O (7 mL) was added 2-(4-(4-bromothiophen-2-yl)pyridin-2-yl)-2-methylpropan-1-ol (0.304 g, 0.9792 mmol) and K₂CO₃ (0.368 g, 2.6706 mmol). The reaction mixture was purged with N₂, Pd(PPh₃)₄ (0.061 g, 0.0534 mmol) was added and the reaction mixture was heated to 80° C. while stirring for 16 h. The reaction mixture was filtered through celite and concentrated under reduced pressure to give a crude product was purified by prep HPLC to give cyclopropyl(3-chloro-4-(5-(2-(1-hydroxy-2-methylpropan-2-yl)pyridin-4-yl)thiophen-3-yl)phenyl)carbamate as white solid (0.1 g:25%). 1HNMR 400 MHz, DMSO-d6: δ 9.98 (s, 1H), 8.53 (d, J=4.80 Hz, 1H), 7.97 (d, J=1.20 Hz, 1H), 7.81 (d, J=1.60 Hz, 1H), 7.73 (d, J=1.60 Hz, 1H), 7.62 (d, J=0.80 Hz, 1H), 7.55 (s, 1H), 7.47-7.45 (m, 2H), 4.68 (t, J=5.20 Hz, 1H), 4.09-4.08 (m, 1H), 3.59 (d, J=5.60 Hz, 2H), 1.29 (s, 6H), 0.71-0.70 (m, 4H); LCMS: 99.43% (M+H=443.48.

### Synthesis Example 25: 2-(4-(4-(2-chloro-4-(((isopropylamino)thio)amino)phenyl)thiophen-2-yl)pyridin-2-yl)-2-methylpropan-1-ol, S,S-dioxide (I-9162)

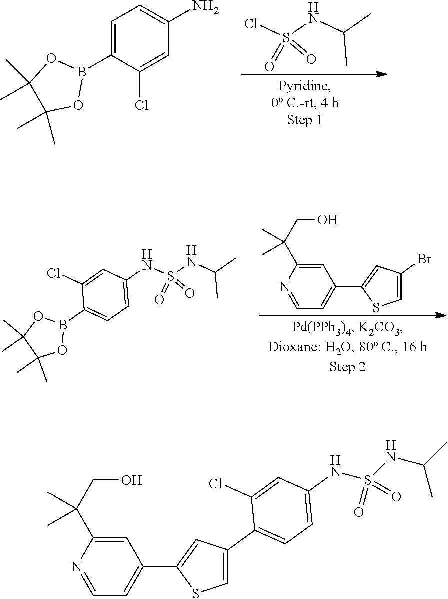

### Step 1: N-(3-chloro-4-(4,4,5,5-tetramethyl-1,3,2-dioxaborolan-2-yl)phenyl)-S-(isopropylamino)thiohydroxylamine S,S-dioxide

A solution of 3-chloro-4-(4,4,5,5-tetramethyl-1,3,2-dioxaborolan-2-yl)aniline (500 mg, 1.97 mmol) in pyridine was cooled to 0-5° C. and isopropylsulfamoyl chloride (341 mg, 2.17 mmol) was added dropwise. After stirring at RT for 4 h the reaction was quenched with water and the product was extracted into DCM. The organic layer was washed with water, dried over sodium sulfate and concentrated under reduced pressure to afford crude N-(3-chloro-4-(4,4,5,5-tetramethyl-1,3,2-dioxaborolan-2-yl)phenyl)-S-(isopropylamino)thiohydroxylamine S,S-dioxide (360 mg; 49%) as a yellow gum.

### Step 2: 2-(4-(4-(2-chloro-4-(((isopropylamino)thio)amino)phenyl)thiophen-2-yl)pyridin-2-yl)-2-methylpropan-1-ol, S,S-dioxide (I-9162)

A stirred solution of the product of Step 1 (360 mg, 0.96 mmol), 2-(4-(4-bromothiophen-2-yl)pyridin-2-yl)-2-methylpropan-1-ol (250 mg, 0.80 mmol), potassium carbonate (332 mg, 2.41 mmol) in 1,4-dioxane (8 mL) and water (2 mL) was purged with nitrogen gas for 5 minutes. After adding palladium Pd(PPh₃)₄ (55 mg, 0.04 mmol), the reaction mass was heated to 80° C. for 16 h. The reaction mixture was cooled to 25° C.-30° C., filtered through a celite bed and concentrated under reduced pressure to give a crude product which was purified by prep HPLC to afford 2-(4-(4-(2-chloro-4-(((isopropylamino)thio)amino)phenyl)thiophen-2-yl)pyridin-2-yl)-2-methylpropan-1-ol, S,S-dioxide (42 mg; 11%) as an off white solid. 1HNMR 400 MHz, DMSO-d6: δ 9.82 (br s, 1H), 8.52 (d, J=5.20 Hz, 1H), 7.97 (d, J=1.60 Hz, 1H), 7.79 (d, J=1.20 Hz, 1H), 7.63 (d, J=0.80 Hz, 1H), 7.47-7.48 (m, 3H), 7.31 (d, J=2.40 Hz, 1H), 7.13 (dd, J=2.00, 8.40 Hz, 1H), 4.68 (t, J=5.20 Hz, 1H), 3.59 (d, J=5.60 Hz, 2H), 3.41 (s, 1H), 1.29 (s, 6H), 1.03 (d, J=6.40 Hz, 6H); LCMS: 99.68% ((M+H) 480.20).

### Biological Example 1: Gene Expression, Reporter Assay, and Liver Microsomes

Gene Expression:

The effect of selected compounds on the gene expression of HepG2 cells was evaluated. HepG2 cells (P2) were seeded in 24 well plate (80,000 cells/well) for RNA extraction and in a 96 well plate (10,000 cells/well) for Cell Titer Glow (CTG). The media used was DMEM and contained 10% FBS. Each compound was evaluated at 500 Mm for 48 hours. Two biological replicates per experimental group were evaluated for RNA. For gene analysis, RNA was harvested with RNEasy kit and 20-100 ng used to synthesize cDNA with random primers. Quantitative PCR was performed on 1 pg to 100 ng cDNA for the following genes: ACACA, ACLY, FASN, LSS, PNPLA3. Gene expression levels were normalized with housekeeping gene, β-Actin, and relative expression levels determined using ΔΔCT method comparing treated to mock or vehicle treated cells as a baseline. “Total” gene expression refers to the average of the values of the 5 genes listed above. Results are presented in Table 1 below.

Reporter Screening Assay: This assay was used to evaluate the effect on transcriptional activity SREBP of selected compounds using an SRE-luciferase reporter construct. On day 1, 10,000 cells were seeded in a 96 well (white) plate as per the plate map in Growth media without antibiotics. Cells were incubated at 37° C. for 8 hours. After 8 hours, cells were washed with DPBS for complete removal of FBS. DPBS was completely removed and Growth media was replaced with phenol free treatment medium (90 μl) with different FBS concentrations. The cells were then incubated at 37° C. for 24 hours with varying doses (0.01 uM to 10 uM) of compounds. Then a Luciferase assay was performed.

Reagents for performing Luciferase assay were stored at −20° C. To a tube of lyophilized assay substrate was added 1 mL Substrate Solvent and mixed well. The Substrate tube after reconstitution was covered with aluminum foil so as to keep it protected from light. The assay buffer was thawed to room temperature. To 20 mL Assay Buffer was added 200 μL of reconstituted 100× Substrate and mixed well. The reconstituted substrate as well as the assay solution (buffer+substrate) was protected from light throughout the procedure by keeping it covered with aluminum foil. Using a multi-channel pipette, 100 μL Assay Solution (buffer+substrate) was added directly to each sample well in Plate 1, which was incubated for 30 min (plate was covered with aluminum foil). After 30 min incubation, the plate was read for total luminescence. Each well was read for 2 seconds in a plate luminometer. (Microplate reader Envision Microplate reader from Perkin Elmer). Precaution was taken to incubate plate exactly for 30 min prior to reading on the plate reader. Results are presented in Table 1 below.

Reporter Assay Materials:

SREBPv1 Reporter cell line: HepG2—#32251. Growth Medium: MEM (Corning 10-010), 10% FBS, 1% GlutaMax (Invitrogen Catalog #35050061), μg/ml Puromycin (Invitrogen Catalog # A1113803) and 1% Penicillin-Streptomycin (Pen-Strep). Treatment Media: Phenol-free MEM (Invitrogen Catalog #51200-038) and 1% GlutaMax (Invitrogen Catalog #35050061). Luciferase Assay: LightSwitch Luciferase Assay Kit (Catalog #32032). LDH assay: Pierce LDH Cytotoxicity Assay Kit (Catalog # SD249616).

Half-Life Human Microsomes:

Compounds were evaluated for stability in human liver microsomes. A 10 mM stock solution of the compound being evaluated was prepared in DMSO and diluted with water:acetonitrile (1:1) to a concentration of 1 mM. A working concentration of 100 μM was prepared by further dilution with water:acetonitrile (1:1). To make the preincubation mixture, 2.5 μL of the diluted compound was combined with 75 μL of human liver microsomes at 3.33 mg/mL, and 85 μL of 100 mM potassium phosphate buffer, and this mixture was pre-incubated for 10 min at 37° C. To make the 60 minute mixture without cofactor, 32.5 μL of the preincubation mixture was combined with 17.5 μL of 100 mM potassium phosphate buffer and incubated for 60 min at 37° C. To make the 0 min sample with cofactor (NADPH), 16.25 μL of the preincubation mixture was combined with 200 μL of acetonitrile containing internal standard and 8.75 μL of cofactor (NADPH). To make the incubation mixture, 62 μL of cofactor (2.85 mM) was combined with the remaining incubation mixture, and incubated for 60 min at 37° C. To prepare the sample mixture to be evaluated, 25 μL incubation mixture was combined with 200 μL of acetonitrile containing internal standard and vortexed for 5 min at 1200 rpm, then centrifuged for 10 min at 4000 rpm. The supernatant was diluted 2 fold with water and injected on LC-MS/MS. The sample mixture was evaluated by LC-MS/MS using 10 mM ammonium acetate with 0.100 FA as the aqueous mobile phase, and methanol as the organic mobile phase.

Half-Life Mouse Microsomes:

Compounds were evaluated in mouse liver microsomes following a similar procedure as described above for human liver microsomes. A similar procedure could be used to evaluate compounds in rat liver microsomes. Results are presented in Table 1 below.

TABLE 1
Data for selected compounds. Entries A-F are effect of compounds on gene
expression of HepG2 cells. A: ACACA; B: ACLY; C: FASN; D: LSS; E: PNPLA3; F:
Total. For gene expression at the tested dose, 0-0.309 = +++, 0.31-0.7509 = ++, >0.751 = +.
Entries G and H are the half-life of liver microsomes (percent rem @60 min; G is human, H
is mouse), where 0-30 = +; 30.1-60 = ++; >60.1 = +++. Entry I is for the Reporter
Screening Assay (Ave EC50 (nM)), where 0-249 = +++, 250-500 = ++, and >500 = +.

| Compound | A | B | C | D | E | F | G | H | I |
|---|---|---|---|---|---|---|---|---|---|
| 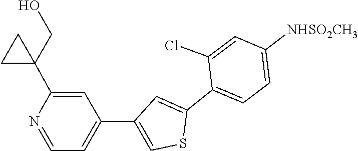 | ++ | ++ | ++ | ++ | ++ | ++ | +++ | +++ | +++ |
| I-9002 | | | | | | | | | |
| 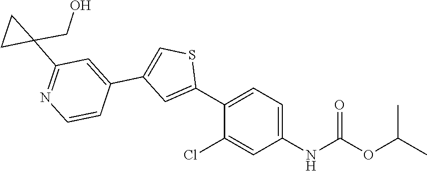 [SMILES: CC(C)OC(=O)Nc1ccc(-c2cc(-c3ccnc(C4(CO)CC4)c3)cs2)c(Cl)c1] | ++ | ++ | ++ | +++ | +++ | ++ | +++ | +++ | +++ |
| I-8980 | | | | | | | | | |
| 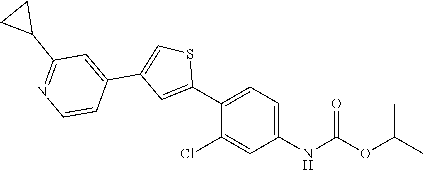 [SMILES: CC(C)OC(=O)Nc1ccc(-c2cc(-c3ccnc(C4CC4)c3)cs2)c(Cl)c1] | ++ | ++ | ++ | ++ | ++ | ++ | +++ | +++ | +++ |
| I-8966 | | | | | | | | | |
| 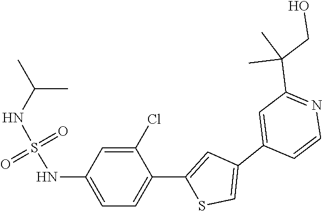 [SMILES: CC(C)NS(=O)(=O)Nc1ccc(-c2cc(-c3ccnc(C(C)(C)CO)c3)cs2)c(Cl)c1] | ++ | ++ | ++ | +++ | +++ | ++ | ++ | ++ | +++ |
| I-8941 | | | | | | | | | |
| 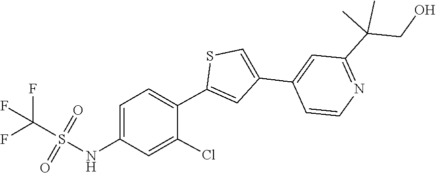 [SMILES: CC(C)(CO)c1cc(-c2csc(-c3ccc(NS(=O)(=O)C(F)(F)F)cc3Cl)c2)ccn1] | + | + | + | + | + | + | + | ++ | +++ |
| I-8940 | | | | | | | | | |
| 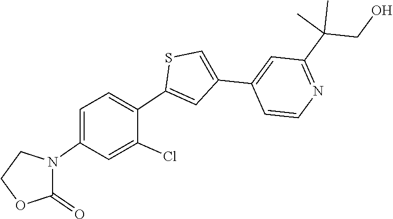 [SMILES: CC(C)(CO)c1cc(-c2csc(-c3ccc(N4CCOC4=O)cc3Cl)c2)ccn1] | + | ++ | ++ | ++ | ++ | ++ | ++ | +++ | ++ |
| I-8939 | | | | | | | | | |
| 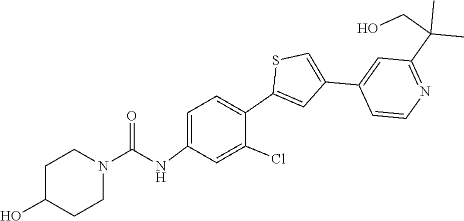 [SMILES: CC(C)(CO)c1cc(-c2csc(-c3ccc(NC(=O)N4CCC(O)CC4)cc3Cl)c2)ccn1] | + | + | + | + | + | + | ++ | +++ | +++ |
| I-8926 | | | | | | | | | |
| 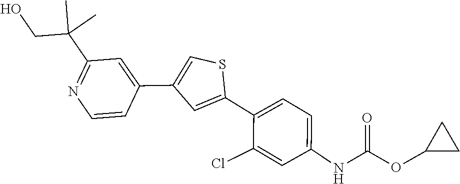 [SMILES: CC(C)(CO)c1cc(-c2csc(-c3ccc(NC(=O)OC4CC4)cc3Cl)c2)ccn1] | ++ | ++ | ++ | ++ | ++ | ++ | +++ | ++ | ++ |
| I-8918 | | | | | | | | | |
| 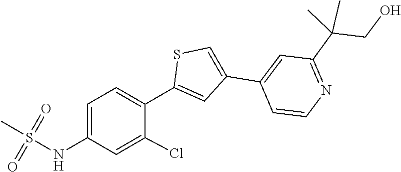 [SMILES: CC(C)(CO)c1cc(-c2csc(-c3ccc(NS(C)(=O)=O)cc3Cl)c2)ccn1] | ++ | ++ | ++ | +++ | +++ | ++ | ++ | +++ | +++ |
| I-8909 | | | | | | | | | |
| 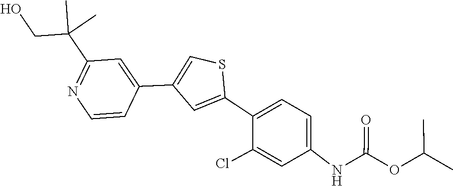 [SMILES: CC(C)OC(=O)Nc1ccc(-c2cc(-c3ccnc(C(C)(C)CO)c3)cs2)c(Cl)c1] | + | ++ | ++ | +++ | +++ | ++ | +++ | +++ | ++ |
| I-8907 | | | | | | | | | |
| 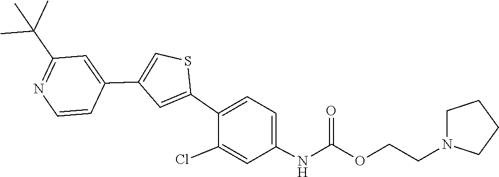 [SMILES: CC(C)(C)c1cc(-c2csc(-c3ccc(NC(=O)OCCN4CCCC4)cc3Cl)c2)ccn1] | + | ++ | + | + | ++ | ++ | +++ | + | + |
| I-8762 | | | | | | | | | |
| 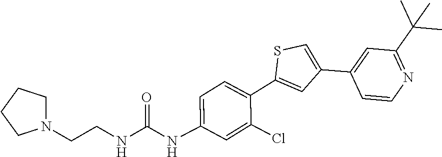 [SMILES: CC(C)(C)c1cc(-c2csc(-c3ccc(NC(=O)NCCN4CCCC4)cc3Cl)c2)ccn1] | + | + | + | + | ++ | + | +++ | +++ | + |
| I-8761 | | | | | | | | | |
| 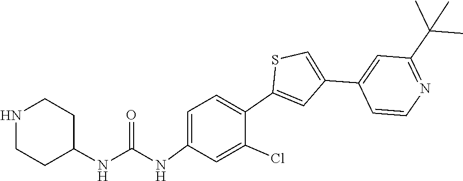 [SMILES: CC(C)(C)c1cc(-c2csc(-c3ccc(NC(=O)NC4CCNCC4)cc3Cl)c2)ccn1] | + | + | + | + | + | + | +++ | +++ | + |
| I-8755 | | | | | | | | | |
| 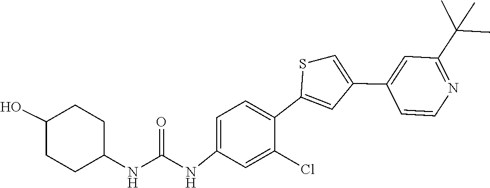 [SMILES: CC(C)(C)c1cc(-c2csc(-c3ccc(NC(=O)NC4CCC(O)CC4)cc3Cl)c2)ccn1] | + | + | + | + | + | + | +++ | +++ | + |
| I-8742 | | | | | | | | | |
| 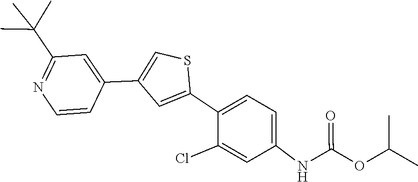 [SMILES: CC(C)OC(=O)Nc1ccc(-c2cc(-c3ccnc(C(C)(C)C)c3)cs2)c(Cl)c1] | ++ | ++ | ++ | +++ | ++ | ++ | +++ | +++ | +++ |
| I-8741 | | | | | | | | | |
| 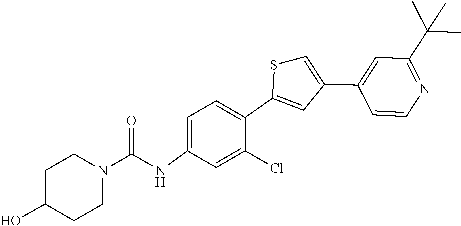 [SMILES: CC(C)(C)c1cc(-c2csc(-c3ccc(NC(=O)N4CCC(O)CC4)cc3Cl)c2)ccn1] | ++ | ++ | +++ | +++ | +++ | +++ | +++ | +++ | ++ |
| I-8736 | | | | | | | | | |
| 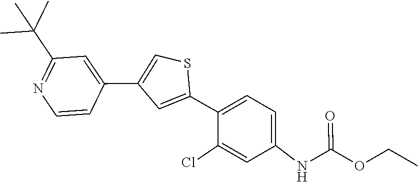 [SMILES: CCOC(=O)Nc1ccc(-c2cc(-c3ccnc(C(C)(C)C)c3)cs2)c(Cl)c1] | ++ | ++ | +++ | +++ | +++ | +++ | +++ | +++ | ++ |
| I-8735 | | | | | | | | | |
| 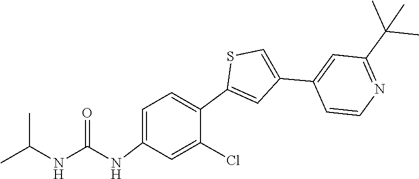 [SMILES: CC(C)NC(=O)Nc1ccc(-c2cc(-c3ccnc(C(C)(C)C)c3)cs2)c(Cl)c1] | ++ | ++ | ++ | ++ | ++ | ++ | ++ | ++ | +++ |
| I-8731 | | | | | | | | | |
| 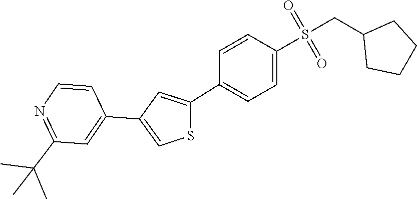 [SMILES: CC(C)(C)c1cc(-c2csc(-c3ccc(S(=O)(=O)CC4CCCC4)cc3)c2)ccn1] | + | + | + | + | + | + | + | + | + |
| I-8729 | | | | | | | | | |
| 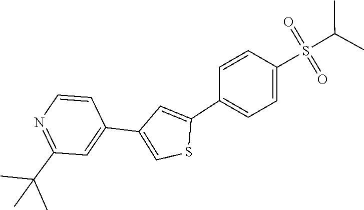 [SMILES: CC(C)S(=O)(=O)c1ccc(-c2cc(-c3ccnc(C(C)(C)C)c3)cs2)cc1] | + | ++ | + | + | ++ | + | ++ | +++ | ++ |
| I-8728 | | | | | | | | | |
| 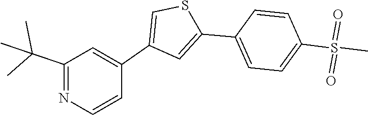 [SMILES: CC(C)(C)c1cc(-c2csc(-c3ccc(S(C)(=O)=O)cc3)c2)ccn1] | | | | | | | +++ | +++ | + |
| I-8723 | | | | | | | | | |
| 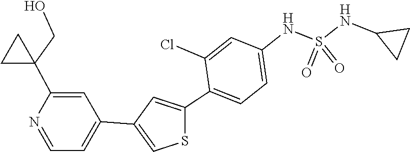 [SMILES: O=S(=O)(Nc1ccc(-c2cc(-c3ccnc(C4(CO)CC4)c3)cs2)c(Cl)c1)NC1CC1] | | | | | | | ++ | ++ | |
| I-9090 | | | | | | | | | |
| 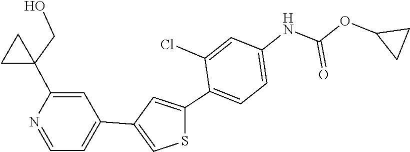 [SMILES: O=C(Nc1ccc(-c2cc(-c3ccnc(C4(CO)CC4)c3)cs2)c(Cl)c1)OC1CC1] | + | + | ++ | ++ | ++ | + | +++ | ++ | + |
| I-9082 | | | | | | | | | |
| 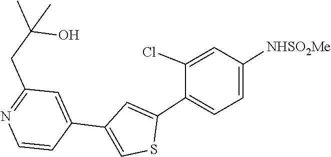 | ++ | ++ | ++ | ++ | ++ | ++ | +++ | +++ | +++ |
| I-9004 | | | | | | | | | |
| 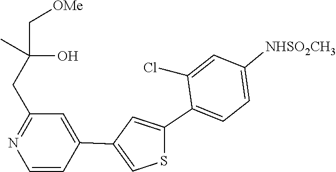 | | | | | | | | | |
| I-9129 | | | | | | | | | |
| 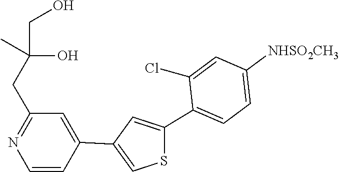 | | | | | | | | | |
| I-9119 | | | | | | | | | |
| 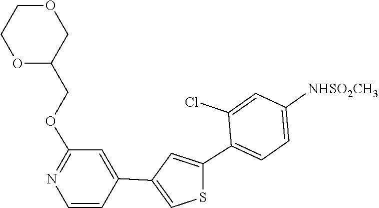 | | | | | | | ++ | +++ | |
| I-9113 | | | | | | | | | |
| 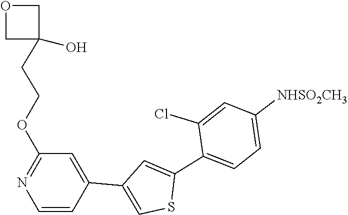 | | | | | | | +++ | +++ | |
| I-9112 | | | | | | | | | |
| 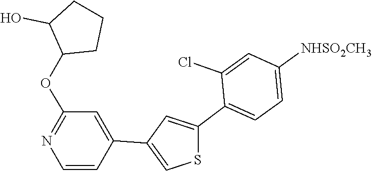 | | | | | | | +++ | +++ | |
| I-9111 | | | | | | | | | |
| 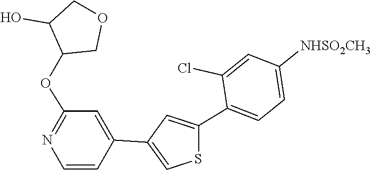 | | | | | | | +++ | +++ | + |
| I-9108 | | | | | | | | | |
| 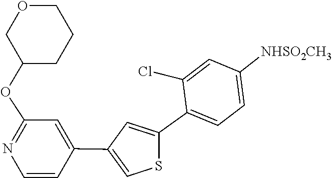 | | | | | | | +++ | ++ | + |
| I-9107 | | | | | | | | | |
| 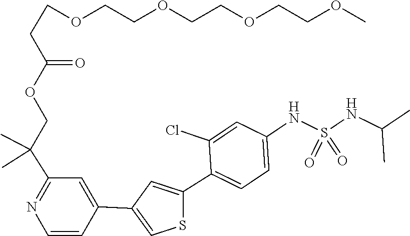 [SMILES: COCCOCCOCCOCCC(=O)OCC(C)(C)c1cc(-c2csc(-c3ccc(NS(=O)(=O)NC(C)C)cc3Cl)c2)ccn1] | | | | | | | | | |
| I-9104 | | | | | | | | | |
| 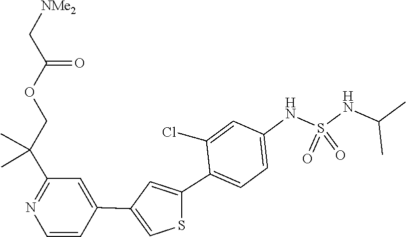 [SMILES: CC(C)NS(=O)(=O)Nc1ccc(-c2cc(-c3ccnc(C(C)(C)COC(=O)CN(C)C)c3)cs2)c(Cl)c1] | | | | | | | | | |
| I-9095 | | | | | | | | | |
| 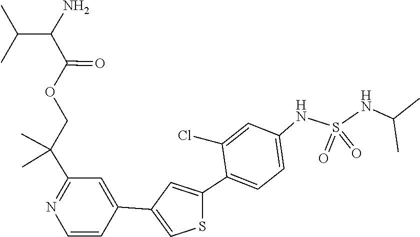 [SMILES: CC(C)NS(=O)(=O)Nc1ccc(-c2cc(-c3ccnc(C(C)(C)COC(=O)C(N)C(C)C)c3)cs2)c(Cl)c1] | | | | | | | | | |
| I-9094 | | | | | | | | | |
| 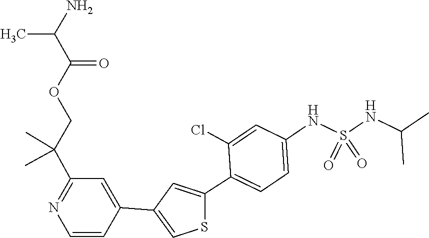 [SMILES: CCC(N)C(=O)OCC(C)(C)c1cc(-c2csc(-c3ccc(NS(=O)(=O)NC(C)C)cc3Cl)c2)ccn1] | | | | | | | | | |
| I-9093 | | | | | | | | | |
| 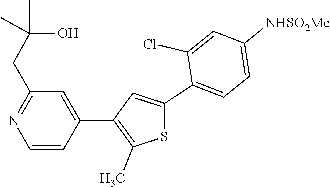 | | | | | | | +++ | +++ | + |
| I-9091 | | | | | | | | | |
| 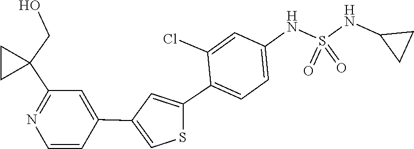 [SMILES: O=S(=O)(Nc1ccc(-c2cc(-c3ccnc(C4(CO)CC4)c3)cs2)c(Cl)c1)NC1CC1] | | | | | | | ++ | ++ | |
| I-9090 | | | | | | | | | |
| 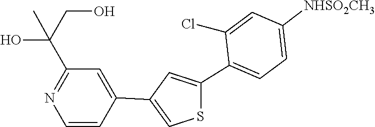 | | | | | | | +++ | +++ | + |
| I-9088 | | | | | | | | | |
| 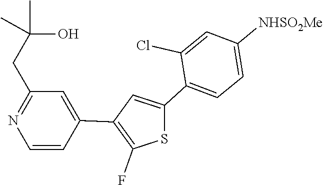 | + | + | + | + | + | + | +++ | +++ | + |
| I-9085 | | | | | | | | | |
| 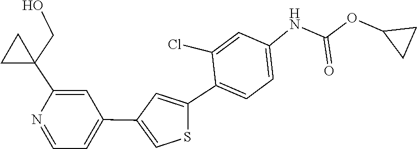 [SMILES: O=C(Nc1ccc(-c2cc(-c3ccnc(C4(CO)CC4)c3)cs2)c(Cl)c1)OC1CC1] | + | + | ++ | ++ | ++ | + | +++ | ++ | + |
| I-9082 | | | | | | | | | |
| 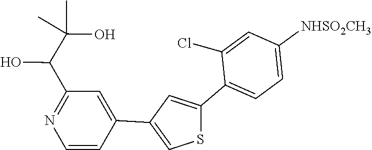 | + | + | + | + | + | + | +++ | +++ | + |
| I-9075 | | | | | | | | | |
| 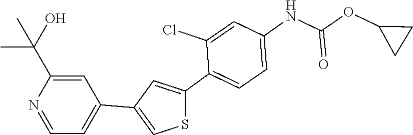 [SMILES: CC(C)(O)c1cc(-c2csc(-c3ccc(NC(=O)OC4CC4)cc3Cl)c2)ccn1] | + | + | + | + | ++ | + | +++ | +++ | + |
| I-9065 | | | | | | | | | |
| 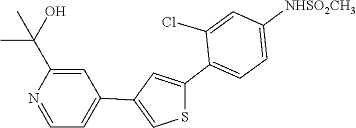 | + | ++ | + | ++ | ++ | ++ | +++ | +++ | +++ |
| I-9064 | | | | | | | | | |
| 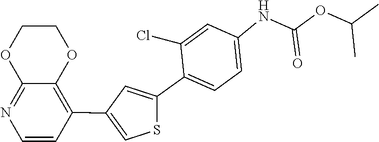 [SMILES: CC(C)OC(=O)Nc1ccc(-c2cc(-c3ccnc4c3OCCO4)cs2)c(Cl)c1] | ++ | ++ | ++ | +++ | +++ | ++ | +++ | +++ | +++ |
| I-9062 | | | | | | | | | |
| 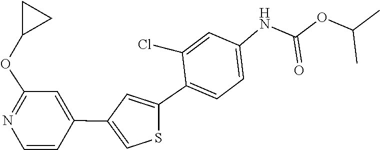 [SMILES: CC(C)OC(=O)Nc1ccc(-c2cc(-c3ccnc(OC4CC4)c3)cs2)c(Cl)c1] | ++ | ++ | ++ | ++ | +++ | ++ | +++ | +++ | +++ |
| I-9060 | | | | | | | | | |
| 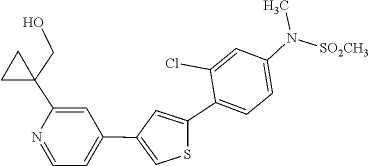 [SMILES: CN(c1ccc(-c2cc(-c3ccnc(C4(CO)CC4)c3)cs2)c(Cl)c1)S(C)(=O)=O] | ++ | ++ | ++ | ++ | +++ | ++ | ++ | +++ | +++ |
| I-9056 | | | | | | | | | |
| 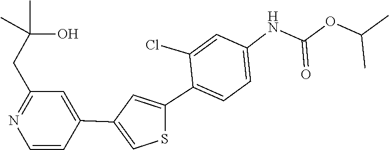 [SMILES: CC(C)OC(=O)Nc1ccc(-c2cc(-c3ccnc(CC(C)(C)O)c3)cs2)c(Cl)c1] | ++ | ++ | ++ | +++ | +++ | ++ | +++ | +++ | + |
| I-9051 | | | | | | | | | |
| 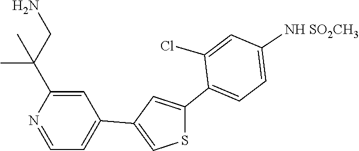 | + | + | + | ++ | ++ | + | +++ | +++ | + |
| I-9049 | | | | | | | | | |
| 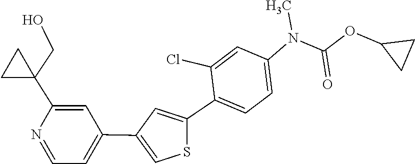 [SMILES: CN(C(=O)OC1CC1)c1ccc(-c2cc(-c3ccnc(C4(CO)CC4)c3)cs2)c(Cl)c1] | ++ | ++ | ++ | ++ | +++ | ++ | ++ | +++ | +++ |
| I-9042 | | | | | | | | | |
| 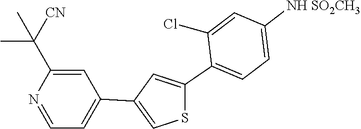 | + | + | + | + | + | + | +++ | +++ | + |
| I-9039 | | | | | | | | | |
| 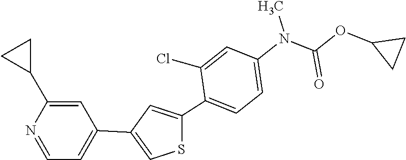 [SMILES: CN(C(=O)OC1CC1)c1ccc(-c2cc(-c3ccnc(C4CC4)c3)cs2)c(Cl)c1] | ++ | ++ | ++ | +++ | +++ | ++ | +++ | +++ | +++ |
| I-9038 | | | | | | | | | |
| 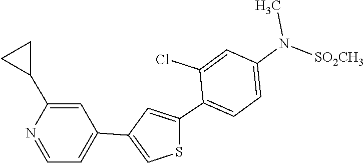 [SMILES: CN(c1ccc(-c2cc(-c3ccnc(C4CC4)c3)cs2)c(Cl)c1)S(C)(=O)=O] | +++ | ++ | +++ | +++ | +++ | +++ | +++ | +++ | +++ |
| I-9037 | | | | | | | | | |
| 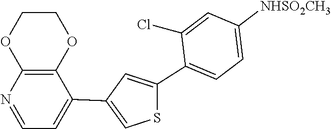 | ++ | ++ | ++ | +++ | +++ | ++ | +++ | +++ | +++ |
| I-9017 | | | | | | | | | |
| 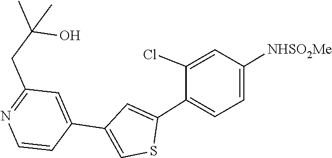 | ++ | ++ | ++ | ++ | ++ | ++ | +++ | +++ | +++ |
| I-9004 | | | | | | | | | |
| 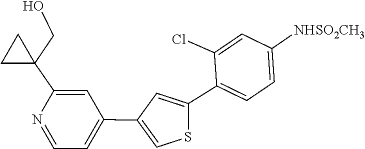 | ++ | ++ | ++ | ++ | ++ | ++ | +++ | +++ | +++ |
| I-9002 | | | | | | | | | |
| 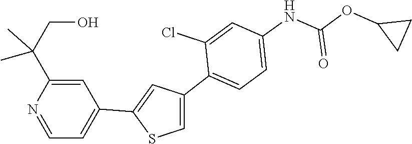 [SMILES: CC(C)(CO)c1cc(-c2cc(-c3ccc(NC(=O)OC4CC4)cc3Cl)cs2)ccn1] | | | | | | | ++ | ++ | |
| I-9161 | | | | | | | | | |
| 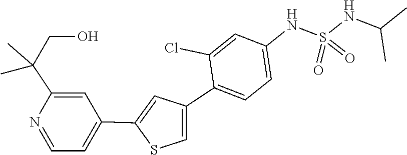 [SMILES: CC(C)NS(=O)(=O)Nc1ccc(-c2csc(-c3ccnc(C(C)(C)CO)c3)c2)c(Cl)c1] | | | | | | | + | +++ | |
| I-9162 | | | | | | | | | |

### Biological Example 2: Kinetic Solubility

Kinetic Solubility Procedure:

A 10 mM stock solution of a compound is prepared in DMSO, then 4 μL of the stock is added to a deep well plate containing 396 μL of pH 7.4 buffer. The sample plate is vortexed at 800 rpm for 24 h on thermomixer at room temperature. The plate is sealed well during the incubation process. The dimethylsulfoxide (DMSO) content in the sample is 1.0%. The concentration of the evaluated compound in the final incubation is 100 μM. At the end of the incubation period, the sample plate is centrifuged at 4000 rpm for 10 min and analyzed in LC-UV against a calibration curve (CC).

### Biological Example 3: Western Blotting of SREBP Processing

The effect of selected compounds on SREBP processing and activation is evaluated in HepG2 cells via Western blotting. Cells are seeded at a density of 8e⁶ in 150 mm plate in DMEM (Dulbecco's modified Eagle's medium) supplemented with 10% (V/V) heat-inactivated FBS (fetal bovine serum), penicillin G (100 units/ml) and gentamycin (0.2 mg/ml). After overnight incubation, they are washed twice in PBS, and then DMEM media with 0% FBS with 500 nM of compound is added to the plate. Cells are incubated at 37° C. After 48 hours, the cells are washed and lysed to obtain cytoplasmic and nuclear extracts for Western blotting to measure SREBP expression along with topoisomerase I as loading control.

### Biological Example 4: Adipocyte Differentiation and Oil Red-O Staining

The effect of selected compounds on adipocyte differentiation in human pre-adipocyte and 3T3-L1 cells is evaluated.

Human Pre-Adipocyte Differentiation:

Cells are thawed and seeded at 40,625 cells/cm² in pre-adipocyte media (ZenBio) as per manufacturer's direction. The cells are allowed to reach confluence for 48 hours, and media switched to Adipocyte Differentiation Media (ZenBio) for 7 days. The media is then switched to Adipocyte Maintenance Media (ZenBio) for additional 7 days. The compound being evaluated is added to the cells for day 1-7 during differentiation, or day 7-14 during maturation. Cells are then stained with oil red-O as described below.

NIH 3T3-L1 Cell Differentiation:

Cells are thawed into Pre-Adipocyte Media (ZenBio) and grown to 80-85% confluence. Cells are seeded 50,000 cells/well into 96-wp in Pre-Adipocyte Media (ZenBio) and allowed to reach confluence for 48-72 hours. They are grown an additional 48 hours after reaching confluence, then the media is changed to Differentiation Media (Zen Bio) and incubated for 72 hours. The media is changed to Adipocyte Differentiation Media (ZenBio) using 150 microliters/well in 96-wp for 72 hours, then media is removed and replaced with 150 microliters of Adipocyte Maintenance Media for an additional 8-14 days, feeding cells every 2-3 days. The compound being evaluated is added to the cells for day 3-6 during differentiation, or day 7-14 during maturation. Cells are then stained with oil red-O as described below.

Oil Red-O Staining:

After maturation, the cells are washed, then fixed in 10% Formalin for 30-60 minutes. The formalin is removed, the cells are washed in water twice, and then the cells are incubated in 60% isopropanol for 5 minutes. The isopropanol is removed and Oil Red-O solution added for 20 minutes with gentle rotation of plate. The stain is removed, the cells washed twice with water, and Hematoxylin added for 1 minute. The cells are washed twice with water and air dried, then images are acquired.

### Biological Example 5: Log D of Compounds

The Log D of selected compounds is evaluated by octanol/aqueous buffer partitioning. 500 μL of organic phase (1-octanol) is added to each well of a 2 mL deep well plate, followed by 500 μL of buffer and 15 μL of test compound in DMSO (0.15 mM). The plate is vortexed for 10 seconds and incubated at room temperature for 1 hr on a plate shaker at 200 rpm. After incubation, the samples are allowed to equilibrate for 20 min and then centrifuged at 4000 rpm for 30 min for complete phase separation. The distribution of test compound in buffer and octanol phase was analyzed by HPLC-UV. Log D=Log (Area of Octanol/Area of Buffer).

### Biological Example 6: In Vivo Activity Assay

The in vivo effect of selected compounds may be assessed using the ob/ob mouse model. The ob/ob mouse is a well characterized model of obesity, fatty liver, and diabetes, which are exhibited due to a mutation in the ob gene, which encodes for leptin.

Compounds are administered by the oral route once or twice daily for 4 weeks in male ob/ob mice. Body weight and food and water intake are assessed daily, and improvements in glucose control are assessed by plasma glucose and insulin measurement. Upon completion of the test period, terminal blood samples are taken and analyzed for triglyceride, cholesterol (total, HDL-C and LDL-C), blood urea nitrogen (BUN), alanine aminotransferase (ALT), and aspartate aminotransferase (AST) levels. Liver and fat pad weights are determined and liver tissue is processed for histological determination of NASH activity scores (NAS: ballooning, inflammation, steatosis and fibrosis). Liver levels of triglycerides, cholesterol, and non-esterified fatty acids (NEFA) are also determined.

### Biological Example 7: Evaluation of In Vivo Pharmacokinetic Properties of Compounds

The in vivo pharmacokinetic properties of compounds by both intravenous and oral administration is evaluated in male Sprague Dawley rats or C57BL/6J mice.

Animals are housed in cages with clean bedding. Certified rodent diet is provided. Water was available ad libitum. Environmental controls for the animal room are set to maintain a temperature of 22° C. to 25° C., humidity of 40-70% RH, and a 12-hour light/12-hour dark cycle. Normal healthy animals certified by the attending veterinarian are selected and acclimatized for minimum three days prior to initiation of study.

Surgical Procedure for Jugular Vein Cannulation of Rats:

Rats are anaesthetized with a single dose of ketamine 50 mg/kg i.p.+xylazine 6 mg/kg i.p. The right jugular vein is exposed, a loose ligature is placed caudally, and the cranial end of vein is ligated. A small incision is made between the ligatures into which the catheter (polyethylene 50 tubing of internal diameter 0.58 mm and outer diameter 0.96 mm) is inserted. The catheter is secured in place by tying the loose ligature around the catheterized vessel. A small incision is made in the scapular region to serve as the exit site of the catheter. The catheter is subcutaneously tunneled and exteriorized through scapular incision. A stay suture is placed in the scapular area. Patency is tested, and catheter is filled with a locking solution (heparinized saline) and sealed with a stainless steel plug. The incision is then sutured with sterile suturing material. Anti-septic solution is applied to the sutured site and animal is placed back in the home cage.

To evaluate pharmacokinetic properties of intravenous delivery, male Sprague Dawley rats are administered 2.00 mg compound/kg animal weight through the tail vein. The concentration of the compound in the plasma of the animals is evaluated at 0.083, 0.25, 0.5, 1, 2, 4, 8, 12 and 24 hr by taking blood samples from the cannulated jugular vein.

To evaluate pharmacokinetic properties of oral delivery, rats (male Sprague Dawley rats) or mice (C5Bl/6J) are administered 10 mg compound/kg animal weight by mouth. The concentration of compound in the plasma of the animals is evaluated at 0.25, 0.5, 1, 2, 4, 6, 8, 12 and 24 hr by taking blood samples from the cannulated jugular vein (rats) or through a capillary, guided in retro-orbital plexus (mice).

### Biological Example 8: Evaluation of Compounds on Liver Gene Expression in Mice

The pharmacodynamic properties of selected compounds are evaluated in mice. The animals are housed in cages with clean bedding, and maintained and monitored for good health in accordance with Test Facility SOPs and at the discretion of the laboratory animal veterinarian. Certified rodent diet is provided. Food and water is available ad libitum. Environmental controls for the animal room are set to maintain a temperature of 22° C. to 25° C., humidity of 40-70% RH, and a 12-hour light/12-hour dark cycle. Normal healthy animals certified by the attending veterinarian are selected and acclimatized for minimum three days prior to initiation of study. Animals are identified with body markings.

Aliquots of the compounds being evaluated are weighed and triturated with 0.5% methylcellulose (with the addition of 5% N-methyl pyrrolidone when required to remove clumping) to an appropriate dose concentration. Vials are labeled with the information about study number, test item, concentration and date of preparation. A description of appearance of formulation is recorded (e.g., color, turbidity, etc.). The composition of formulation vehicle is recorded. An aliquot of each dose solution was taken before the dosing began and after dosing is finished, and stored at approximately −20° C. or below for subsequent analysis. The animals are dosed orally through oral gavage needle, and time of dosing is recorded.

After dosing, the mice are anesthetized using gaseous anesthesia. Blood samples are collected through a capillary, guided in retro-orbital plexus, at 6 h or at 24 h. Approximately 100 uL of blood is collected from each mouse, in pre-labeled tubes. The collected blood is stored on ice prior to centrifugation. Blood samples are then centrifuged within 1 hour of collection to separate plasma. Centrifugation was conducted at 2500×g for 15 minutes at 4° C. The plasma is separated and transferred to pre-labeled micro-centrifuge tubes and promptly frozen at −80±10° C. until bioanalysis.

### Liver Collection at 6 h or 24 h:

Immediately after blood withdrawal for pharmacokinetic evaluation (at 6 or at 24 h), liver tissue is collected without perfusion. Animals are euthanized using carbon dioxide gas in a CO₂ chamber. The whole blood is drained by cutting the both side jugular vein and abdominal aorta. The liver is separated out. All the liver samples are divided in two parts. The first part (200 mg approx.) is snap frozen using liquid nitrogen as soon as possible. These samples are immediately transferred to −80° C. for storage. The remaining part was weighed and used for bioanalysis.

RNA Processing and Gene Expression Analysis:

Liver Tissue RNA is harvested with the RNEasy kit and 20-100 ng used to synthesize cDNA with random primers following the manufacturer's protocol. Quantitative PCR was performed on 1 pg to 100 ng cDNA for the following genes: ACACA, ACLY, FASN, LSS, PNPLA3. Gene expression levels are determined using ΔΔCT method comparing treated to vehicle treated samples as a baseline, and fold change calculated. The average value for all 5 genes above is averaged and termed to Total Fold Change.

### Biological Example 9: Evaluation of Compounds on Cancer Cell Growth Inhibition

The ability of select compounds to inhibit cell growth in various cancer cell lines are evaluated. Cells are treated with compound at doses ranging from 10 uM to 1 pM to generate IC₅₀ curves of cell line growth inhibition. The cell lines shown in Table 2 have reduced growth by at least 50% at 10 uM or lower over 72 hr treatment in growth media. The cell lines shown in Table 3 have reduced growth by at least 50% with an IC₅₀ greater than 10 uM.

Six 10-fold compound dilutions are prepared in DMSO (e.g. 10 mM, 1 mM, 100 uM, 10 uM, 1 uM, and 0.1 uM). Single data points are acquired for each concentration. The final concentration of DMSO is 0.1%. The duration of the treatment is 72 hr. Growth inhibition is measured using Sulforhodamine B in a protein staining assay. Activity of the agents is determined by evaluation the following parameters: IC₅₀, GI₅₀, IC₁₀, TGI, LC₅₀, IC₉₀, and GI₉₀ (where these values can be calculated).

TABLE 2
Responder Cell Lines with IC50 < 10 uM

| Compound I-9017 | | Compound I-9004 | | Compound I-8909 | |
|---|---|---|---|---|---|
| Cell line | Cell origin | Cell line | Cell origin | Cell line | Cell origin |
| A2780 | ovary | A2780 | ovary | PLCPRF5 | liver |
| PLCPRF5 | liver | PLCPRF5 | liver | A2780 | ovary |
| ACHN | kidney | ACHN | kidney | ACHN | kidney |
| C33A | endometrial | C33A | endometrial | C33A | endometrial |
| SKMEL5 | skin | SKMEL5 | skin | SKMEL5 | skin |
| L-363 | hematological | MCF7 | breast | MCF7 | breast |
| MDAMB468 | breast | L-363 | hematological | L-363 | hematological |
| MCF7 | breast | OVCAR4 | ovary | OVCAR4 | ovary |
| K-562 | hematological | K-562 | hematological | K-562 | hematological |
| DLD1 | colon | OVCAR3 | ovary | MDAMB468 | breast |
| OVCAR4 | ovary | HEPG2 | liver | HEPG2 | liver |
| HEPG2 | liver | MDAMB468 | breast | ASPC1 | pancreas |
| OVCAR3 | ovary | DLD1 | colon | MV4-11 | hematological |
| MV4-11 | hematological | MINO | hematological | OVCAR3 | ovary |
| ASPC1 | pancreas | HL-60 | hematological | SF295 | brain |
| HL-60 | hematological | SF295 | brain | DLD1 | colon |
| MINO | hematological | ASPC1 | pancreas | MINO | hematological |
| MT3 | breast | WSU-NHL | hematological | HL-60 | hematological |
| SU-DHL-10 | hematological | MV4-11 | hematological | MT3 | breast |
| HCT116 | colon | MT3 | breast | CALU6 | lung |
| HEK293 | kidney | CALU6 | lung | HCT116 | colon |
| SKHEP1 | liver | SU-DHL-6 | hematological | WSU-NHL | hematological |
| SU-DHL-6 | hematological | 22RV1 | prostate | RAMOS | hematological |
| 22RV1 | prostate | HCT116 | colon | 22RV1 | prostate |
| RAMOS | hematological | RAMOS | hematological | SU-DHL-6 | hematological |
| SF295 | brain | HEK293 | kidney | SU-DHL-10 | hematological |
| LOVO | colon | SU-DHL-10 | hematological | JAR | placenta |
| HT1080 | connective tissue | EFO21 | ovary | HEK293 | kidney |
| NCIH82 | lung | HT1080 | connective tissue | HT1080 | connective tissue |
| WSU-NHL | hematological | JAR | placenta | LOVO | colon |
| EFO21 | ovary | SKHEP1 | liver | NCIH82 | lung |
| CALU6 | lung | LOVO | colon | SKHEP1 | liver |
| CACO2 | colon | NCIH358M | lung | EFO21 | ovary |
| JAR | placenta | THP-1 | hematological | JIMT1 | breast |
| THP-1 | hematological | NCIH82 | lung | CACO2 | colon |
| A549 | lung | JIMT1 | breast | THP-1 | hematological |
| COLO205 | colon | A549 | lung | COLO678 | colon |
| SKNSH | brain | CACO2 | colon | UMUC3 | bladder |
| COLO678 | colon | COLO678 | colon | NCIH358M | lung |
| JIMT1 | breast | UMUC3 | bladder | A549 | lung |
| SKMEL28 | skin | SKNSH | brain | COLO205 | colon |
| CAKI1 | kidney | PANC1 | pancreas | SKMEL28 | skin |
| 786O | kidney | COLO205 | colon | PANC1 | pancreas |
| UMUC3 | bladder | 786O | kidney | SKNSH | brain |
| HELA | endometrial | SKBR3 | breast | 786O | kidney |
| KASUMI-1 | hematological | SKMEL28 | skin | MHHES1 | bone |
| HT29 | colon | TE671 | muscle | SKBR3 | breast |
| MHHES1 | bone | MHHES1 | bone | A204 | muscle |
| NCIH292 | lung | CAKI1 | kidney | HT29 | colon |
| A204 | muscle | HCT15 | colon | TE671 | muscle |
| SKBR3 | breast | SF268 | brain | NCIH460 | lung |
| SF268 | brain | NCIH292 | lung | NCIH292 | lung |
| SAOS2 | bone | PANC1005 | pancreas | CAKI1 | kidney |
| NCIH460 | lung | MDAMB436 | breast | PANC1005 | pancreas |
| TE671 | muscle | HT29 | colon | SF268 | brain |
| MDAMB436 | breast | MDAMB435 | skin | HELA | endometrial |
| A673 | muscle | JEG3 | placenta | J82 | bladder |
| SKOV3 | ovary | NCIH460 | lung | JEG3 | placenta |
| NCIH358M | lung | HELA | endometrial | 5637 | bladder |
| SW620 | colon | | | | |
| U87MG | brain | | | | |
| GRANTA-519 | hematological | | | | |
| JEG3 | placenta | | | | |
| 5637 | bladder | | | | |
| U2OS | bone | | | | |
| BXPC3 | pancreas | | | | |
| UO31 | kidney | | | | |
| T24 | bladder | | | | |
| DU145 | prostate | | | | |
| RDES | bone | | | | |
| IGROV1 | ovary | | | | |
| CLS439 | bladder | | | | |
| BT20 | breast | | | | |
| MIAPACA2 | pancreas | | | | |
| PANC1 | pancreas | | | | |

TABLE 3
Non-Responder Cell Lines with IC50 > 10 uM or Not Determined

| Compound I-9017 | | Compound I-9004 | | Compound I-8909 | |
|---|---|---|---|---|---|
| Cell line | Cell origin | Cell line | Cell origin | Cell line | Cell origin |
| SKNAS | brain | SW620 | colon | U2OS | bone |
| EJ28 | bladder | A204 | muscle | KASUMI-1 | hematological |
| HS578T | breast | 5637 | bladder | SW620 | colon |
| PC3 | prostate | BT20 | breast | MDAMB435 | skin |
| A375 | skin | A431 | skin | A431 | skin |
| SKLMS1 | uterus | SAOS2 | bone | BXPC3 | pancreas |
| RD | muscle | U2OS | bone | HCT15 | colon |
| HCT15 | colon | MIAPACA2 | pancreas | SAOS2 | bone |
| MDAMB435 | skin | IGROV1 | ovary | EJ28 | bladder |
| MDAMB231 | breast | KASUMI-1 | hematological | HS578T | breast |
| J82 | bladder | PC3 | prostate | CASKI | endometrial |
| CASKI | endometrial | RDES | bone | BT20 | breast |
| HS729 | muscle | T24 | bladder | MDAMB436 | breast |
| SNB75 | brain | A375 | skin | RDES | bone |
| IMR90 | lung | A673 | muscle | MIAPACA2 | pancreas |
| MG63 | bone | GRANTA-519 | hematological | PC3 | prostate |
| A431 | skin | SKOV3 | ovary | DU145 | prostate |
| PANC1005 | pancreas | EJ28 | bladder | SKOV3 | ovary |
| PBMC | hematological | CLS439 | bladder | A375 | skin |
| | | RD | muscle | CLS439 | bladder |
| | | BXPC3 | pancreas | T24 | bladder |
| | | DU145 | prostate | A673 | muscle |
| | | IMR90 | lung | IGROV1 | ovary |
| | | SKLMS1 | uterus | IMR90 | lung |
| | | UO31 | kidney | SKLMS1 | uterus |
| | | MG63 | bone | GRANTA-519 | hematological |
| | | CASKI | endometrial | SKNAS | brain |
| | | HS729 | muscle | UO31 | kidney |
| | | HS578T | breast | U87MG | brain |
| | | J82 | bladder | RD | muscle |
| | | SKNAS | brain | MG63 | bone |
| | | MDAMB231 | breast | HS729 | muscle |
| | | SNB75 | brain | SNB75 | brain |
| | | PBMC | hematological | MDAMB231 | breast |
| | | U87MG | brain | PBMC | hematological |

1: A compound of Formula (II): 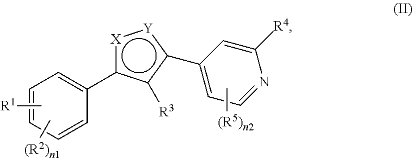
or a pharmaceutically acceptable salt, solvate, or isotope thereof, wherein:
X is S and Y is —CR^{6a}, or Y is S and X is —CR^{6b};
wherein when X is S and Y is —CR^{6a}, R¹ is —NR⁷C(O)NR⁸R⁹, —NR⁷S(O)₂NR⁸R⁹, —NR⁷C(O)OR⁹, —NR⁷S(O)₂R⁹, —SR⁹, —S(O)R⁹, —S(O)₂R⁹, —NR⁷C(S)NR⁸R⁹, —NR⁷C(O)SR⁹, or —NR⁸R⁹; and
wherein when Y is S and X is —CR^{6b}, R¹ is —NR⁷C(O)NR⁸R⁹, —NR⁷S(O)₂NR⁸R⁹, —NR⁷C(O)OR⁹, —SR⁹, —S(O)R⁹, —S(O)₂R⁹, —NR⁷C(S)NR⁸R⁹, —NR⁷C(O)SR⁹, or —NR⁸R⁹;
R⁷, R⁸, and R⁹ are independently selected from the group consisting of hydrogen, alkyl, cycloalkyl, cycloalkyl-alkyl, heterocycloalkyl, heterocycloalkyl-alkyl, aryl, aryl-alkyl, heteroaryl, and heteroaryl-alkyl; wherein each alkyl, cycloalkyl, cycloalkyl-alkyl, heterocycloalkyl, heterocycloalkyl-alkyl, aryl, aryl-alkyl, heteroaryl, and heteroaryl-alkyl of R⁷, R⁸, and R⁹ is independently unsubstituted or substituted with one or more substituents independently selected from the group consisting of halo, alkyl, haloalkyl, cyano, oxo, —OR¹⁰, —C(O)NR¹⁰R¹⁰, —NR¹⁰C(O)R¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR²¹)ₙ₆OR¹⁰;
or R⁸ and R⁹, together with the nitrogen atom to which they are attached, form a heterocycloalkyl, which is unsubstituted or substituted with one or more substituents independently selected from the group consisting of halo, cyano, oxo, alkyl, alkenyl, alkynyl, cycloalkyl, cycloalkyl-alkyl, heterocycloalkyl, heterocycloalkyl-alkyl, aryl, aryl-alkyl, heteroaryl, heteroaryl-alkyl, —OR¹⁰, —C(O)NR¹⁰R¹⁰, —NR¹⁰C(O)R¹⁰, —NR¹⁰C(O)OR¹⁰, —NR¹⁰C(O)NR¹⁰R¹⁰, —NR¹⁰R¹⁰, —S(O)₂NR¹⁰R¹⁰, —NR¹⁰S(O)₂R¹⁰, —S(O)ₘ₁R¹⁰, —C(O)OR¹⁰, —C(O)R¹⁰, and —(OR¹¹))ₙ₆OR¹⁰;
wherein each alkyl, alkenyl, alkynyl, cycloalkyl, cycloalkyl-alkyl, heterocycloalkyl, heterocycloalkyl-alkyl, aryl, aryl-alkyl, heteroaryl, and heteroaryl-alkyl is independently unsubstituted or substituted with one or more substituents independently selected from the group consisting of halo, oxo, alkyl, haloalkyl, —OR¹⁶, —C(O)NR¹⁶R¹⁶, —NR¹⁶C(O)R¹⁶, —NR¹⁶C(O)OR¹⁶, —NR¹⁶C(O)NR¹⁶R¹⁶, —NR¹⁶S(O)₂R¹⁶, and —S(O)ₙ₃R¹⁶; wherein each R¹⁶ is independently hydrogen, alkyl, cycloalkyl, cycloalkyl-alkyl, or heterocycloalkyl, each of which is independently unsubstituted or substituted with one or more halo; and each n3 is independently 0, 1, or 2;
n1 is 0, 1, or 2;
each R² is independently selected from the group consisting of halo, cyano, alkyl, cycloalkyl, cycloalkyl-alkyl, —OR¹¹, —C(O)NR¹¹R¹¹, —NR¹¹C(O)R¹¹, —NR¹¹C(O)NR¹¹R¹¹, —NR¹¹R¹¹, —S(O)₂NR¹¹R¹¹, —NR¹¹S(O)₂R¹¹, —S(O)ₘ₂R¹¹, —NR¹¹C(O)OR¹¹, —C(O)OR¹¹, and —C(O)R¹¹, wherein each alkyl, cycloalkyl, and cycloalkyl-alkyl is independently unsubstituted or substituted with one or more halo;
R⁴ is alkyl, alkenyl, cycloalkyl, cycloalkyl-alkyl, cycloalkenyl, heterocycloalkyl, heterocycloalkyl-alkyl, heterocycloalkenyl, —OR¹², —C(O)NR¹²R¹², —NR¹²C(O)NR¹²R¹², —S(O)₂NR¹²R¹², —S(O)ₘ₃R¹², or —C(O)R¹²;
n2 is 0, 1, 2, or 3;
each R⁵ is independently halo, alkyl, cycloalkyl, cycloalkyl-alkyl, heterocycloalkyl, heterocycloalkyl-alkyl, —OR¹³, —C(O)NR¹³R¹³, —S(O)₂NR¹³R¹³, —S(O)ₘ₄R¹³, or —C(O)R¹³;
or R⁴ and one R⁵, together with the atoms to which they are attached, form a carbocyclyl or heterocyclyl;
wherein each alkyl, alkenyl, cycloalkyl, cycloalkyl-alkyl, cycloalkenyl, heterocycloalkyl, heterocycloalkyl-alkyl, and heterocycloalkenyl of R⁴; alkyl, cycloalkyl, cycloalkyl-alkyl, heterocycloalkyl, heterocycloalkyl-alkyl of R⁵; and the carbocyclyl or heterocyclyl formed by R⁴ and one R⁵ is independently unsubstituted or substituted with one or more substituents independently selected from the group consisting of halo, cyano, oxo, alkyl, cycloalkyl, cycloalkyl-alkyl, heterocycloalkyl, —OR¹⁴, —C(O)OR¹⁴, —C(O)NR¹⁴R¹⁴, —NR¹⁴C(O)R¹⁴, —NR¹⁴C(O)NR¹⁴R¹⁴, —NR¹⁴R¹⁴, —S(O)₂NR¹⁴R¹⁴, —NR¹⁴S(O)₂R¹⁴, —S(O)ₘ₄R¹⁴, —C(O)R¹⁴, and —OC(O)R²²,
wherein each alkyl, cycloalkyl, cycloalkyl-alkyl, and heterocycloalkyl is independently unsubstituted or substituted with one or more substituents independently selected from the group consisting of halo, cyano, oxo, —C(O)OR¹⁷, —C(O)NR¹⁷R¹⁷, —NR¹⁷C(O)R¹⁷, —NR¹⁷C(O)NR¹⁷R¹⁷, —NR¹⁷R¹⁷, —S(O)₂NR¹⁷R¹⁷, —NR¹⁷S(O)₂R¹⁷, —S(O)ₙ₄R¹⁷, —C(O)R¹⁷, and —(OR¹⁸)ₙ₅OR¹⁷, wherein each R¹⁷ is independently hydrogen, alkyl, or haloalkyl; each n4 is independently 0, 1, or 2; each n5 is independently an integer from 0 to 5; and each R¹⁸ is independently alkylene or haloalkylene;
R²² is independently —R²³N(R²⁴)₂ or —(CH₂CH₂—O—)ₙ₈CH₃,
wherein each R²³ is (C₁-C₆)alkylene; each R²⁴ is independently H or —CH₃; and each n8 is independently an integer from 2 to 8;
R³, R^{6a}, and R^{6b} are independently selected from the group consisting of hydrogen, halo, cyano, alkyl, cycloalkyl, cycloalkyl-alkyl, heterocycloalkyl, heterocycloalkyl-alkyl, and —OR¹⁵, wherein each alkyl, cycloalkyl, cycloalkyl-alkyl, heterocycloalkyl, and heterocycloalkyl-alkyl is independently unsubstituted or substituted with one or more halo;
each R¹⁰, R¹¹, R¹⁴, and R¹⁵ is independently hydrogen, alkyl, cycloalkyl, cycloalkyl-alkyl, or heterocycloalkyl; two R¹⁰ together with the nitrogen atom to which they are attached may form a heterocycloalkyl; two R¹¹ together with the nitrogen atom to which they are attached may form a heterocycloalkyl; two R¹⁴ together with the nitrogen atom to which they are attached may form a heterocycloalkyl; and wherein each of the foregoing moieties is independently unsubstituted or substituted with one or more halo;
each R¹² and R¹³ is independently hydrogen, alkyl, cycloalkyl, cycloalkyl-alkyl, heterocycloalkyl, or heterocycloalkyl-alkyl, or two R¹² together with the nitrogen atom to which they are attached may form a heterocycloalkyl, or two R¹³ together with the nitrogen atom to which they are attached may form a heterocycloalkyl, wherein each of the foregoing is independently unsubstituted or substituted with one or more substituents independently selected from the group consisting of halo, cyano, oxo, alkyl, haloalkyl, —C(O)OR¹⁹,
—C(O)NR¹⁹R¹⁹, —NR¹⁹C(O)R¹⁹, —NR¹⁹C(O)NR¹⁹R¹⁹, —NR¹⁹R¹⁹, —S(O)₂NR¹⁹R¹⁹, —NR¹⁹S(O)₂R¹⁹, —S(O)ₙ₆R¹⁹, —C(O)R¹⁹, and —(OR²⁰)ₙ₇OR¹⁹, wherein each R¹⁹ is independently hydrogen, alkyl, or haloalkyl; each n6 is independently 0, 1, or 2; each n7 is independently an integer from 0 to 5; and each R²⁰ is independently alkylene or haloalkylene;
each R²¹ is independently alkylene or haloalkylene;
each n6 is independently an integer from 1 to 5; and
each m1, m2, m3, and m4 is independently 0, 1, or 2.

2. (canceled)

3: The compound of claim 1, wherein the compound is of Formula (II-A): 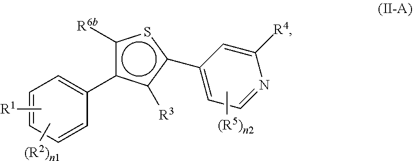
or a pharmaceutically acceptable salt, solvate, or isotope thereof.

4-6. (canceled)

7: The compound of claim 1, wherein the compound is of Formula (II-B): 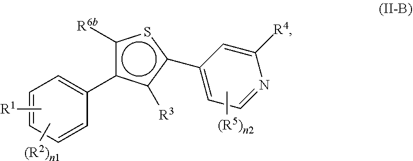
or a pharmaceutically acceptable salt, solvate, or isotope thereof.

8-10. (canceled)

11: The compound of claim 1, or a pharmaceutically acceptable salt, solvate, or isotope thereof, wherein X is S and Y is CR^{6a}; and wherein R¹ is —NR⁷C(O)NR⁸R⁹, —NR⁷S(O)₂NR⁸R⁹, —NR⁷C(O)OR⁹, —S(O)₂R⁹, —NR⁷(SO)₂R⁹, or —NR⁸R⁹.

12: The compound of claim 1 or a pharmaceutically acceptable salt, solvate, or isotope thereof, wherein R¹ is —NR⁷C(O)NR⁸R⁹, —NR⁷S(O)₂NR⁸R⁹, —NR⁷C(O)OR⁹, —S(O)₂R⁹, or —NR⁸R⁹.

13-14. (canceled)

15: The compound of claim 1, or a pharmaceutically acceptable salt, solvate, or isotope thereof, wherein R⁷ and R⁸ are both hydrogen, and R⁹ is alkyl, cycloalkyl, cycloalkyl-alkyl, heterocycloalkyl, or heterocycloalkyl-alkyl, wherein the alkyl, cycloalkyl, cycloalkyl-alkyl, heterocycloalkyl, or heterocycloalkyl-alkyl is unsubstituted or substituted with one or more substituents independently selected from the group consisting of halo and —OR¹⁰, wherein each R¹⁰ is independently hydrogen, alkyl, or haloalkyl.

16: The compound of claim 1, or a pharmaceutically acceptable salt, solvate, or isotope thereof, wherein R⁸ and R⁹, together with the nitrogen atom to which they are attached, form a heterocycloalkyl, wherein the heterocycloalkyl is unsubstituted or substituted with 1 to 3 substituents independently selected from the group consisting of halo, oxo, and —OR¹⁰, wherein each R¹⁰ is independently hydrogen, unsubstituted alkyl, or haloalkyl.

17: The compound of claim 1, or a pharmaceutically acceptable salt, solvate, or isotope thereof, wherein n1 is 0 or 1.

18: The compound of any one of claim 1, or a pharmaceutically acceptable salt, solvate, or isotope thereof, wherein each R² is independently cyano, halo, alkyl or —OR¹¹, wherein each R¹¹ is independently hydrogen, unsubstituted alkyl, or haloalkyl.

19. (canceled)

20: The compound of claim 1, or a pharmaceutically acceptable salt, solvate, or isotope thereof, wherein each R² is chloro.

21: The compound of claim 1, or a pharmaceutically acceptable salt, solvate, or isotope thereof, wherein R³ and R^{6a} or R^{6b} are both hydrogen.

22: The compound of claim 1, or a pharmaceutically acceptable salt, solvate, or isotope thereof, wherein n2 is 0.

23: The compound of claim 1, or a pharmaceutically acceptable salt, solvate, or isotope thereof, wherein R⁴ is alkyl or cycloalkyl, wherein the alkyl or cycloalkyl is unsubstituted or substituted with 1 to 3 substituents independently selected from the group consisting of halo, alkyl, alkyl substituted with —(OR¹⁸)ₙ₅OR¹⁷, haloalkyl, haloalkyl substituted with —(OR¹⁸)ₙ₅OR¹⁷, cycloalkyl, and —OR¹⁴, wherein each R¹⁴ and R¹⁷ is independently hydrogen, unsubstituted alkyl, or haloalkyl, and each R¹⁸ is independently alkylene.

24: The compound of claim 1, or a pharmaceutically acceptable salt, solvate, or isotope thereof, wherein R⁴ is —OR¹² and R¹² is heterocycloalkyl-alkyl.

25: The compound of claim 1, or a pharmaceutically acceptable salt, solvate, or isotope thereof, wherein R⁴ is alkyl substituted with one or more —OC(O)R²²; wherein R²² is —R²³N(R²⁴)₂ or —(CH₂CH₂—O—)ₙ₈CH₃.

26-27. (canceled)

28: The compound of claim 1, selected from the group consisting of: 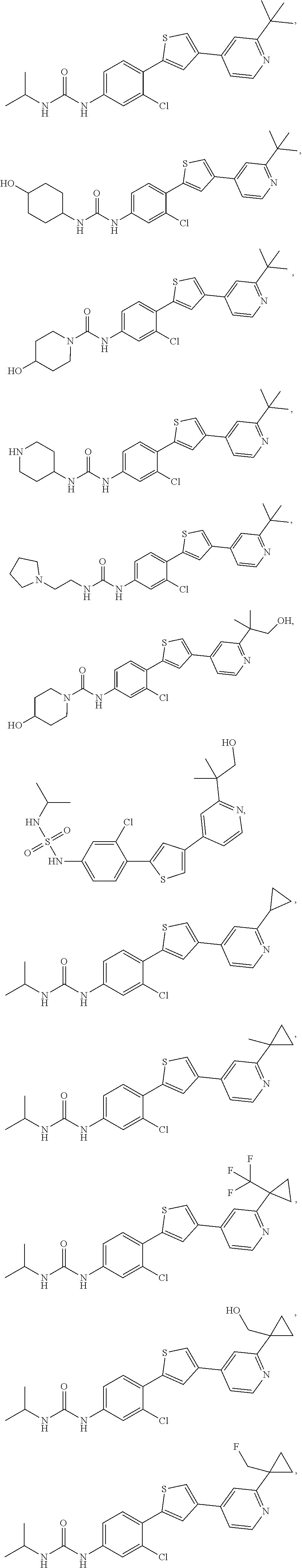 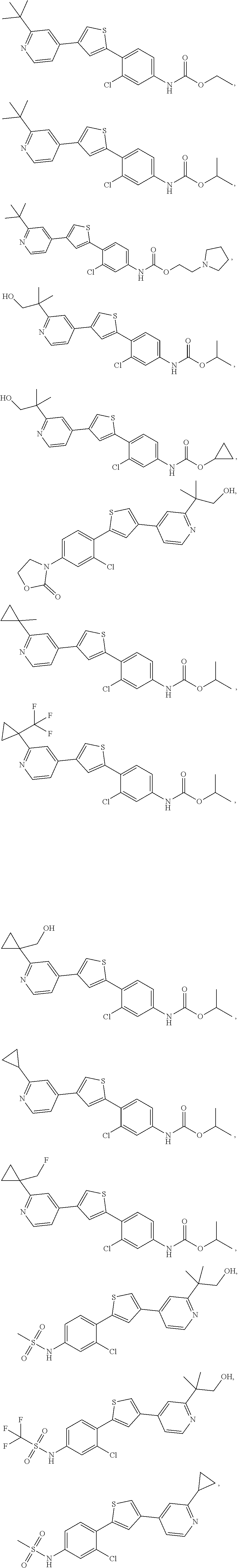 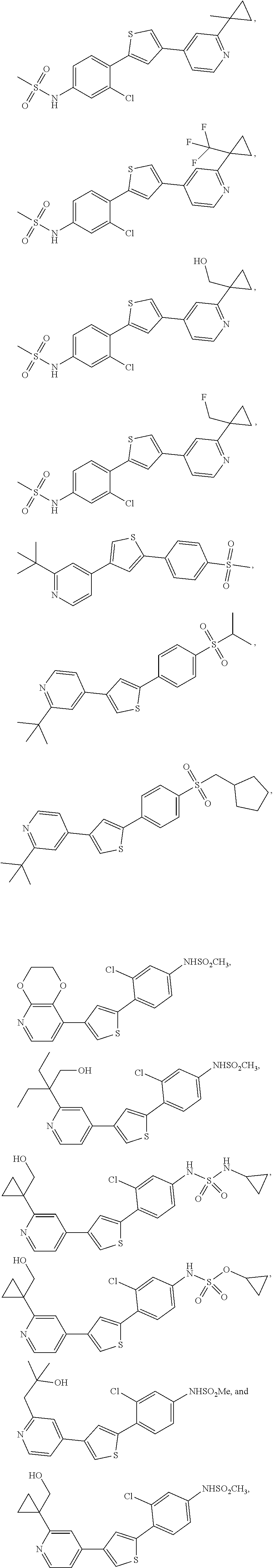
or a pharmaceutically acceptable salt, solvate, or isotope of any of the foregoing.

29: The compound of claim 1, selected from the group consisting of: 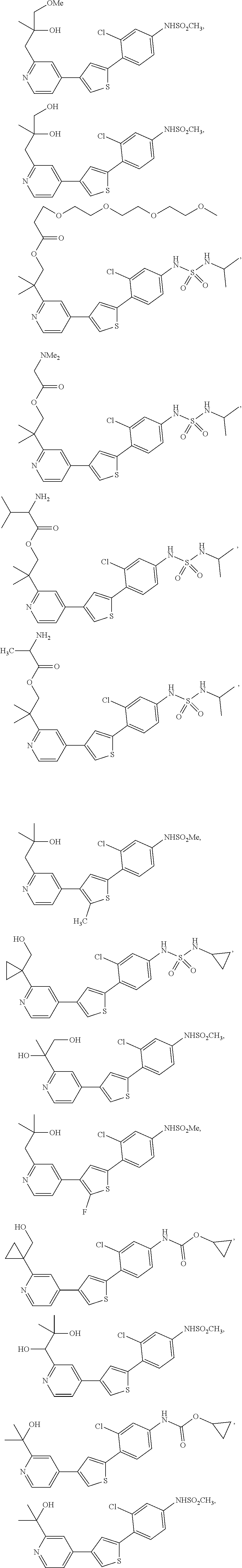 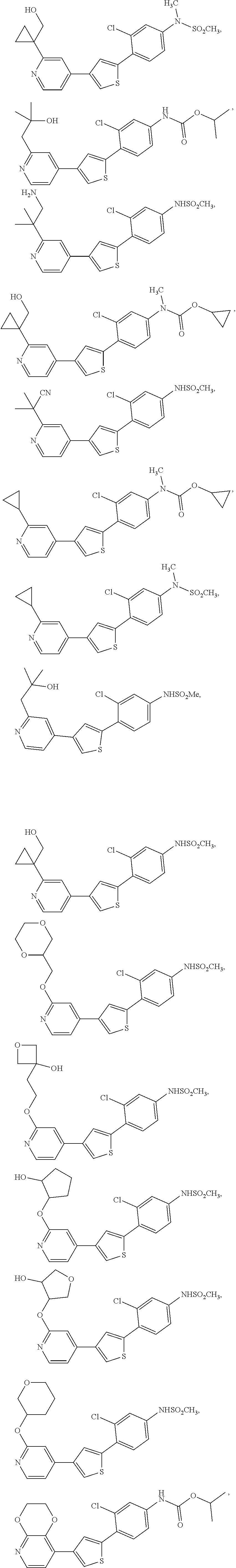 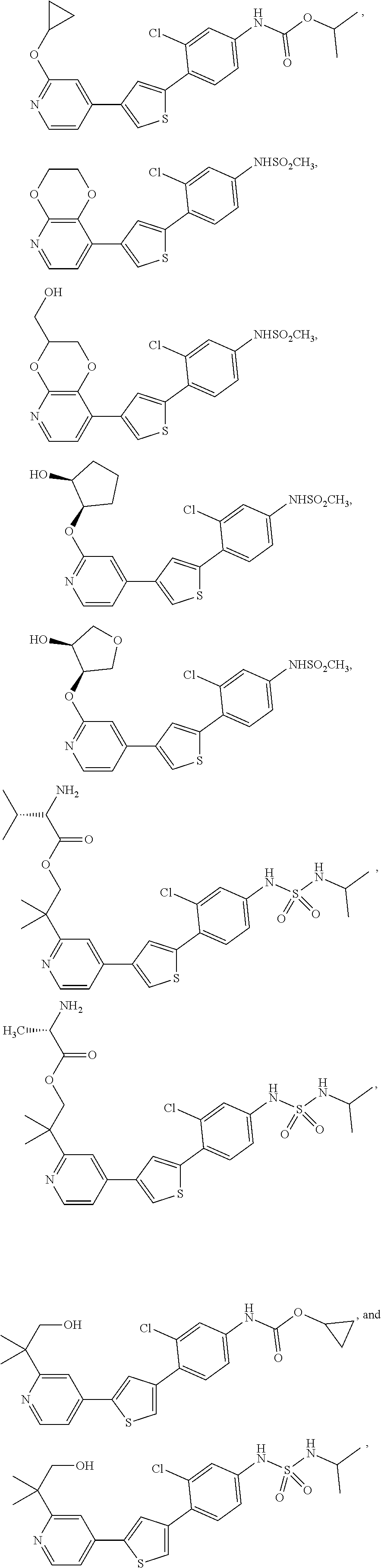
or a pharmaceutically acceptable salt, solvate, or isotope of any of the foregoing.

30: The compound of claim 1, selected from the group consisting of: 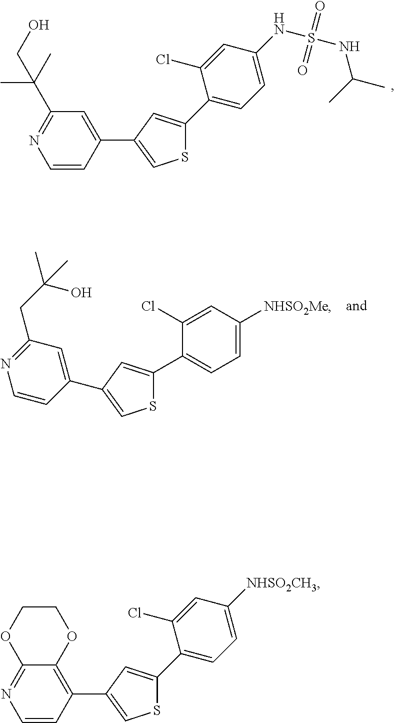
or a pharmaceutically acceptable salt, solvate, or isotope of any of the foregoing.

31: A pharmaceutical composition, comprising the compound of claim 1, or a pharmaceutically acceptable salt, solvate, or isotope thereof, and a pharmaceutically acceptable excipient.

32: A method of inhibiting a sterol regulatory element-binding protein (SREBP), comprising contacting the SREBP or contacting an SREBP cleavage activating-protein (SCAP) with an effective amount of a compound of claim 1, or a pharmaceutically acceptable salt, solvate, or isotope thereof.

33: A method of inhibiting the proteolytic activation of a sterol regulatory element-binding protein (SREBP), comprising contacting an SREBP cleavage activating-protein (SCAP) with an effective amount of a compound of claim 1, or a pharmaceutically acceptable salt, solvate, or isotope thereof.

34-41. (canceled)

42: A method of treating a disorder in a subject in need thereof, wherein the disorder is Metabolic Syndrome, type 2 diabetes, obesity, liver disease, insulin resistance, adiposopathy, or dyslipidemia, a hyperproliferative disorder, endotoxic shock, systemic inflammation, or atherosclerosis, comprising administering to the subject in need thereof an effective amount of a compound of claim 1, or a pharmaceutically acceptable salt, solvate, or isotope thereof.

43-44. (canceled)

45: The method of claim 42, wherein the liver disease is nonalcoholic steatohepatitis, liver fibrosis, or liver inflammation, or a combination thereof.

46. (canceled)

47. The method of claim 42, wherein the hyperproliferative disorder is cancer.

48: The method of claim 47, wherein the cancer is breast cancer, liver cancer, ovarian cancer, pancreatic cancer, prostate cancer, soft tissue sarcoma, bladder cancer, endometrial cancer, skin cancer, colon cancer, hematologic cancer, placenta cancer, brain cancer, kidney cancer, lung cancer, or bone cancer.

49-85. (canceled)

86. The compound of claim 1 selected from the group consisting of: 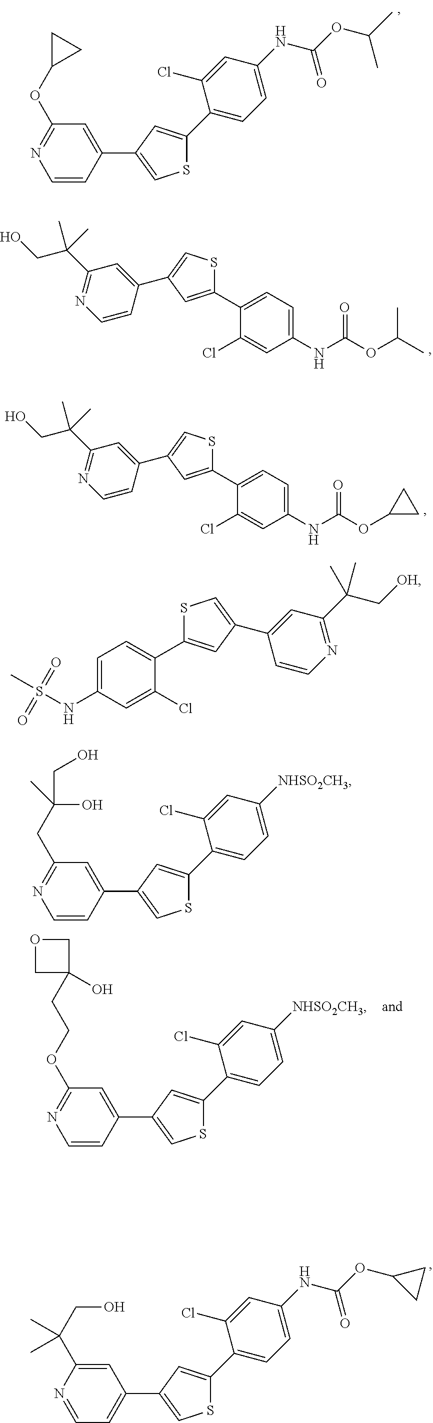
or a pharmaceutically acceptable salt, solvate, or isotope of any of the foregoing.
